(19) United States
(12) Patent Application Publication
(10) Pub. No.: US 2026/0150574 A1
(43) Pub. Date: May 28, 2026

(54) COMPOSITION FOR ORGANIC OPTOELECTRONIC DIODE, ORGANIC OPTOELECTRONIC DIODE, AND DISPLAY DEVICE

(71) Applicant: SAMSUNG SDI CO., LTD., Yongin-si, Gyeonggi-do (KR)

(72) Inventors: Dong Min KANG, Suwon-si, Gyeonggi-do (KR); Changwoo KIM, Suwon-si, Gyeonggi-do (KR); Yunsoo KIM, Suwon-si, Gyeonggi-do (KR); Mijin LEE, Suwon-si, Gyeonggi-do (KR); Seungjae LEE, Suwon-si, Gyeonggi-do (KR); Byoungkwan LEE, Suwon-si, Gyeonggi-do (KR); Kipo JANG, Suwon-si, Gyeonggi-do (KR); Ho Kuk JUNG, Suwon-si, Gyeonggi-do (KR); Hyung Sun KIM, Suwon-si, Gyeonggi-do (KR); Sunghyun JUNG, Suwon-si, Gyeonggi-do (KR); Chanwoo KIM, Suwon-si, Gyeonggi-do (KR); Jonghoon KO, Suwon-si, Gyeonggi-do (KR)

(21) Appl. No.: 19/123,453

(22) PCT Filed: Dec. 8, 2023
(86) PCT No.: PCT/KR2023/020210

§ 371 Date: Apr. 23, 2025

(30) Foreign Application Priority Data
Dec. 12, 2022 (KR) 10-2022-0173083

(51) Int. Cl. H10K 85/60; C07B 59/00; C07D 403/14; C07D 405/14; C07D 409/14; C09K 11/02; H10K 50/12

(52) U.S. Cl. CPC H10K 85/654; C07B 59/002; C07D 403/14; C07D 405/14; C07D 409/14; C09K 11/02; H10K 85/6572; H10K 85/6574; H10K 50/12

(57) ABSTRACT

Disclosed are a composition for an organic optoelectronic device, and an organic optoelectronic device including the same and a display device, the composition for an organic optoelectronic device including a first compound represented by Chemical Formula 1, and a second compound represented by Chemical Formula 2.

### TECHNICAL FIELD

A composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric element, an organic light emitting diode, an organic solar cell, and an organic photo conductor drum.

Of these, an organic light emitting diode (OLED) has recently drawn attention due to an increase in demand for flat panel displays. The organic light emitting diode is a device that converts electrical energy into light, and the performance of the organic light emitting diode is greatly influenced by an organic material between electrodes.

### DISCLOSURE

### Technical Problem

An embodiment provides a composition for an organic optoelectronic device capable of realizing an organic optoelectronic device having a high-efficiency and long life-span.

Another embodiment provides an organic optoelectronic device including the composition for the organic optoelectronic device.

Another embodiment provides a display device including the organic optoelectronic device.

### Technical Solution

According to an embodiment, a composition for an organic optoelectronic device includes a first compound represented by Chemical Formula 1 and a second compound represented by

### Chemical Formula 2.

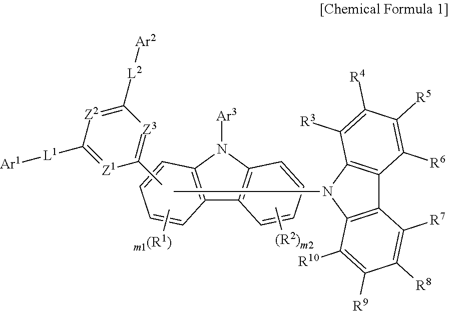

In Chemical Formula 1,
Z¹ to Z³ are each independently N or C—R^{a},
at least two of Z¹ to Z³ are N,
L¹ and L² are each independently a single bond or a substituted or unsubstituted C6 to C20 arylene group,
Ar¹ and Ar² are each independently a substituted or unsubstituted C6 to C30 aryl group or a substituted or unsubstituted C2 to C30 heterocyclic group,

Ar³ is a substituted or unsubstituted C6 to C20 aryl group,

R¹ and R² are each independently hydrogen, deuterium, a substituted or unsubstituted C1 to C10 alkyl group, a substituted or unsubstituted C6 to C20 aryl group, or a combination thereof,
m1 is an integer from 1 to 3,
m2 is an integer from 1 to 4, and

R^{a} and R³ to R¹⁰ are each independently hydrogen, deuterium, a substituted or unsubstituted C1 to C10 alkyl group, a substituted or unsubstituted C6 to C30 aryl group, a substituted or unsubstituted C2 to C30 heterocyclic group, or a combination thereof;

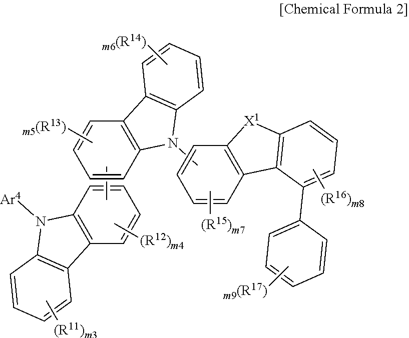

wherein, in Chemical Formula 2,
X¹ is O or S,
Ar⁴ is a substituted or unsubstituted C6 to C30 aryl group, a substituted or unsubstituted C2 to C30 heterocyclic group, or a combination thereof,
R¹¹ to R¹⁴ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
R¹⁵ to R¹⁷ are each independently hydrogen, deuterium, or a substituted or unsubstituted phenyl group,
m4, m5, m7, and m8 are each independently an integer from 1 to 3, and
m3, m6, and m9 are each independently an integer from 1 to 4.

According to another embodiment, an organic optoelectronic device includes an anode and a cathode facing each other, and at least one organic layer between the anode and the cathode, wherein the organic layer includes the composition for an organic optoelectronic device.

According to another embodiment, a display device including the organic optoelectronic device is provided.

### Advantageous Effects

High-efficiency, long life-span, and low-driving organic optoelectronic devices may be implemented.

### DESCRIPTION OF THE DRAWINGS

FIG. 1 is cross-sectional view illustrating an organic light emitting diode according to embodiments.

### DESCRIPTION OF SYMBOLS

100: organic light emitting diode
105: organic layer
110: cathode
120: anode
130: light emitting layer
140: hole transport region
150: electron transport region

### BEST MODE

Hereinafter, embodiments of the present invention are described in detail. However, these embodiments are exemplary, the present invention is not limited thereto and the present invention is defined by the scope of claims.

In the present specification, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, or a combination thereof.

In one example of the present invention, the “substituted” refers to replacement of at least one hydrogen of a substituent or a compound by deuterium, a cyano group, a C1 to C30 alkyl group, a C1 to C10 alkylsilyl group, a C6 to C30 arylamine group, a C6 to C30 arylsilyl group, a C3 to C30 cycloalkyl group, a C3 to C30 heterocycloalkyl group, a C6 to C30 aryl group, or a C2 to C30 heteroaryl group. In specific example of the present invention, the “substituted” refers to replacement of at least one hydrogen of a substituent or a compound by deuterium, a cyano group, a C1 to C20 alkyl group, a C6 to C30 arylamine group, a C6 to C30 aryl group, or a C2 to C30 heteroaryl group. In specific example of the present invention, the “substituted” refers to replacement of at least one hydrogen of a substituent or a compound by deuterium, a cyano group, a C1 to C5 alkyl group, a C6 to C20 arylamine group, a C6 to C18 aryl group, a dibenzofuranyl group, a dibenzothiophenyl group, a carbazolyl group, or a pyridinyl group. In specific example of the present invention, the “substituted” refers to replacement of at least one hydrogen of a substituent or a compound by deuterium, a cyano group, a methyl group, an ethyl group, a propyl group, a butyl group, a C6 to C20 arylamine group, a phenyl group, a biphenyl group, a terphenyl group, a naphthyl group, triphenyl group, fluorenyl group, dibenzofuranyl group, a dibenzothiophenyl group, a carbazolyl group, or a pyridinyl group.

In the present specification, “unsubstituted” refers to non-replacement of a hydrogen atom by another substituent and remaining of the hydrogen atom.

In this specification, “deuterium substitution (-D)” may include “tritium substitution (-T)”.

In the present specification, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quaterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

In the present specification, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a composition for an organic optoelectronic device according to an embodiment is described.

The composition for an organic optoelectronic device according to an embodiment includes a first compound represented by Chemical Formula 1 and a second compound represented by Chemical Formula 2.

The first compound may be represented by Chemical Formula 1.

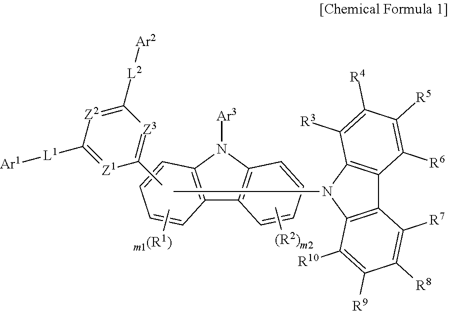

In Chemical Formula 1,
Z¹ to Z³ are each independently N or C—R^{a},
at least two of Z¹ to Z³ are N,
L¹ and L² are each independently a single bond or a substituted or unsubstituted C6 to C20 arylene group,
Ar¹ and Ar² are each independently a substituted or unsubstituted C6 to C30 aryl group or a substituted or unsubstituted C2 to C30 heterocyclic group,
Ar³ is a substituted or unsubstituted C6 to C20 aryl group,
R¹ and R² are each independently hydrogen, deuterium, a substituted or unsubstituted C1 to C10 alkyl group, a substituted or unsubstituted C6 to C20 aryl group, or a combination thereof,
m1 is an integer from 1 to 3,
m2 is an integer from 1 to 4, and
R^{a} and R³ to R¹⁰ are each independently hydrogen, deuterium, a substituted or unsubstituted C1 to C10 alkyl group, a substituted or unsubstituted C6 to C30 aryl group, a substituted or unsubstituted C2 to C30 heterocyclic group, or a combination thereof.

The first compound represented by Chemical Formula 1 has a structure in which carbazole is a basic skeleton, and at least one nitrogen-containing ring (nitrogen-containing 6-membered ring) and 9-carbazole are substituted at 1st to 8th positions of the carbazole.

The compound includes at least one nitrogen-containing ring and thus, may have a structure easily accepting the electrons when an electric field is applied thereto and accordingly, decrease a driving voltage of an organic optoelectronic device manufactured by using the compound.

In addition, the first compound can form a bipolar structure by including carbazole, which is prone to receiving holes, to properly balance flow of holes and electrons, thereby improving efficiency of organic optoelectronic devices including the compound.

In particular, by linking to the nitrogen-containing 6-membered ring in 1st to 8th directions of carbazole, LUMO electron cloud expands from the nitrogen-containing 6-membered ring to the carbazole linker, increasing electron transport capability, and additional carbazole substitution in the 9th direction (N-direction) can separate the HOMO electron cloud and enhance hole characteristics. Therefore, by separating the electron cloud between HOMO-LUMO into a hole transport part and an electron transport part, the efficiency and life-span of the organic light emitting diode including the same can be further improved.

In Chemical Formula 1, when m1 is greater than or equal to 2, each R¹ may be the same or different from each other.

In Chemical Formula 1, when m2 is greater than or equal to 2, each R² may be the same or different from each other.

For example, Chemical Formula 1 may be represented by Chemical Formula 1A or Chemical Formula 1B.

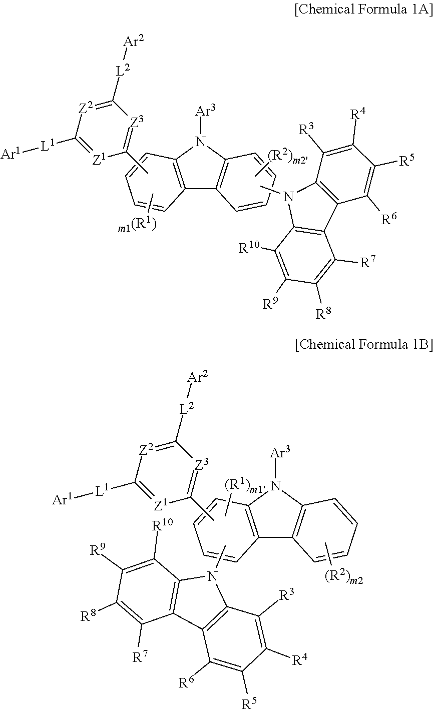

In Chemical Formula 1A and Chemical Formula 1B,
Z¹ to Z³, L¹, L², Ar¹ to Ar³, R¹ to R¹⁰, m1, and m2 are the same as described above,
m1′ is an integer of 1 or 2, and
m2′ is an integer of 1 to 3.

As a specific example, Chemical Formula 1A may be represented by Chemical Formula 1A-1 to Chemical Formula 1A-4.

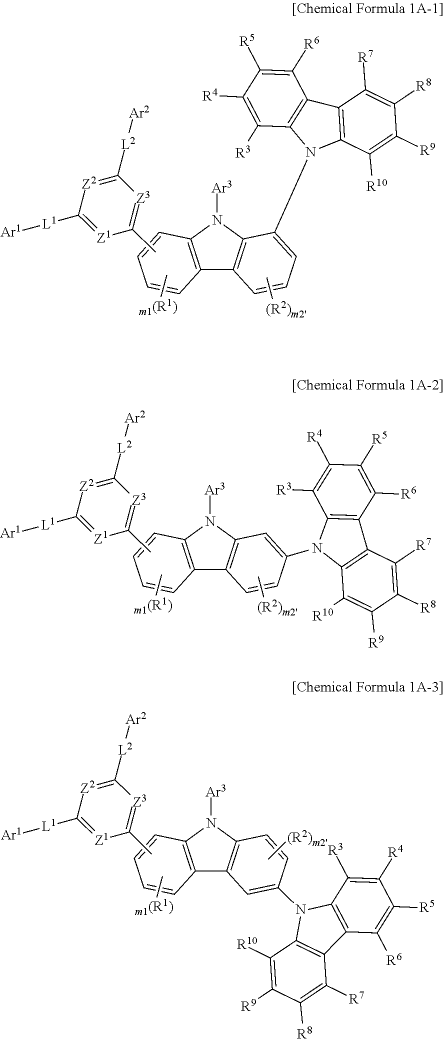

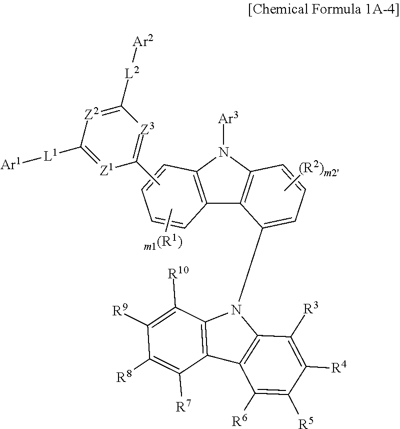

In Chemical Formula 1A-1 to Chemical Formula 1A-4,
Z¹ to Z³, L¹, L², Ar¹ to Ar³, R¹ to R¹⁰, m1, and m2′ are the same as described above.

As a specific example, Chemical Formula 1B may be represented by any of Chemical Formula 1B-1 to Chemical Formula 1B-4.

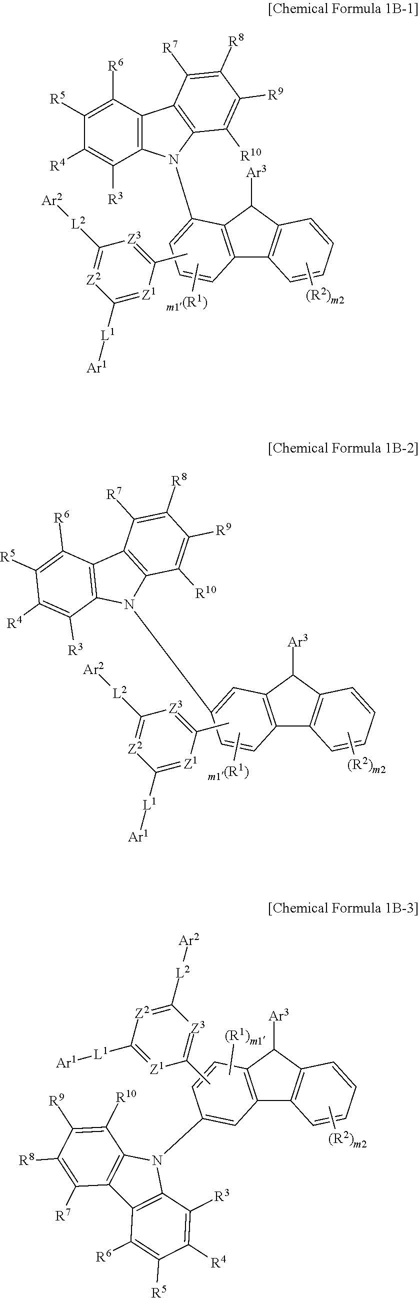

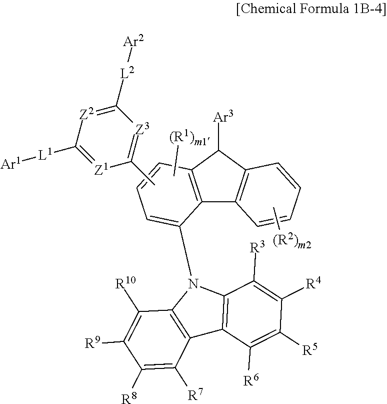

In Chemical Formula 1B-1 to Chemical Formula 1B-4,
Z¹ to Z³, L¹, L², Ar¹ to Ar³, R¹ to R¹⁰, m1′, and m2 are the same as described above.

For example, Chemical Formula 1A-1 may be represented by any of Chemical Formula 1A-1-1 to Chemical Formula 1A-1-4.

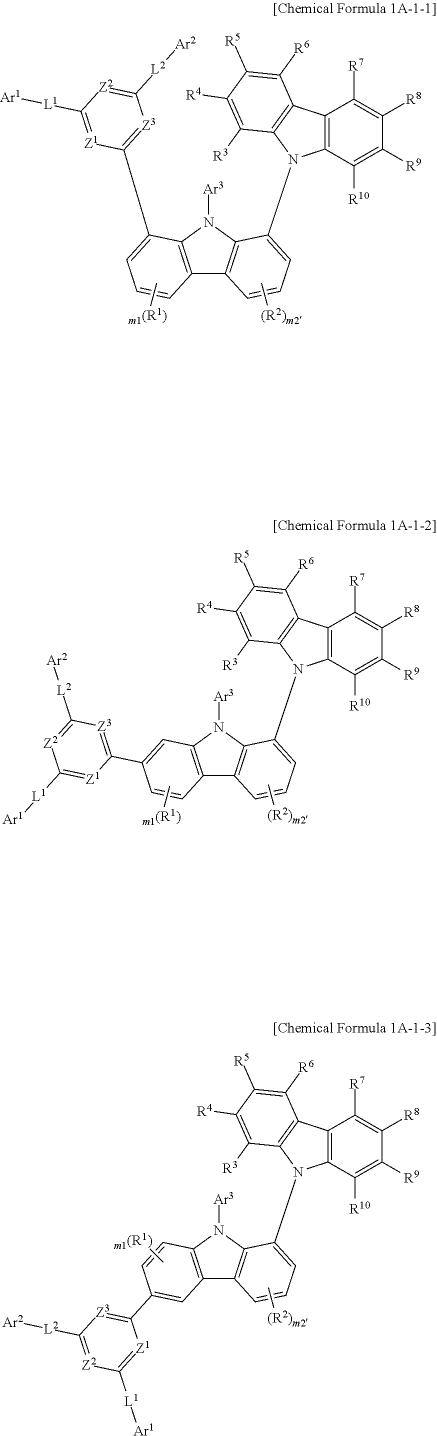

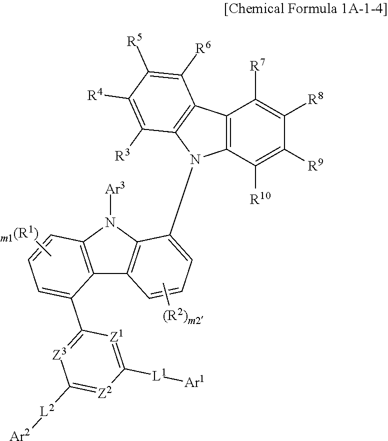

In Chemical Formula 1A-1-1 to Chemical Formula 1A-1-4,
Z¹ to Z³, L¹, L², Ar¹ to Ar³, R¹ to R¹⁰, m1, and m2′ are the same as described above.

For example, Chemical Formula 1A-2 may be represented by Chemical Formula 1A-2-1 to Chemical Formula 1A-2-4.

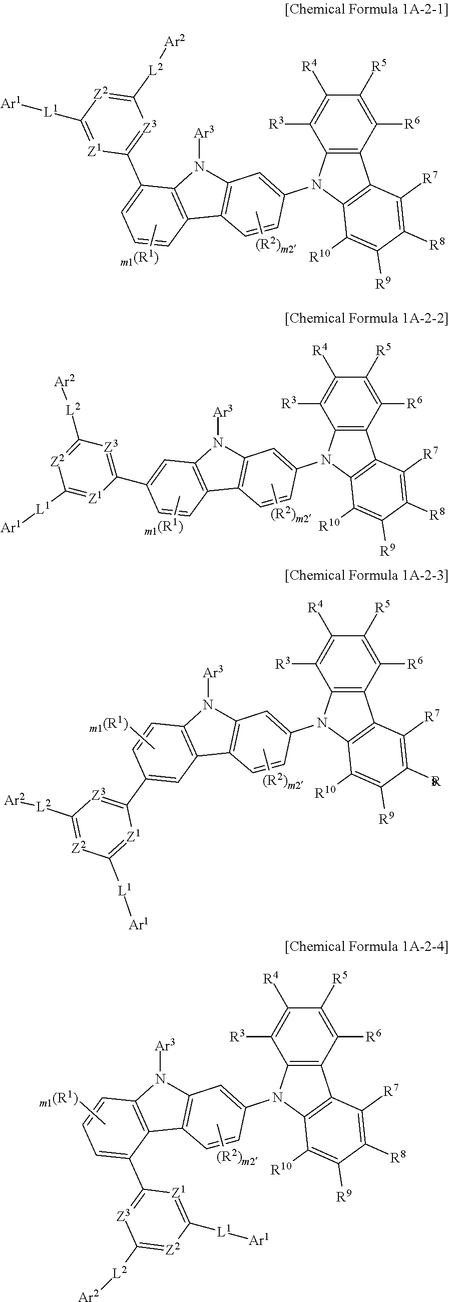

In Chemical Formula 1A-2-1 to Chemical Formula 1A-2-4, Z¹ to Z³, L¹, L², Ar¹ to Ar³, R¹ to R¹⁰, m1, and m2′ are the same as described above.

For example, Chemical Formula 1A-3 may be represented by any of Chemical Formula 1A-3-1 to Chemical Formula 1A-3-4.

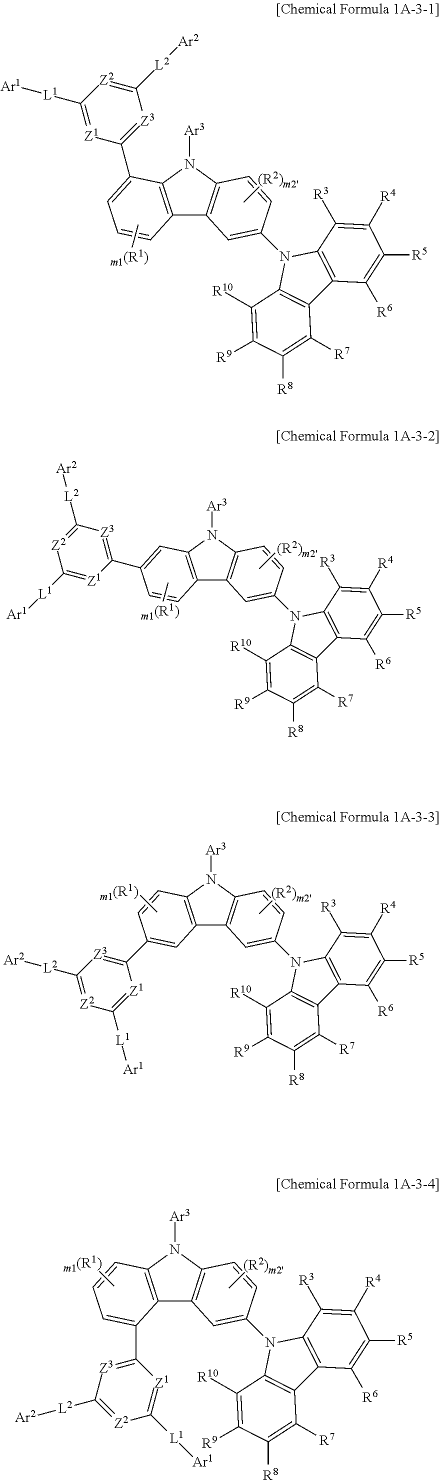

In Chemical Formula 1A-3-1 to Chemical Formula 1A-3-4, Z¹ to Z³, L¹, L², Ar¹ to Ar³, R¹ to R¹⁰, m1, and m2′ are the same as described above.

For example, Chemical Formula 1A-4 may be represented by any of Chemical Formula 1A-4-1 to Chemical Formula 1A-4-4.

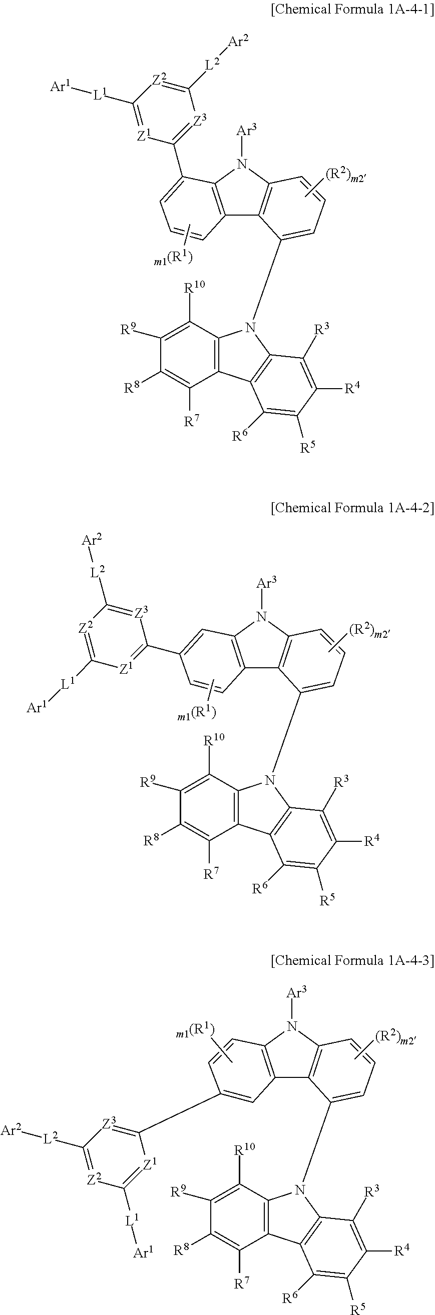

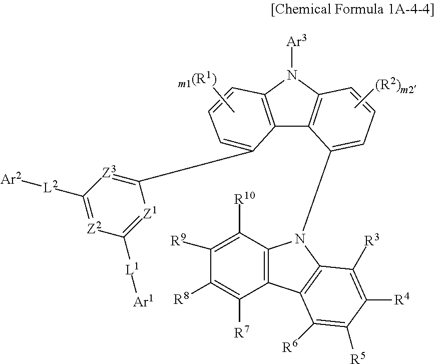

In Chemical Formula 1A-4-1 to Chemical Formula 1A-4-4, Z¹ to Z³, L¹, L², Ar¹ to Ar³, R¹ to R¹⁰, m1, and m2′ are the same as described above.

For example, Chemical Formula 1B-1 may be represented by any of Chemical Formula 1B-1-2 to Chemical Formula 1B-1-4.

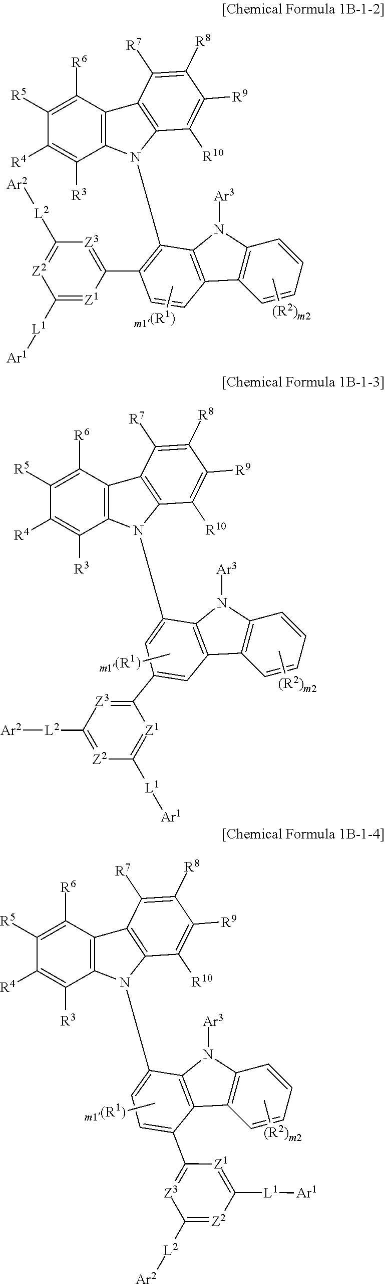

In Chemical Formula 1B-1-2 to Chemical Formula 1B-1-4, Z¹ to Z³, L¹, L², Ar¹ to Ar³, R¹ to R¹⁰, m1′, and m2 are the same as described above.

For example, Chemical Formula 1B-2 may be represented by any one of Chemical Formula 1B-2-1, Chemical Formula 1B-2-3 and Chemical Formula 1B-2-4.

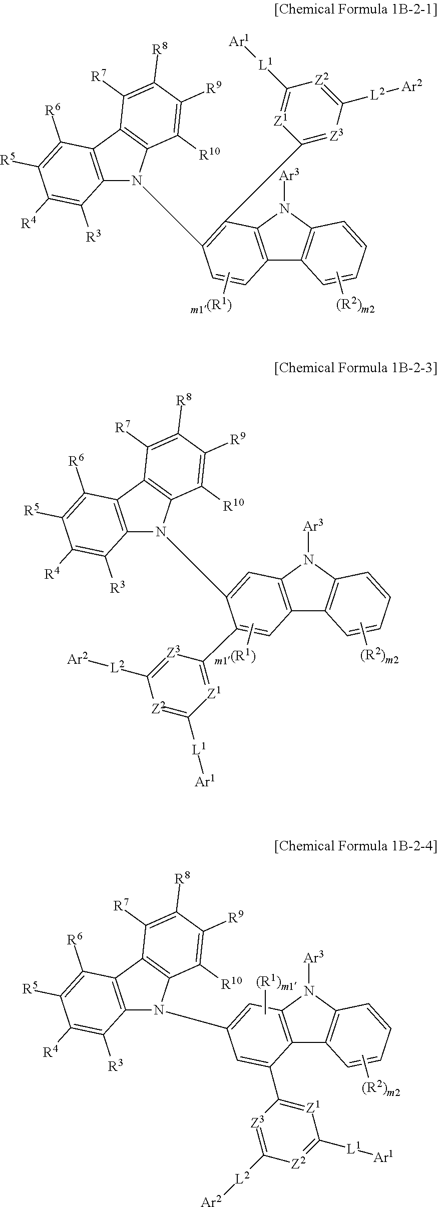

In Chemical Formula 1B-2-1, Chemical Formula 1B-2-3 and Chemical Formula 1B-2-4, z¹ to Z³, L¹, L², Ar¹ to Ar³, R¹ to R¹⁰, m1′, and m2 are the same as described above.

For example, Chemical Formula 1B-3 may be represented by any one of Chemical Formula 1B-3-1, Chemical Formula 1B-3-2 and Chemical Formula 1B-3-4.

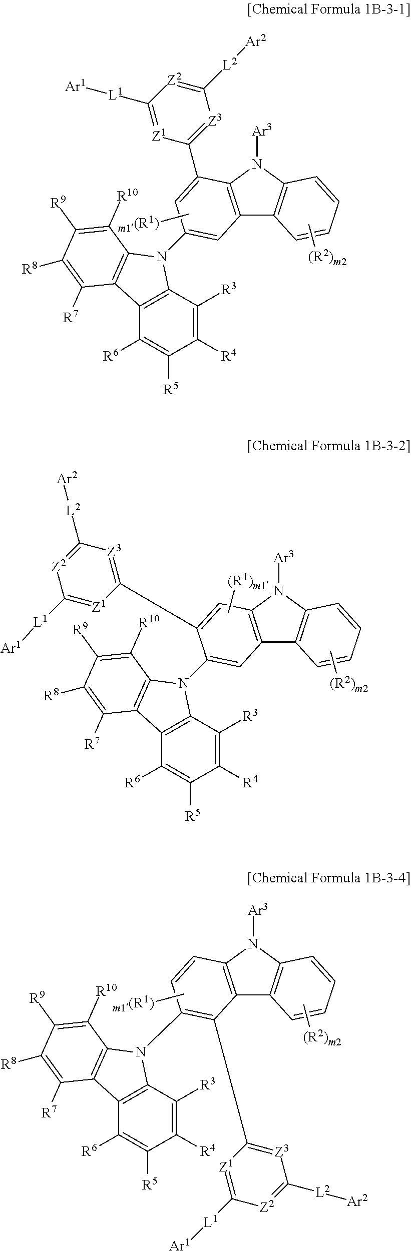

In Chemical Formula 1B-3-1, Chemical Formula 1B-3-2 and Chemical Formula 1B-3-4, Z¹ to Z³, L¹, L², Ar¹ to Ar³, R¹ to R¹⁰, m1′, and m2 are the same as described above.

For example, Chemical Formula 1B-4 may be represented by any one of Chemical Formula 1B-4-1 to Chemical Formula 1B-4-3.

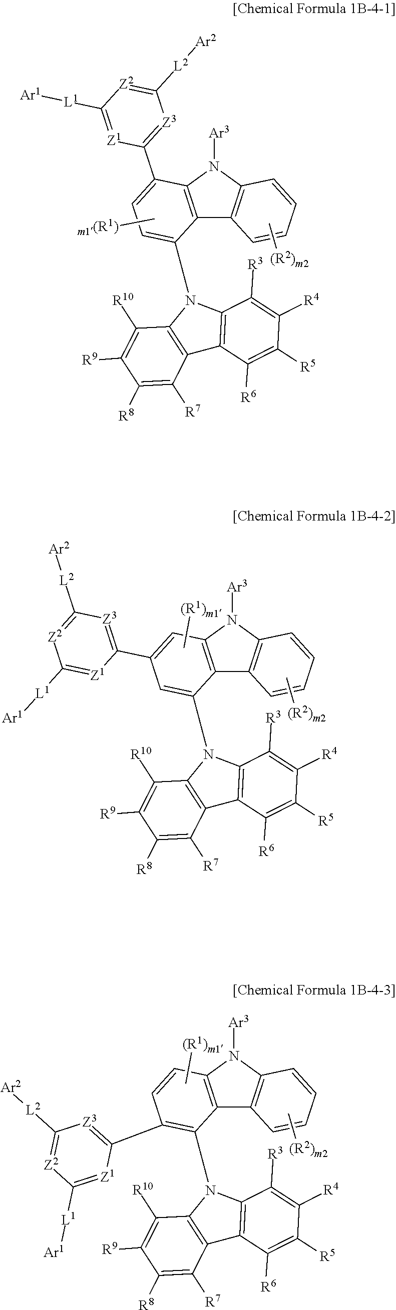

In Chemical Formula 1B-4-1 to Chemical Formula 1B-4-3, Z¹ to Z³, L¹, L², Ar¹ to Ar³, R¹ to R¹⁰, m1′, and m2 are the same as described above.

In an embodiment, the first compound may be represented by any one of Chemical Formula 1A-1-2, Chemical Formula 1A-2-2, Chemical Formula 1A-3-2, Chemical Formula 1A-4-2, Chemical Formula 1B-1-2, Chemical Formula 1B-2-2, Chemical Formula 1B-3-2, and Chemical Formula 1B-4-2.

In a specific embodiment, the first compound may be represented by any one of Chemical Formula 1A-1-2, Chemical Formula 1A-4-2, Chemical Formula 1B-1-2 and Chemical Formula 1B-4-2.

For example, in Chemical Formula 1, Ar¹ and Ar² may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, or a substituted or unsubstituted dibenzothiophenyl group.

As a specific example, in Chemical Formula 1, Ar¹ and Ar² may each independently be a substituted or unsubstituted phenyl group, or a substituted or unsubstituted biphenyl group.

For example, in Chemical Formula 1, L¹ and L² may each independently be a single bond, or a substituted or unsubstituted phenylene group.

For example, L¹-Ar¹ and L²-Ar² in Chemical Formula 1 may each be independently selected from the substituents listed in Group I.

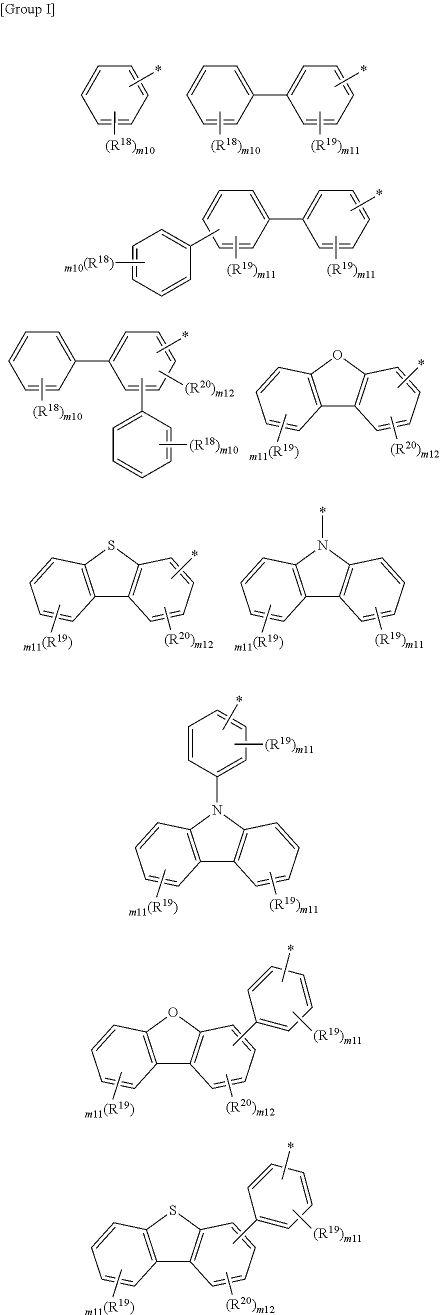

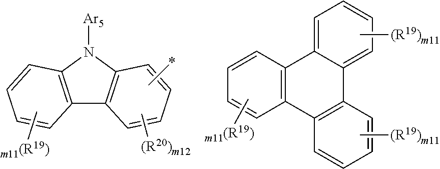

In Group I,
R¹⁸ to R²⁰ are each independently hydrogen, deuterium, a halogen, a cyano group, a substituted or unsubstituted C1 to C10 alkyl group, or a substituted or unsubstituted C6 to C12 aryl group,
Ar⁵ is a substituted or unsubstituted C6 to C12 aryl group,
m10 is an integer of 1 to 5,
m11 is an integer of 1 to 4,
m12 is an integer of 1 to 3, and
* is a linking point.

For example, in Chemical Formula 1, Ar³ may be a substituted or unsubstituted C6 to C12 aryl group.

As a specific example, in Chemical Formula 1, Ar³ may be a substituted or unsubstituted phenyl group, a substituted or unsubstituted biphenyl group, or a substituted or unsubstituted naphthyl group.

For example, in Chemical Formula 1, R¹ and R² may each independently be hydrogen, deuterium or a substituted or unsubstituted C6 to C12 aryl group.

For example, in Chemical Formula 1, R³ to R⁸ may each independently be hydrogen, deuterium, or a substituted or unsubstituted C6 to C20 aryl group.

As a specific example, in Chemical Formula 1, R¹ to R⁸ may each independently hydrogen, deuterium, a substituted or unsubstituted phenyl group, a substituted or unsubstituted biphenyl group, or a substituted or unsubstituted naphthyl group.

The first compound may be, for example, one selected from the compounds listed in Group 1.

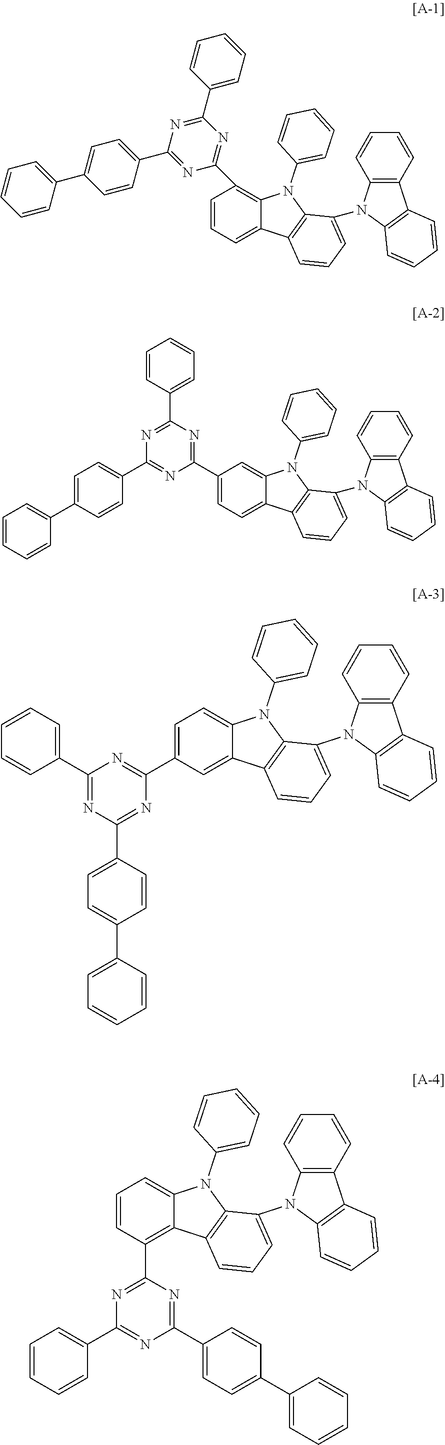 [SMILES: c1ccc(-c2ccc(-c3nc(-c4ccccc4)nc(-c4cccc5c6cccc(-n7c8ccccc8c8ccccc87)c6n(-c6ccccc6)c45)n3)cc2)cc1 ; c1ccc(-c2ccc(-c3nc(-c4ccccc4)nc(-c4ccc5c6cccc(-n7c8ccccc8c8ccccc87)c6n(-c6ccccc6)c5c4)n3)cc2)cc1 ; c1ccc(-c2ccc(-c3nc(-c4ccccc4)nc(-c4ccc5c(c4)c4cccc(-n6c7ccccc7c7ccccc76)c4n5-c4ccccc4)n3)cc2)cc1 ; c1ccc(-c2ccc(-c3nc(-c4ccccc4)nc(-c4cccc5c4c4cccc(-n6c7ccccc7c7ccccc76)c4n5-c4ccccc4)n3)cc2)cc1]

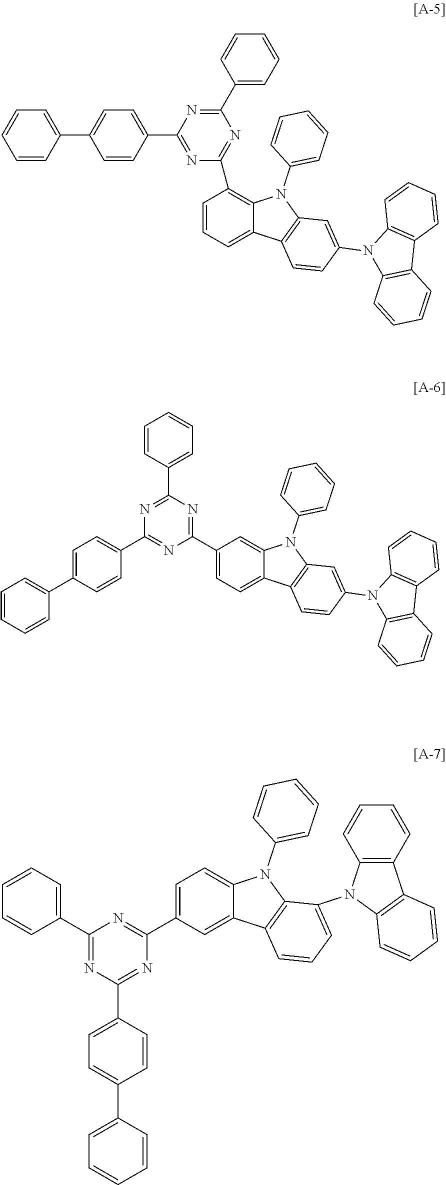 [SMILES: c1ccc(-c2ccc(-c3nc(-c4ccccc4)nc(-c4cccc5c6ccc(-n7c8ccccc8c8ccccc87)cc6n(-c6ccccc6)c45)n3)cc2)cc1 ; c1ccc(-c2ccc(-c3nc(-c4ccccc4)nc(-c4ccc5c6ccc(-n7c8ccccc8c8ccccc87)cc6n(-c6ccccc6)c5c4)n3)cc2)cc1 ; c1ccc(-c2ccc(-c3nc(-c4ccccc4)nc(-c4ccc5c(c4)c4cccc(-n6c7ccccc7c7ccccc76)c4n5-c4ccccc4)n3)cc2)cc1]

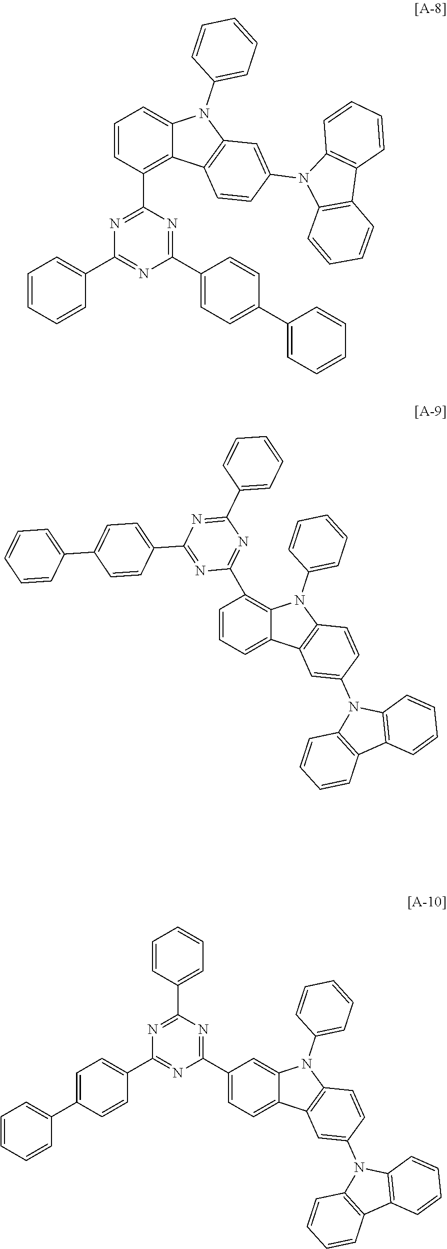 [SMILES: c1ccc(-c2ccc(-c3nc(-c4ccccc4)nc(-c4cccc5c4c4ccc(-n6c7ccccc7c7ccccc76)cc4n5-c4ccccc4)n3)cc2)cc1 ; c1ccc(-c2ccc(-c3nc(-c4ccccc4)nc(-c4cccc5c6cc(-n7c8ccccc8c8ccccc87)ccc6n(-c6ccccc6)c45)n3)cc2)cc1 ; c1ccc(-c2ccc(-c3nc(-c4ccccc4)nc(-c4ccc5c6cc(-n7c8ccccc8c8ccccc87)ccc6n(-c6ccccc6)c5c4)n3)cc2)cc1]

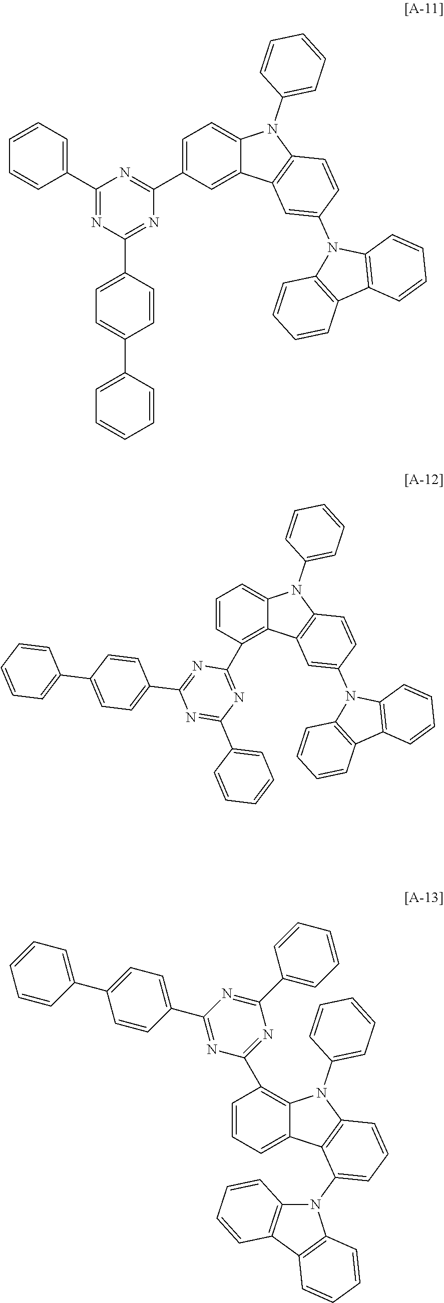 [SMILES: c1ccc(-c2ccc(-c3nc(-c4ccccc4)nc(-c4ccc5c(c4)c4cc(-n6c7ccccc7c7ccccc76)ccc4n5-c4ccccc4)n3)cc2)cc1 ; c1ccc(-c2ccc(-c3nc(-c4ccccc4)nc(-c4cccc5c4c4cc(-n6c7ccccc7c7ccccc76)ccc4n5-c4ccccc4)n3)cc2)cc1 ; c1ccc(-c2ccc(-c3nc(-c4ccccc4)nc(-c4cccc5c6c(-n7c8ccccc8c8ccccc87)cccc6n(-c6ccccc6)c45)n3)cc2)cc1]

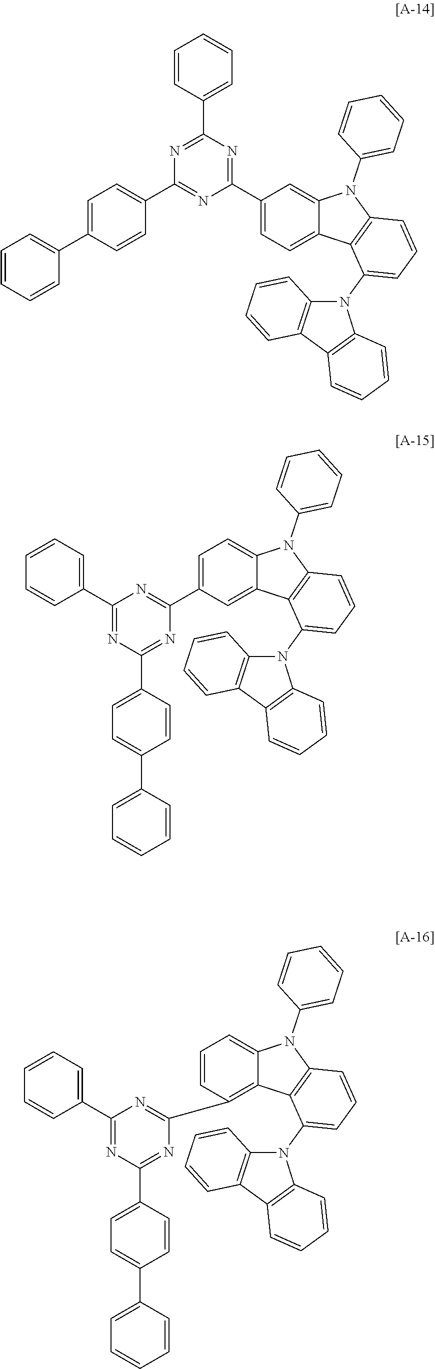 [SMILES: c1ccc(-c2ccc(-c3nc(-c4ccccc4)nc(-c4ccc5c6c(-n7c8ccccc8c8ccccc87)cccc6n(-c6ccccc6)c5c4)n3)cc2)cc1 ; c1ccc(-c2ccc(-c3nc(-c4ccccc4)nc(-c4ccc5c(c4)c4c(-n6c7ccccc7c7ccccc76)cccc4n5-c4ccccc4)n3)cc2)cc1 ; c1ccc(-c2ccc(-c3nc(-c4ccccc4)nc(-c4cccc5c4c4c(-n6c7ccccc7c7ccccc76)cccc4n5-c4ccccc4)n3)cc2)cc1]

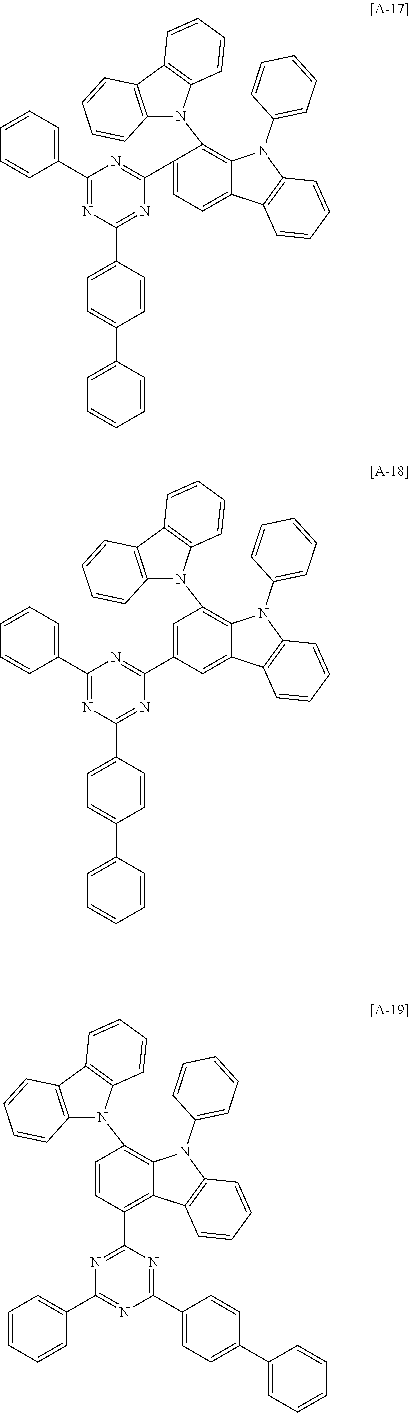 [SMILES: c1ccc(-c2ccc(-c3nc(-c4ccccc4)nc(-c4ccc5c6ccccc6n(-c6ccccc6)c5c4-n4c5ccccc5c5ccccc54)n3)cc2)cc1 ; c1ccc(-c2ccc(-c3nc(-c4ccccc4)nc(-c4cc(-n5c6ccccc6c6ccccc65)c5c(c4)c4ccccc4n5-c4ccccc4)n3)cc2)cc1 ; c1ccc(-c2ccc(-c3nc(-c4ccccc4)nc(-c4ccc(-n5c6ccccc6c6ccccc65)c5c4c4ccccc4n5-c4ccccc4)n3)cc2)cc1]

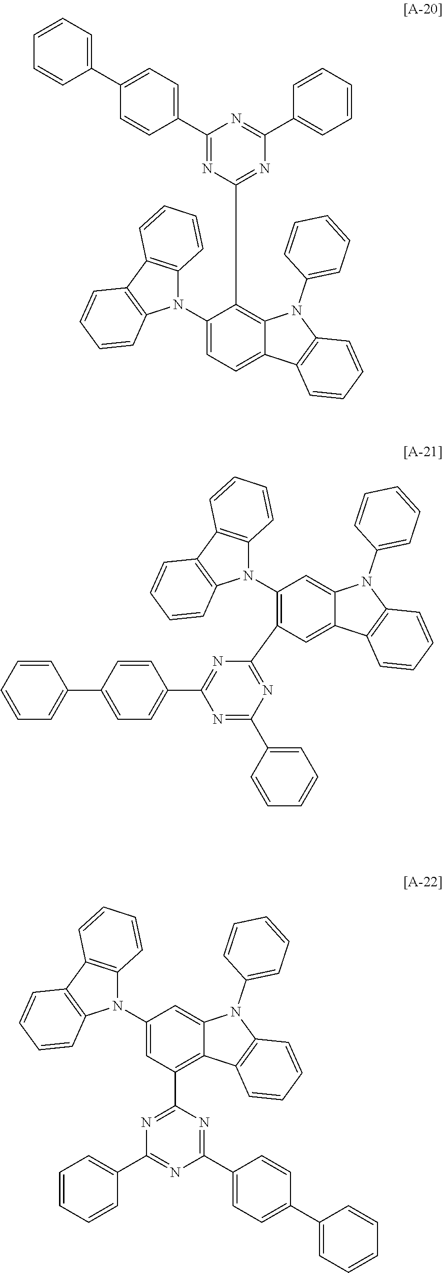 [SMILES: c1ccc(-c2ccc(-c3nc(-c4ccccc4)nc(-c4c(-n5c6ccccc6c6ccccc65)ccc5c6ccccc6n(-c6ccccc6)c45)n3)cc2)cc1 ; c1ccc(-c2ccc(-c3nc(-c4ccccc4)nc(-c4cc5c6ccccc6n(-c6ccccc6)c5cc4-n4c5ccccc5c5ccccc54)n3)cc2)cc1 ; c1ccc(-c2ccc(-c3nc(-c4ccccc4)nc(-c4cc(-n5c6ccccc6c6ccccc65)cc5c4c4ccccc4n5-c4ccccc4)n3)cc2)cc1]

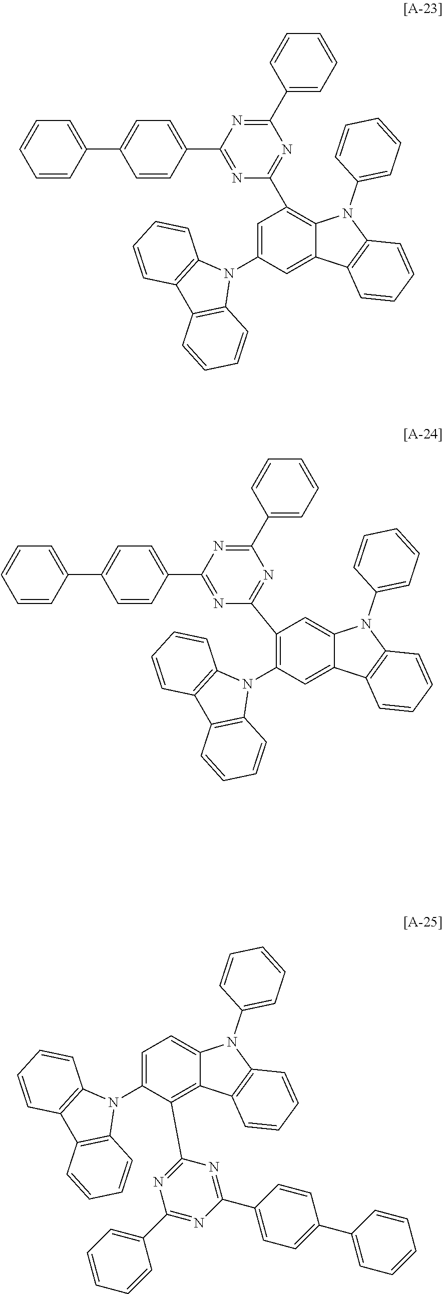 [SMILES: c1ccc(-c2ccc(-c3nc(-c4ccccc4)nc(-c4cc(-n5c6ccccc6c6ccccc65)cc5c6ccccc6n(-c6ccccc6)c45)n3)cc2)cc1 ; c1ccc(-c2ccc(-c3nc(-c4ccccc4)nc(-c4cc5c(cc4-n4c6ccccc6c6ccccc64)c4ccccc4n5-c4ccccc4)n3)cc2)cc1 ; c1ccc(-c2ccc(-c3nc(-c4ccccc4)nc(-c4c(-n5c6ccccc6c6ccccc65)ccc5c4c4ccccc4n5-c4ccccc4)n3)cc2)cc1]

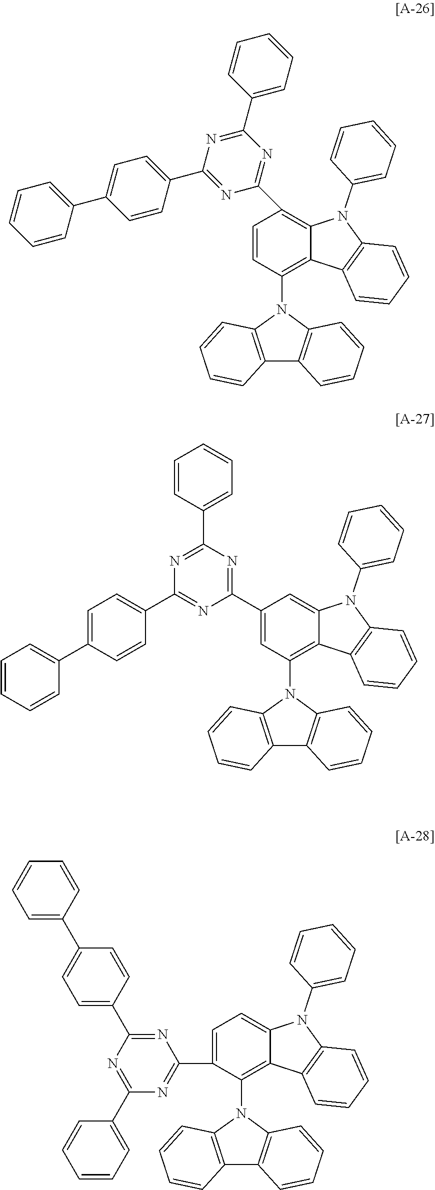 [SMILES: c1ccc(-c2ccc(-c3nc(-c4ccccc4)nc(-c4ccc(-n5c6ccccc6c6ccccc65)c5c6ccccc6n(-c6ccccc6)c45)n3)cc2)cc1 ; c1ccc(-c2ccc(-c3nc(-c4ccccc4)nc(-c4cc(-n5c6ccccc6c6ccccc65)c5c6ccccc6n(-c6ccccc6)c5c4)n3)cc2)cc1 ; c1ccc(-c2ccc(-c3nc(-c4ccccc4)nc(-c4ccc5c(c4-n4c6ccccc6c6ccccc64)c4ccccc4n5-c4ccccc4)n3)cc2)cc1]

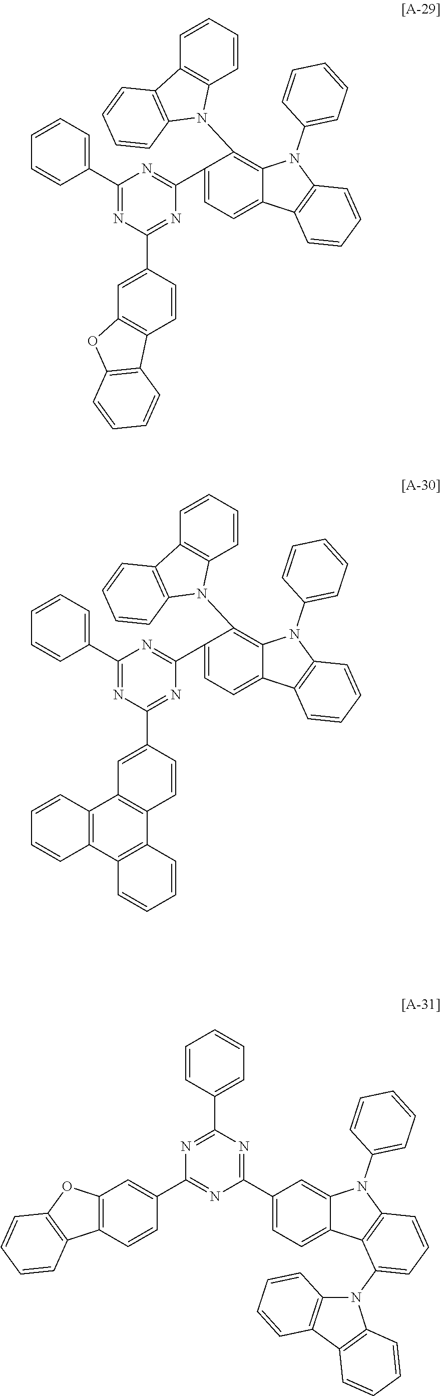 [SMILES: c1ccc(-c2nc(-c3ccc4c(c3)oc3ccccc34)nc(-c3ccc4c5ccccc5n(-c5ccccc5)c4c3-n3c4ccccc4c4ccccc43)n2)cc1 ; c1ccc(-c2nc(-c3ccc4c5ccccc5c5ccccc5c4c3)nc(-c3ccc4c5ccccc5n(-c5ccccc5)c4c3-n3c4ccccc4c4ccccc43)n2)cc1 ; c1ccc(-c2nc(-c3ccc4c(c3)oc3ccccc34)nc(-c3ccc4c5c(-n6c7ccccc7c7ccccc76)cccc5n(-c5ccccc5)c4c3)n2)cc1]

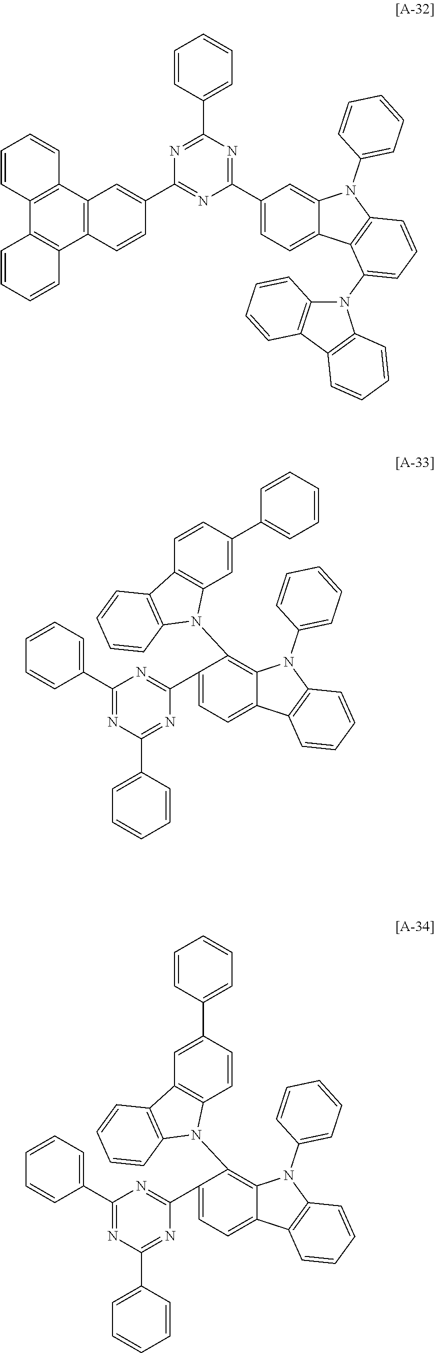 [SMILES: c1ccc(-c2nc(-c3ccc4c5ccccc5c5ccccc5c4c3)nc(-c3ccc4c5c(-n6c7ccccc7c7ccccc76)cccc5n(-c5ccccc5)c4c3)n2)cc1 ; c1ccc(-c2ccc3c4ccccc4n(-c4c(-c5nc(-c6ccccc6)nc(-c6ccccc6)n5)ccc5c6ccccc6n(-c6ccccc6)c45)c3c2)cc1 ; c1ccc(-c2ccc3c(c2)c2ccccc2n3-c2c(-c3nc(-c4ccccc4)nc(-c4ccccc4)n3)ccc3c4ccccc4n(-c4ccccc4)c23)cc1]

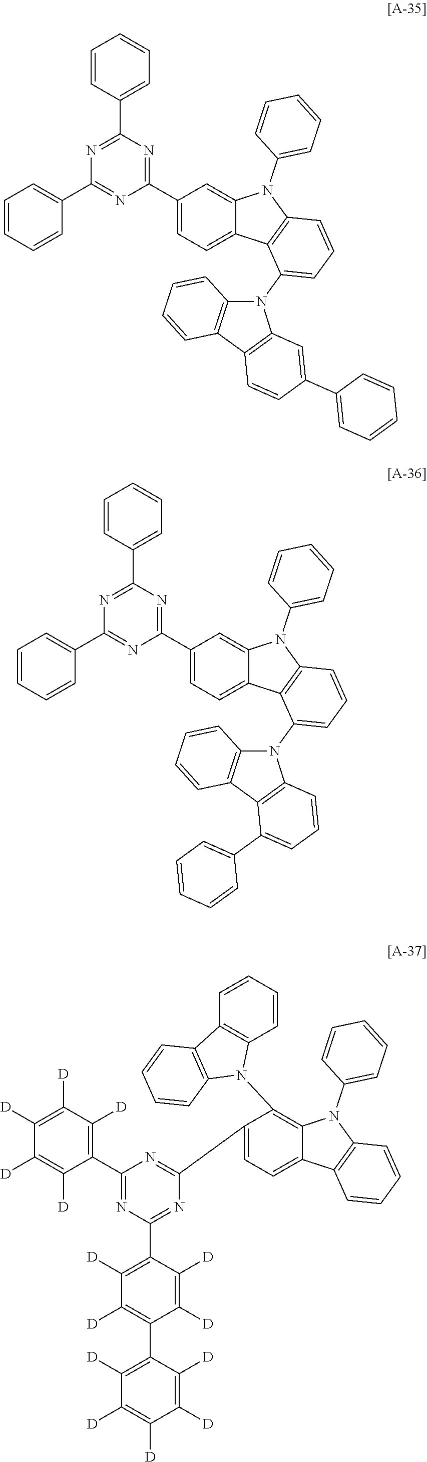 [SMILES: c1ccc(-c2ccc3c4ccccc4n(-c4cccc5c4c4ccc(-c6nc(-c7ccccc7)nc(-c7ccccc7)n6)cc4n5-c4ccccc4)c3c2)cc1 ; c1ccc(-c2nc(-c3ccccc3)nc(-c3ccc4c5c(-n6c7ccccc7c7c(-c8ccccc8)cccc76)cccc5n(-c5ccccc5)c4c3)n2)cc1 ; [2H]c1c([2H])c([2H])c(-c2nc(-c3ccc4c5ccccc5n(-c5ccccc5)c4c3-n3c4ccccc4c4ccccc43)nc(-c3c([2H])c([2H])c(-c4c([2H])c([2H])c([2H])c([2H])c4[2H])c([2H])c3[2H])n2)c([2H])c1[2H]]

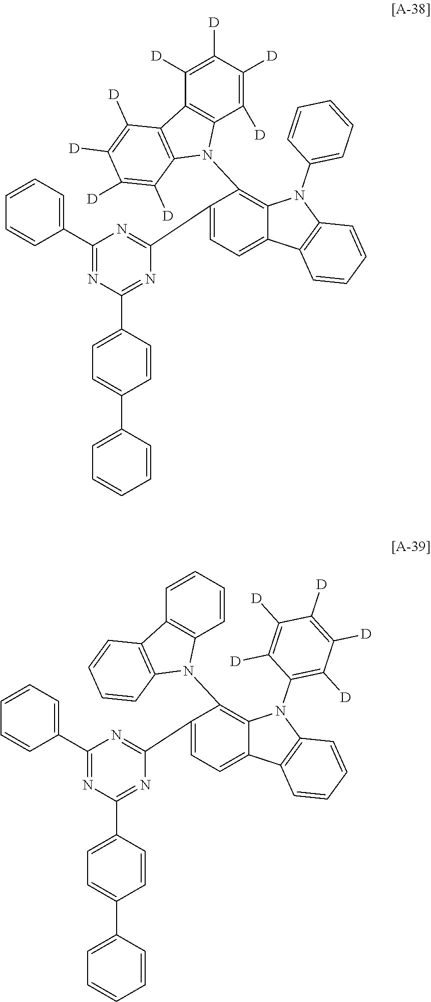 [SMILES: [2H]c1c([2H])c([2H])c2c(c1[2H])c1c([2H])c([2H])c([2H])c([2H])c1n2-c1c(-c2nc(-c3ccccc3)nc(-c3ccc(-c4ccccc4)cc3)n2)ccc2c3ccccc3n(-c3ccccc3)c12 ; [2H]c1c([2H])c([2H])c(-n2c3ccccc3c3ccc(-c4nc(-c5ccccc5)nc(-c5ccc(-c6ccccc6)cc5)n4)c(-n4c5ccccc5c5ccccc54)c32)c([2H])c1[2H]]

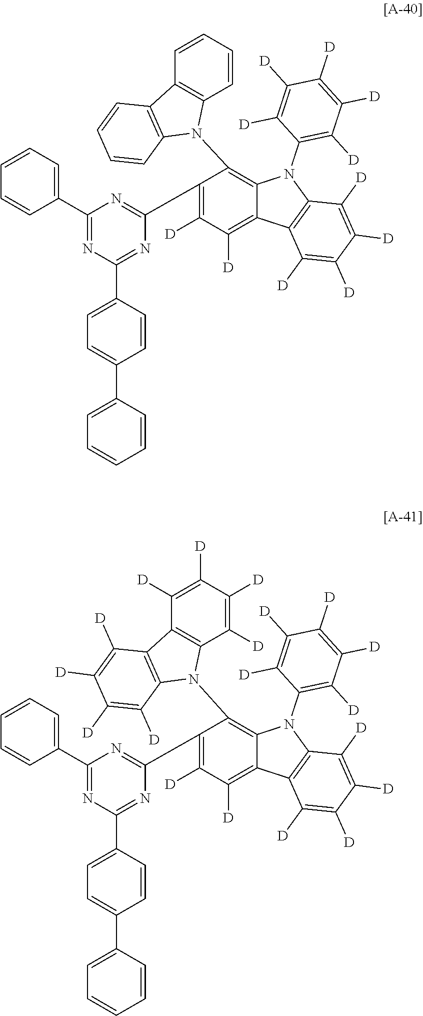 [SMILES: [2H]c1c([2H])c([2H])c(-n2c3c([2H])c([2H])c([2H])c([2H])c3c3c([2H])c([2H])c(-c4nc(-c5ccccc5)nc(-c5ccc(-c6ccccc6)cc5)n4)c(-n4c5ccccc5c5ccccc54)c32)c([2H])c1[2H] ; [2H]c1c([2H])c([2H])c(-n2c3c([2H])c([2H])c([2H])c([2H])c3c3c([2H])c([2H])c(-c4nc(-c5ccccc5)nc(-c5ccc(-c6ccccc6)cc5)n4)c(-n4c5c([2H])c([2H])c([2H])c([2H])c5c5c([2H])c([2H])c([2H])c([2H])c54)c32)c([2H])c1[2H]]

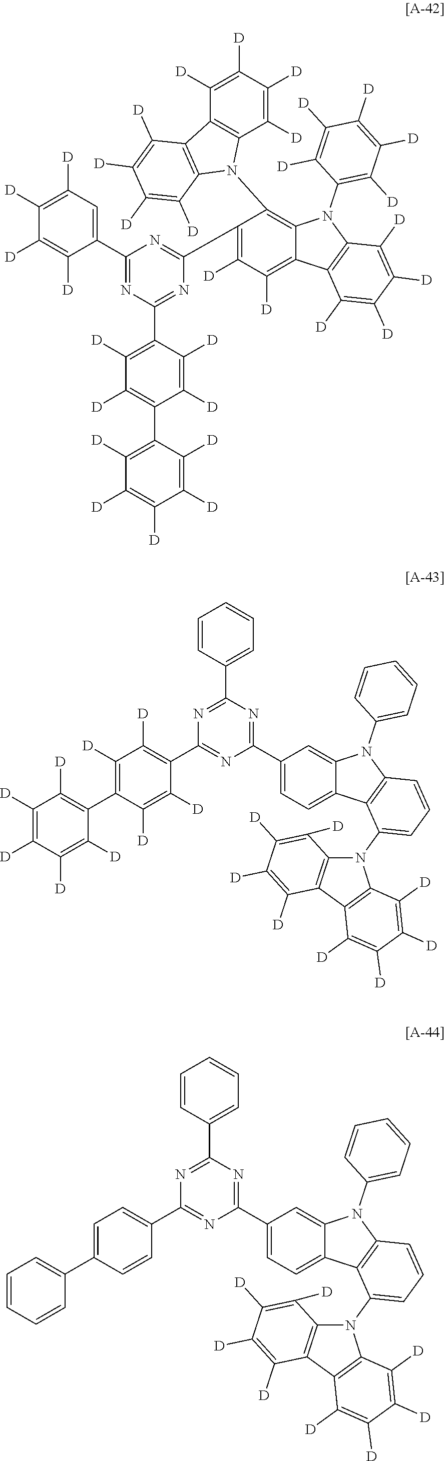

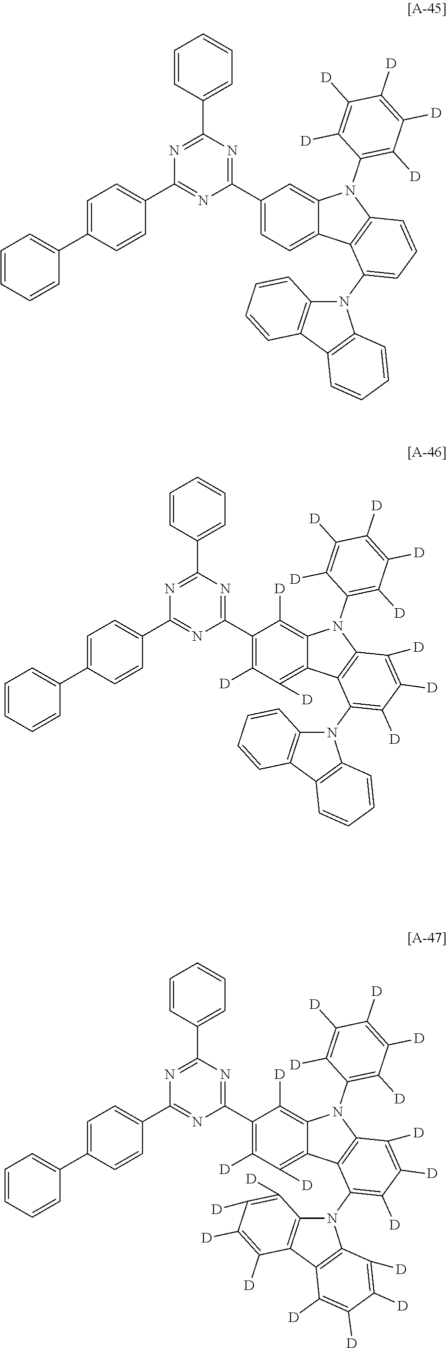 [SMILES: [2H]c1c([2H])c([2H])c(-n2c3cc(-c4nc(-c5ccccc5)nc(-c5ccc(-c6ccccc6)cc5)n4)ccc3c3c(-n4c5ccccc5c5ccccc54)cccc32)c([2H])c1[2H] ; [2H]c1c([2H])c([2H])c(-n2c3c([2H])c(-c4nc(-c5ccccc5)nc(-c5ccc(-c6ccccc6)cc5)n4)c([2H])c([2H])c3c3c(-n4c5ccccc5c5ccccc54)c([2H])c([2H])c([2H])c32)c([2H])c1[2H] ; [2H]c1c([2H])c([2H])c(-n2c3c([2H])c(-c4nc(-c5ccccc5)nc(-c5ccc(-c6ccccc6)cc5)n4)c([2H])c([2H])c3c3c(-n4c5c([2H])c([2H])c([2H])c([2H])c5c5c([2H])c([2H])c([2H])c([2H])c54)c([2H])c([2H])c([2H])c32)c([2H])c1[2H]]

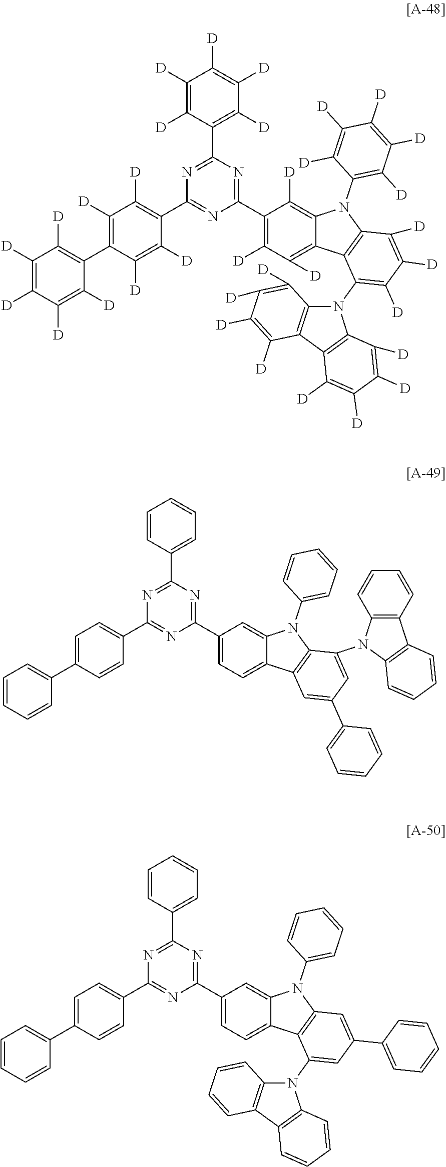

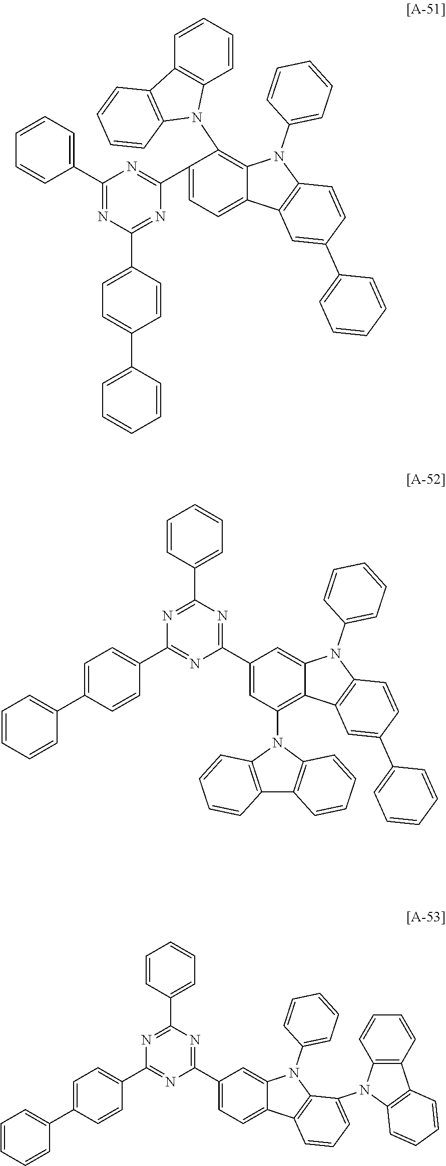 [SMILES: c1ccc(-c2ccc(-c3nc(-c4ccccc4)nc(-c4ccc5c6cc(-c7ccccc7)ccc6n(-c6ccccc6)c5c4-n4c5ccccc5c5ccccc54)n3)cc2)cc1 ; c1ccc(-c2ccc(-c3nc(-c4ccccc4)nc(-c4cc(-n5c6ccccc6c6ccccc65)c5c6cc(-c7ccccc7)ccc6n(-c6ccccc6)c5c4)n3)cc2)cc1 ; c1ccc(-c2ccc(-c3nc(-c4ccccc4)nc(-c4ccc5c6cccc(-n7c8ccccc8c8ccccc87)c6n(-c6ccccc6)c5c4)n3)cc2)cc1]

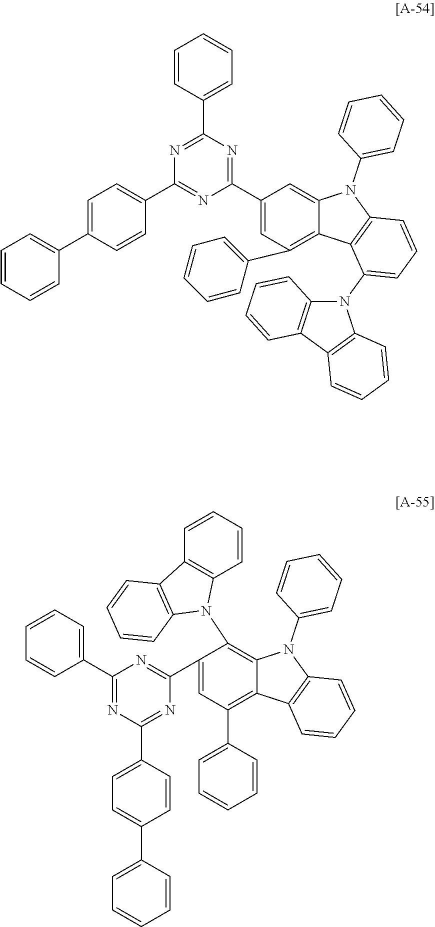 [SMILES: c1ccc(-c2ccc(-c3nc(-c4ccccc4)nc(-c4cc(-c5ccccc5)c5c6c(-n7c8ccccc8c8ccccc87)cccc6n(-c6ccccc6)c5c4)n3)cc2)cc1 ; c1ccc(-c2ccc(-c3nc(-c4ccccc4)nc(-c4cc(-c5ccccc5)c5c6ccccc6n(-c6ccccc6)c5c4-n4c5ccccc5c5ccccc54)n3)cc2)cc1]

The second compound may be represented by Chemical Formula 2.

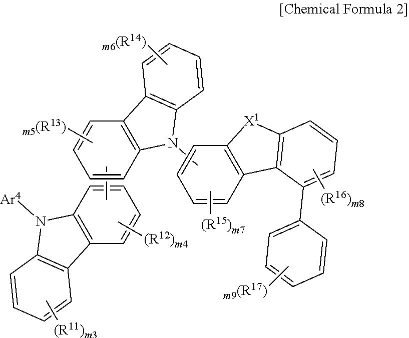

In Chemical Formula 2,
X¹ is O or S,
Ar⁴ is a substituted or unsubstituted C6 to C30 aryl group, a substituted or unsubstituted C2 to C30 heterocyclic group, or a combination thereof,
R¹¹ to R¹⁴ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
R¹⁵ to R¹⁷ are each independently hydrogen, deuterium, or a substituted or unsubstituted phenyl group,
m4, m5, m7, and m8 are each independently an integer from 1 to 3, and
m3, m6, and m9 are each independently an integer from 1 to 4.

The bicarbazole, which has dibenzofuran (or dibenzothiophene) substituted with phenyl at the 9th position, has a property of shortening a distance between molecules of the hole characteristic compound and the electron characteristic compound. In particular, the LUMO of the hole characteristic compound and the LUMO of the electronic characteristic compound are arranged closely, so that the LUMO of the electronic characteristic compound extends to the LUMO of the hole characteristic compound, and is deposited in an arrangement that is advantageous for electron transfer. Due to the above structural arrangement, the low-driving/high-efficiency characteristics of the organic light emitting diode using the same can be realized.

In particular, when used together with the above-described first compound, the charge balance is appropriately maintained and exciton generation is advantageous, thereby enabling the implementation of high-efficiency device characteristics.

In Chemical Formula 2, when m3 is greater than or equal to 2, each R¹¹ may be the same or different from each other.

In Chemical Formula 2, when m4 is greater than or equal to 2, each R¹² may be the same or different from each other.

In Chemical Formula 2, when m5 is greater than or equal to 2, each R¹³ may be the same or different from each other.

In Chemical Formula 2, when m6 is greater than or equal to 2, each R¹⁴ may be the same or different from each other.

In Chemical Formula 2, when m7 is greater than or equal to 2, each R¹⁵ may be the same or different from each other.

In Chemical Formula 2, when m8 is greater than or equal to 2, each R¹⁶ may be the same or different from each other.

In Chemical Formula 2, when m9 is greater than or equal to 2, each R¹⁷ may be the same or different from each other.

For example, the second compound may be represented by any one of Chemical Formula 2-1 to Chemical Formula 2-4.

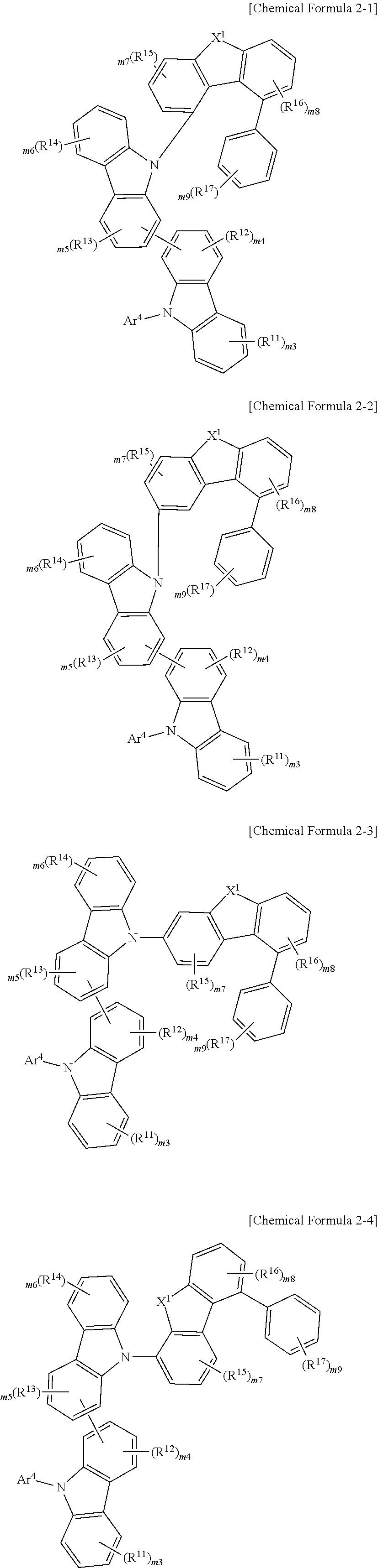

In Chemical Formula 2-1 to Chemical Formula 2-4, X¹, Art, R¹¹ to R¹⁷, and m3 to m9 are the same as described above.

As a specific example, Chemical Formula 2-1 may be represented by any one of Chemical Formula 2-1-1 to Chemical Formula 2-1-16.

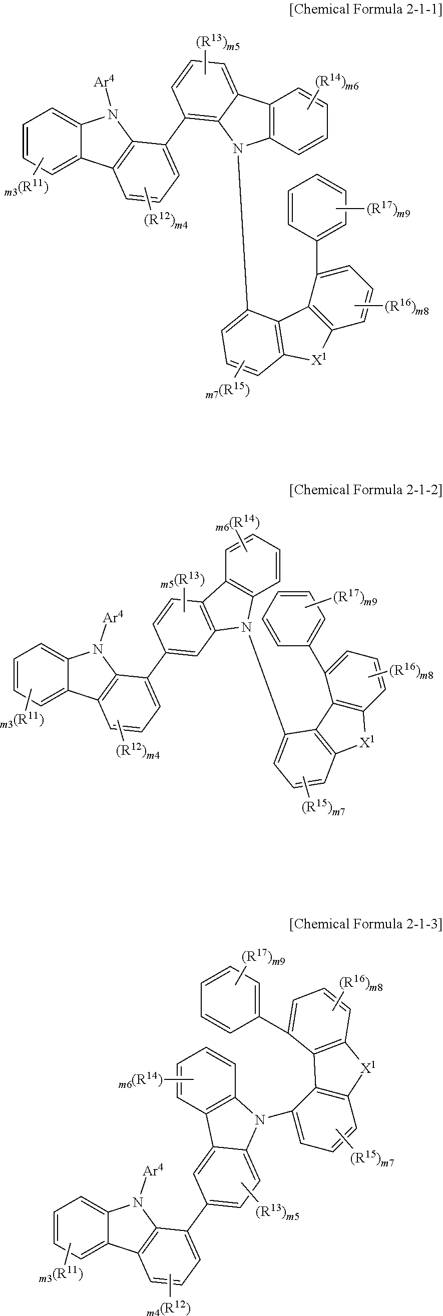

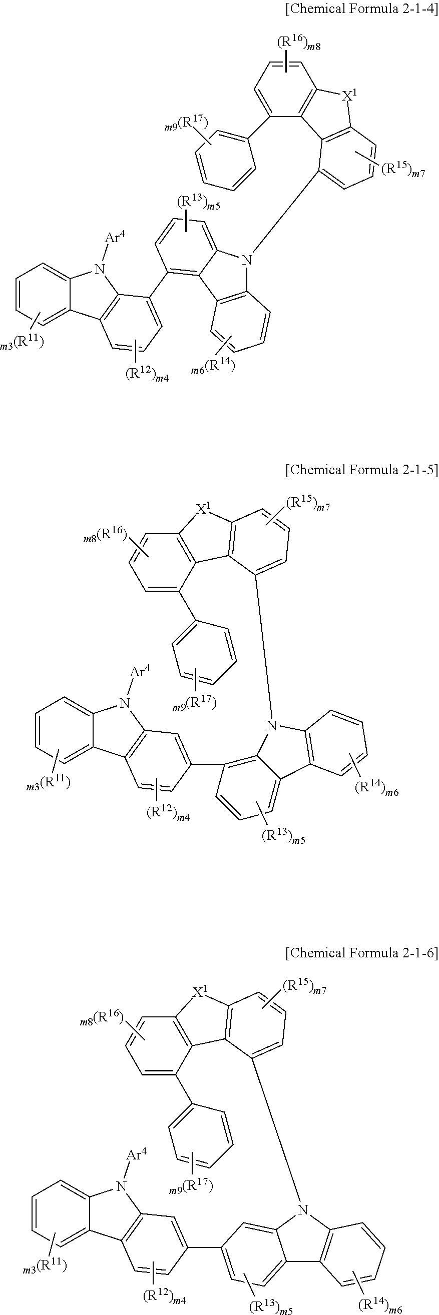

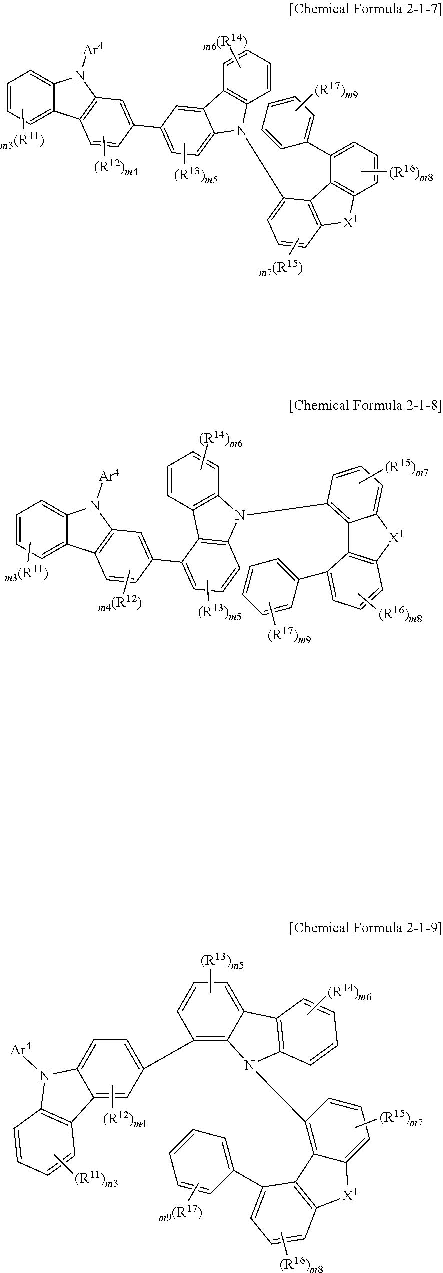

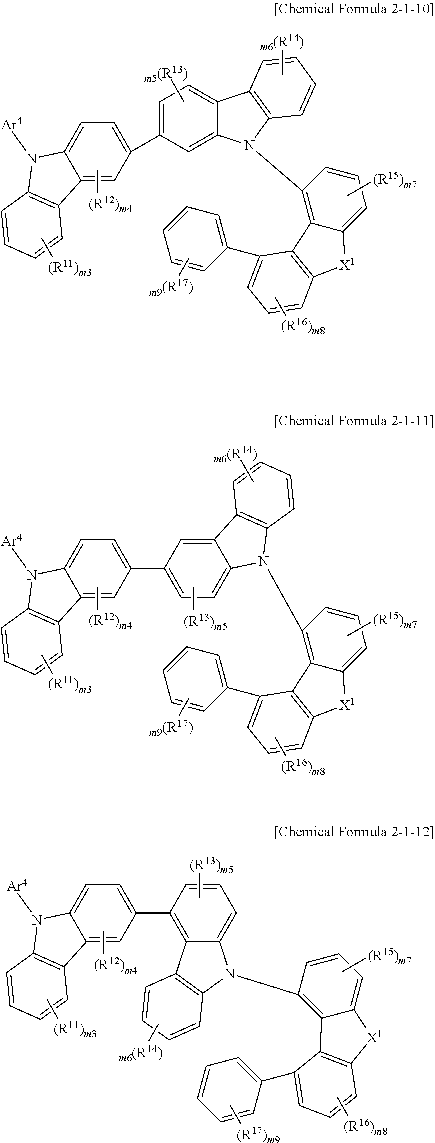

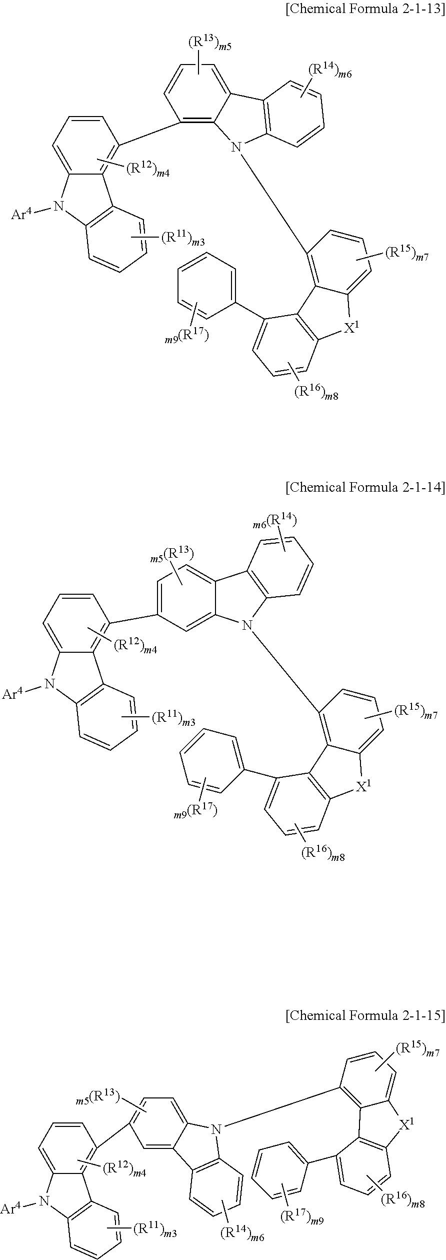

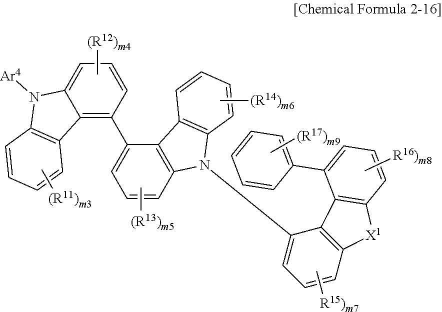

In Chemical Formula 2-1-1 to Chemical Formula 2-1-16, X¹, Art, R¹¹ to R¹⁷, and m3 to m9 are the same as described above.

As a specific example, Chemical Formula 2-1 may be represented by any one of Chemical Formula 2-2-1 to Chemical Formula 2-2-16.

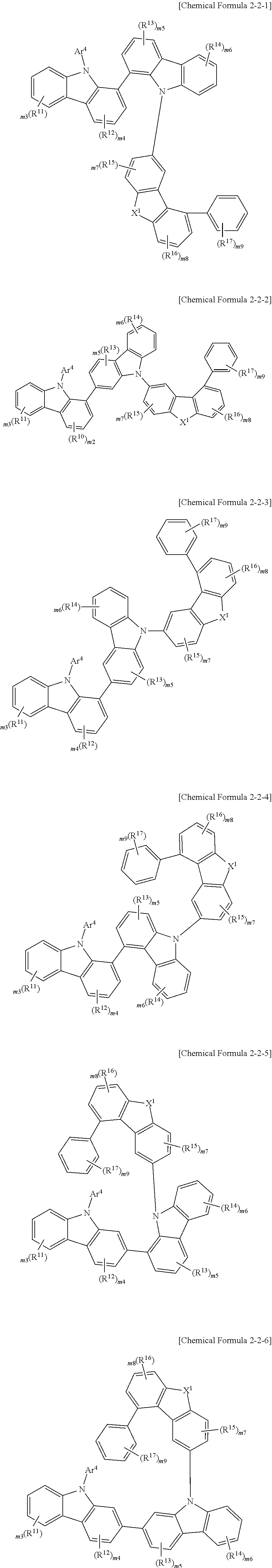

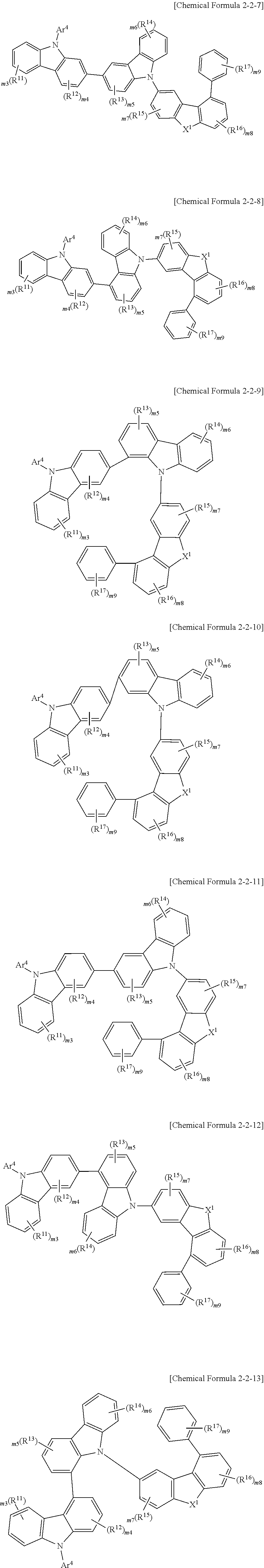

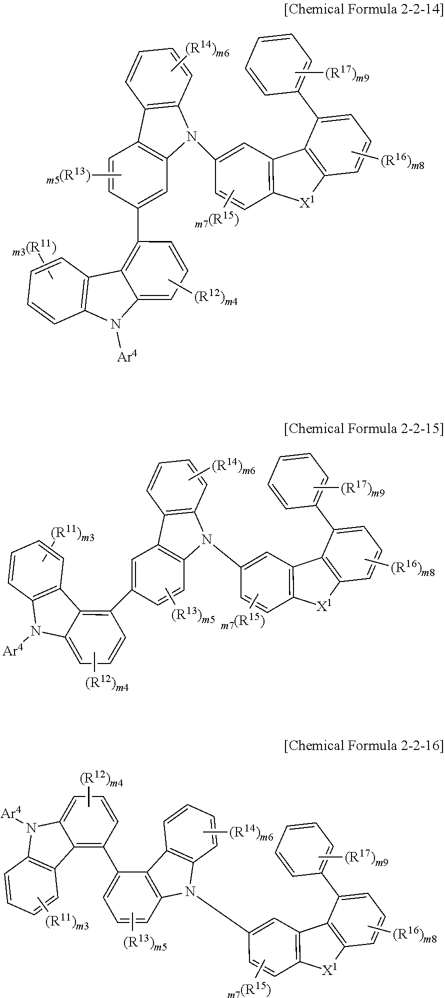

In Chemical Formula 2-2-1 to Chemical Formula 2-2-16, X¹, Art, R¹¹ to R¹⁷, and m3 to m9 are the same as described above.

As a specific example, Chemical Formula 2-3 may be represented by any one of Chemical Formula 2-3-1 to Chemical Formula 2-3-16.

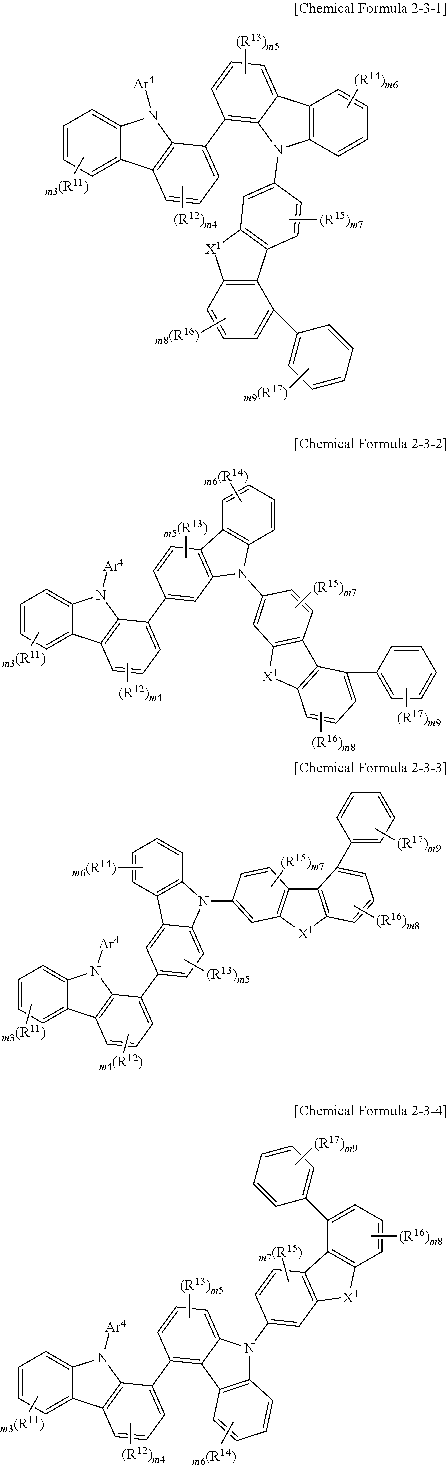

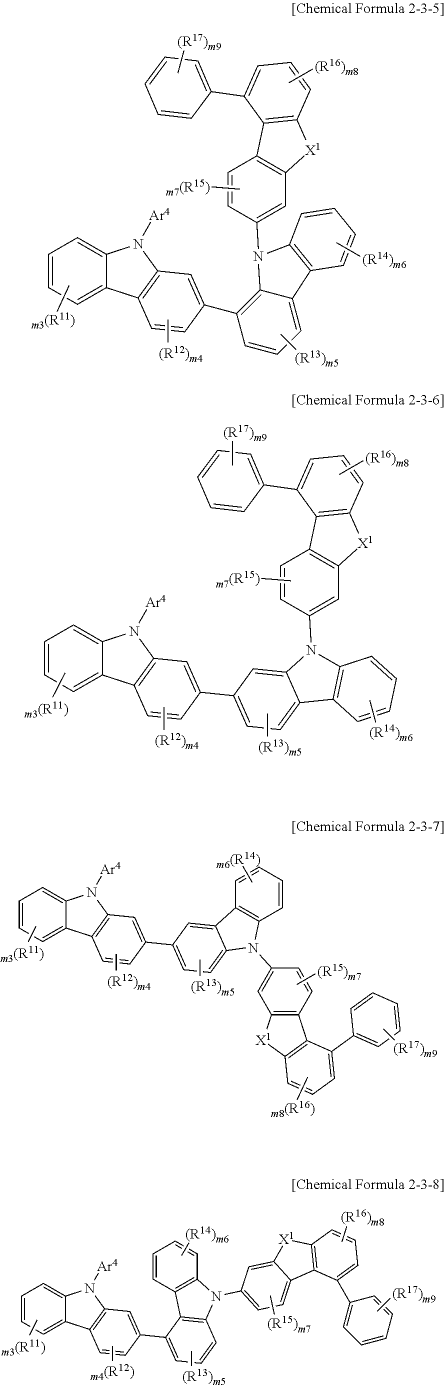

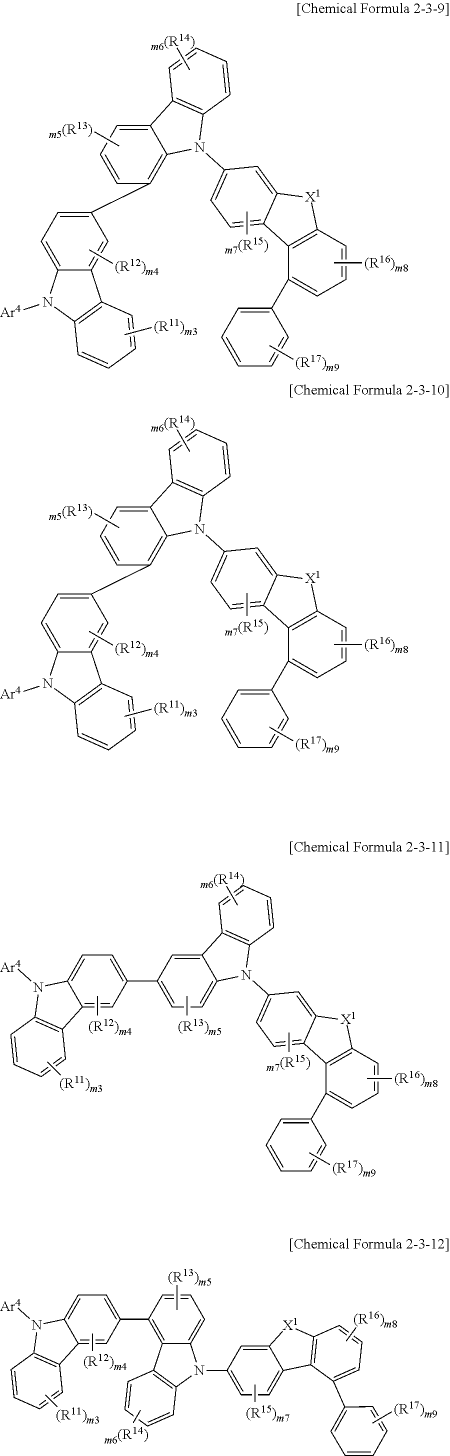

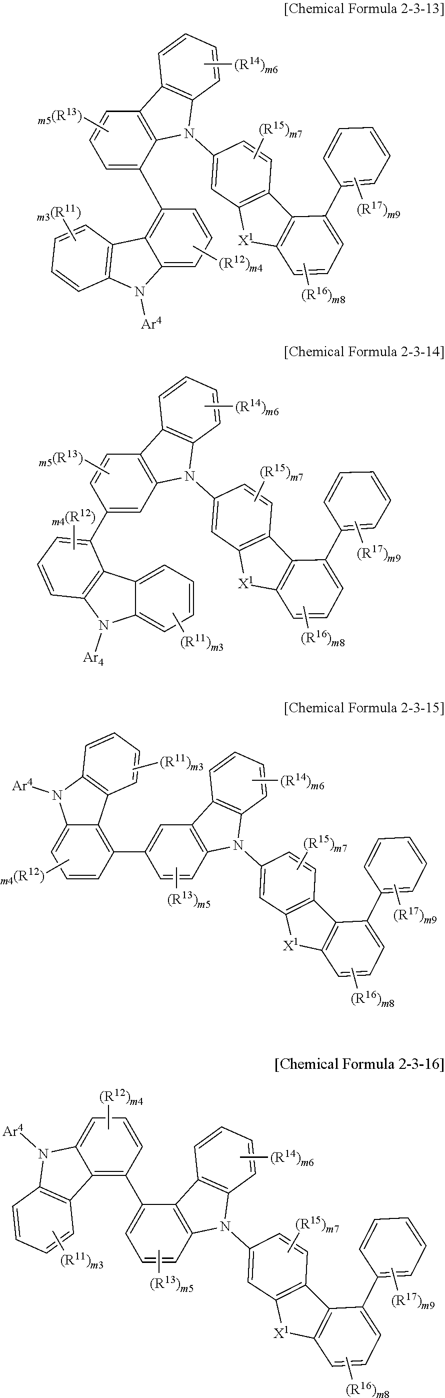

In Chemical Formula 2-3-1 to Chemical Formula 2-3-16, X¹, Art, R¹¹ to R¹⁷, and m3 to m9 are the same as described above.

As a specific example, Chemical Formula 2-4 may be represented by any one of Chemical Formula 2-4-1 to Chemical Formula 2-4-10.

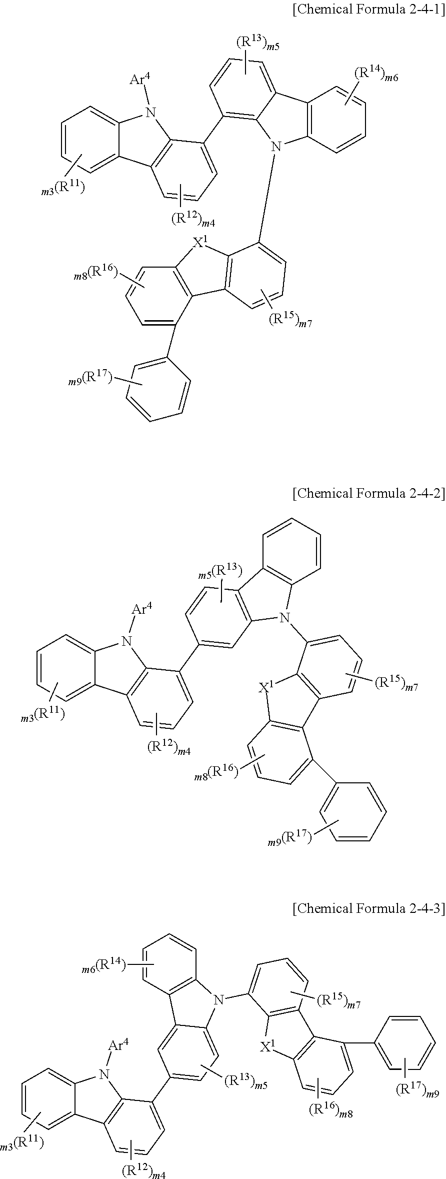

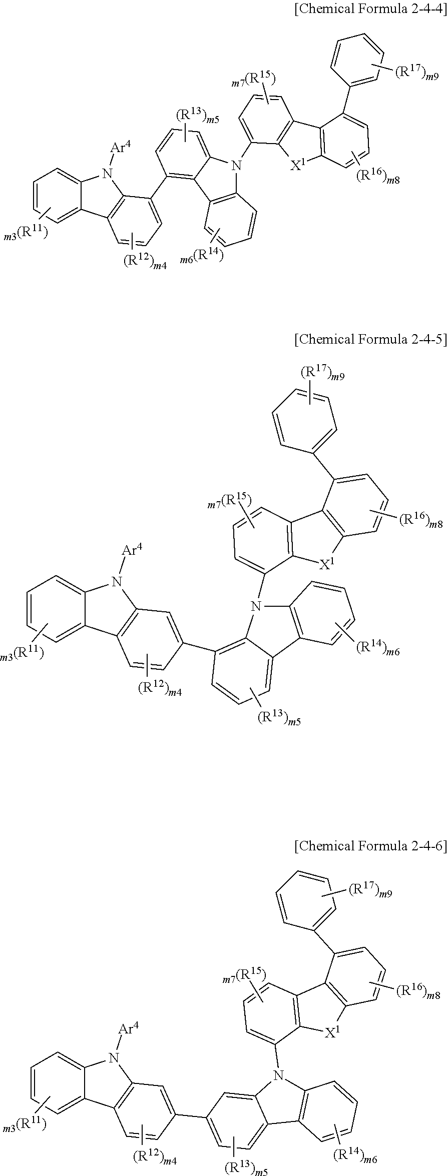

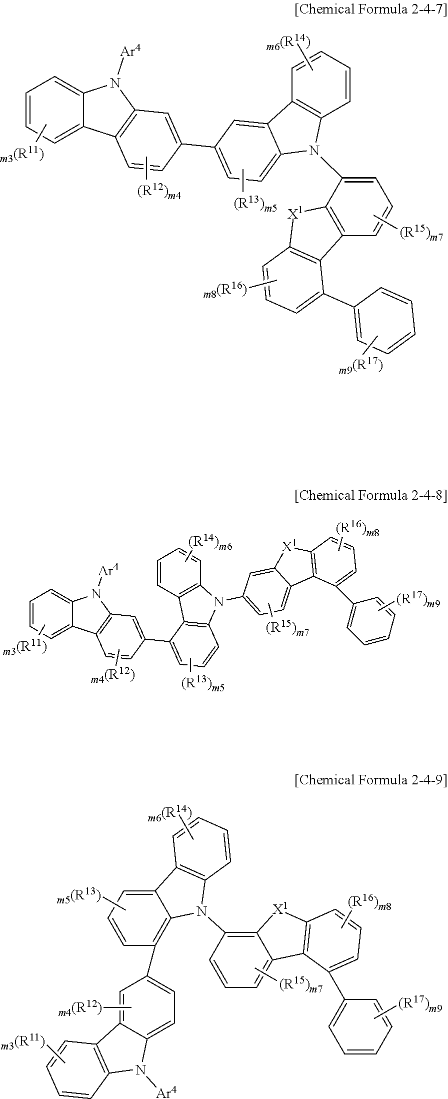

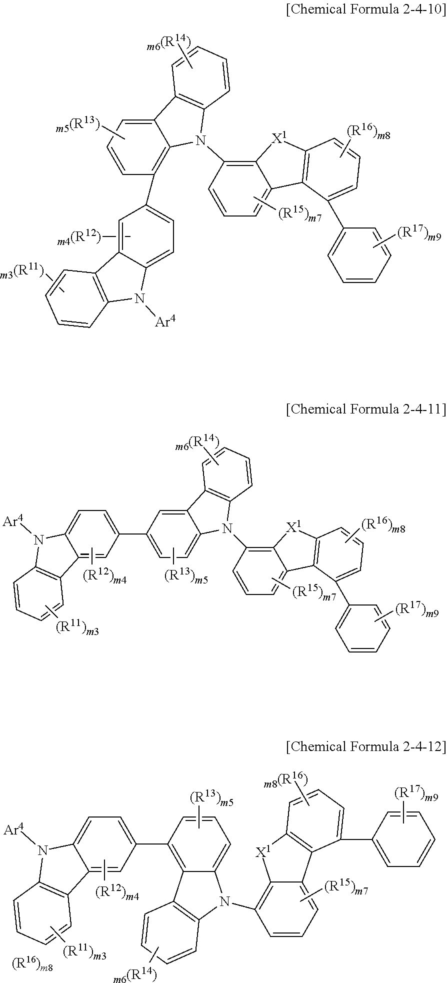

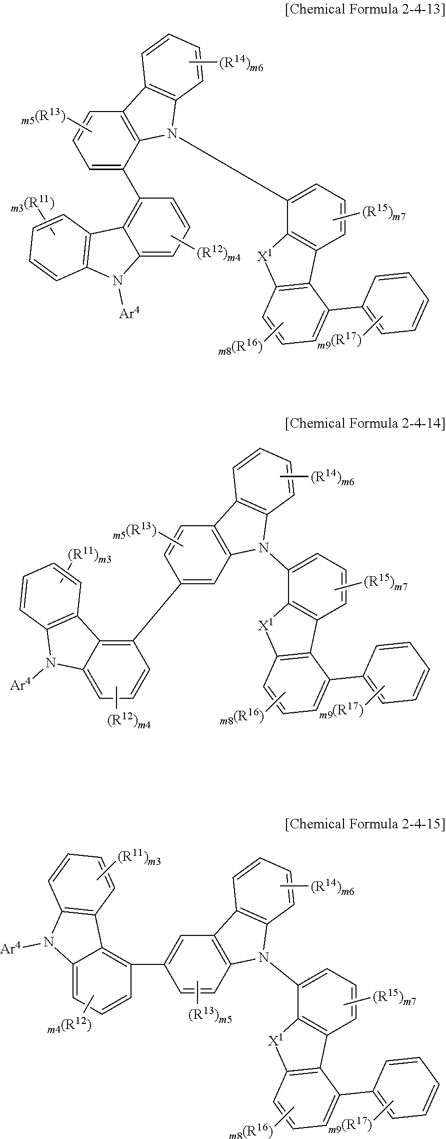

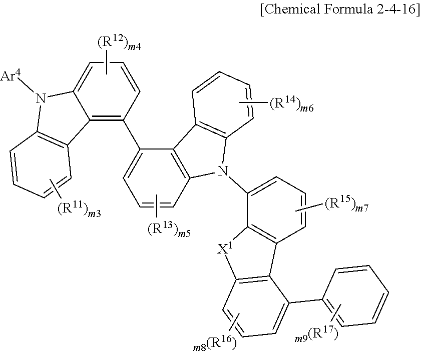

In Chemical Formula 2-4-1 to Chemical Formula 2-4-16, X¹, Art, R¹¹ to R¹⁷, and m3 to m9 are the same as described above.

For example, the second compound may be represented by any one of Chemical Formula Feb. 1, 2011, Chemical Formula 2-2-12, Chemical Formula 2-3-13, and Chemical Formula 2-4-8.

For example, in Chemical Formula 2, Ar⁴ may be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, or a substituted or unsubstituted fluorenyl group.

As a specific example, in Chemical Formula 2, Ar⁴ may be a substituted or unsubstituted phenyl group, or a substituted or unsubstituted biphenyl group.

For example, in Chemical Formula 2, R¹¹ to R¹⁴ may each independently be hydrogen, deuterium, a substituted or unsubstituted C1 to C10 alkyl group, a substituted or unsubstituted C6 to C20 aryl group or a substituted or unsubstituted C2 to C20 heterocyclic group.

As a specific example, in Chemical Formula 2, R¹¹ to R¹⁴ may each independently be hydrogen, deuterium, a substituted or unsubstituted C6 to C12 aryl group, a substituted or unsubstituted carbazolyl group, a substituted or unsubstituted dibenzofuranyl group, or a substituted or unsubstituted dibenzothiophenyl group.

As a more specific example, in Chemical Formula 2, R¹¹ to R¹⁴ may each independently be hydrogen, deuterium, a substituted or unsubstituted phenyl group, a substituted or unsubstituted biphenyl group, a substituted or unsubstituted naphthyl group, a substituted or unsubstituted carbazolyl group, a substituted or unsubstituted dibenzofuranyl group, or a substituted or unsubstituted dibenzothiophenyl group.

For example, the second compound may be one selected from the compounds listed in Group 2, but is not limited thereto.

[chemical structure]

[chemical structure]

[chemical structure]

[chemical structure]

[chemical structure]

[chemical structure]

[chemical structure]

[chemical structure]

[chemical structure]

[chemical structure]

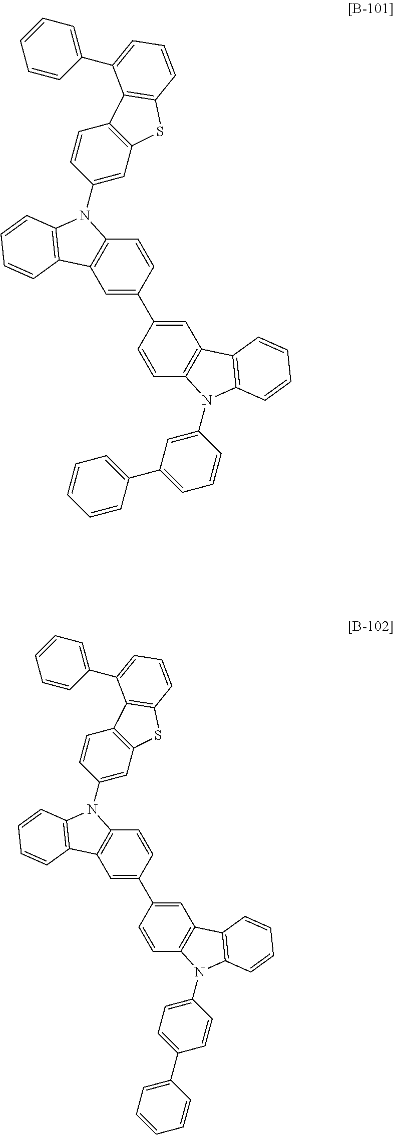 [SMILES: c1ccc(-c2cccc(-n3c4ccccc4c4cc(-c5ccc6c(c5)c5ccccc5n6-c5ccc6c(c5)sc5cccc(-c7ccccc7)c56)ccc43)c2)cc1 ; c1ccc(-c2ccc(-n3c4ccccc4c4cc(-c5ccc6c(c5)c5ccccc5n6-c5ccc6c(c5)sc5cccc(-c7ccccc7)c56)ccc43)cc2)cc1]

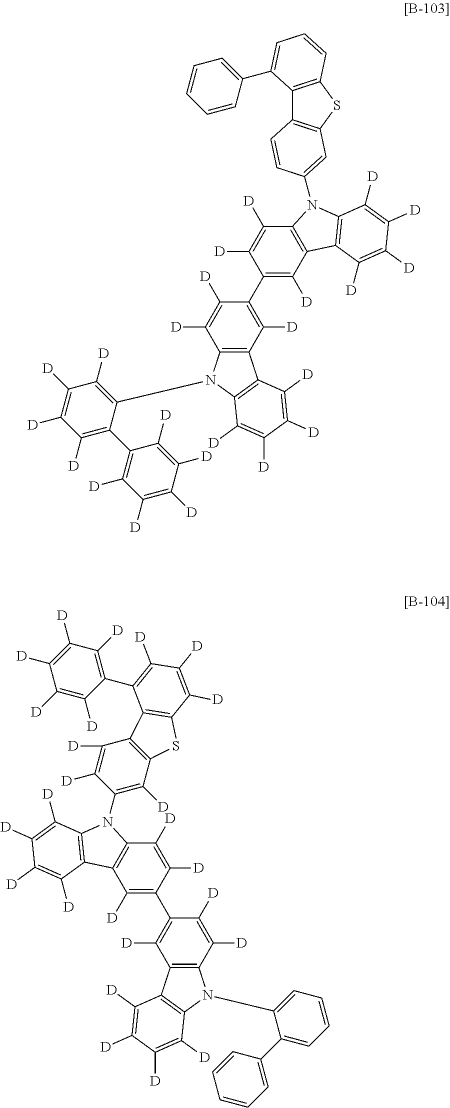 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)sc4cccc(-c6ccccc6)c45)c([2H])c([2H])c32)c([2H])c1[2H] ; [2H]c1c([2H])c([2H])c(-c2c([2H])c([2H])c([2H])c3sc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c([2H])c4c23)c([2H])c1[2H]]

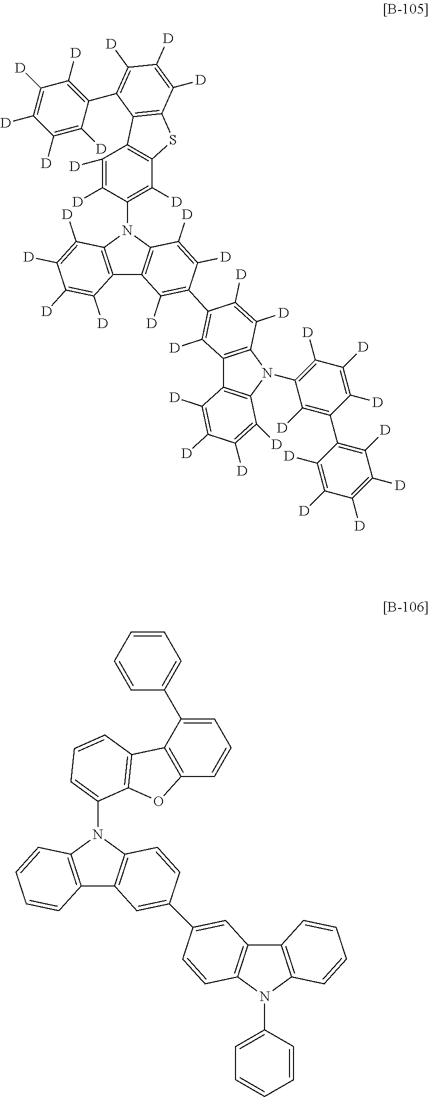 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(sc7c([2H])c([2H])c([2H])c(-c8c([2H])c([2H])c([2H])c([2H])c8[2H])c76)c5[2H])c([2H])c([2H])c43)c2[2H])c([2H])c1[2H] ; c1ccc(-c2cccc3oc4c(-n5c6ccccc6c6cc(-c7ccc8c(c7)c7ccccc7n8-c7ccccc7)ccc65)cccc4c23)cc1]

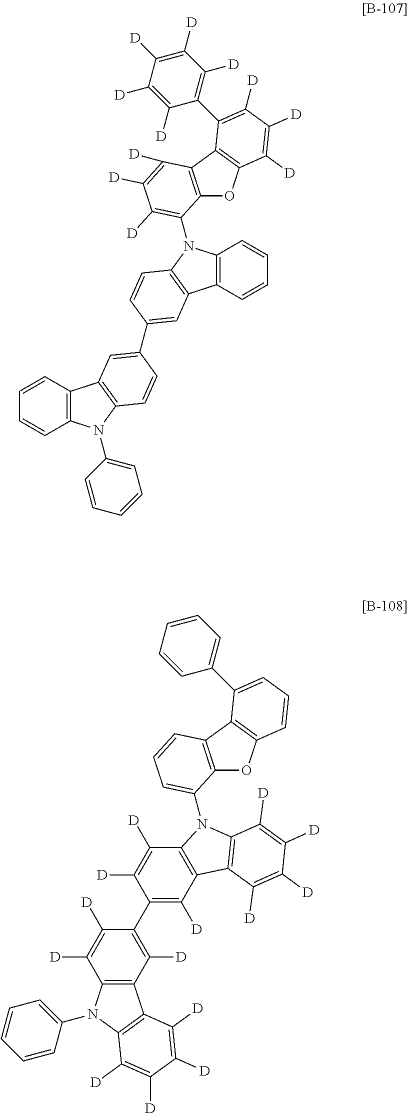 [SMILES: [2H]c1c([2H])c([2H])c(-c2c([2H])c([2H])c([2H])c3oc4c(-n5c6ccccc6c6cc(-c7ccc8c(c7)c7ccccc7n8-c7ccccc7)ccc65)c([2H])c([2H])c([2H])c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cccc(-c5ccccc5)c34)c([2H])c([2H])c1n2-c1ccccc1]

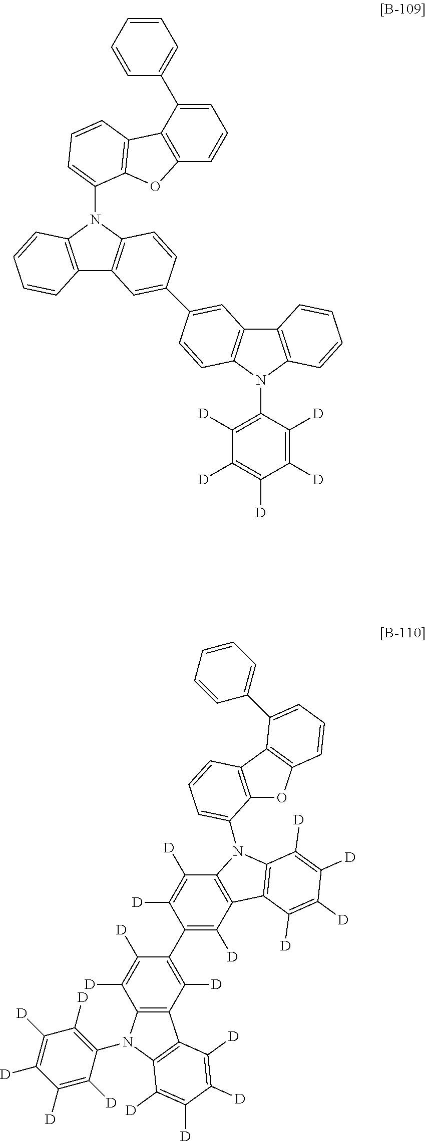 [SMILES: [2H]c1c([2H])c([2H])c(-n2c3ccccc3c3cc(-c4ccc5c(c4)c4ccccc4n5-c4cccc5c4oc4cccc(-c6ccccc6)c45)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4oc4cccc(-c6ccccc6)c45)c([2H])c([2H])c32)c([2H])c1[2H]]

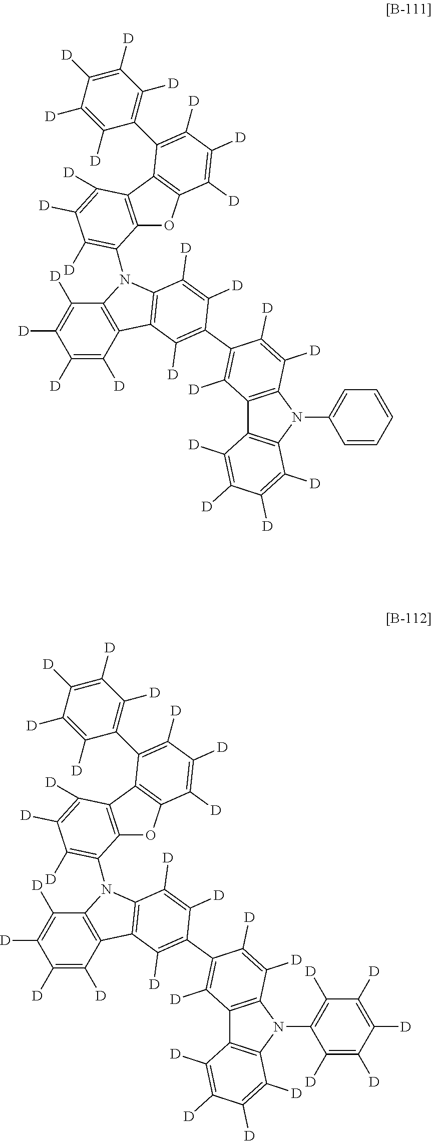 [SMILES: [2H]c1c([2H])c([2H])c(-c2c([2H])c([2H])c([2H])c3oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c([2H])c([2H])c([2H])c4c23)c([2H])c1[2H] ; [2H]c1c([2H])c([2H])c(-c2c([2H])c([2H])c([2H])c3oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c([2H])c([2H])c4c23)c([2H])c1[2H]]

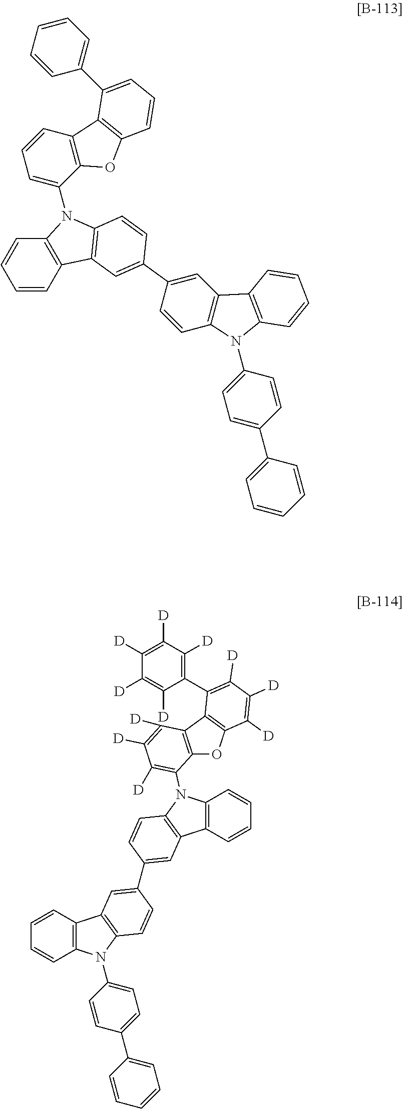 [SMILES: c1ccc(-c2ccc(-n3c4ccccc4c4cc(-c5ccc6c(c5)c5ccccc5n6-c5cccc6c5oc5cccc(-c7ccccc7)c56)ccc43)cc2)cc1 ; [2H]c1c([2H])c([2H])c(-c2c([2H])c([2H])c([2H])c3oc4c(-n5c6ccccc6c6cc(-c7ccc8c(c7)c7ccccc7n8-c7ccc(-c8ccccc8)cc7)ccc65)c([2H])c([2H])c([2H])c4c23)c([2H])c1[2H]]

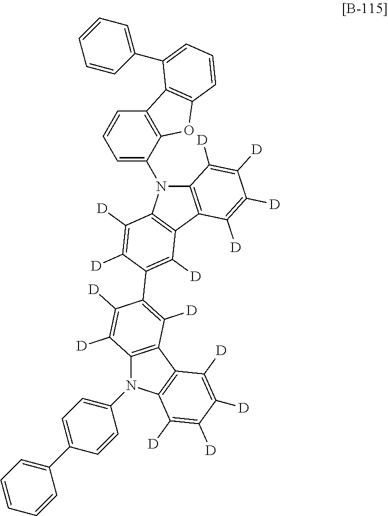 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cccc(-c5ccccc5)c34)c([2H])c([2H])c1n2-c1ccc(-c2ccccc2)cc1]

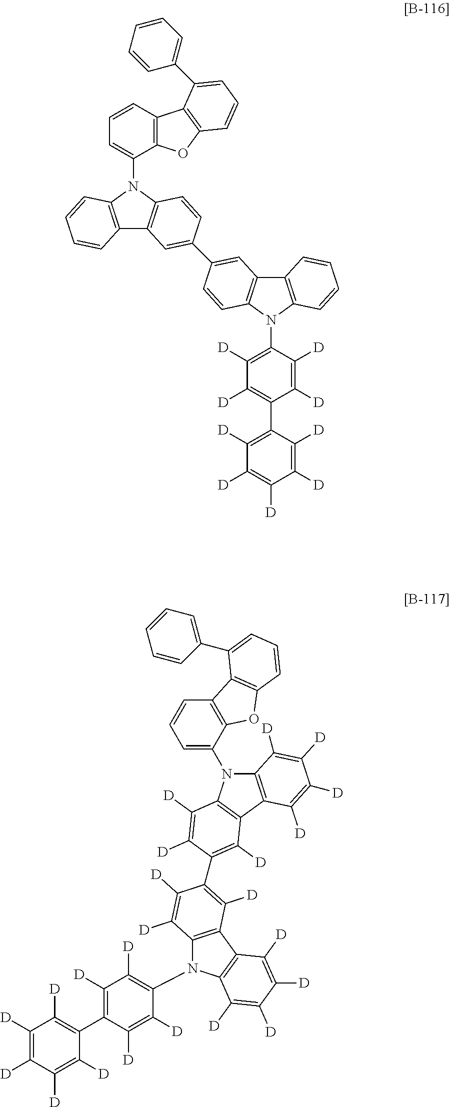 [SMILES: [2H]c1c([2H])c([2H])c(-c2c([2H])c([2H])c(-n3c4ccccc4c4cc(-c5ccc6c(c5)c5ccccc5n6-c5cccc6c5oc5cccc(-c7ccccc7)c56)ccc43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5oc5cccc(-c7ccccc7)c56)c([2H])c([2H])c43)c([2H])c2[2H])c([2H])c1[2H]]

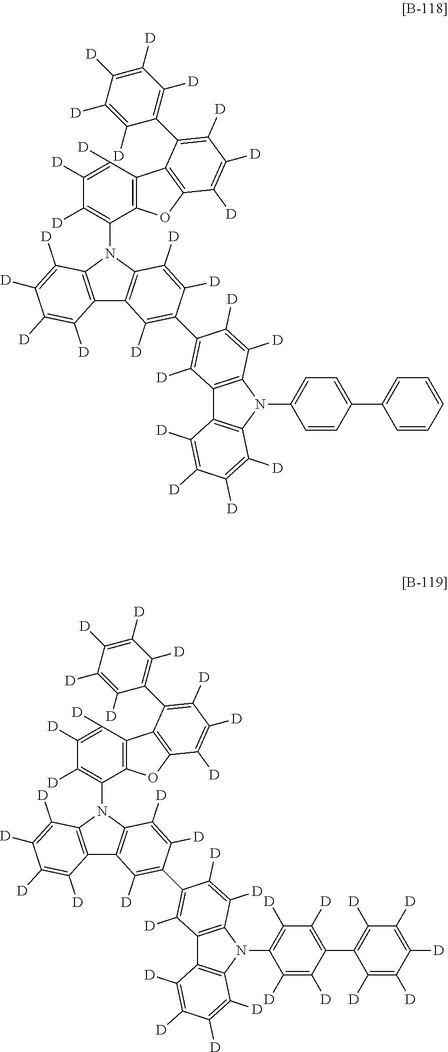

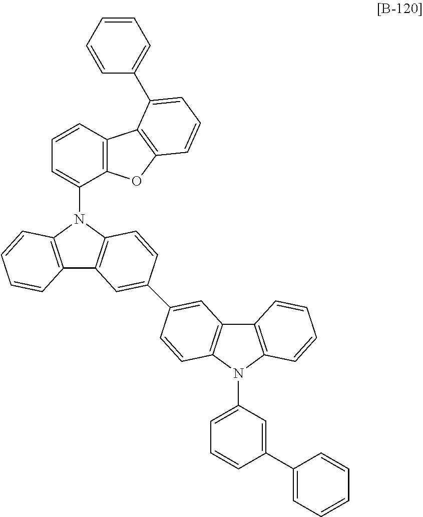 [SMILES: c1ccc(-c2cccc(-n3c4ccccc4c4cc(-c5ccc6c(c5)c5ccccc5n6-c5cccc6c5oc5cccc(-c7ccccc7)c56)ccc43)c2)cc1]

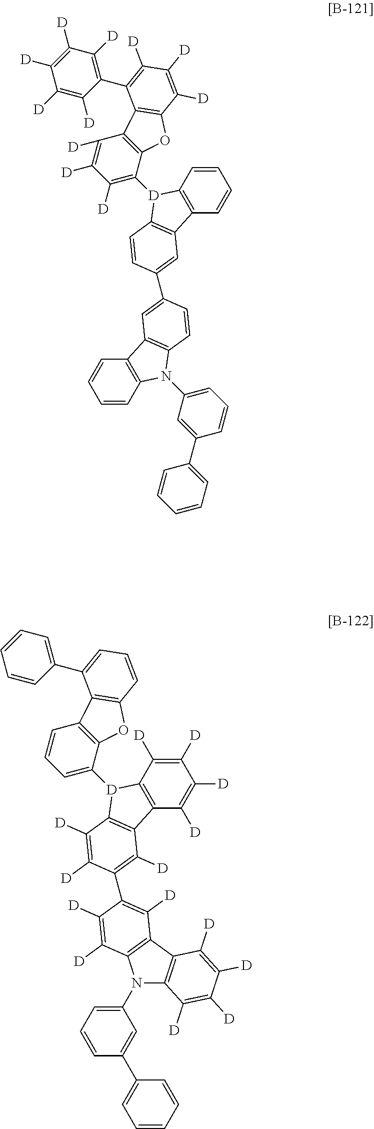

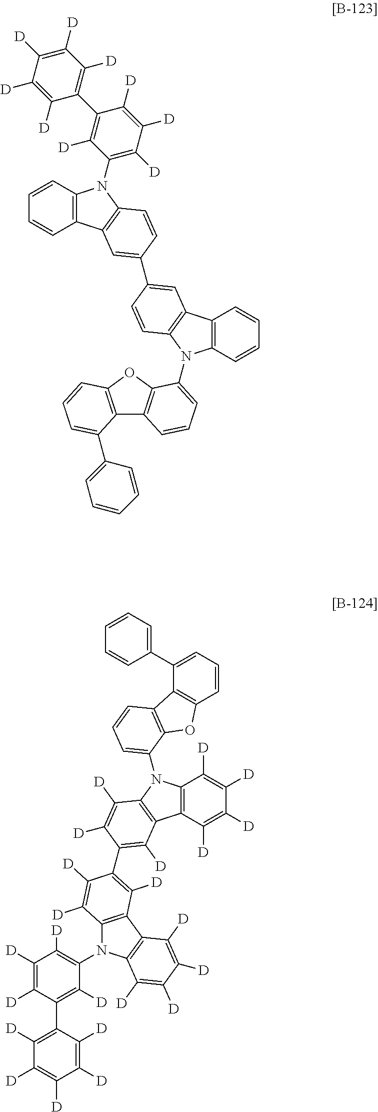 [SMILES: [2H]c1c([2H])c([2H])c(-c2c([2H])c([2H])c([2H])c(-n3c4ccccc4c4cc(-c5ccc6c(c5)c5ccccc5n6-c5cccc6c5oc5cccc(-c7ccccc7)c56)ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5oc5cccc(-c7ccccc7)c56)c([2H])c([2H])c43)c2[2H])c([2H])c1[2H]]

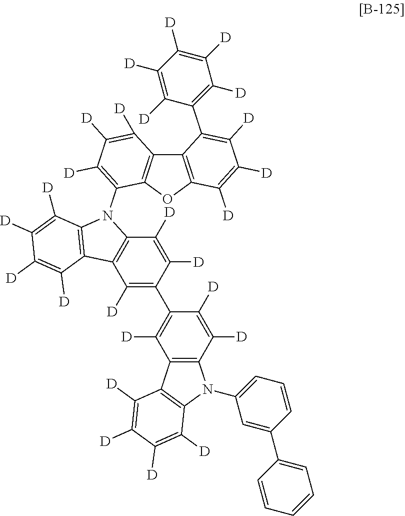 [SMILES: [2H]c1c([2H])c([2H])c(-c2c([2H])c([2H])c([2H])c3oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7cccc(-c8ccccc8)c7)c([2H])c([2H])c65)c([2H])c([2H])c([2H])c4c23)c([2H])c1[2H]]

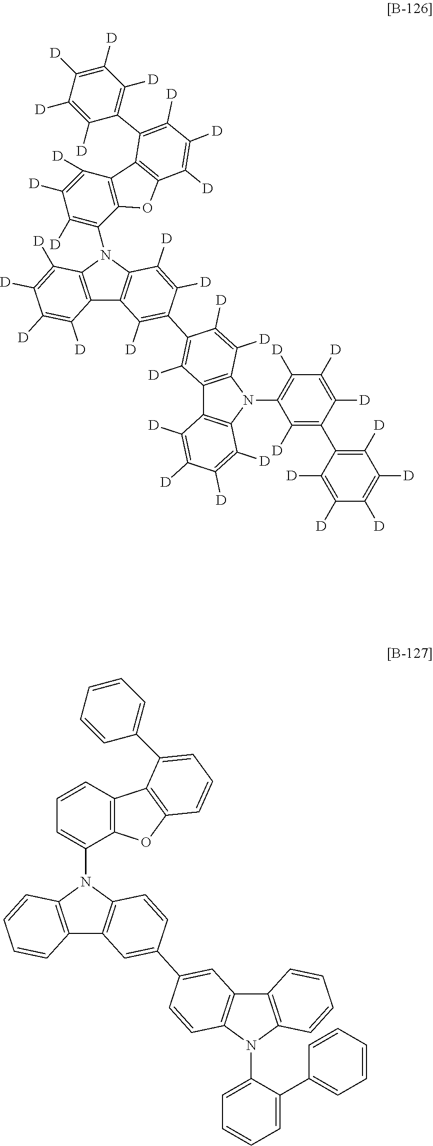 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c5oc5c([2H])c([2H])c([2H])c(-c7c([2H])c([2H])c([2H])c([2H])c7[2H])c56)c([2H])c([2H])c43)c2[2H])c([2H])c1[2H] ; c1ccc(-c2ccccc2-n2c3ccccc3c3cc(-c4ccc5c(c4)c4ccccc4n5-c4cccc5c4oc4cccc(-c6ccccc6)c45)ccc32)cc1]

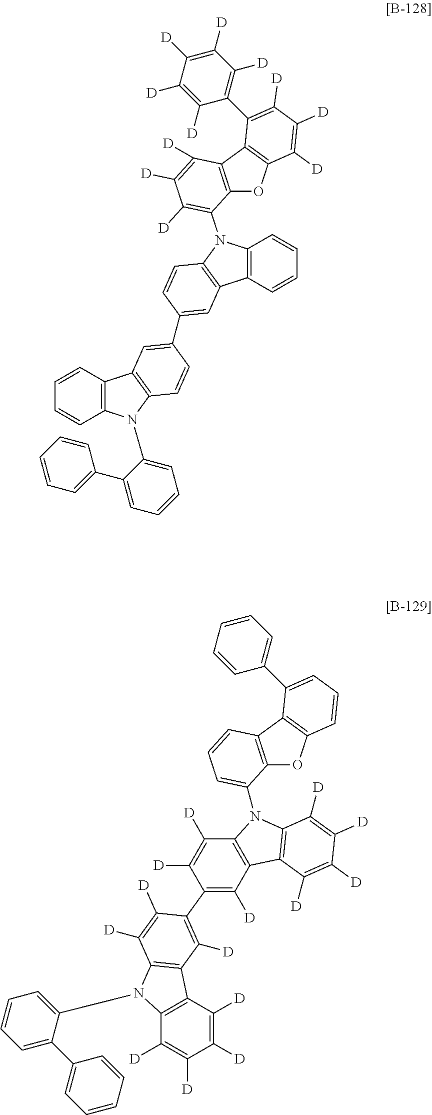 [SMILES: [2H]c1c([2H])c([2H])c(-c2c([2H])c([2H])c([2H])c3oc4c(-n5c6ccccc6c6cc(-c7ccc8c(c7)c7ccccc7n8-c7ccccc7-c7ccccc7)ccc65)c([2H])c([2H])c([2H])c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cccc(-c5ccccc5)c34)c([2H])c([2H])c1n2-c1ccccc1-c1ccccc1]

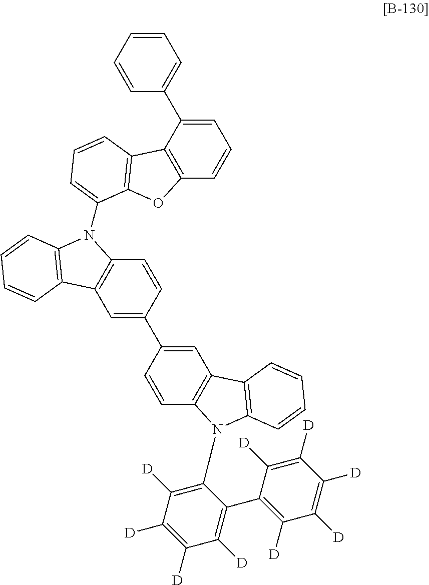 [SMILES: [2H]c1c([2H])c([2H])c(-c2c([2H])c([2H])c([2H])c([2H])c2-n2c3ccccc3c3cc(-c4ccc5c(c4)c4ccccc4n5-c4cccc5c4oc4cccc(-c6ccccc6)c45)ccc32)c([2H])c1[2H]]

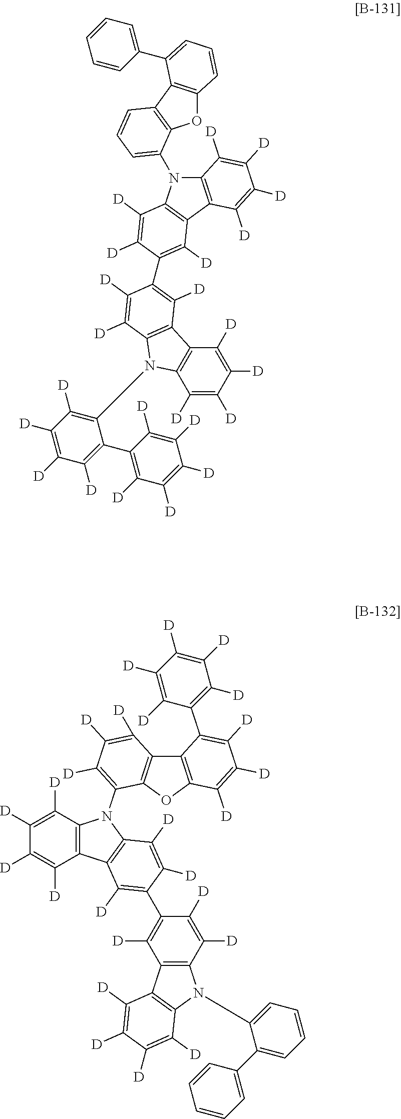 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4oc4cccc(-c6ccccc6)c45)c([2H])c([2H])c32)c([2H])c1[2H] ; [2H]c1c([2H])c([2H])c(-c2c([2H])c([2H])c([2H])c3oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c([2H])c([2H])c4c23)c([2H])c1[2H]]

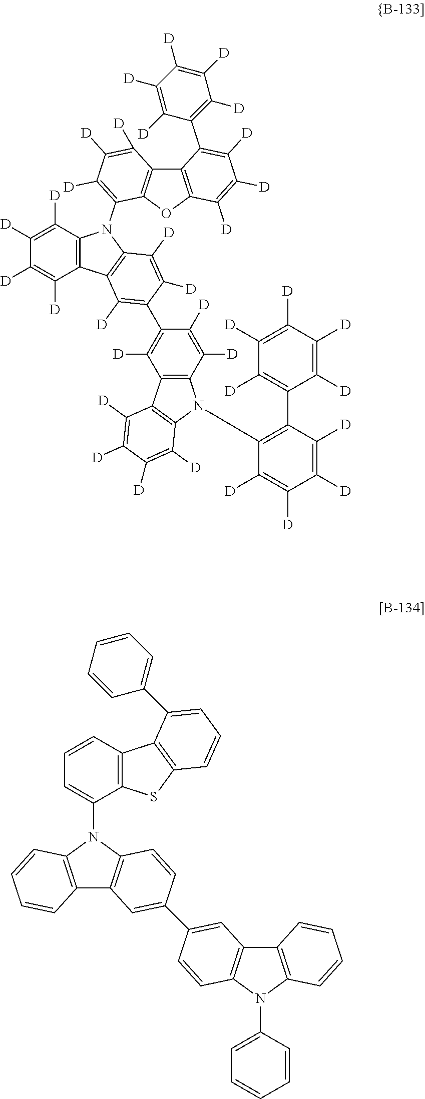 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c4oc4c([2H])c([2H])c([2H])c(-c6c([2H])c([2H])c([2H])c([2H])c6[2H])c45)c([2H])c([2H])c32)c([2H])c1[2H] ; c1ccc(-c2cccc3sc4c(-n5c6ccccc6c6cc(-c7ccc8c(c7)c7ccccc7n8-c7ccccc7)ccc65)cccc4c23)cc1]

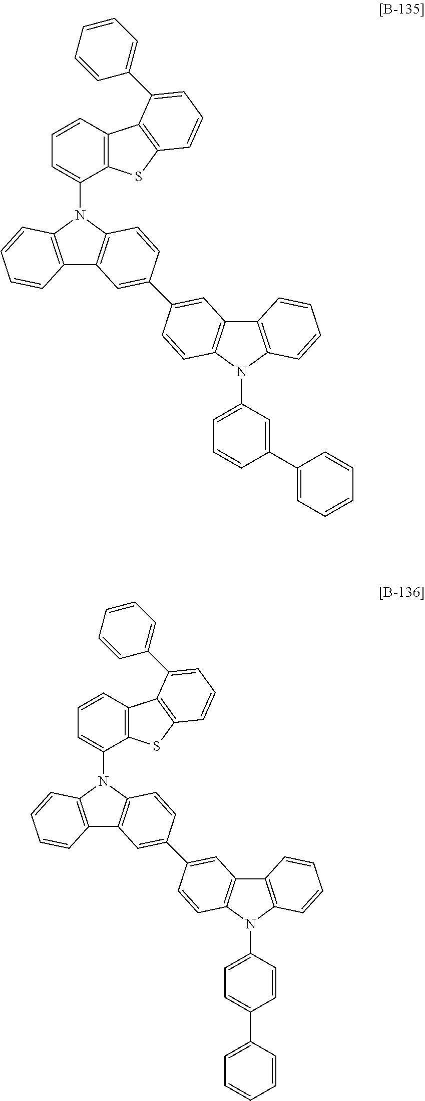 [SMILES: c1ccc(-c2cccc(-n3c4ccccc4c4cc(-c5ccc6c(c5)c5ccccc5n6-c5cccc6c5sc5cccc(-c7ccccc7)c56)ccc43)c2)cc1 ; c1ccc(-c2ccc(-n3c4ccccc4c4cc(-c5ccc6c(c5)c5ccccc5n6-c5cccc6c5sc5cccc(-c7ccccc7)c56)ccc43)cc2)cc1]

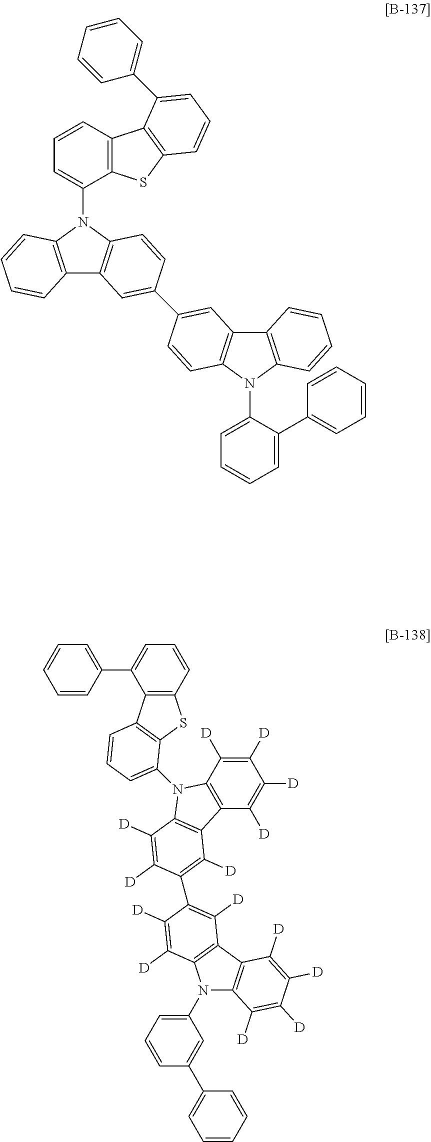 [SMILES: c1ccc(-c2ccccc2-n2c3ccccc3c3cc(-c4ccc5c(c4)c4ccccc4n5-c4cccc5c4sc4cccc(-c6ccccc6)c45)ccc32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3cccc(-c5ccccc5)c34)c([2H])c([2H])c1n2-c1cccc(-c2ccccc2)c1]

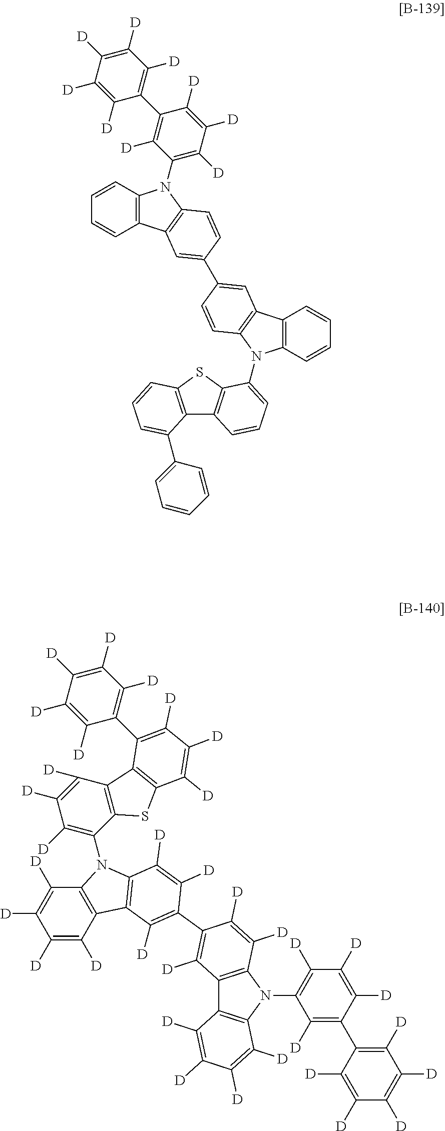 [SMILES: [2H]c1c([2H])c([2H])c(-c2c([2H])c([2H])c([2H])c(-n3c4ccccc4c4cc(-c5ccc6c(c5)c5ccccc5n6-c5cccc6c5sc5cccc(-c7ccccc7)c56)ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c5sc5c([2H])c([2H])c([2H])c(-c7c([2H])c([2H])c([2H])c([2H])c7[2H])c56)c([2H])c([2H])c43)c2[2H])c([2H])c1[2H]]

The first compound and the second compound may be included in a weight ratio of, for example, 1:99 to 99:1. Within the range, a desirable weight ratio may be adjusted using an electron transport capability of the first compound and a hole transport capability of the second compound to realize bipolar characteristics and thus to improve efficiency and life-span. Within the range, they may be for example included in a weight ratio of 10:90 to 90:10, 20:80 to 80:20, for example, 20:80 to 70:30, 20:80 to 60:40, or 30:70 to 60:40. As a specific example, it may be included in a weight ratio of 40:60, 50:50, or 60:40.

In addition to the first and second compounds described above, one or more compounds may be further included.

For example, the composition for an aforementioned organic optoelectronic device may further include a dopant.

The dopant may be, for example, a phosphorescent dopant, for example a phosphorescent dopant of red, green or blue, and may be, for example, a red phosphorescent dopant.

The dopant is a material mixed with the composition for an organic optoelectronic device in a small amount to cause light emission and may be generally a material such as a metal complex that emits light by multiple excitation into a triplet or more. The dopant may be, for example, an inorganic, organic, or organic-inorganic compound, and may include one or two or more types.

An example of the dopant may be a phosphorescent dopant, and examples of the phosphorescent dopant may include an organometallic compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z, but is not limited thereto.

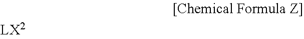

In Chemical Formula Z, M is a metal, and L and X² are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof, and L and X² may be, for example a bidentate ligand.

Examples of the ligands represented by L and X² may be selected from the chemical formulas of Group A, but are not limited thereto.

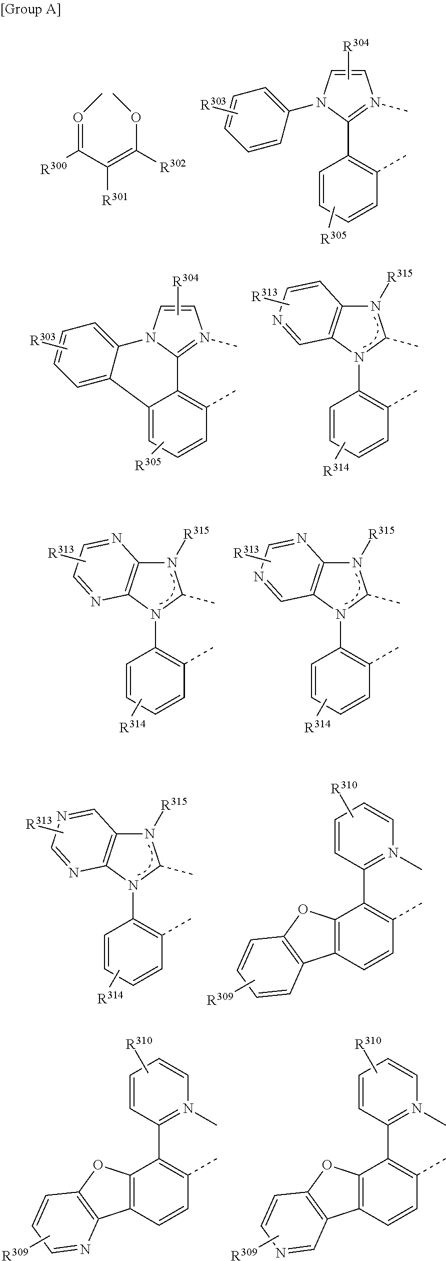

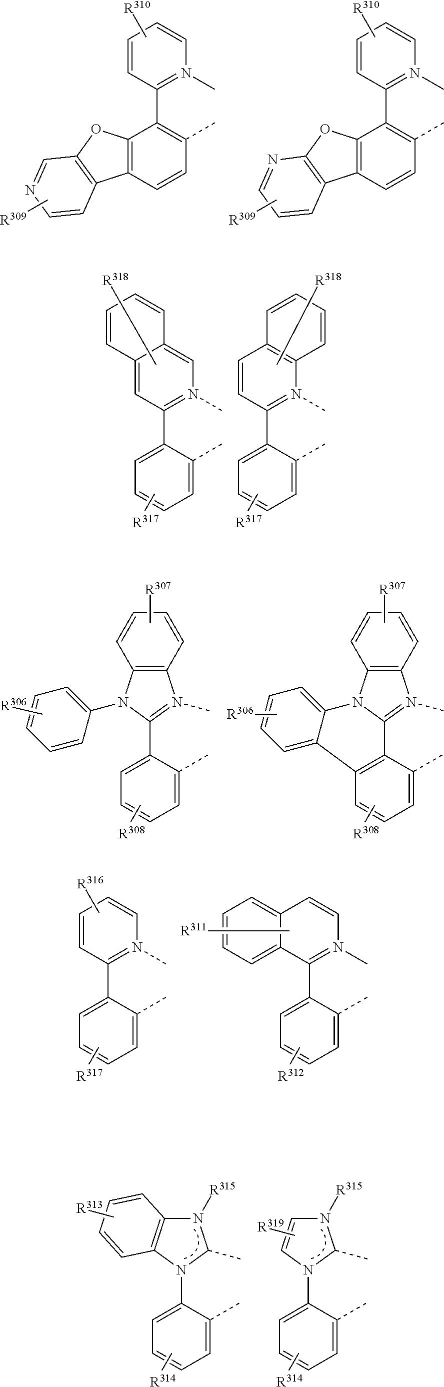

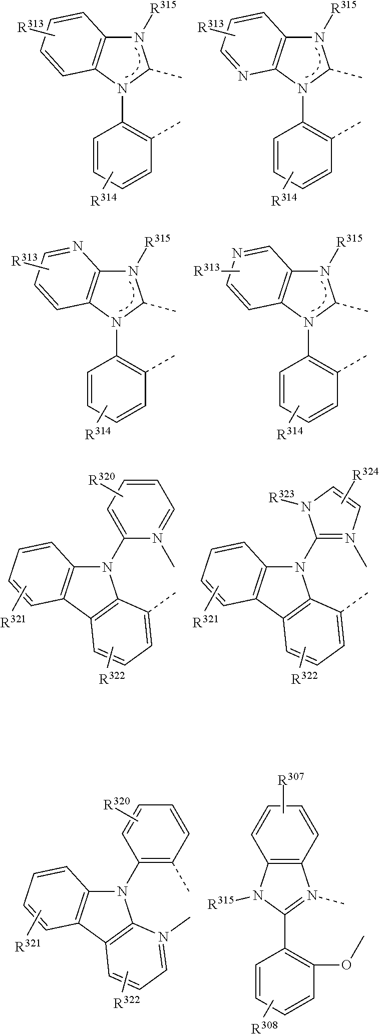

In Group A,
R³⁰⁰ to R³⁰² are each independently hydrogen, deuterium a C1 to C30 alkyl group that is substituted or unsubstituted with a halogen, a C6 to C30 aryl group that is substituted or unsubstituted with a C1 to C30 alkyl, or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SFs, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

For example, a dopant represented by Chemical Formula IV may be included.

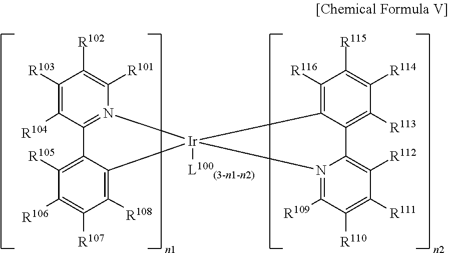

In Chemical Formula IV,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently C1 to C6 alkyl group,
at least one of R¹⁰¹ to R¹¹⁶ is a functional group represented by Chemical Formula IV-1, L¹⁰⁰ is a bidentate ligand of a monovalent anion, and is a ligand that coordinates to iridium through a lone pair of carbons or heteroatoms, and
n1 and n2 are each independently any one of integers of 0 to 3, and n1+n2 is any one of integers of 1 to 3,

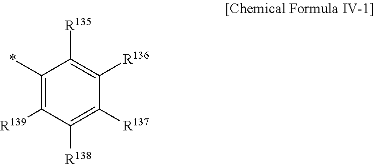

wherein, in Chemical Formula IV-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* means a part connected to a carbon atom.

For example, the dopant represented by Chemical Formula Z-1 may be included.

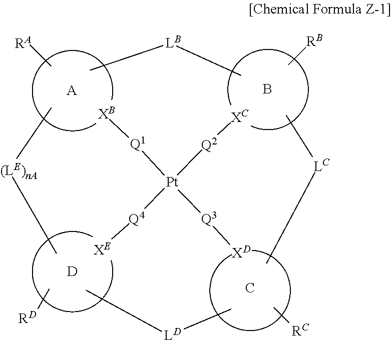

In Chemical Formula Z-1, rings A, B, C, and D may each independently be a 5-membered or 6-membered carbocyclic or heterocyclic ring;
R^{A}, R^{B}, R^{C}, and R^{D} may each independently be mono-, di-, tri-, or tetra-substitution, or unsubstitution;
L^{B}, L^{C}, and L^{D} may each independently be a direct bond, BR, NR, PR, O, S, Se, C═O, S═O, SO₂, CRR′, SiRR′, GeRR′, or a combination thereof. When nA is 1, L^{E} may be a direct bond, BR, NR, PR, O, S, Se, C═O, S═O, SO₂, CRR′, SiRR′, GeRR′, or a combination thereof; and when nA is 0, L^{E} does not exist;

R^{A}, R^{B}, R^{C}, R^{D}, R, and R′ may each independently be hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an embodiment may be a platinum complex, and may be represented by Chemical Formula V.

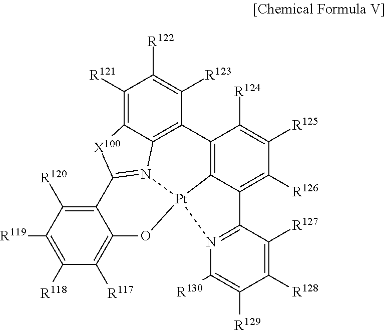

In Chemical Formula V,
X¹⁰⁰ may be O, S, or NR¹³¹,
R¹¹⁷ to R¹³¹ may each independently be hydrogen, deuterium, a substituted or unsubstituted C1 to C10 alkyl group, a substituted or unsubstituted C6 to C20 aryl group, or —SiR¹³²R¹³³R¹³⁴
R¹³² to R¹³⁴ may each independently be a C1 to C6 alkyl group, and
at least one of R¹¹⁷ to R¹³¹ may be —SiR¹³²R¹³³R¹³⁴ or a tert-butyl group.

Hereinafter, an organic optoelectronic device including the aforementioned composition for the organic optoelectronic device is described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

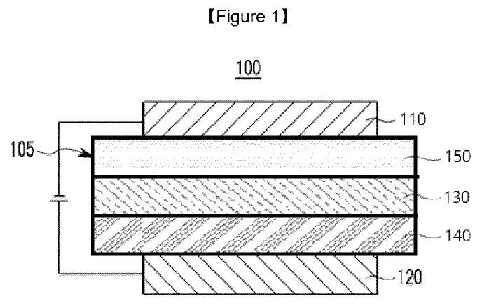

The FIG. 1 is a cross-sectional view illustrating an organic light emitting diode according to an embodiment.

Referring to the FIG. 1, an organic light emitting diode 100 according to an embodiment may include an anode 120 and a cathode 110 facing each other and an organic layer 105 between the anode 120 and cathode 110.

The anode 120 may be made of a conductor having a large work function to help hole injection, and may be a metal, a metal oxide, or a conductive polymer. The anode 120 may be a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be a metal, a metal oxide, or a conductive polymer. The cathode 110 may be a metal such as magnesium, calcium, sodium, potassium, titanium, indium, yttrium, lithium, gadolinium, aluminum silver, tin, lead, cesium, barium, or the like, or an alloy thereof; or a multi-layer structure material such as LiF/Al, LiO₂/Al, LiF/Ca, or BaF₂/Ca.

The organic layer 105 may include the aforementioned composition for an organic optoelectronic device.

The organic layer 105 may include the light emitting layer 130, and the light emitting layer 130 may include the aforementioned composition for an organic optoelectronic device.

The composition for an organic optoelectronic device further including a dopant may be a green light-emitting composition.

The light emitting layer 130 may include the aforementioned composition for an organic optoelectronic device as a phosphorescent host.

The organic layer may further include a charge transport region in addition to the light emitting layer.

The charge transport region may be the hole transport region 140.

The hole transport region 140 may help further increase hole injection and/or hole mobility between the anode 120 and the light emitting layer 130 and block electrons.

In an implementation, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

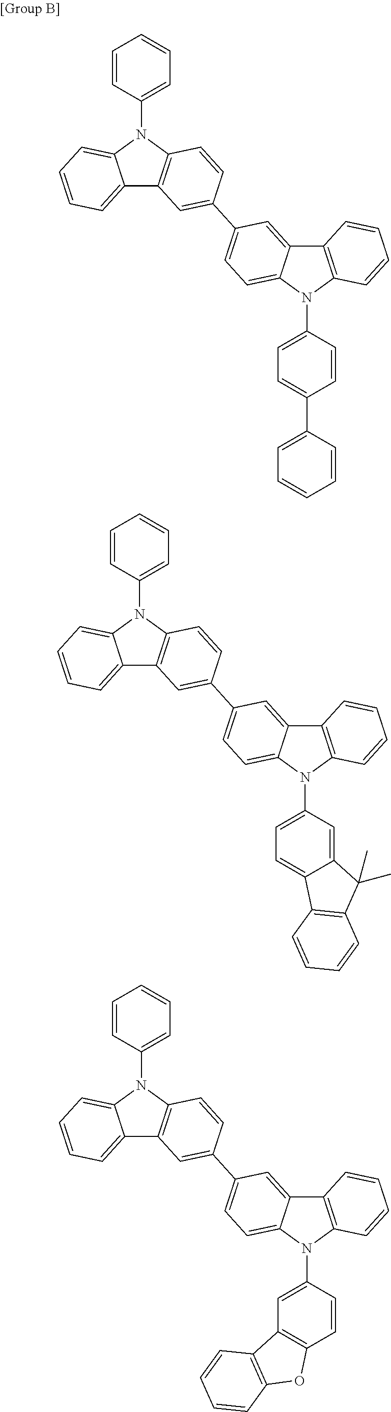 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

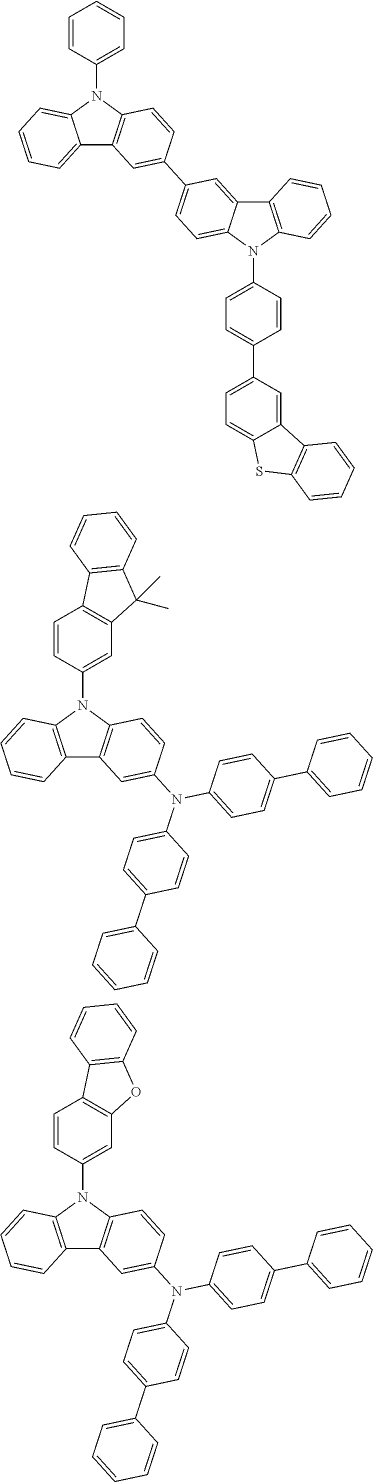

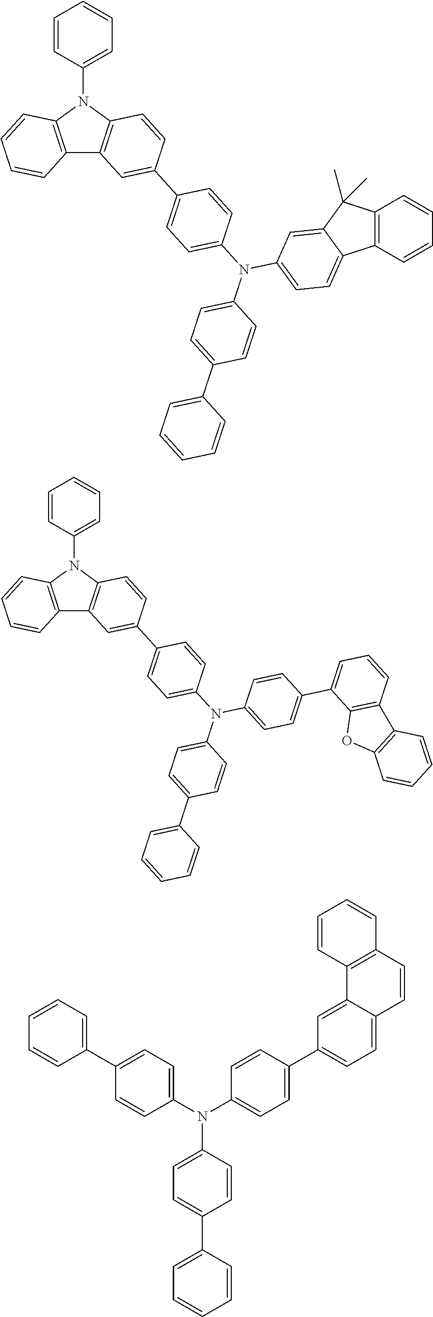 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

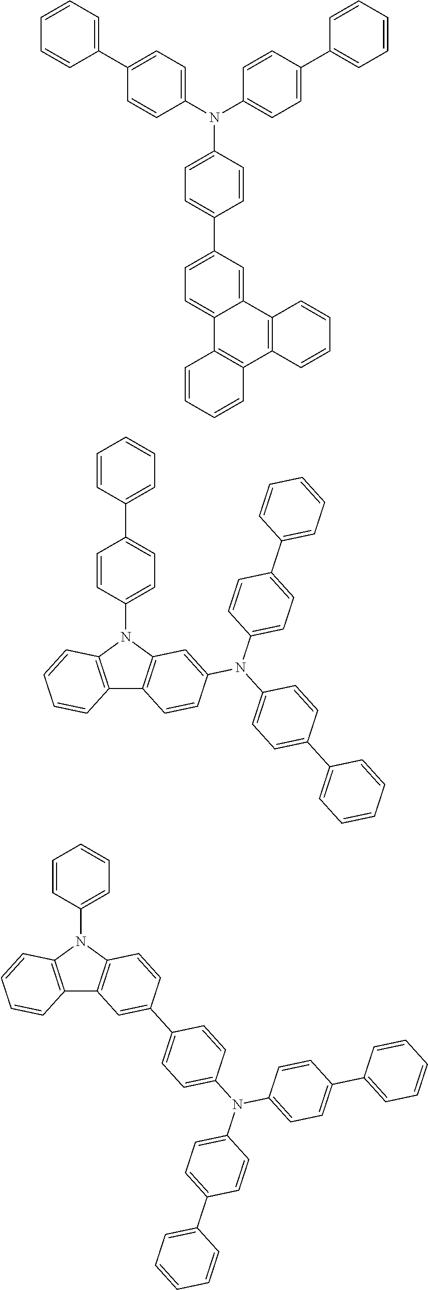 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

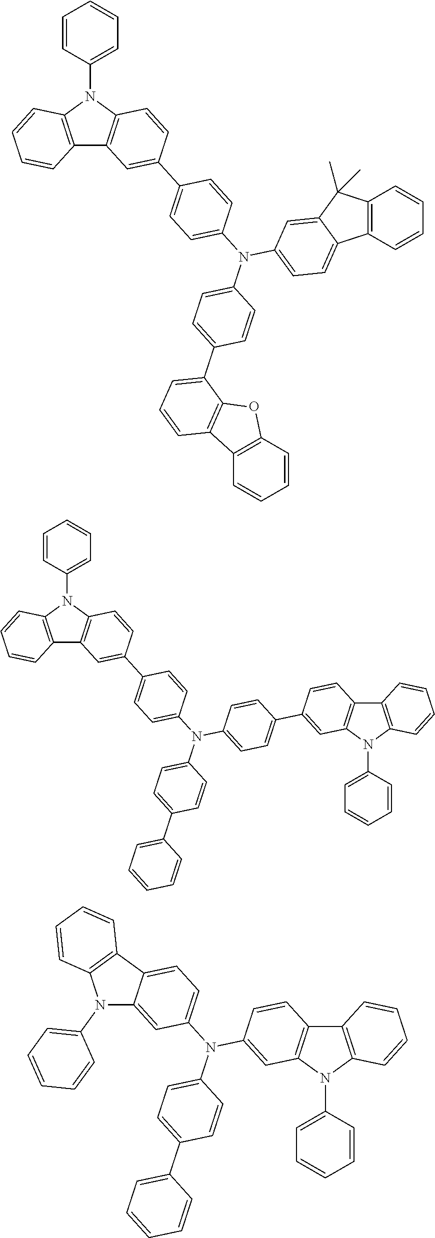 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1]

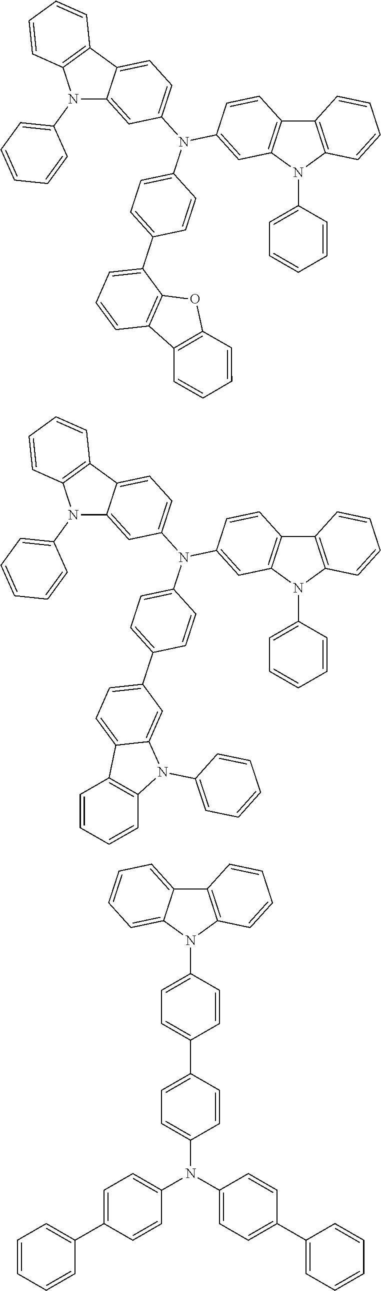

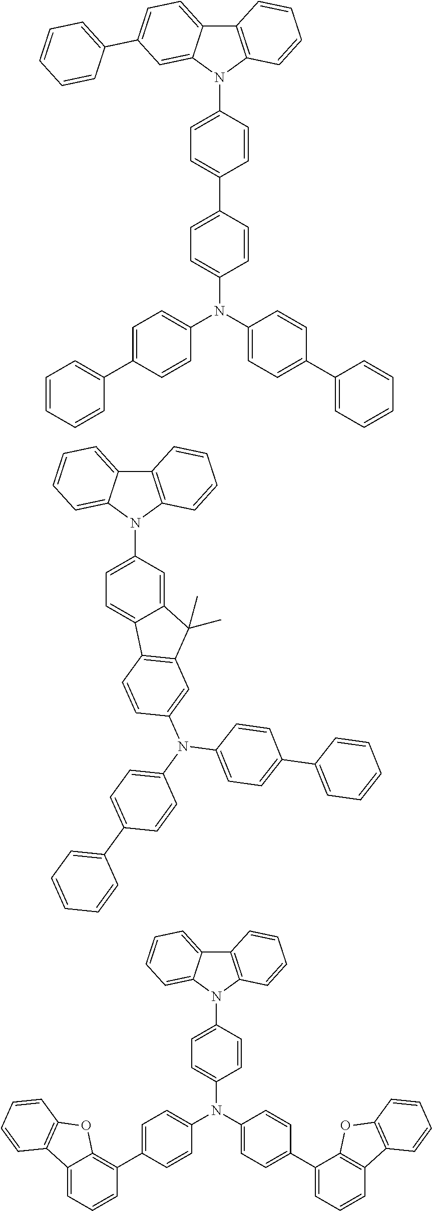

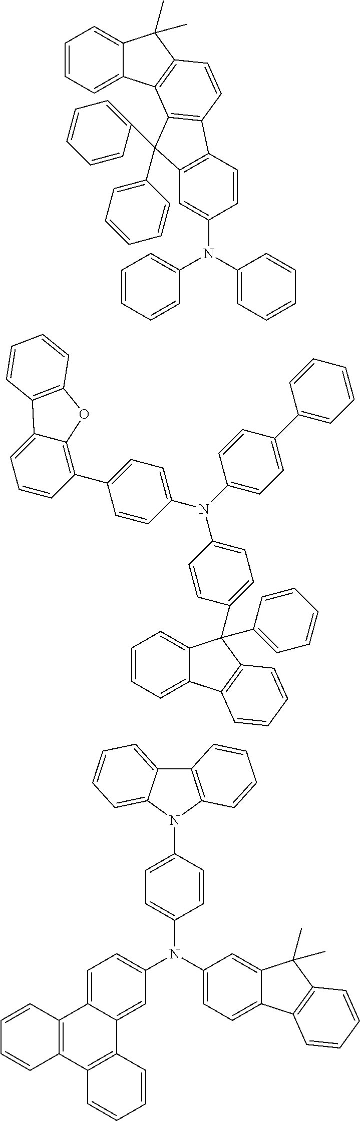

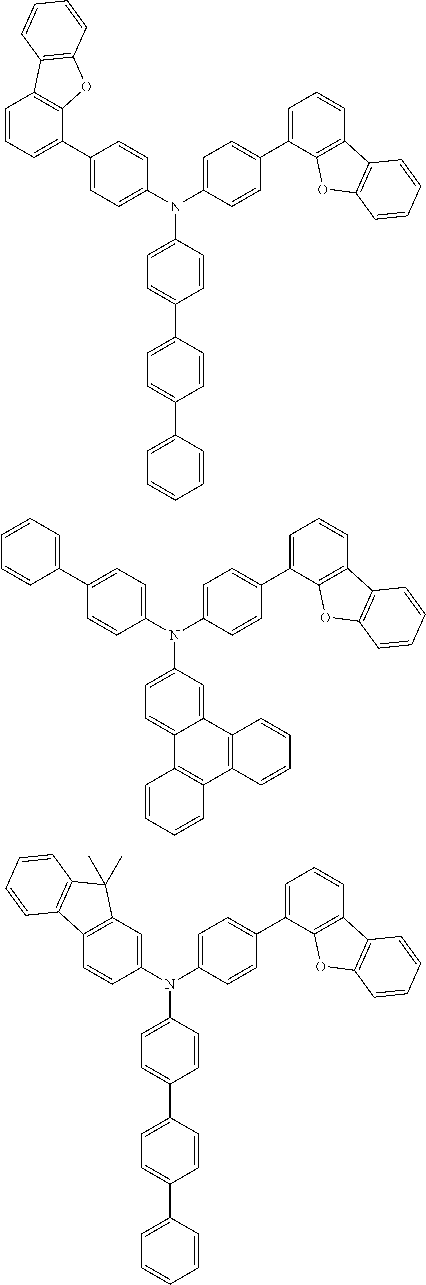

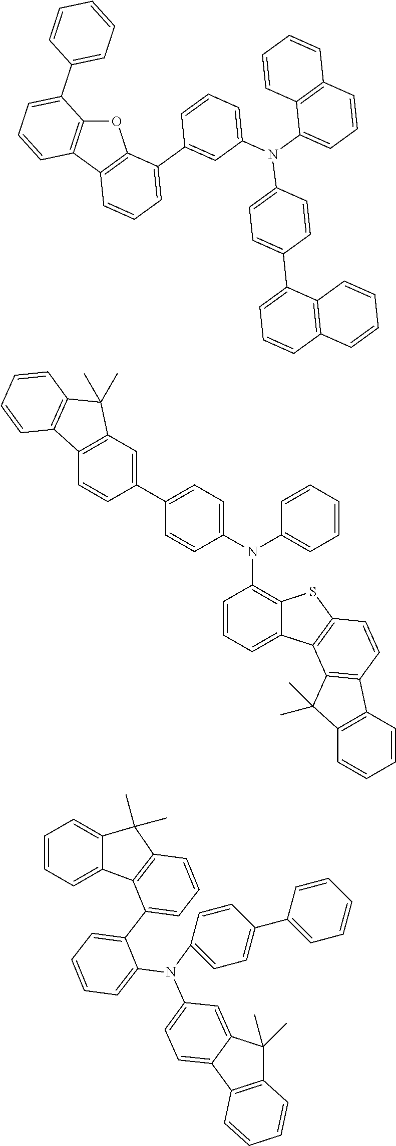 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

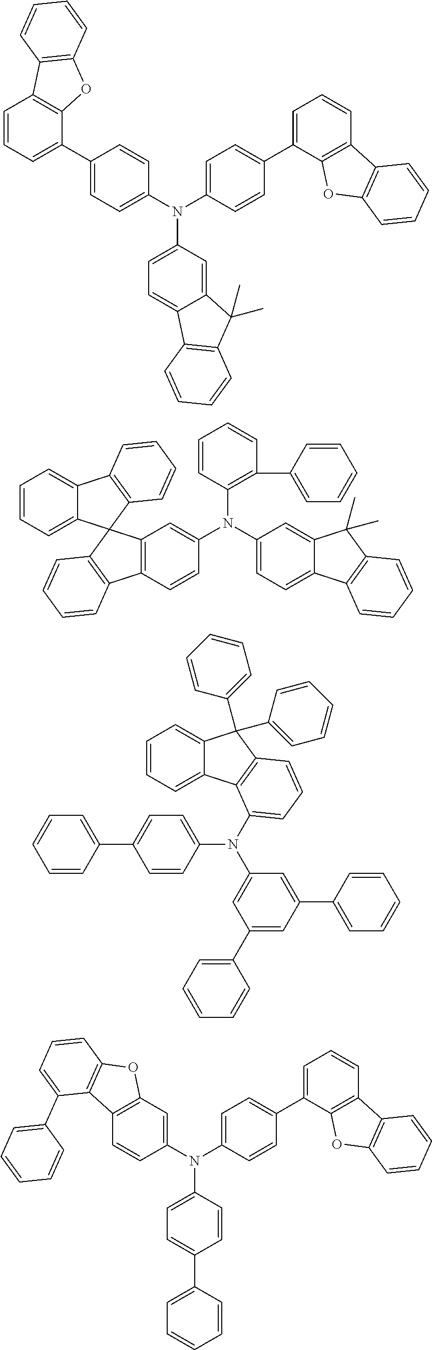

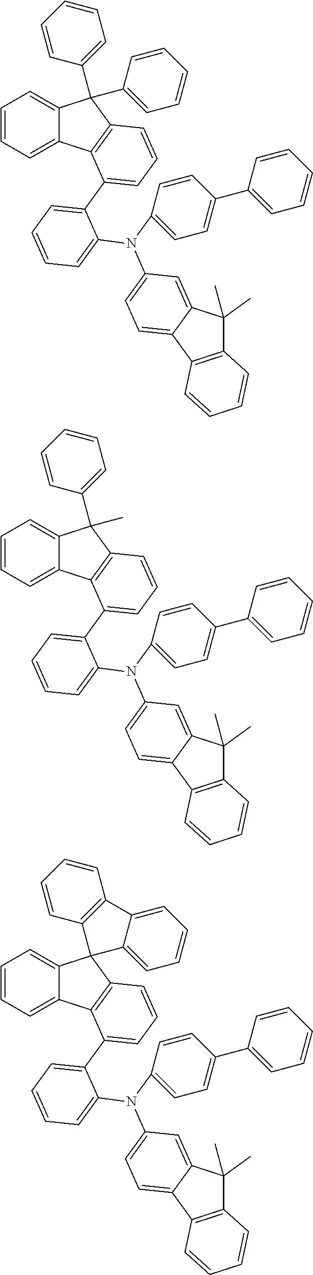 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

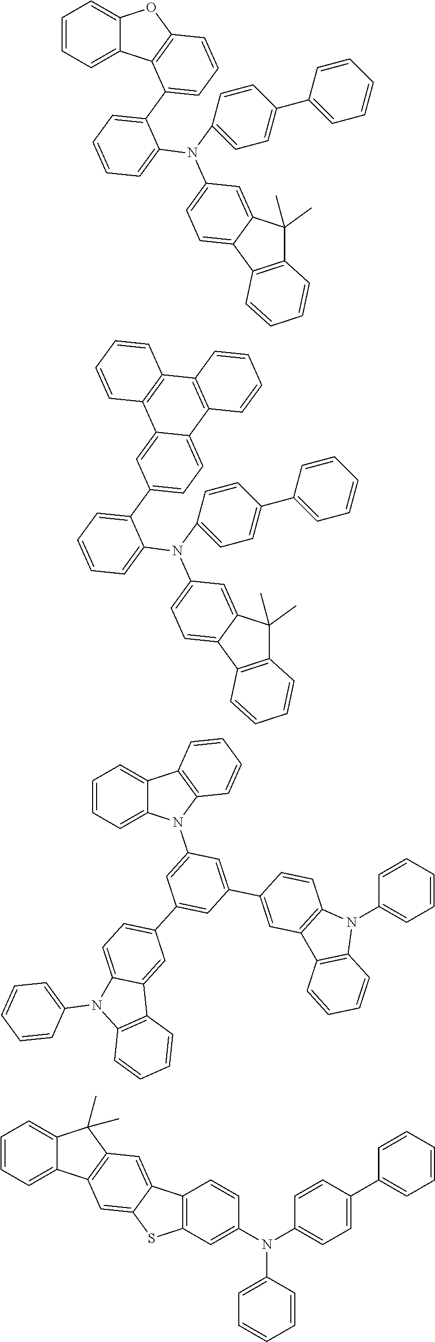

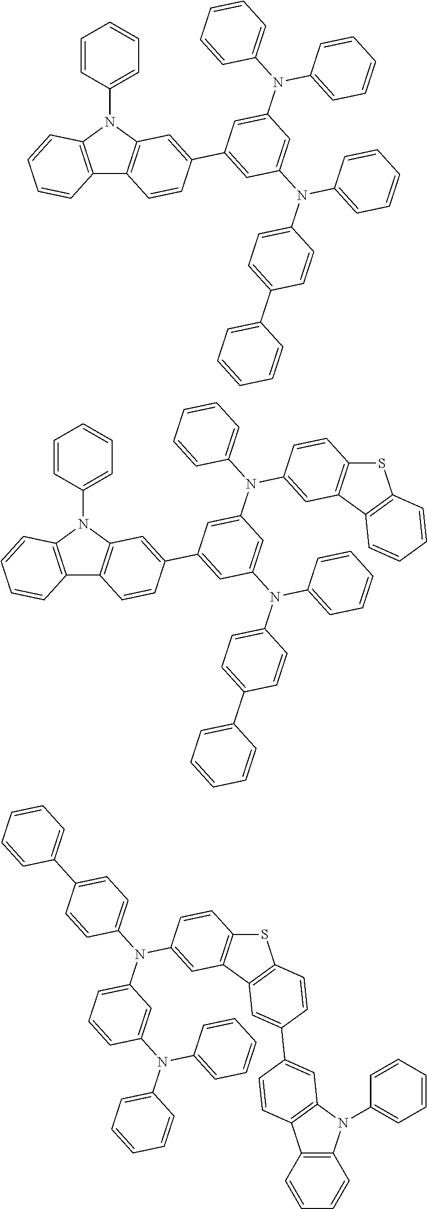

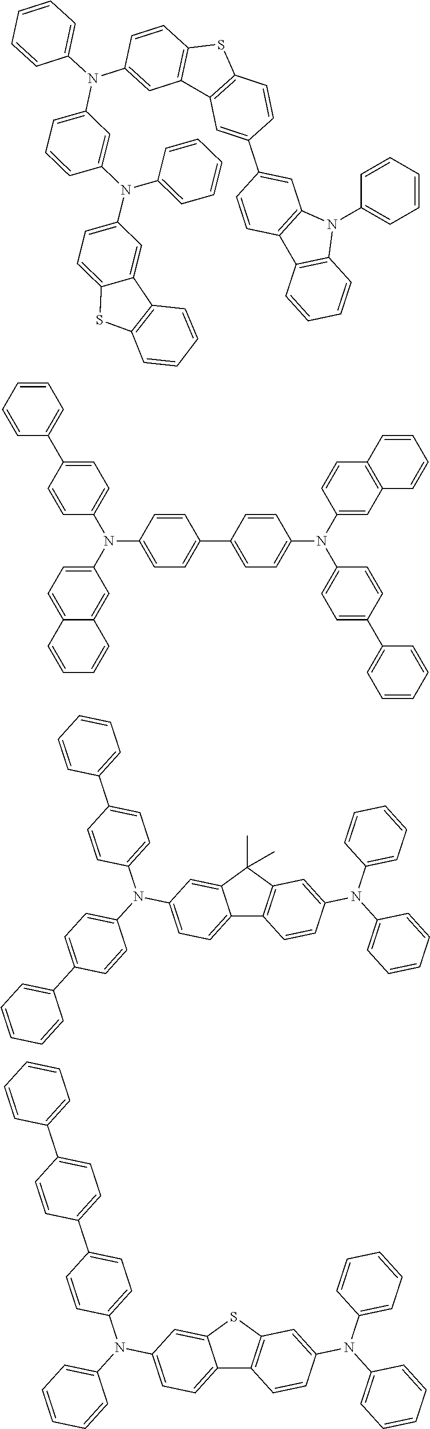

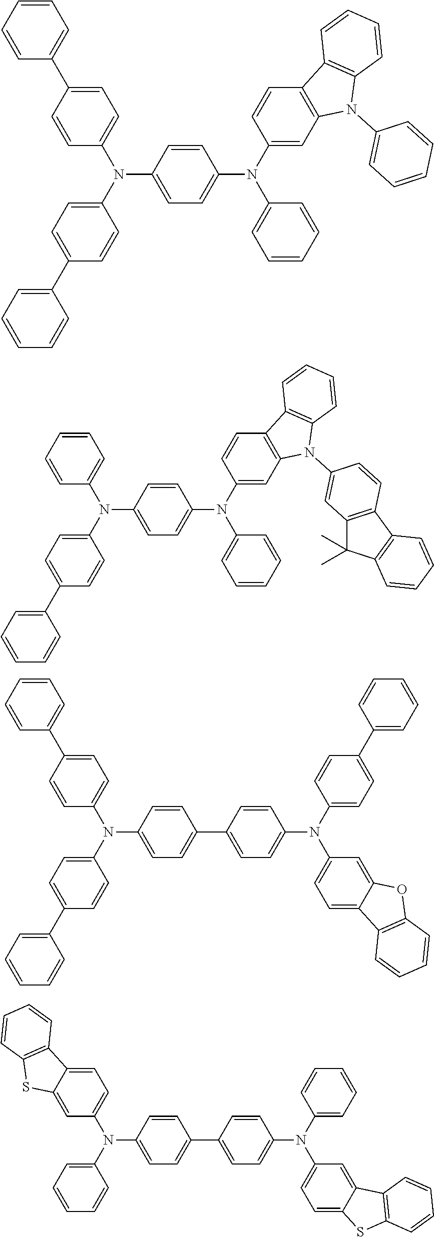

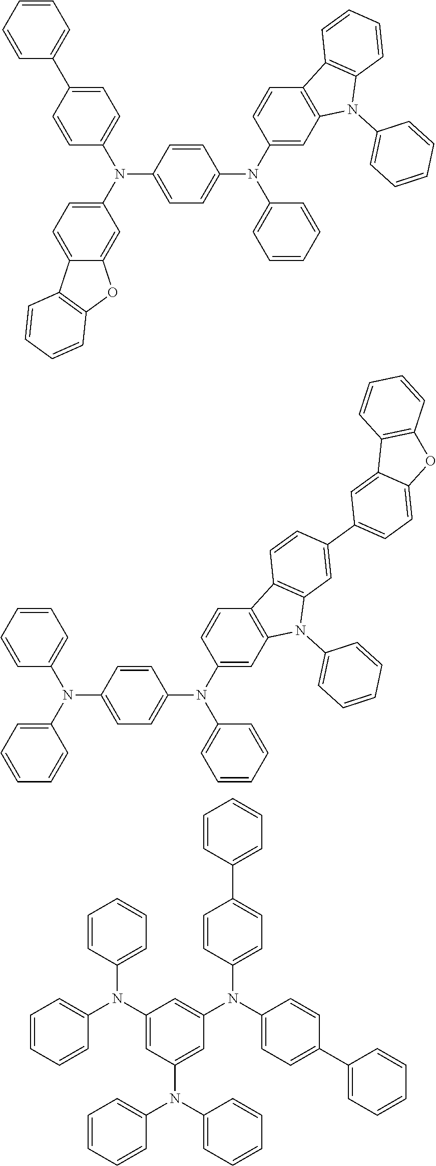

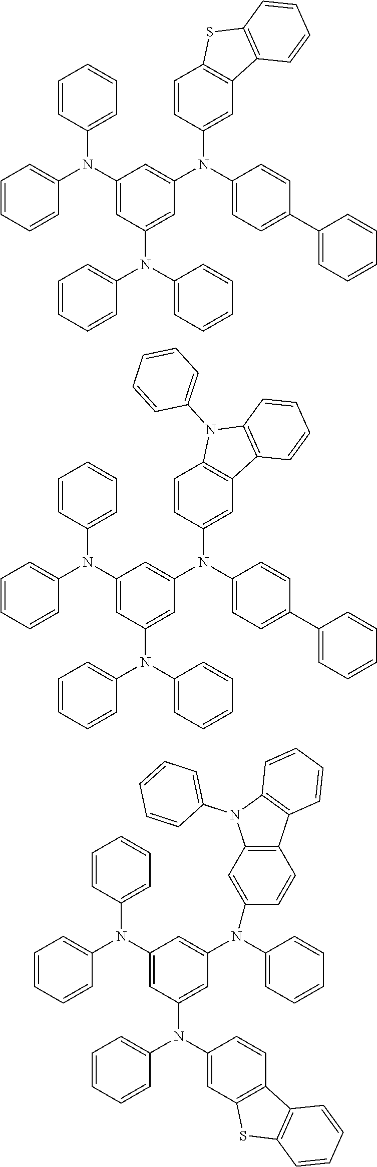

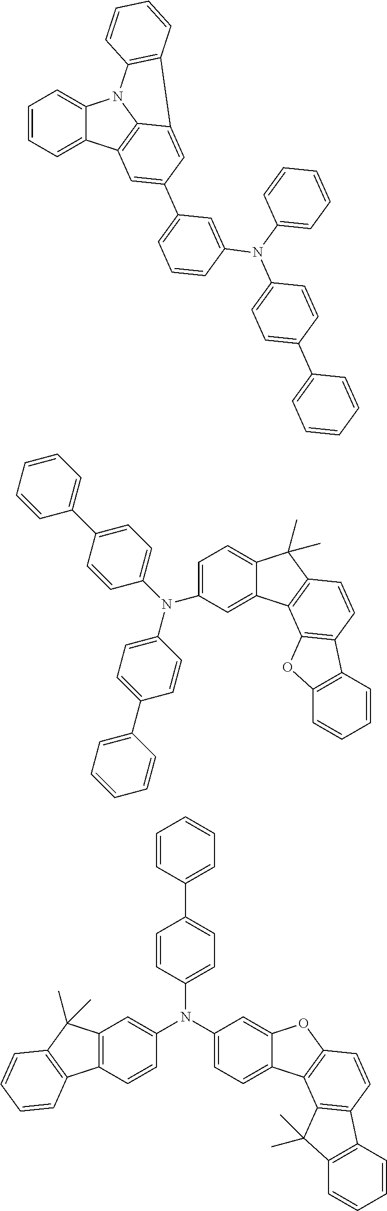

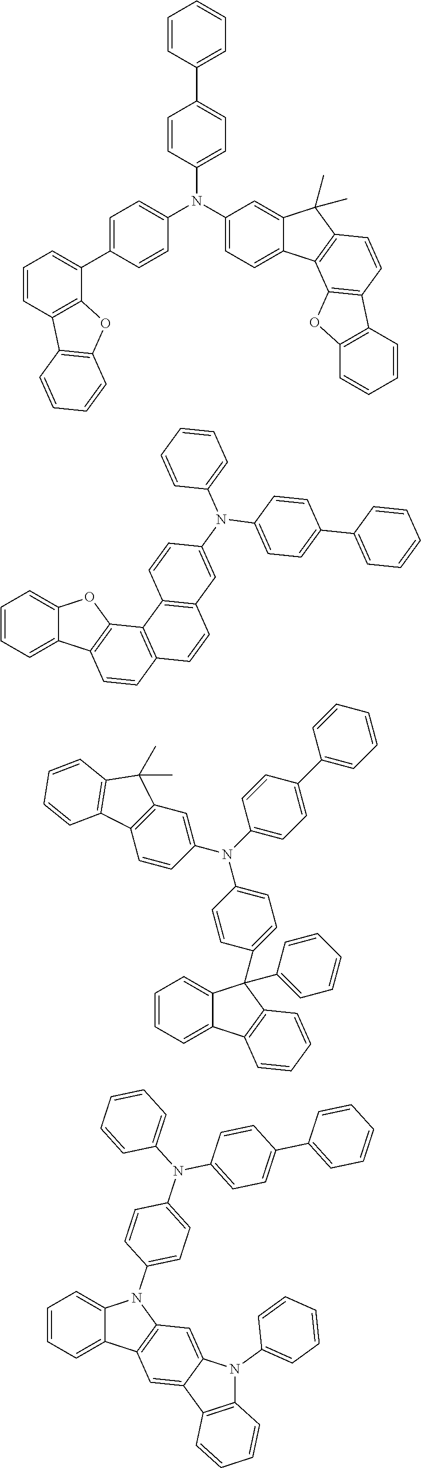 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

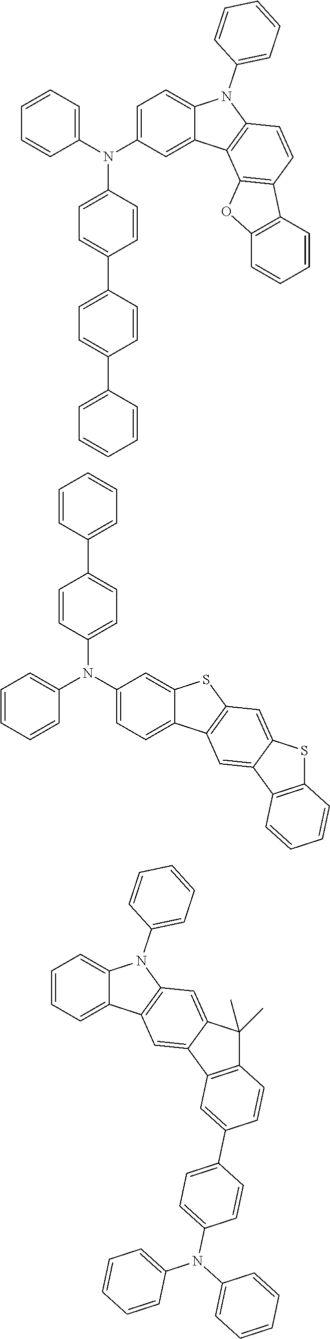

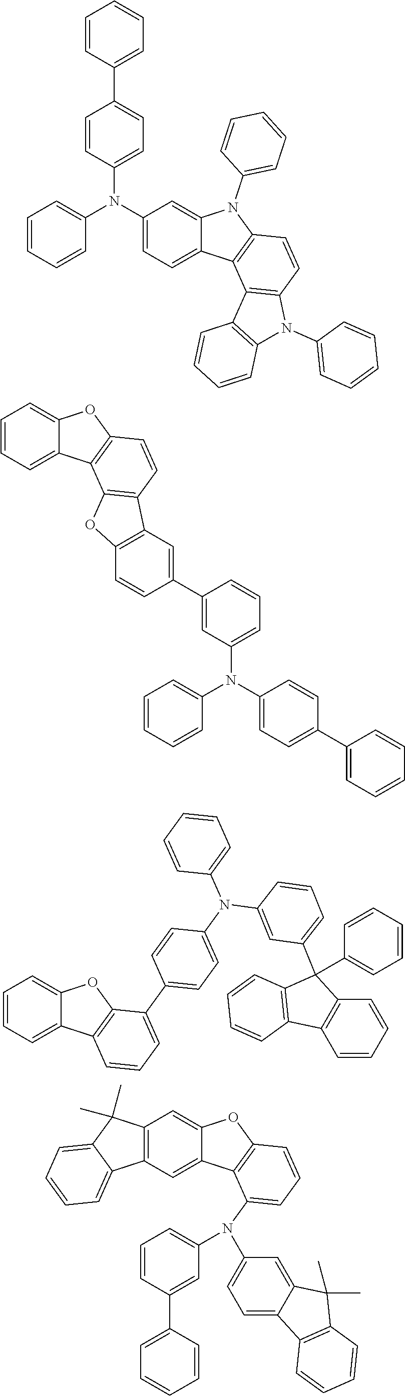

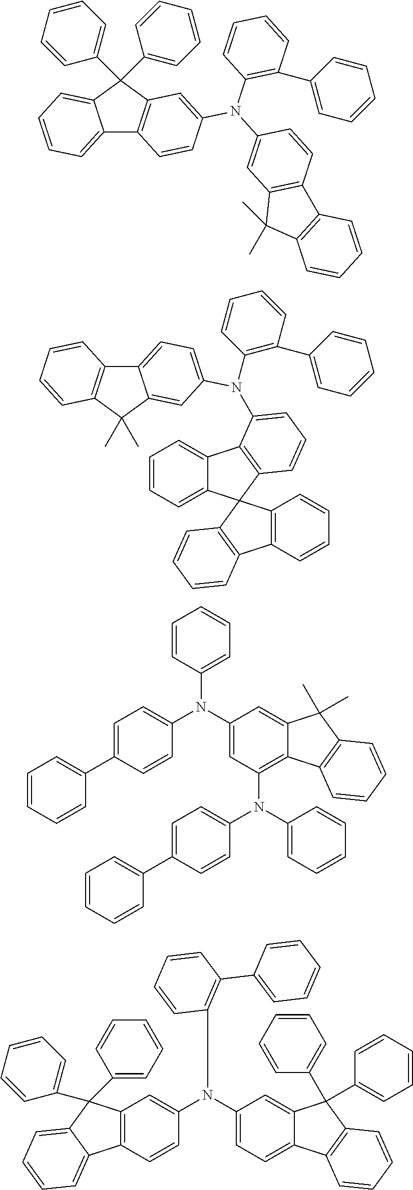

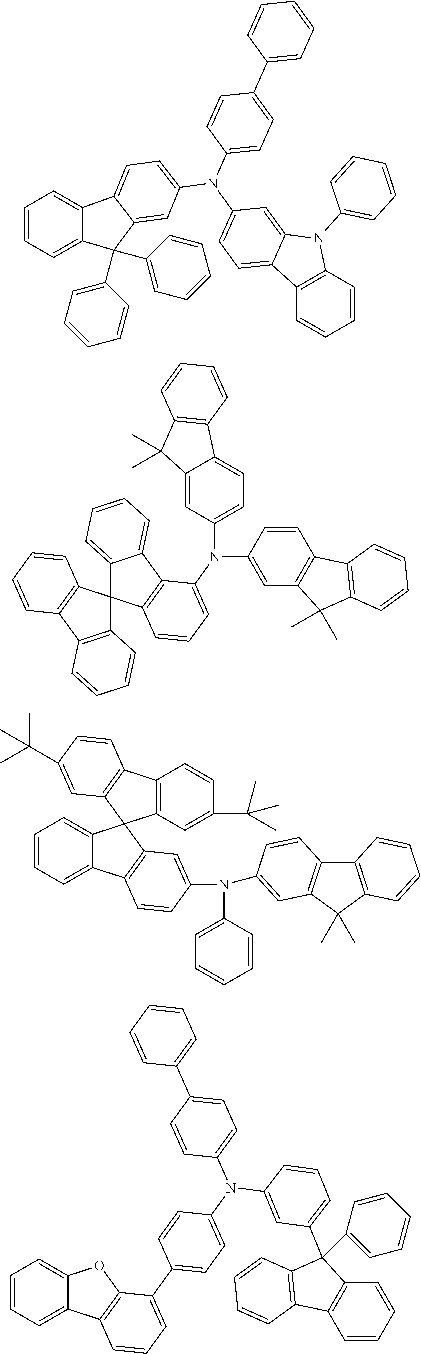

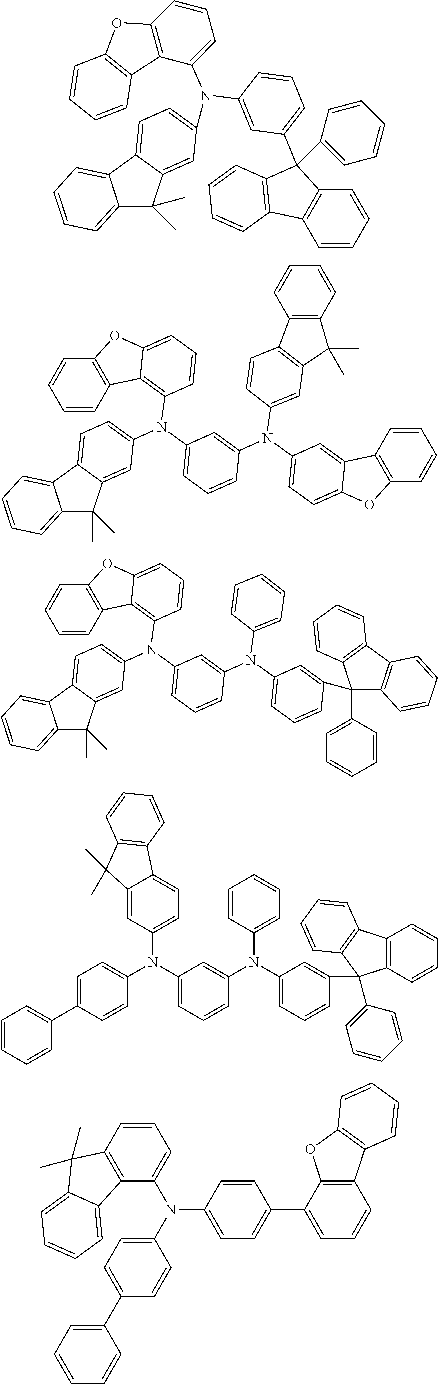

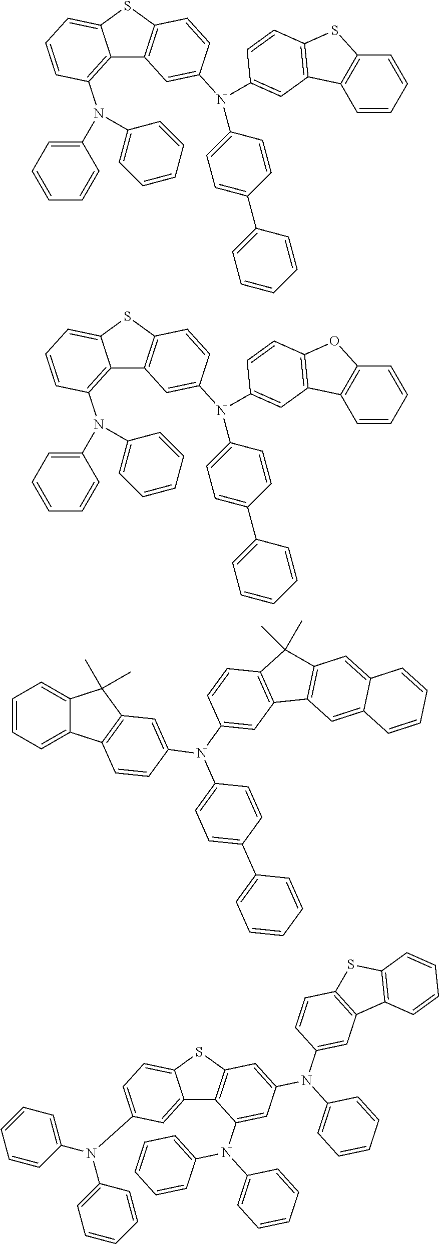

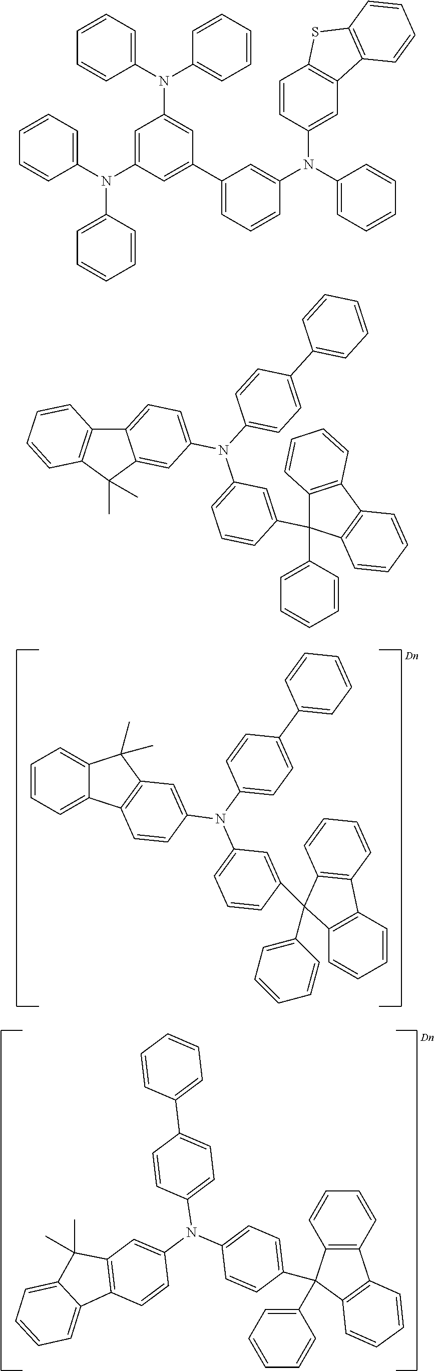

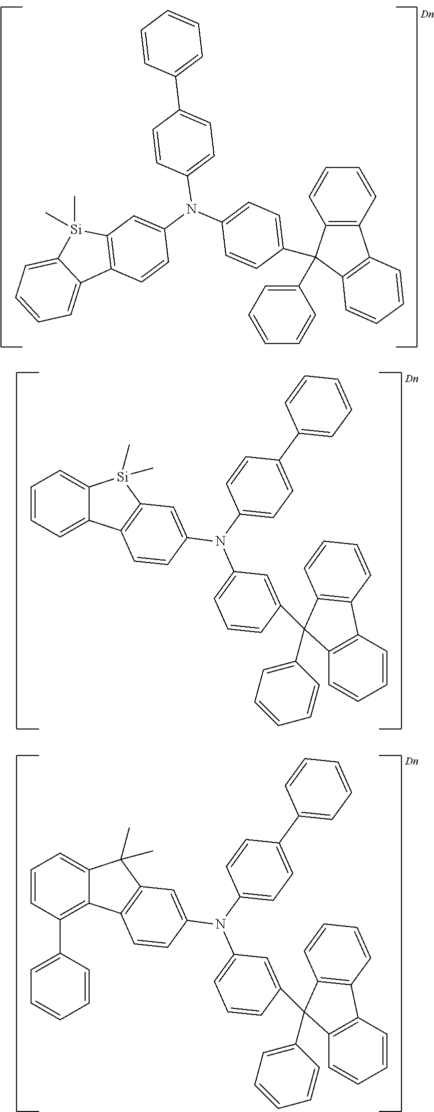

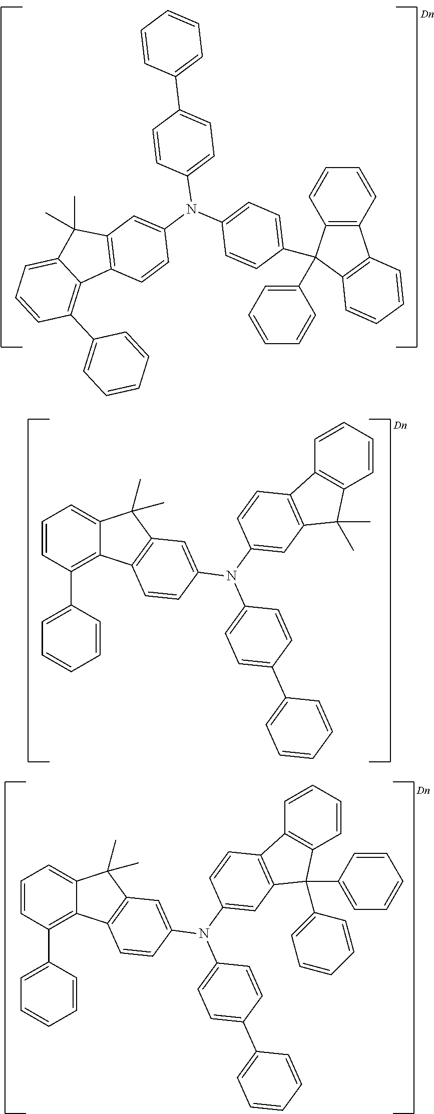

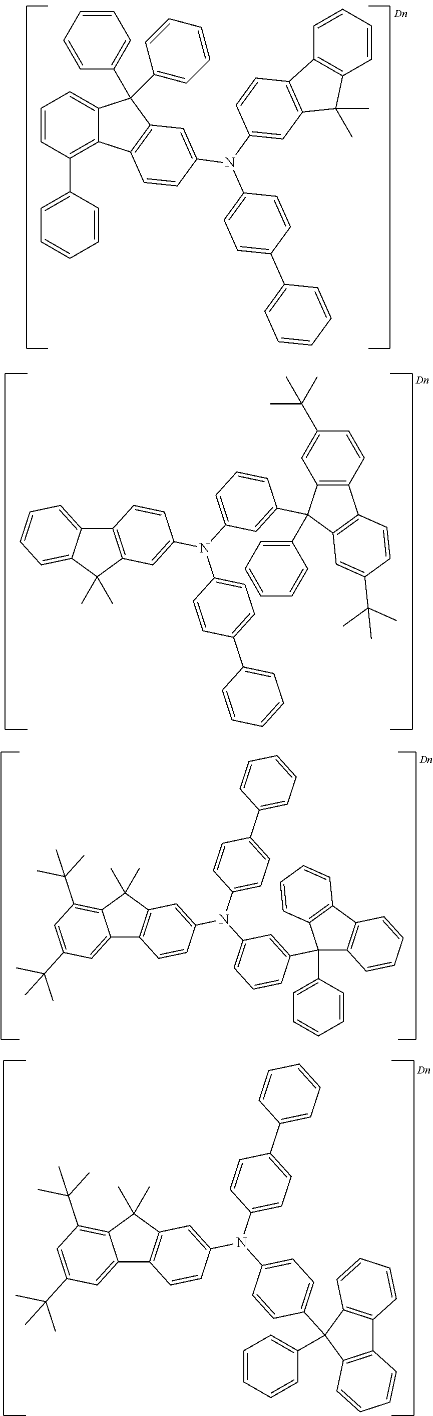

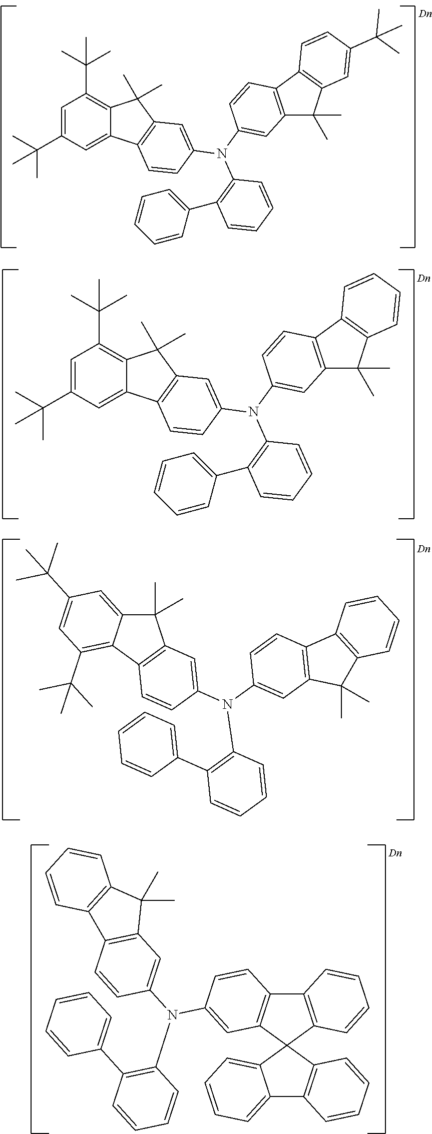

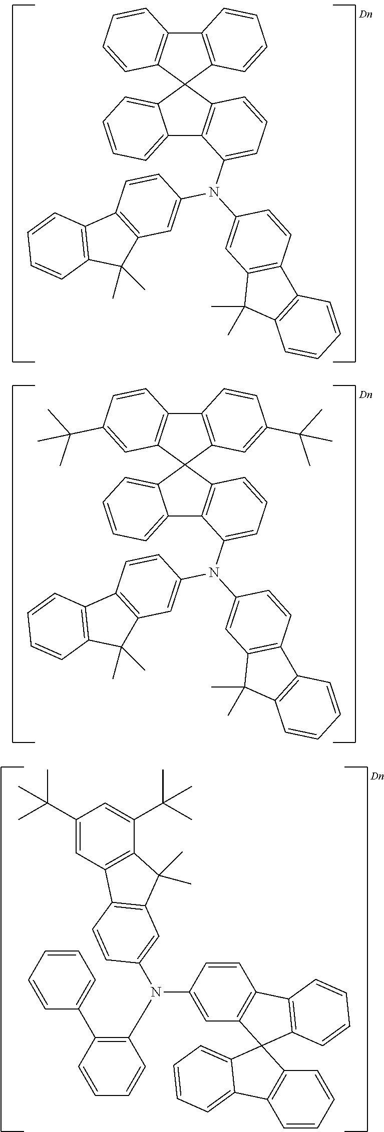

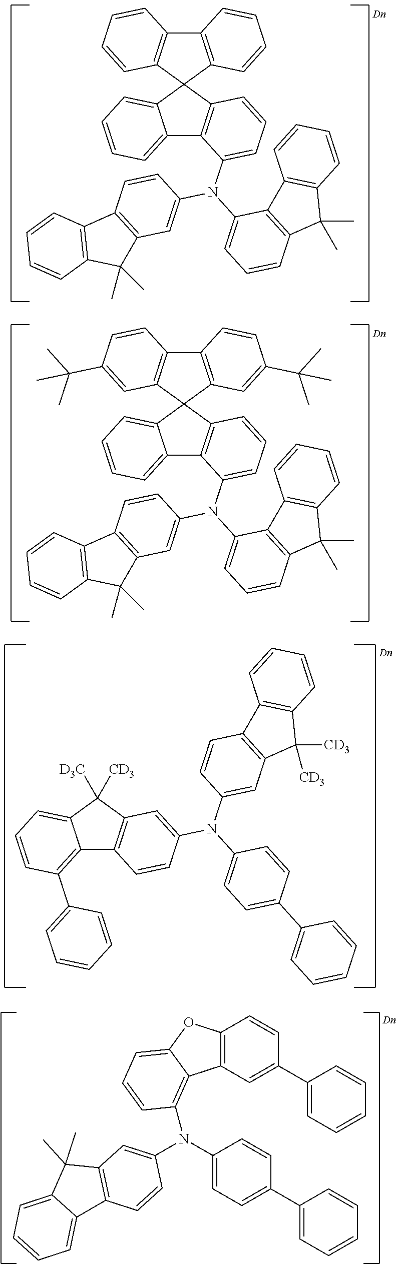

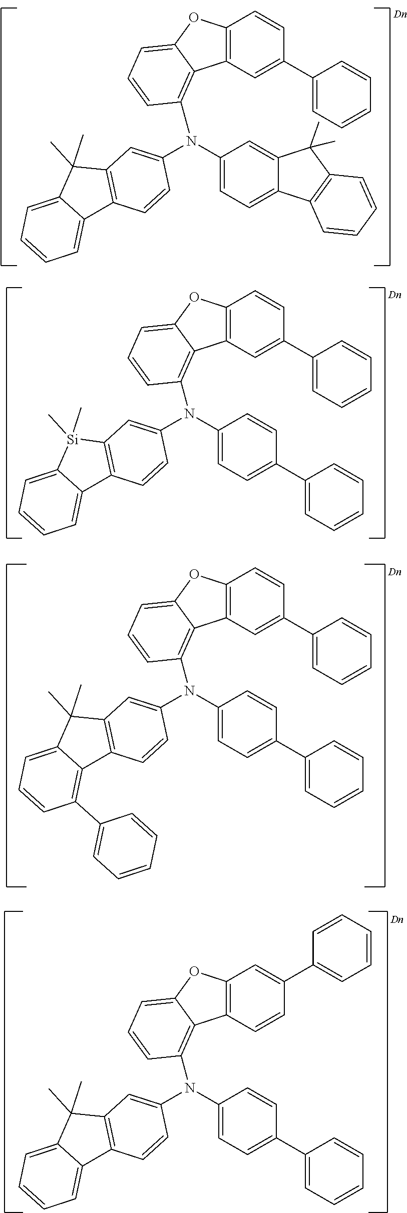

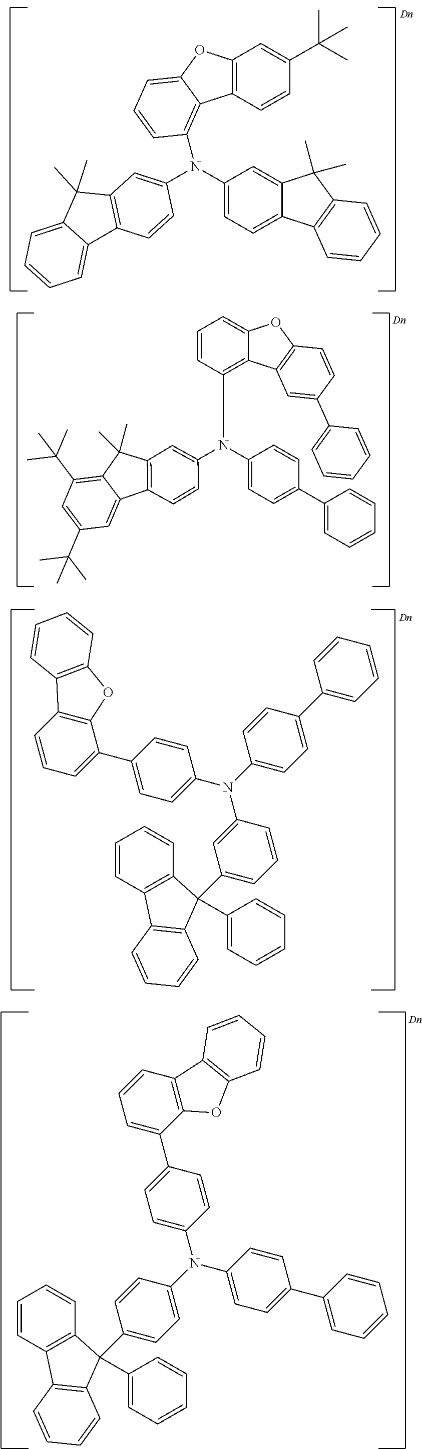

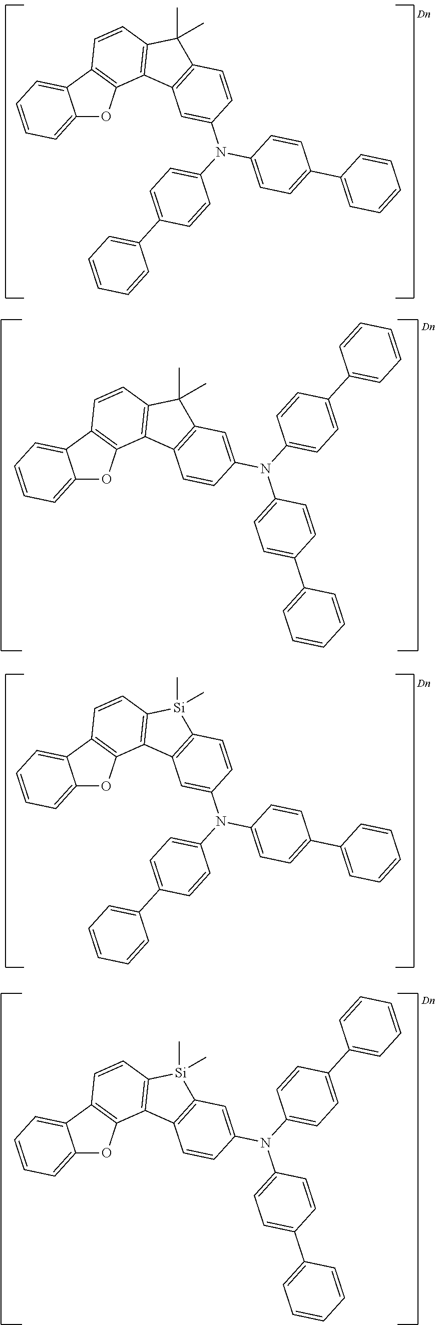

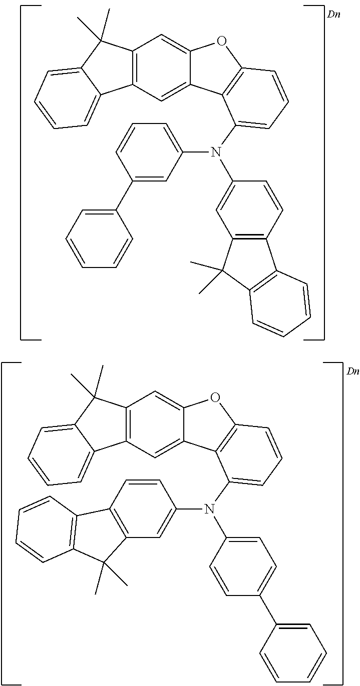

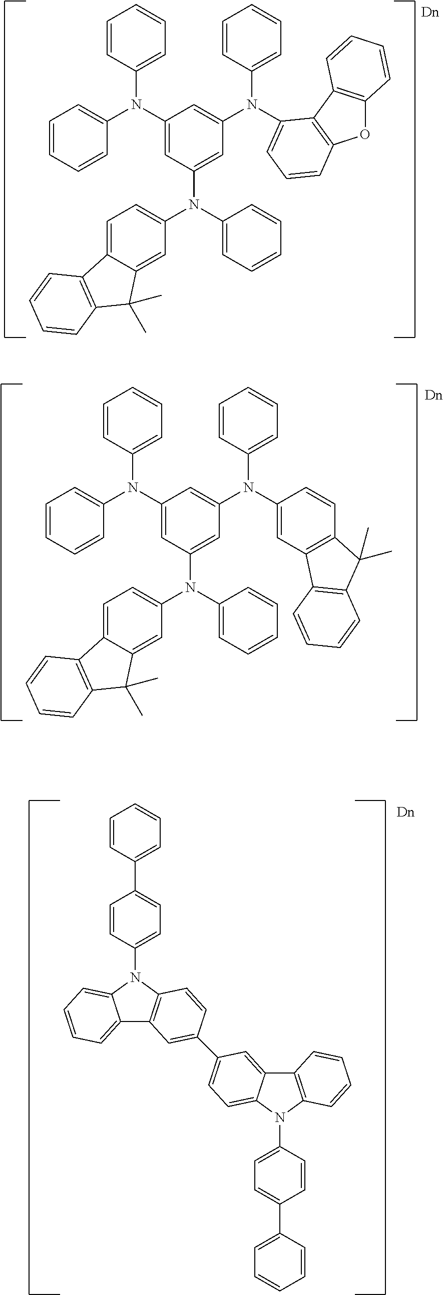

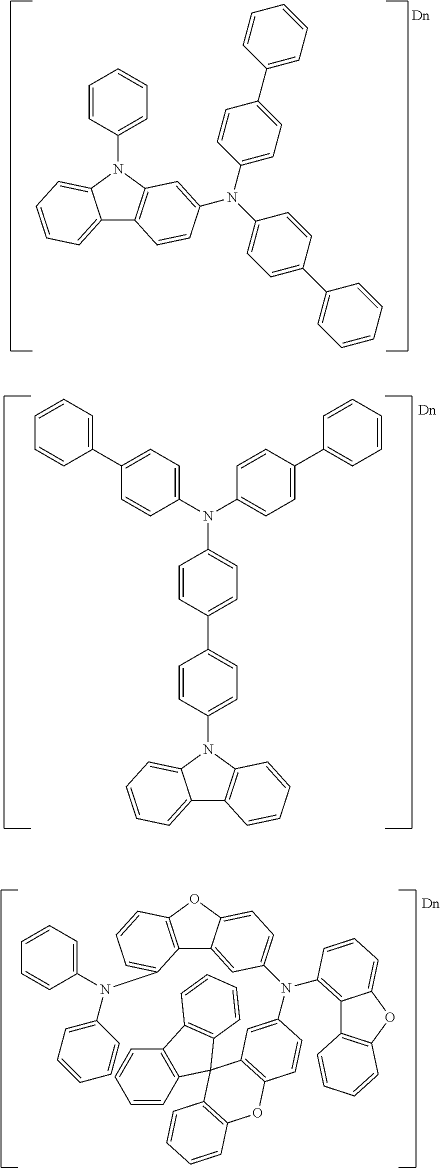

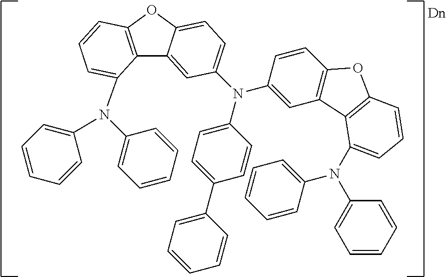

(Dn refers to the number of deuterium substitutions and indicates a structure with one or more deuterium substitutions)

In the hole transport region, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

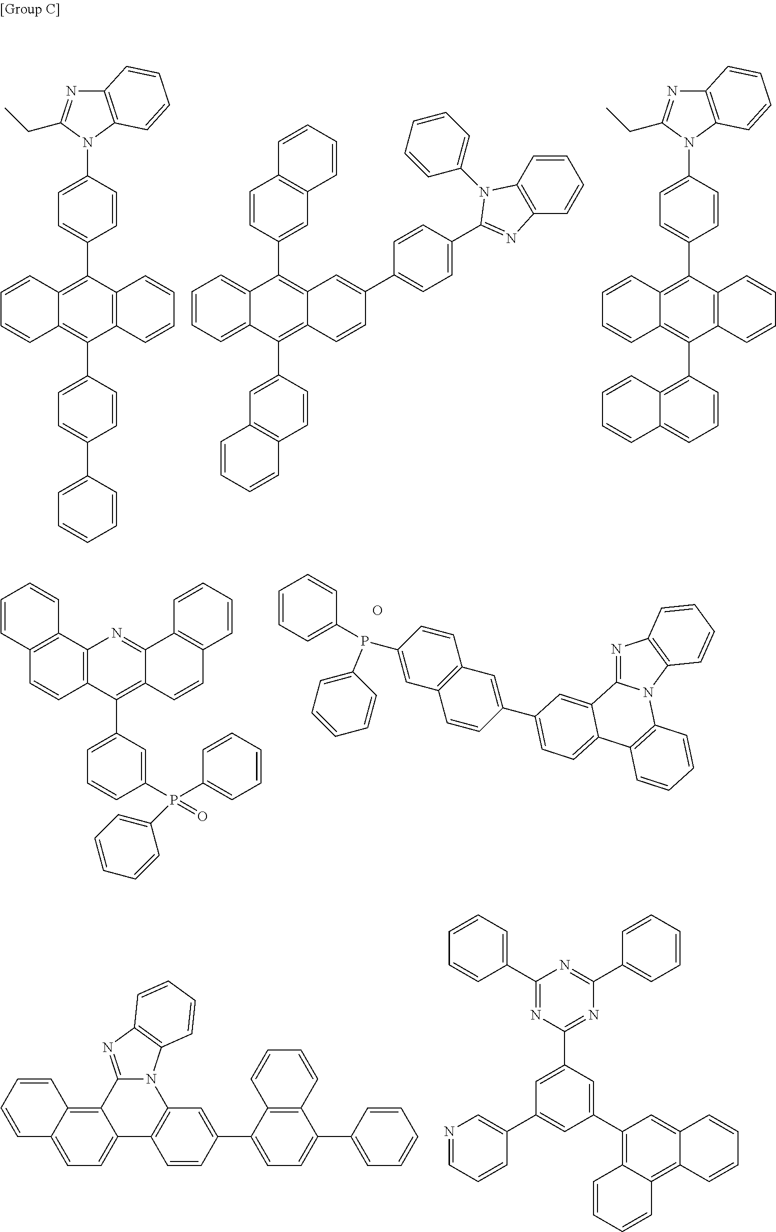

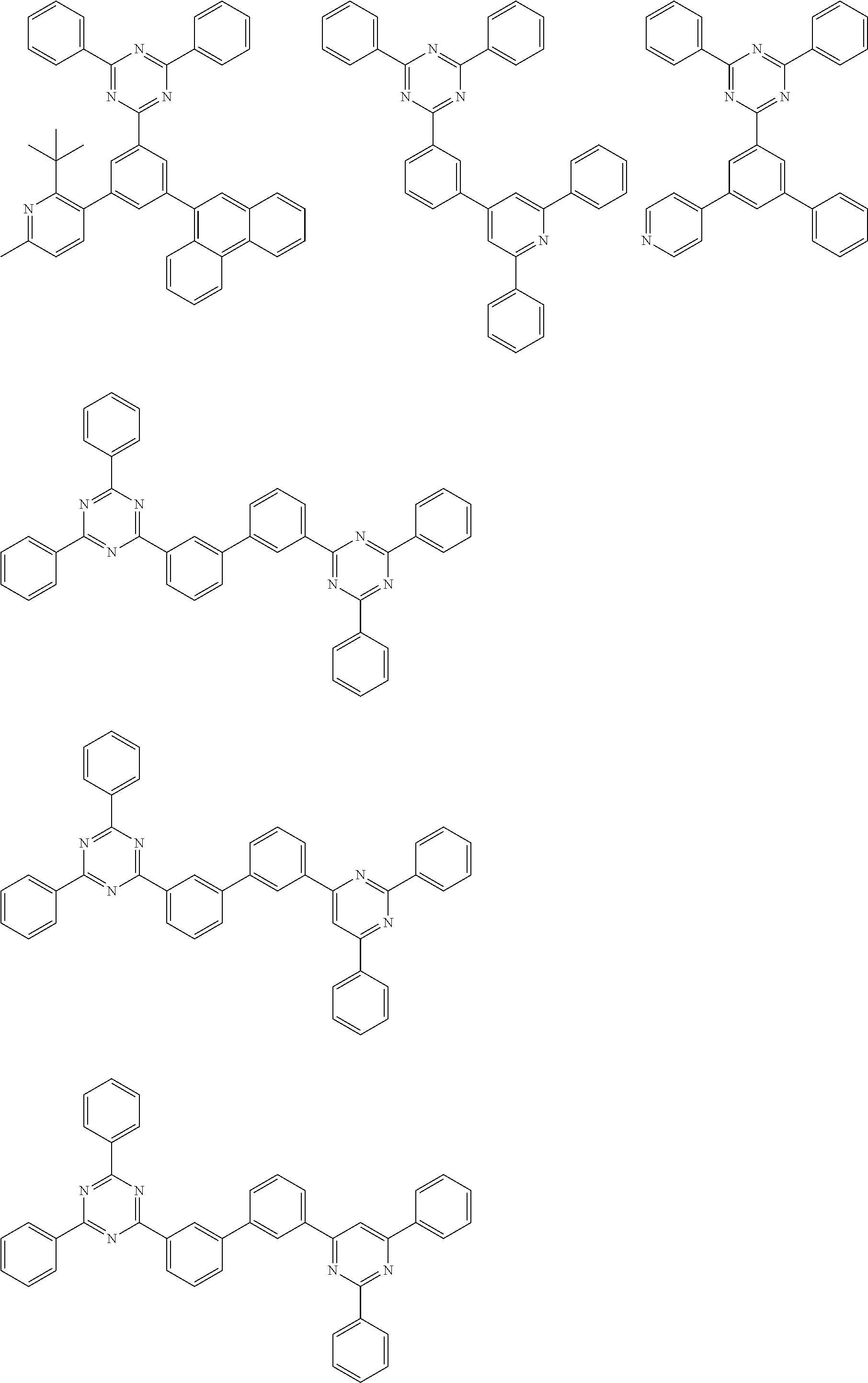

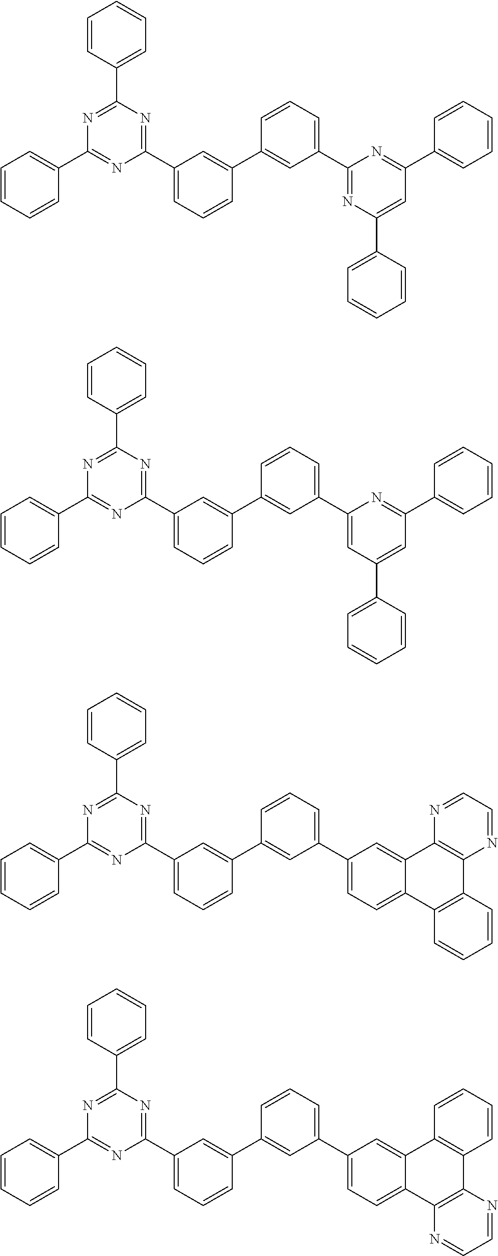 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

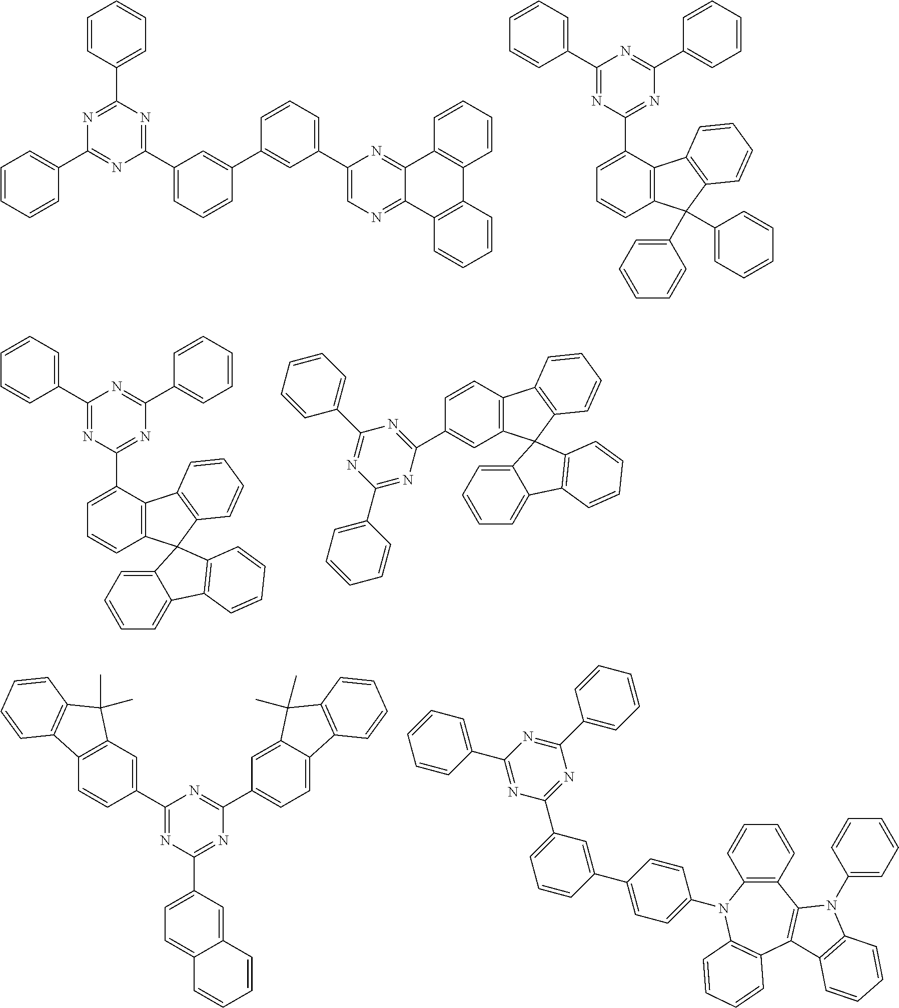

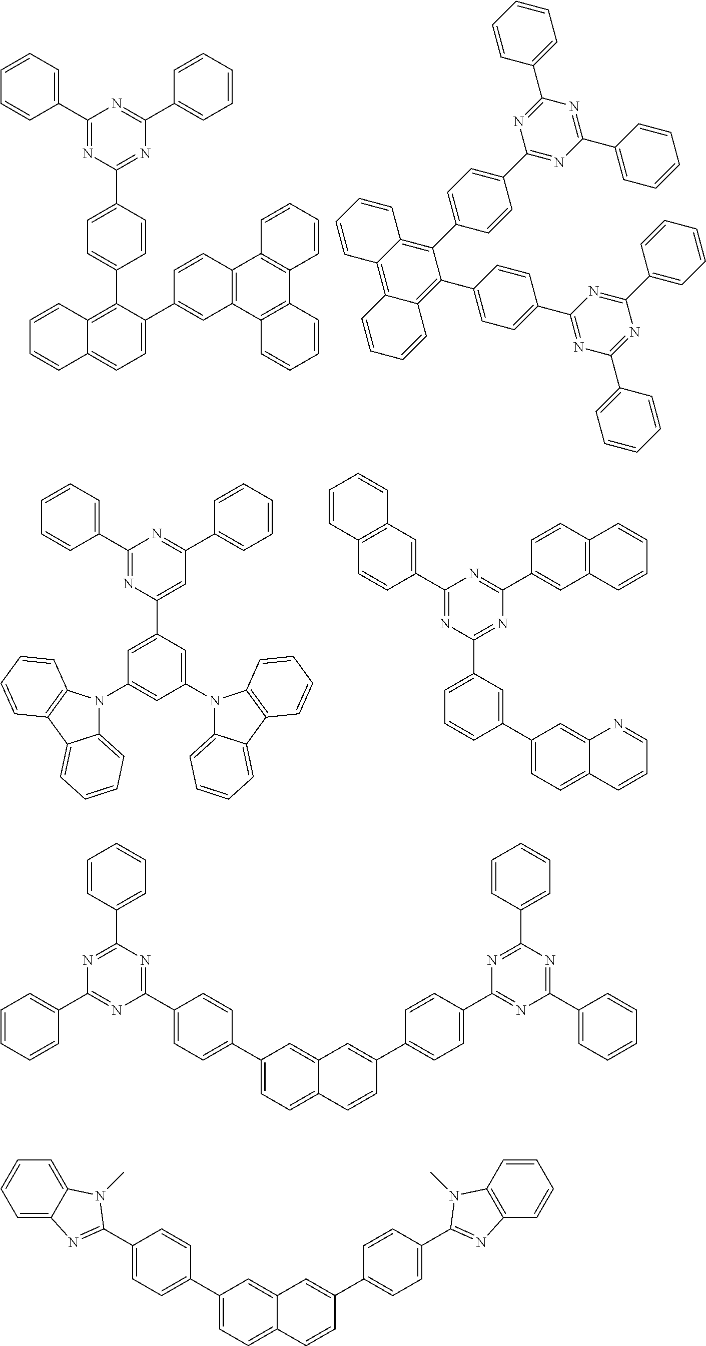

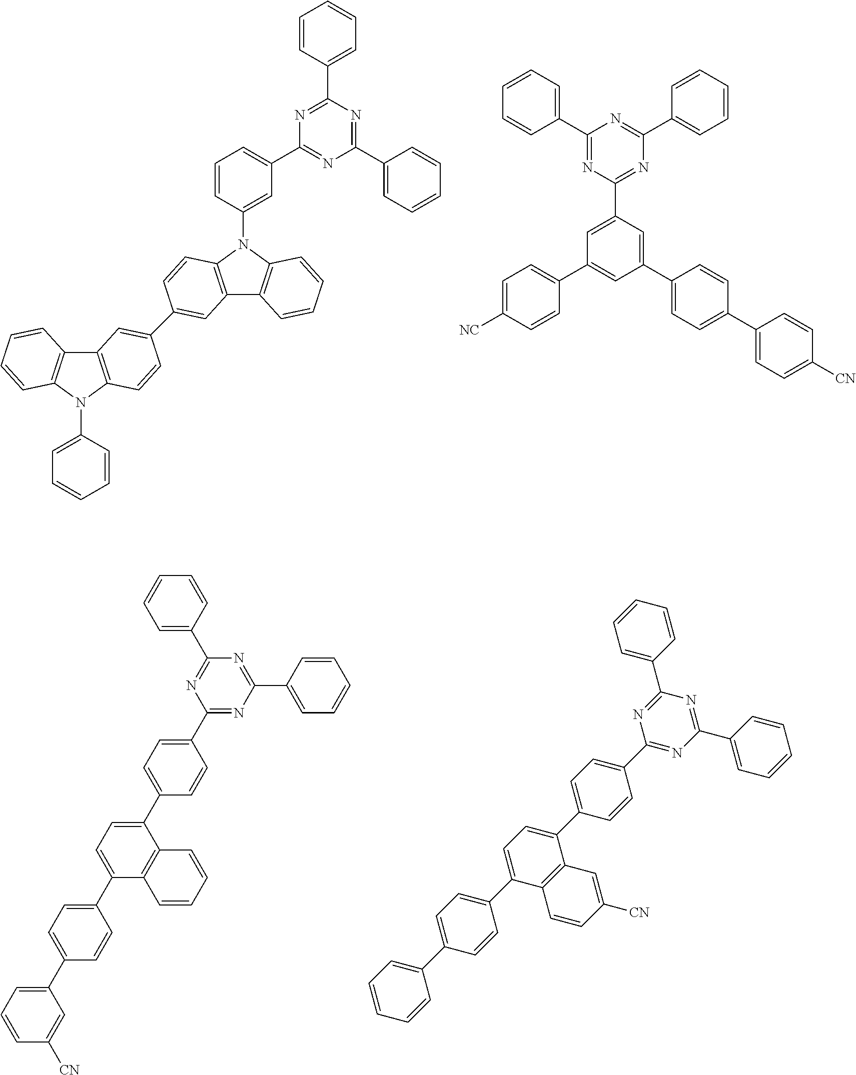 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

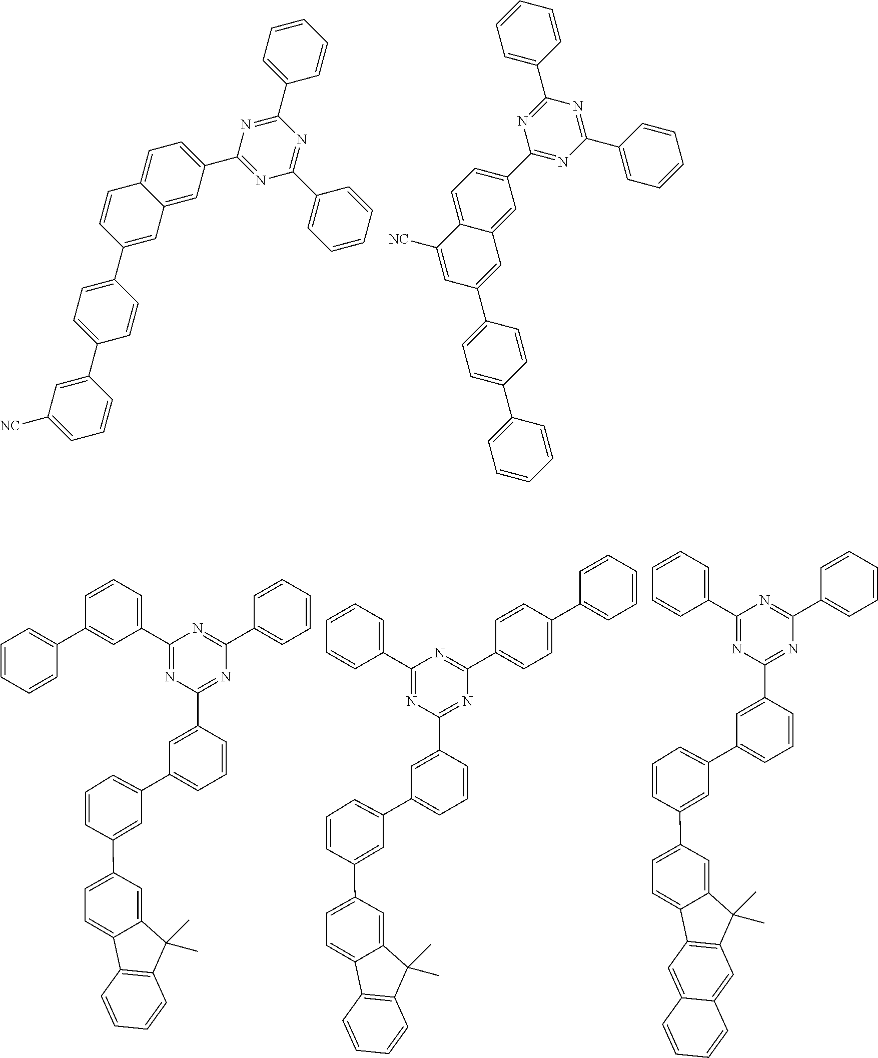

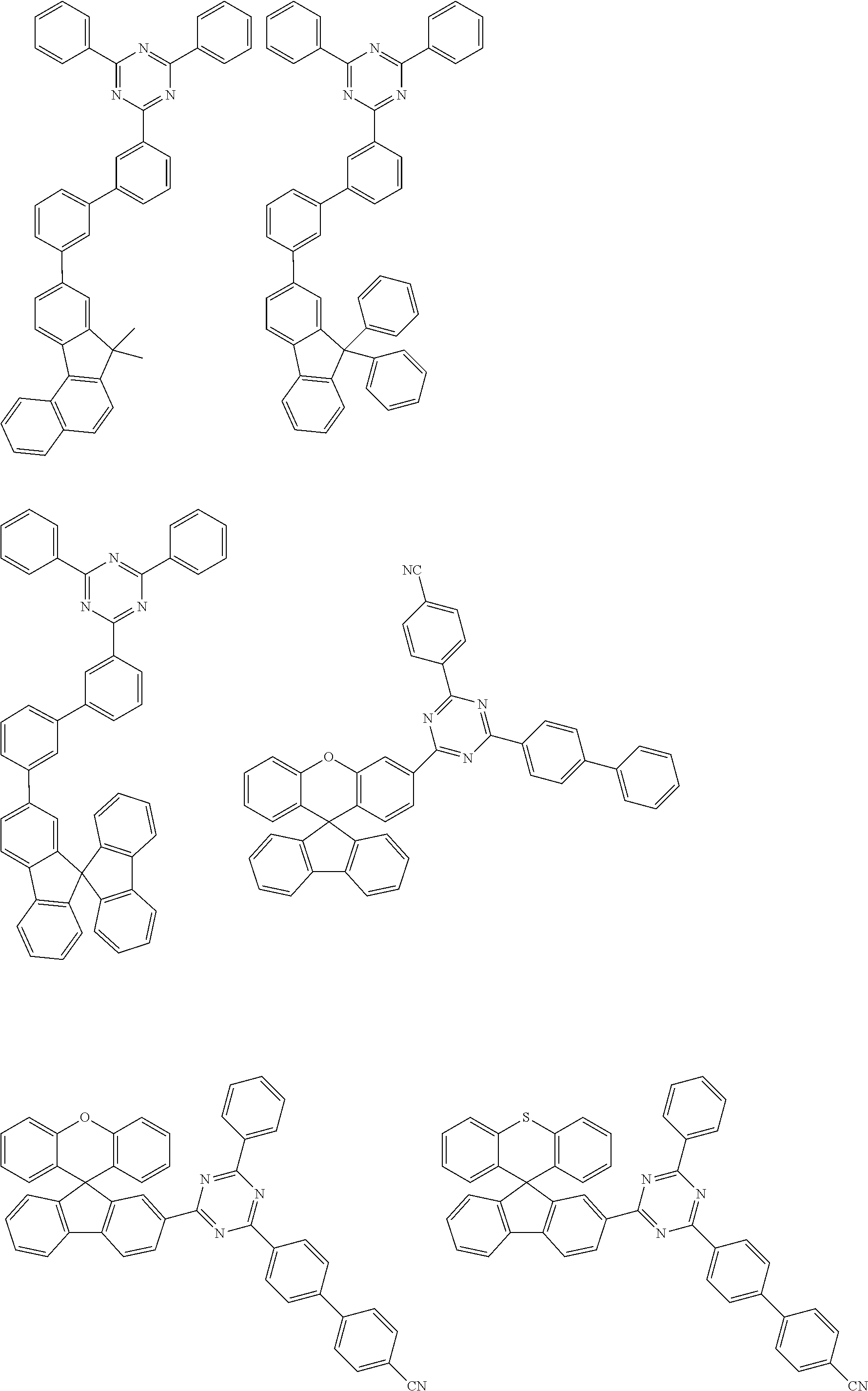

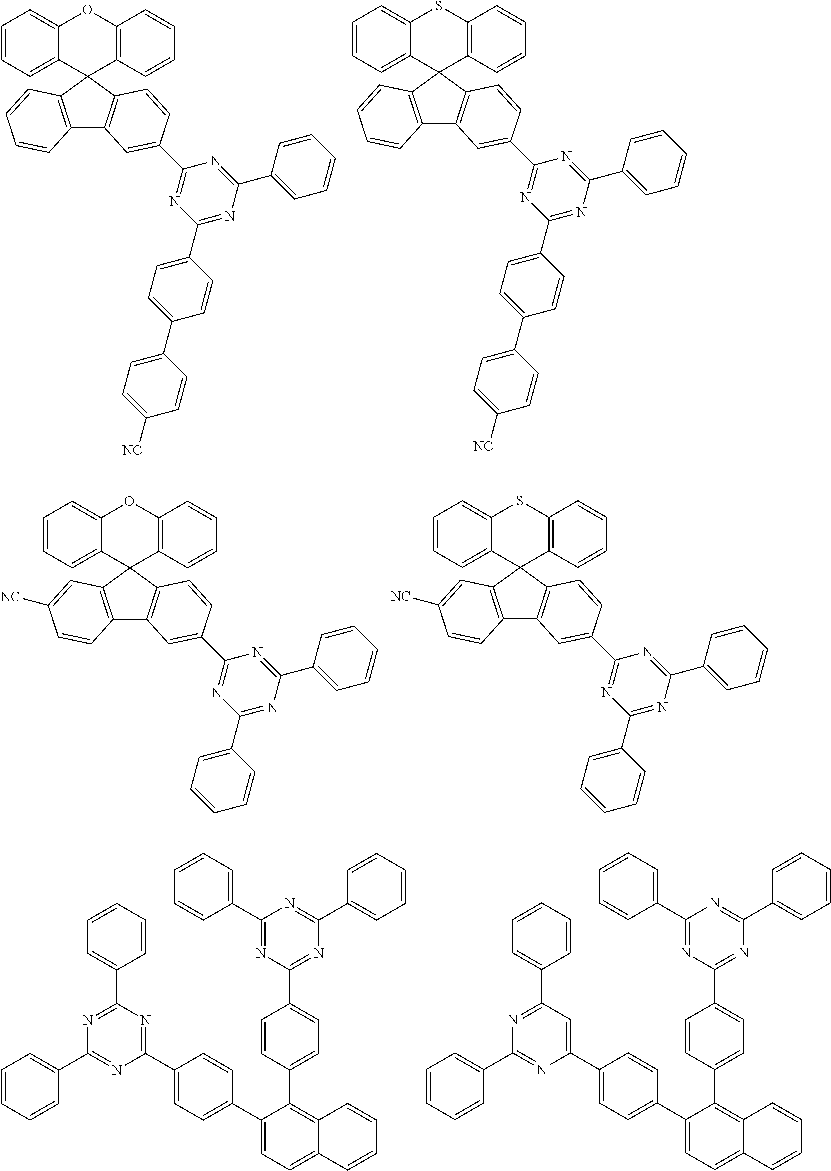

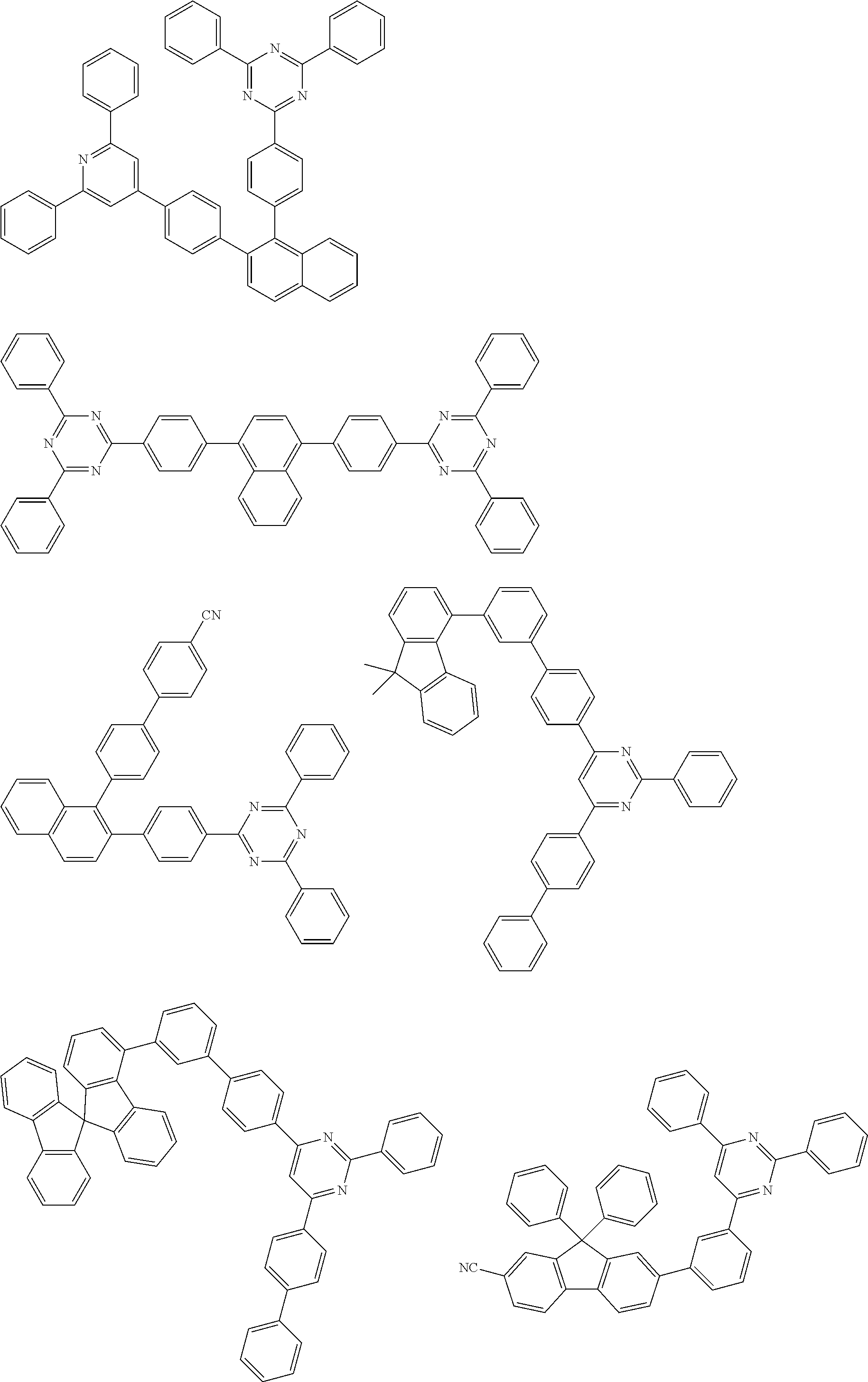

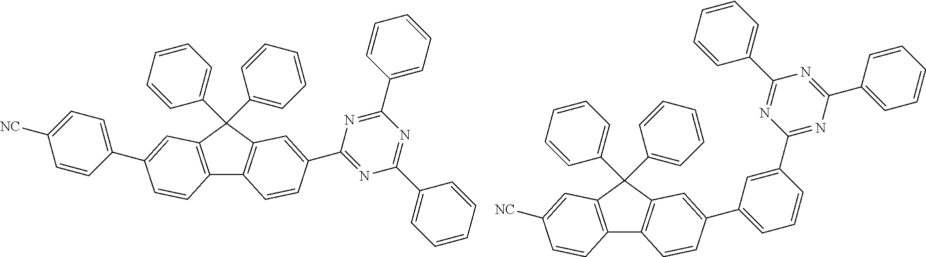

An embodiment of the present invention may provide an organic light emitting diode including the light emitting layer as the organic layer.

Another embodiment of the present invention may provide an organic light emitting diode including a hole transport region and the light emitting layer as the organic layer.

Another embodiment of the present invention may provide an organic light emitting diode including an electron transport region and the light emitting layer as the organic layer.

An embodiment of the present invention may provide an organic light emitting diode including a hole transport region 140 and an electron transport region 150 in addition to the light emitting layer 130 as the organic layer 105, as shown in FIG. 1.

In another embodiment of the present invention, an organic light emitting diode may further include an electron injection layer (not shown), a hole injection layer (not shown), etc. in addition to the light emitting layer as the organic layer.

The organic light emitting diodes may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

### MODE FOR INVENTION

Hereinafter, the embodiments are illustrated in more detail with reference to examples. However, these examples are exemplary, and the present scope is not limited thereto.

### (Synthesis of First Compound)

### Synthesis Example 1: Synthesis of Compound A-17

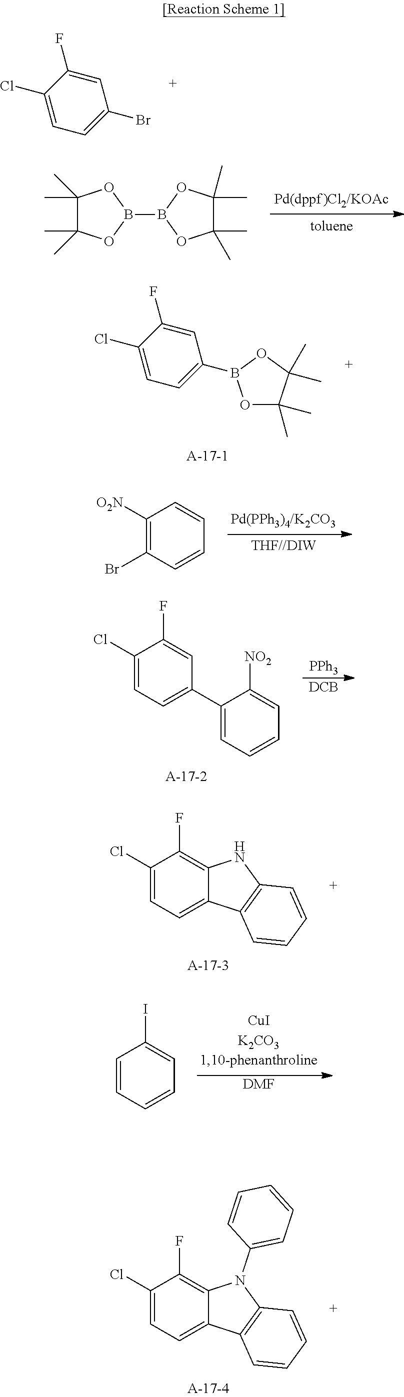

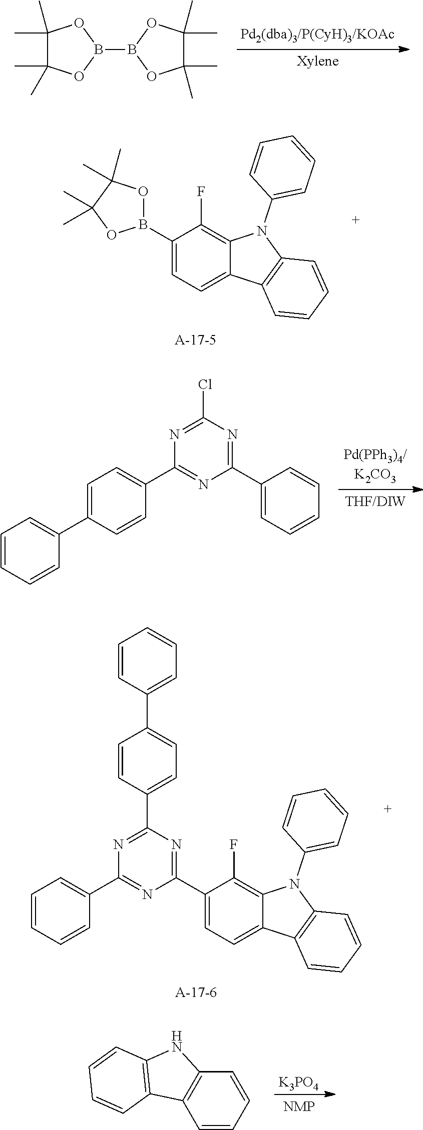

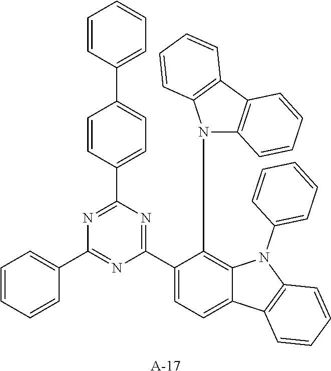 [SMILES: c1ccc(-c2ccc(-c3nc(-c4ccccc4)nc(-c4ccc5c6ccccc6n(-c6ccccc6)c5c4-n4c5ccccc5c5ccccc54)n3)cc2)cc1 ; "A-17"]

1st step: Synthesis of Intermediate A-17-1

An intermediate of 4-bromo-1-chloro-2-fluorobenzene (75 g, 358.1 mmol), bis(pinacolato)diboron (109.1 g, 429.7 mmol), potassium acetate (105.4 g, 1074.3 mmol), and Pd(dppf)Cl₂ (14.6 g, 17.9 mmol) were put in a round bottom flask and dissolved in 900 m1 of toluene. The mixture was stirred under reflux at 125° C. for 8 hours. When a reaction was completed, the resultant was cooled to room temperature, after removing a salt by filtration, an excessive amount of DCM and distilled water were added thereto for extraction. A product therefrom was treated through column chromatography (hexane:DCM (40%)) to obtain 70.0 g (76%) of Intermediate A-17-1.

2nd step: Synthesis of Intermediate A-17-2

Intermediate A-17-1 (67.7 g, 263.8 mmol), 1-bromo-2-nitrobenzene (41.0 g, 202.9 mmol), K₂CO₃ (56.1 g, 405.9 mmol), and Pd(PPh₃)₄ (7.0 g, 6.1 mmol) were put in a round bottom flask and then, dissolved in THF (700 ml) and distilled water (350 ml) and then, stirred at 80° C. for 6 hours. When a reaction was completed, after separating an aqueous layer by using a separatory funnel, an organic layer therefrom was distilled under a reduced pressure. A product therefrom was treated through column chromatography (hexane:DCM (40%)) to obtain 37.0 g (72%) of Intermediate A-17-2.

### 3rd Step: Synthesis of Intermediate A-17-3

Intermediate A-17-2 (37.0 g, 148.5 mmol) and PPh₃ (116.8 g, 445.5 mmol) were put in a round bottom flask and dissolved in 500 m1 of DCB and then, stirred at 200° C. for 6 hours. When a reaction was completed, an organic layer therefrom was distilled under a reduced pressure. A product therefrom was treated through column chromatography (hexane:DCM (20%)) to obtain 15.0 g (46%) of Intermediate A-17-3.

4th step: Synthesis of Intermediate A-17-4

Intermediate A-17-3 (11.6 g, 53.0 mmol), iodobenzene (37.9 g, 186.0 mmol), CuI (2.0 g, 11.0 mmol), 1,10-phenanthroline (1.9 g, 11.0 mmol), and K₂CO₃ (11.0 g, 80.0 mmol) were put in a round bottom flask and dissolved in 180 m1 of DMF and then, stirred at 180° C. for 3 hours.

When a reaction was completed, an organic layer therefrom was distilled under a reduced pressure and then, extracted with an excessive amount of DCM and distilled water. A product therefrom was treated through column chromatography (hexane:DCM (10%)) to obtain 13.0 g (83%) of Intermediate A-17-4.

5th step: Synthesis of Intermediate A-17-5

Intermediate A-17-4 (13.0 g, 43.9 mmol), bis(pinacolato)diboron (14.5 g, 57.1 mmol), tricyclohexylphosphine (2.9 g, 10.5 mmol), potassium acetate (12.9 g, 131.8 mmol), and Pd₂(dba)₃ (2.9 g, 10.5 mmol) were put in a round bottom flask and dissolved in 150 m1 of xylene. The mixture was stirred under reflux at 150° C. for 8 hours. When a reaction was completed, the resultant was cooled to room temperature and filtered, and an organic layer therefrom was distilled under a reduced pressure. A product therefrom was treated through column chromatography (hexane:DCM (40%)) to obtain 15.0 g (88%) of Intermediate A-17-5.

6th step: Synthesis of Intermediate A-17-6

Intermediate A-17-5 (12.4 g, 30.5 mmol), 2-([1,1′-biphenyl]-4-yl)-4-chloro-6-phenyl-1,3,5-triazine (10.5 g, 30.5 mmol), K₂CO₃ (12.6 g, 91.6 mmol), and Pd(PPh₃)₄ (1.1 g, 0.9 mmol) were put in a round bottom flask and dissolved in THF (200 ml) and distilled water (100 ml) and then, stirred at 90° C. for 6 hours. When a reaction was completed, after separating an aqueous layer by using a separatory funnel, an organic layer therefrom was distilled under a reduced pressure. A product therefrom was dissolved by heating in toluene, silica-filtered, and recrystallized to obtain 16.8 g (97%) of Intermediate A-17-6.

7 step: Synthesis of Compound A-17

Intermediate A-17-6 (17.0 g, 30.0 mmol), 9H-carbazole (5.5 g, 33.0 mmol), and K₃PO₄ (9.5 g, 45.0 mmol) were put in a round bottom flask and dissolved in 150 m1 of NMP and then, stirred at 200° C. for 6 hours. When a reaction was completed, the resultant was distilled under a reduced pressure and extracted with an excessive amount of DCM and distilled water. A product therefrom was treated through column chromatography (hexane:EA 10%) to obtain 19.4 g (91%) of Compound A-17.

### Synthesis Example 2: Synthesis of Compound A-14

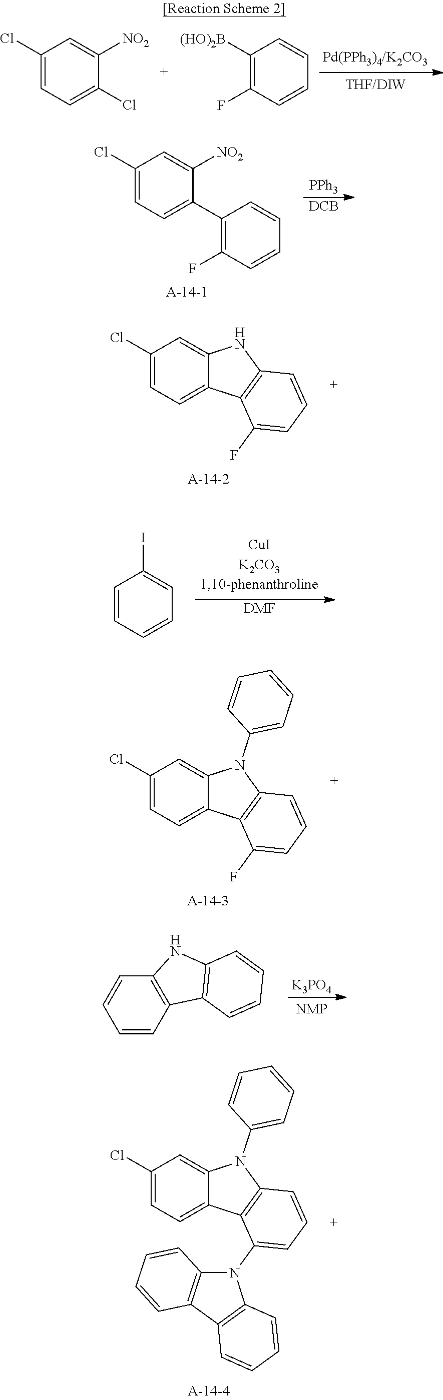

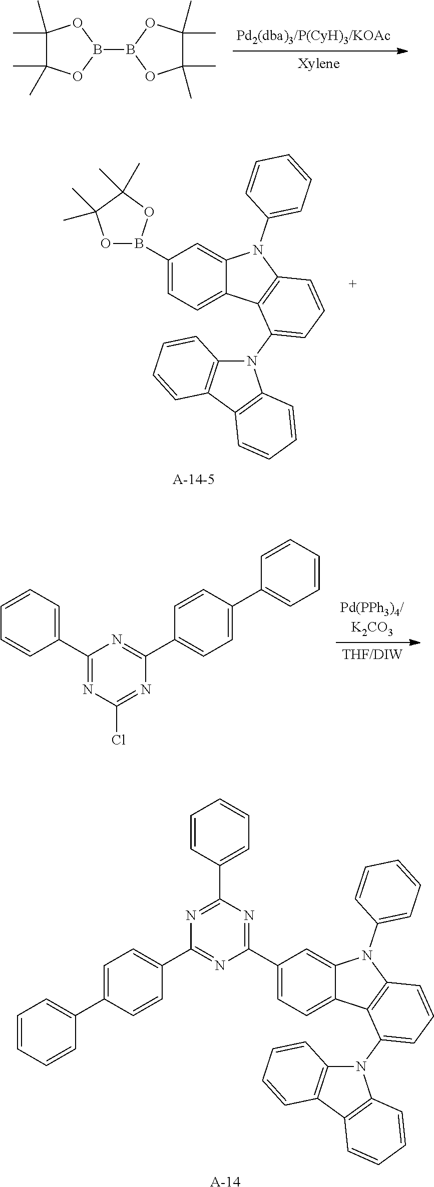

1st step: Synthesis of Intermediate A-14-1

An intermediate of 1,4-dichloro-2-nitrobenzene (37.5 g, 195.3 mmol), (2-fluorophenyl) boronic acid (28.7 g, 205.1 mmol), K₂CO₃ (53.9 g, 390.6 mmol), and Pd(PPh₃)₄ (6.7 g, 5.8 mmol) were put in a round bottom flask and dissolved in 700 m1 of THF and 350 ml of distilled water and then, stirred at 80° C. for 6 hours. When a reaction was completed, after separating an aqueous layer by using a separatory funnel, an organic layer therefrom was distilled under a reduced pressure. A product therefrom was treated through column chromatography (hexane:DCM (40%)) to obtain 42.7 g (97%) of Intermediate A-14-1.

2nd step: Synthesis of Intermediate A-14-2

Intermediate A-14-1 (42.7 g, 169.9 mmol) and PPh₃ (133.7 g, 509.7 mmol) were put in a round bottom flask and dissolved in DCB (500 ml) and then, stirred at 200° C. for 6 hours. When a reaction was completed, an organic layer therefrom was distilled under a reduced pressure. A product therefrom was treated through column chromatography (hexane:DCM (50%)) to obtain 27.1 g (73%) of Intermediate A-14-2.

3rd step: Synthesis of Intermediate A-14-3

Intermediate A-14-2 (27.1 g, 123.0 mmol), iodobenzene (88.0 g, 432.0 mmol), CuI (4.7 g, 25.0 mmol), 1,10-phenanthroline (4.4 g, 25.0 mmol), K₂CO₃ (25.6 g, and 185.0 mmol) were put in a round bottom flask and dissolved in DMF (180 ml) and then, stirred at 180° C. for 3 hours. When a reaction was completed, an organic layer therefrom was distilled under a reduced pressure and then, extracted with an excessive amount of DCM and distilled water. A product therefrom was treated through column chromatography (hexane:DCM (10%)) to obtain 34.1 g (94%) of Intermediate A-14-3.

4th step: Synthesis of Intermediate A-14-4

Intermediate A-14-3 (34.2 g, 115.6 mmol), 9H-carbazole (21.3 g, 127.2 mmol), and K₃PO₄ (36.8 g, 173.4 mmol) were put in a round bottom flask and dissolved in NMP (150 ml) and then, stirred at 200° C. for 6 hours. When a reaction was completed, the resultant was distilled under a reduced pressure and then, extracted with an excessive amount of DCM and distilled water. A product therefrom was treated through column chromatography (hexane:MC (30%)) to obtain 40.3 g (79%) of Intermediate A-14-4.

5th step: Synthesis of Intermediate A-14-5

Intermediate A-14-4 (40.3 g, 90.9 mmol), bis(pinacolato)diboron (30.0 g, 118.2 mmol), tricyclohexylphosphine (6.1 g, 21.8 mmol), potassium acetate (26.8 g, 272.7 mmol), and Pd₂(dba)₃ (5.0 g, 5.4 mmol) were put in a round bottom flask and dissolved in 150 m1 of xylene. The mixture was stirred under reflux at 150° C. for 8 hours. When a reaction was completed, the resultant was cooled to room temperature and filtered to remove a salt, and an organic layer therefrom was distilled under a reduced pressure. A product therefrom was treated through column chromatography (hexane:DCM (40%)) to obtain 45.1 g (93%) of Intermediate A-14-5.

6th step: Synthesis of Compound A-14

Intermediate A-14-5 (11.7 g, 19.8 mmol), 2-([1,1′-biphenyl]-4-yl)-4-chloro-6-phenyl-1,3,5-triazine (6.8 g, 19.8 mmol), K₂CO₃ (8.2 g, 59.3 mmol), and Pd(PPh₃)₄ (0.7 g, 0.6 mmol) were put in a round bottom flask and dissolved in THF (130 ml) and distilled water (65 ml) and then, stirred at 90° C. for 6 hours. When a reaction was completed, after separating an aqueous layer by using a separatory funnel, an organic layer therefrom was distilled under a reduced pressure. A product therefrom was dissolved by heating in toluene and then, silica-filtered and recrystallized to obtain 12.0 g (85%) of Compound A-14.

### Synthesis Example 3: Synthesis of Compound A-27

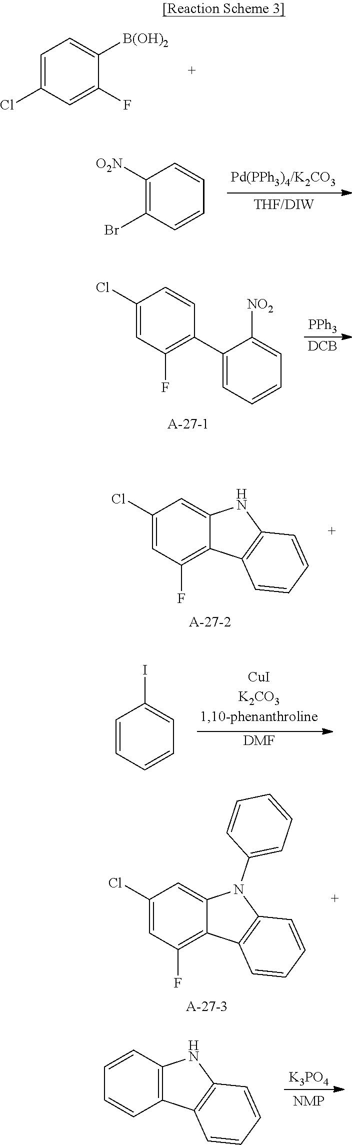

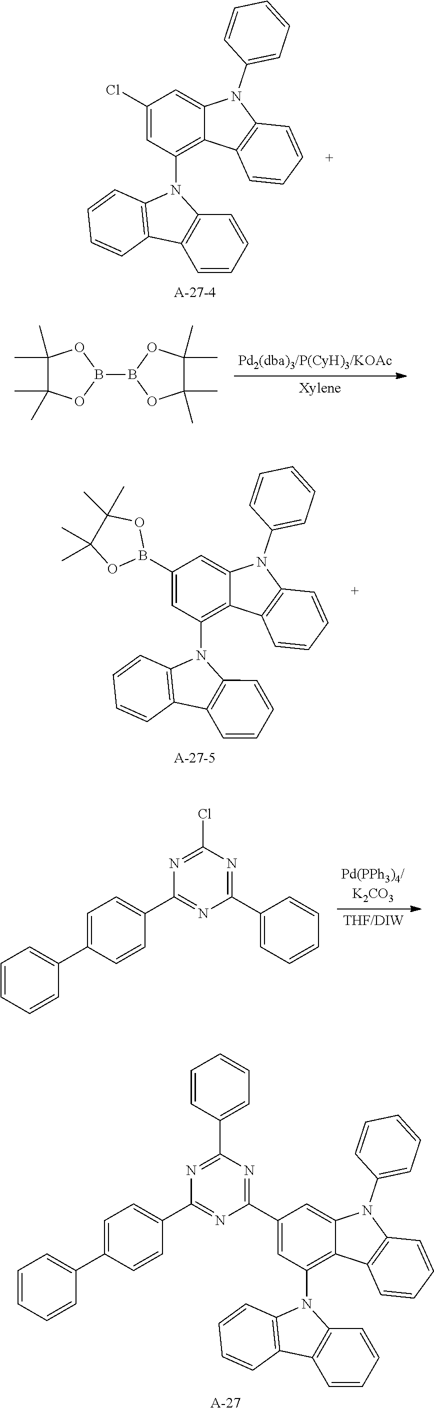

Compound A-27 was synthesized in the same manner as in the 1st step to 6th step of Synthesis Example 2 except that 1-bromo-2-nitrobenzene instead of the 1,4-dichloro-nitrobenzene in the 1st step of Synthesis Example 2 and (4-chloro-2-fluorophenyl) boronic acid instead of the (2-fluorophenyl) boronic acid were used.

### Synthesis Example 4: Synthesis of Compound A-2

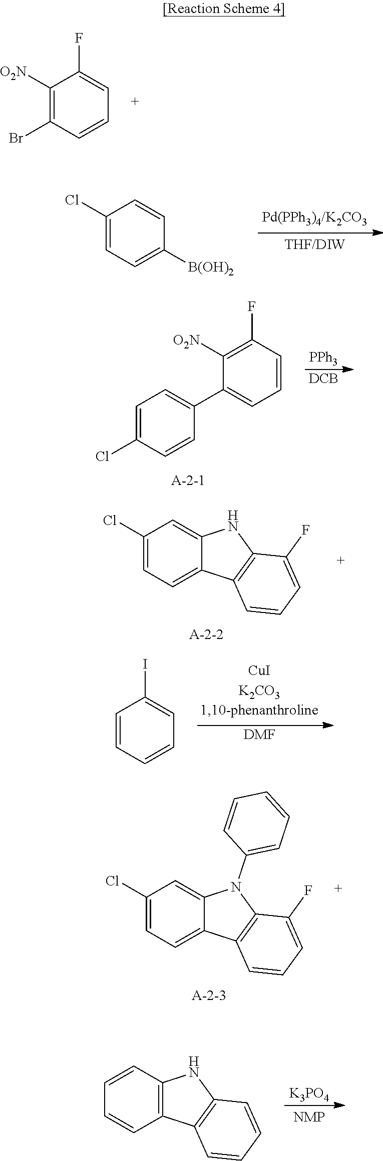

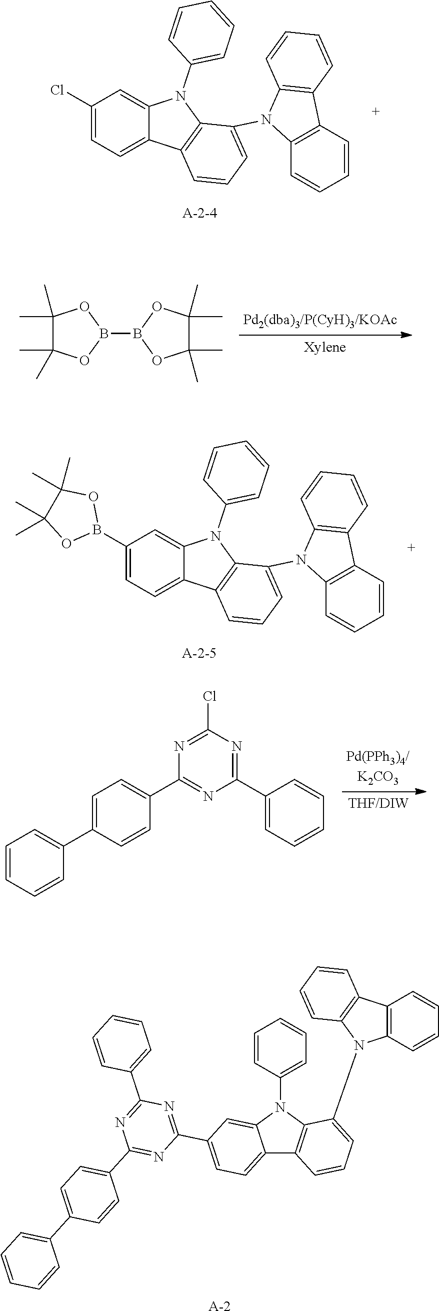

Compound A-2 was synthesized in the same manner as in the 1st step to 6th step of Synthesis Example 2 except that 1-bromo-3-fluoro-2-nitrobenzene instead of the 1,4-dichloro-nitrobenzene in the 1st step of Synthesis Example 2 and (4-chlorophenyl) boronic acid instead of the (2-fluorophenyl) boronic acid were used.

### Synthesis Example 5: Synthesis of Compound B-1

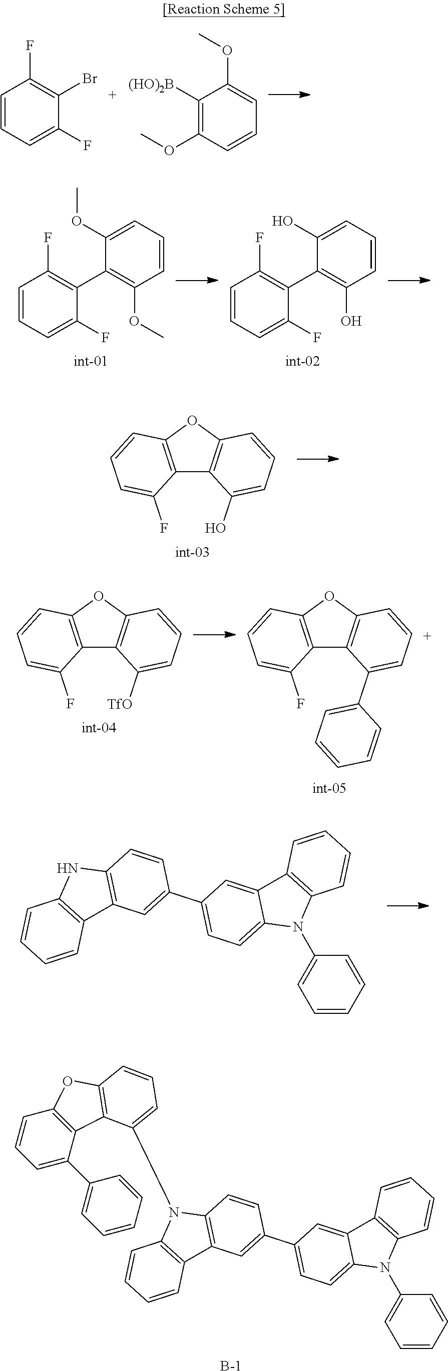

1st step: Synthesis of Intermediate int-01

1-bromo-2,6-difluorobenzene (100 g, 518.2 mmol), 2,6-dimethoxyphenyl boronic acid (99.0 g, 544.1 mmol), K₂CO₃ (179 g, 1295.4 mmol), and Pd(PPh₃)₄ (29.9 g, 25.9 mmol) were put in a round bottom flask and dissolved in THF (1000 ml) and distilled water (500 ml) and then, stirred under reflux at 70° C. for 12 hours. When a reaction was completed, after removing an aqueous layer therefrom, the residue was treated through column chromatography (hexane:DCM (20%)) to obtain 78 g (60%) of Intermediate int-01.

2nd step: Synthesis of Intermediate int-02

Intermediate int-01 (72 g, 287.7 mmol) and pyridine hydrochloride (166.3 g, 1438.6 mmol) were put in a round bottom flask and stirred under reflux at 200° C. for 24 hours. When a reaction was completed, the resultant was cooled to room temperature and slowly poured into distilled water and then, stirred for 1 hour. A solid therefrom was filtered to obtain 60 g (94%) of Intermediate int-02.

3rd step: Synthesis of Intermediate int-03

Intermediate int-02 (64 g, 287.7 mmol) and K₂CO₃ (47.7 g, 345.3 mmol) were put in a round bottom flask and dissolved in NMP (200 ml) and then, stirred under reflux at 180° C. for 12 hours. When a reaction was completed, the mixture was poured into an excessive amount of distilled water. A solid therefrom was filtered, dissolved in ethylacetate, and dried with MgSO₄ to remove an organic layer under a reduced pressure. A product therefrom was treated through column chromatography (hexane:ethyl acetate (30%)) to obtain 51 g (88%) of Intermediate int-03.

4th step: Synthesis of Intermediate int-04

Intermediate int-03 (14 g, 69.2 mmol) and pyridine (8.4 ml, 103.9 mmol) were put in a round bottom flask and dissolved in DCM (150 ml). After cooling to 0° C., trifluoromethanesulfonic anhydride (13.9 ml, 83.1 mmol) was slowly added thereto in a dropwise fashion. After 6 hours, when a reaction was completed, an excessive amount of distilled water was added thereto and then, stirred for 30 minutes and extracted with DCM. After removing an organic solvent therefrom under a reduced pressure, 22 g (95%) of Intermediate int-04 was obtained by vacuum-drying.

5th step: Synthesis of Intermediate int-05

12.5 g (76%) of Intermediate int-05 was synthesized in the same manner as in the 1st step of Synthesis Example 1 except that Intermediate int-04 (21 g, 62.8 mmol), phenylboronic acid (8.4 g, 69.1 mmol), K₂CO₃ (13 g, 94.2 mmol), and Pd(PPh₃)₄ (3.6 g, 3.1 mmol) were used.

6th step: Synthesis of Compound B-1

Intermediate int-05 (12 g, 45.8 mmol), 9-phenyl-3,3′-bicarbazole (22.4 g, 54.9 mmol), and K₃PO₄ (19.4 g, 91.5 mmol) were put in a round bottom flask and dissolved in DMF (130 ml). The solution was stirred under reflux at 160° C. for 6 hours. When a reaction was completed, after filtering a salt, an filtrate therefrom was adsorbed. 19.4 g (65%) of Compound B-1 was obtained by using column chromatography (hexane:DCM (35%)).

### Synthesis Example 6: Synthesis of Compound B-36

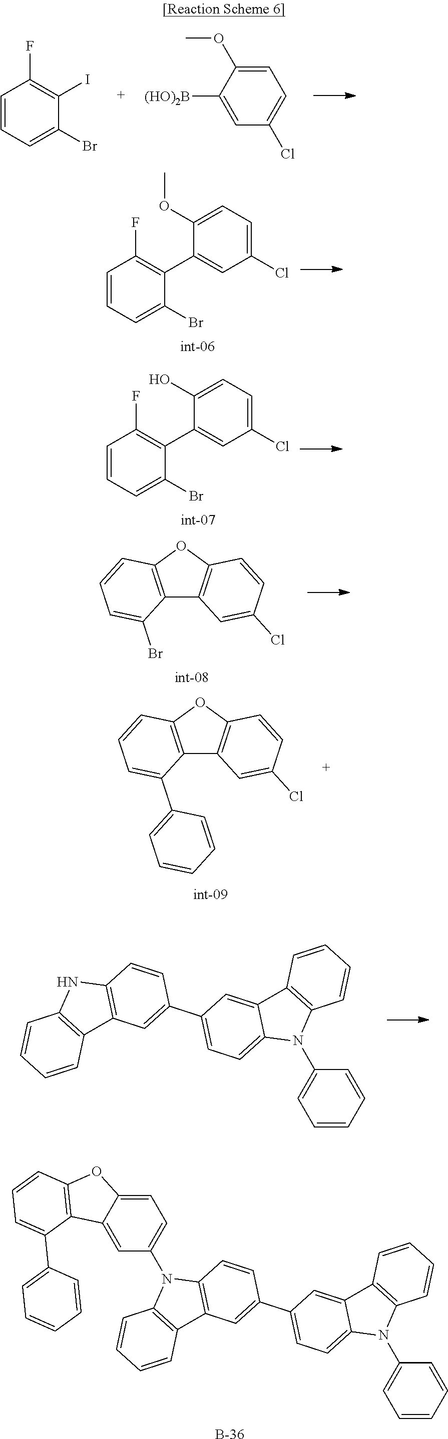

1 st step: Synthesis of Intermediate int-06

1-bromo-3-fluoro-2-iodobenzen (80 g, 265.9 mmol), 5-chloro-2-methoxyphenylboronic acid (54.5 g, 292.5 mmol), K₂CO₃ (73.5 g, 531.8 mmol), and Pd(PPh₃)₄ (15.4 g, 13.3 mmol) were put in a round bottom flask and dissolved in THF (550 ml) and distilled water (250 ml) and then, stirred under reflux at 70° C. for 12 hours. When a reaction was completed, after removing an aqueous layer therefrom, 71.3 g (85%) of Intermediate int-06 was obtained through column chromatography (hexane:DCM (20%)).

2nd step: Synthesis of Intermediate int-07

61.5 g (92%) of Intermediate int-07 was obtained in the same manner as in the 2nd step of Synthesis Example 5 except that Intermediate int-06 (70 g, 287.7 mmol) and pyridine hydrochloride (128.2 g, 1109.1 mmol) were used.

3rd step: Synthesis of Intermediate int-08

48.4 g (85%) of Intermediate int-08 was obtained in the same manner as in the 3rd step of Synthesis Example 5 except that Intermediate int-07 (61 g, 202.3 mmol) and K₂CO₃ (41.9 g, 303.4 mmol) were used.

4th step: Synthesis of Intermediate int-09

Intermediate int-08 (48 g, 170.5 mmol), phenylboronic acid (22.9 g, 187.6 mmol), K₂CO₃ (47.3 g, 341.0 mmol), and Pd(PPh₃)₄ (9.9 g, 8.5 mmol) were put in a round bottom flask and dissolved in THF (560 ml) and distilled water (170 ml) and then, stirred under reflux at 70° C. for 12 hours. When a reaction was completed, after removing an aqueous layer therefrom, 35.2 g (74%) of Intermediate int-09 was obtained through column chromatography (hexane:DCM (20%)).

5th step: Synthesis of Compound B-36

Intermediate int-09 (30 g, 107.6 mmol), 9-phenyl-3,3′-bicarbazole (44 g, 107.6 mmol), sodium tert-butoxide (20.7 g, 215.3 mmol), tri-tert-butylphosphine (2.2 g, 10.8 mmol), and Pd₂(dba)₃ (4.9 g, 5.4 mmol) were put in a round bottom flask and dissolved in xylene (360 ml) and then, stirred under reflux at 150° C. for 6 hours. When a reaction was completed, after removing a salt through filtration, a filtrate therefrom was adsorbed with silica gel. 39.9 g (57%) of Compound B-36 was obtained through column chromatography (hexane:DCM 35%).

### Synthesis Example 7: Synthesis of Compound B-71

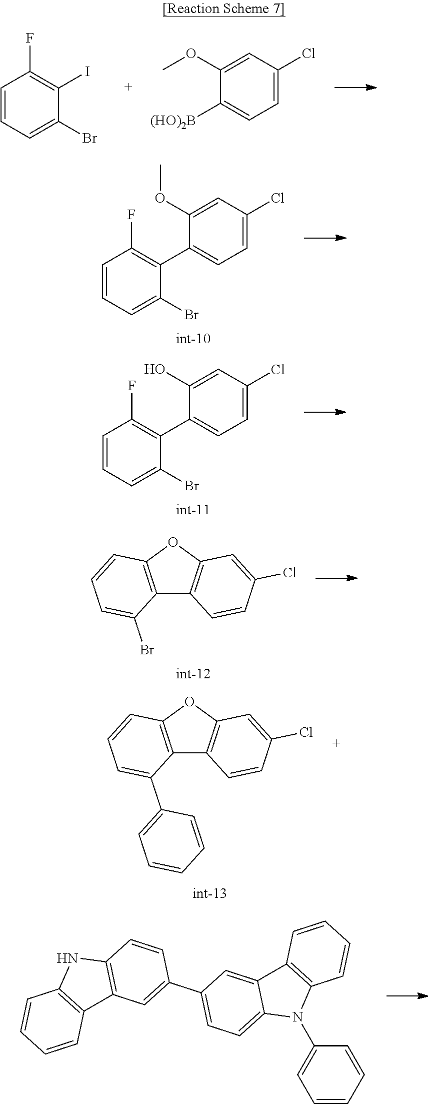

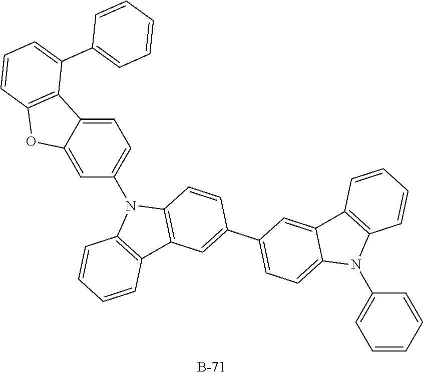

1 st step: Synthesis of Intermediate int-10

Intermediate int-10 was synthesized in the same manner as in the 1st step of Synthesis Example 6 except that 4-chloro-2-methoxyphenylboronic acid instead of the 5-chloro-2-methoxyphenylboronic acid was used.

2nd step: Synthesis of Intermediate int-11

Intermediate int-11 was synthesized in the same manner as in the 2nd step of Synthesis Example 6.

3rd step: Synthesis of Intermediate int-12

Intermediate int-12 was synthesized in the same manner as in the 3rd step of Synthesis Example 6.

4th step: Synthesis of Intermediate int-13

Intermediate int-13 was synthesized in the same manner as in the 4th step of Synthesis Example 6.

5th step: Synthesis of Compound B-71

Compound B-71 was synthesized in the same manner as in the 5th step of Synthesis Example 6.

### Synthesis Example 8: Synthesis of Compound B-106

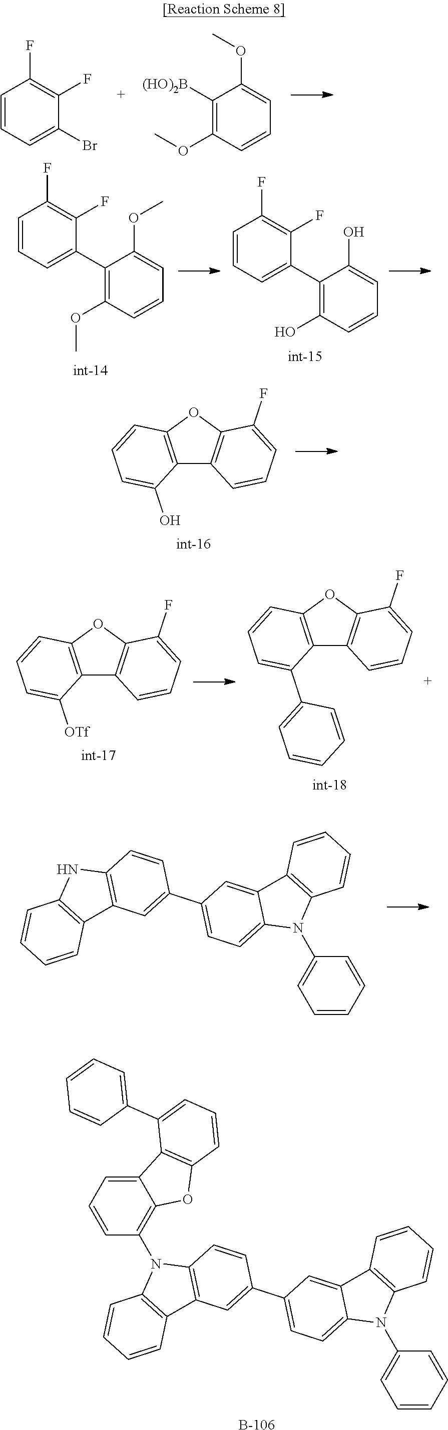

1st step: Synthesis of Intermediate int-14

Intermediate int-14 was synthesized in the same manner as in the 1st step of Synthesis Example 5 except that 1-bromo-2,3-difluorobenzene was used instead of the 1-bromo-2,6-difluorobenzene.

2nd step: Synthesis of Intermediate int-15

Intermediate int-15 was synthesized in the same manner as in the 2nd step of Synthesis Example 5.

3rd step: Synthesis of Intermediate int-16

Intermediate int-16 was synthesized in the same manner as in the 3rd step of Synthesis Example 5.

4th step: Synthesis of Intermediate int-17

Intermediate int-17 was synthesized in the same manner as in the 4th step of Synthesis Example 5.

5th step: Synthesis of Intermediate int-18

Intermediate int-18 was synthesized in the same manner as in the 5th step of Synthesis Example 5.

6th step: Synthesis of Compound B-106

Intermediate int-106 was synthesized in the same manner as in the 6th step of Synthesis Example 5.

### Comparative Synthesis Example 1: Synthesis of Compound C-1

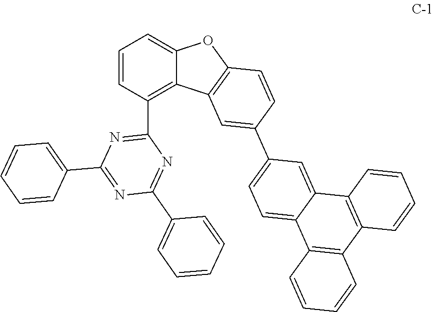 [SMILES: c1ccc(-c2nc(-c3ccccc3)nc(-c3cccc4oc5ccc(-c6ccc7c8ccccc8c8ccccc8c7c6)cc5c34)n2)cc1]

Compound C-1 was synthesized with reference to a synthesis method known in the registered patent KR 1849747 B1.

### Comparative Synthesis Example 2: Synthesis of Compound C-2

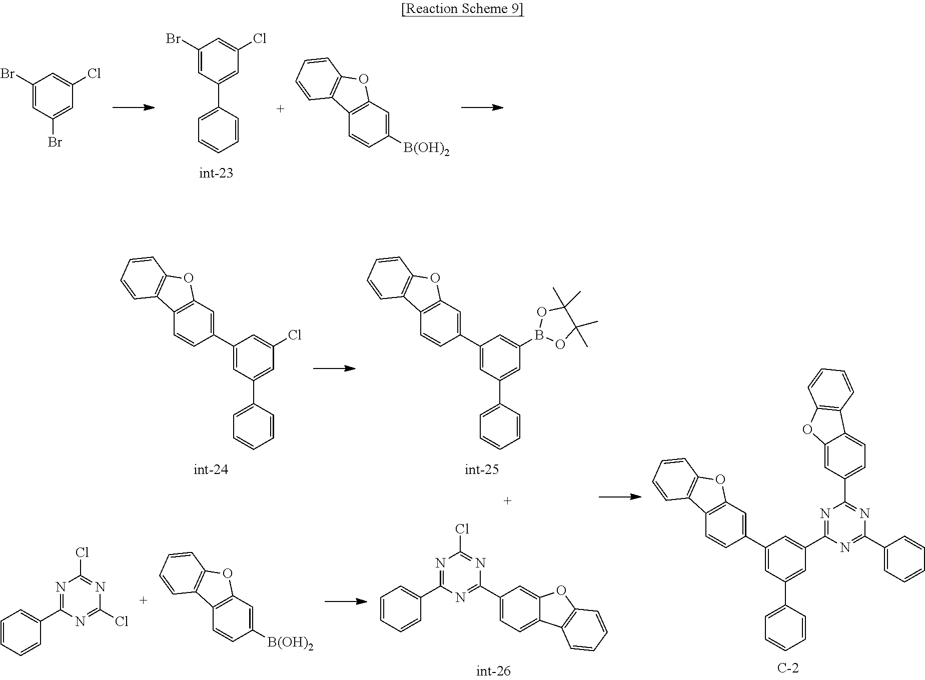

1st step: Synthesis of Intermediate int-23

1,3-dibromo-5-chlorobenzene (45 g, 166.5 mmol), phenylboronic acid (19.3 g, 158.1 mmol), K₂CO₃ (41.4 g, 299.6 mmol), and Pd(PPh₃)₄ (9.6 g, 8.3 mmol) were put in a round bottom flask and dissolved in THF (600 ml) and distilled water (150 ml) and then, stirred under reflux at 70° C. for 8 hours. When a reaction was completed, after removing an aqueous layer, 25 g (59%) of Intermediate int-23 was obtained through column chromatography (hexane:DCM (15%)).

2nd step: Synthesis of Intermediate int-24

Intermediate int-23 (25 g, 93.4 mmol), 3-dibenzofuranylboronic acid (21.8 g, 102.8 mmol), K₂CO₃ (25.8 g, 186.9 mmol), and Pd(PPh₃)₄ (5.4 g, 4.7 mmol) were put in a round bottom flask and dissolved in THF (400 ml) and distilled water (100 ml) and then, stirred under reflux at 70° C. for 8 hours. When a reaction was completed, after removing an aqueous layer therefrom, 24.4 g (67%) of Intermediate int-24 was obtained by using column chromatography (hexane:DCM 30%).

3rd step: Synthesis of Intermediate int-25

Intermediate int-24 (24 g, 67.6 mmol), bis(pinacolato)diboron (20.6 g, 81.2 mmol), tricyclohexylphosphine (3.3 g, 13.5 mmol), potassium acetate (13.3 g, 135.3 mmol), and Pd(dppf)Cl₂ (1.7 g, 2.0 mmol) were put in a round bottom flask and dissolved in xylene (250 ml). The mixture was stirred under reflux at 150° C. for 8 hours. When a reaction was completed, the resultant was cooled to room temperature, filtered to remove a salt, and extracted with an excessive amount of DCM and distilled water. 23.6 g (78%) of Intermediate int-25 was obtained through column chromatography (hexane:DCM (40%)).

4th step: Synthesis of Intermediate int-26

2,4-dichloro-6-phenyl-1,3,5-triazine (20 g, 88.5 mmol), 3-dibenzofuranylboronic acid (17.8 g, 84.1 mmol), K₂CO₃ (24.5 g, 176.9 mmol), and Pd(dppf)Cl₂ (3.6 g, 4.4 mmol) were put in a round bottom flask and dissolved in toluene (250 ml) and distilled water (90 ml) and then, stirred at 60° C. for 6 hours. When a reaction was completed, after removing an aqueous layer by using a separatory funnel, an organic layer therefrom was distilled under a reduced pressure. A product therefrom was dissolved by heating with monochlorobenzene, silica-filtered, and recrystallized to obtain 14.4 g (48%) of Intermediate int-26.

5th step: Synthesis of Compound C-2

Intermediate int-26 (14.1 g, 39.4 mmol), Intermediate int-25 (18.5 g, 14.4 mmol), K₂CO₃ (10.9 g, 78.8 mmol), and Pd(PPh₃)₄ (2.3 g, 2.0 mmol) were put in a round bottom flask and dissolved in THF (200 ml) and distilled water (40 ml) and then, stirred under reflux at 70° C. for 8 hours. When a reaction was completed, after removing an aqueous layer, a solid precipitated therein was filtered. A product therefrom was dissolved by heating with monochlorobenzene and then, silica-filtered and recrystallized to obtain 18.2 g (72%) of Compound C-2.

### Comparative Synthesis Example 3: Synthesis of Compound C-3

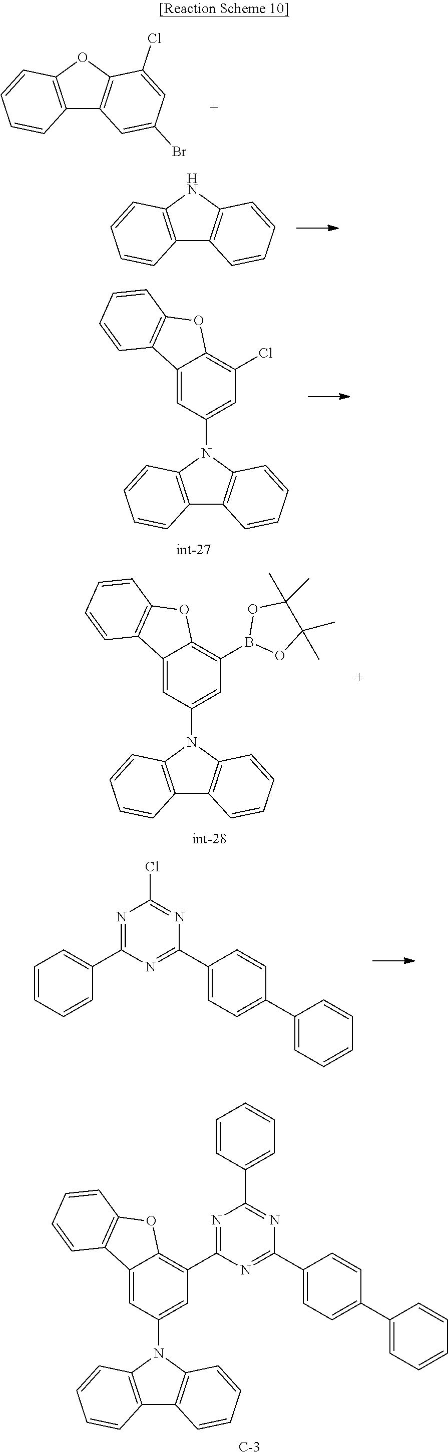

1 st step: Synthesis of Intermediate int-27

2-bromo-4-chlorodibenzofuran (30.3 g, 107.6 mmol), 9H-carbazole (18.0 g, 107.6 mmol), sodium tert-butoxide (20.7 g, 215.3 mmol), tri-tert-butylphosphine (2.2 g, 10.8 mmol), and Pd₂(dba)₃ (4.9 g, 5.4 mmol) were put in a round bottom flask and dissolved in xylene (550 ml) and then, stirred under reflux at 150° C. for 8 hours. When a reaction was completed, after removing a salt by filtration, a filtrate therefrom was adsorbed. 21.4 g (54%) of Intermediate int-27 was obtained through column chromatography (hexane:DCM (40%)).

2nd step: Synthesis of Intermediate int-28

Intermediate int-27 (21.2 g, 57.6 mmol), bis(pinacolato)diboron (17.6 g, 69.2 mmol), tricyclohexylphosphine (2.8 g, 11.5 mmol), potassium acetate (11.3 g, 115.3 mmol), and Pd₂(dba)₃ (1.6 g, 1.7 mmol) were put in a round bottom flask and dissolved in xylene (200 ml). The mixture was stirred under reflux at 160° C. for 8 hours. When a reaction was completed, the resultant was cooled to room temperature, filtered to remove a salt, and extracted with an excessive amount of DCM and distilled water. 23.3 g (88%) of Intermediate int-28 was obtained through column chromatography (hexane:DCM (40%)).

3rd step: Synthesis of Compound C-3

2-chloro-4-(biphenyl-4-yl)-6-phenyl-1,3,5-triazine (13.5 g, 39.3 mmol), Intermediate int-28 (18.9 g, 41.2 mmol), K₂CO₃ (10.9 g, 78.5 mmol), and Pd(PPh₃)₄ (2.3 g, 2.0 mmol) were put in a round bottom flask and dissolved in THF (150 ml) and distilled water (40 ml) and then, stirred under reflux at 70° C. for 12 hours. When a reaction was completed, a solid was separated therefrom by filtration and recrystallized with monochlorobenzene to obtain 18.1 g (72%) of Compound C-3.

### Comparative Synthesis Example 4: Synthesis of Compound C-4

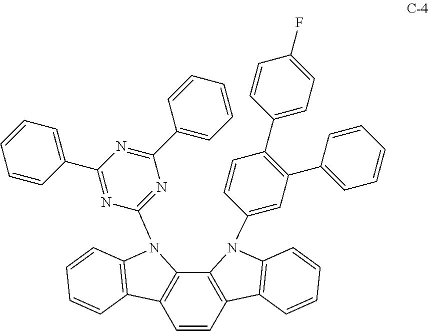 [SMILES: Fc1ccc(-c2ccc(-n3c4ccccc4c4ccc5c6ccccc6n(-c6nc(-c7ccccc7)nc(-c7ccccc7)n6)c5c43)cc2-c2ccccc2)cc1]

Compound C-4 was synthesized by referring to the synthesis method known in the CN 114075204 A published patent.

### Comparative Synthesis Example 5: Synthesis of Compound C-5

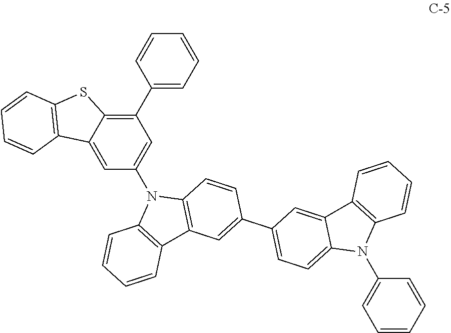 [SMILES: c1ccc(-c2cc(-n3c4ccccc4c4cc(-c5ccc6c(c5)c5ccccc5n6-c5ccccc5)ccc43)cc3c2sc2ccccc23)cc1]

Compound C-5 was synthesized by referring to the synthesis method known in the KR 2290362 B1 registered patent.

### Comparative Synthesis Example 6: Synthesis of Compound C-6

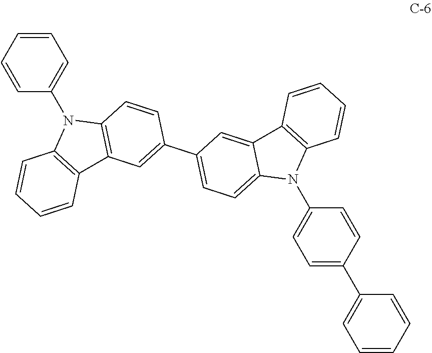 [SMILES: c1ccc(-c2ccc(-n3c4ccccc4c4cc(-c5ccc6c(c5)c5ccccc5n6-c5ccccc5)ccc43)cc2)cc1]

Compound C-6 was synthesized by referring to the synthesis method known in the KR 2022-0087827 published patent.

### (Manufacture of Organic Light Emitting Diode)

### Example 1

The glass substrate coated with ITO (Indium tin oxide) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited on the hole injection layer to form a 1350 Å-thick hole transport layer. On the hole transport layer, Compound B was deposited at a thickness of 350 Å to form a hole transport auxiliary layer. On the hole transport auxiliary layer, 330 Å-thick light emitting layer was formed by using Compound A-17 obtained in Synthesis Example 1 and Compound B-1 obtained in Synthesis Example 5 simultaneously as a host in a weight ratio of 4:6 and doping 7 wt % of PhGD as a dopant by vacuum-deposition.

Subsequently, on the light emitting layer, Compound C was deposited at a thickness of 50 Å to form an electron transport auxiliary layer and Compound D and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

ITO/Compound A (3% NDP-9 doping, 100 Å)/Compound A (1350 Å)/Compound B (350 Å)/EML [93 wt % of host (Compound A-17: Compound B-1=4:6 w/w): 7 wt % of PhGD] (330 Å)/Compound C (50 Å)/Compound D: LiQ (300 Å)/LiQ (15 Å)/Al (1200 Å)

### Compound A: N-(biphenyl-4-yl)-9,9-dimethyl-N-(4-(9-phenyl-9H-carbazol-3-yl)phenyl)-9H-fluoren-2-amine

### Compound B: N,N-bis(9,9-dimethyl-9H-fluoren-4-yl)-9,9-spirobi (fluorene)-2-amine

### Compound C: 2-[3′-(9,9-Dimethyl-9H-fluoren-2-yl) [1,1′-biphenyl]-3-yl]-4,6-diphenyl-1,3,5-triazine

### Compound D: 2-[4-[4-(4′-Cyano-1,1′-biphenyl-4-yl)-1-naphthyl]phenyl]-4,6-diphenyl-1,3,5-triazine

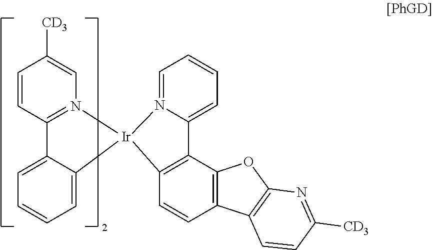

### Examples 2 to 8 and Comparative Examples 1 to 6

The diodes of Examples 2 to 8 and Comparative Examples 1 to 6 were manufactured in the same manner as Example 1, except that the host was changed as shown in Table 1.

### Evaluation

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Using the luminance and current density measured from (1) and (2) above, the luminous efficiency (cd/A) at the same current density (10 mA/cm²) was calculated.

The luminous efficiencies of Examples 1 to 8 and Comparative Examples 1 to 6 were calculated as relative values based on Comparative Example 1 and are listed in Table 1.

### (4) Measurement of Driving Voltage

The results were obtained by measuring the driving voltage of each diode at 15 mA/cm² using a current-voltage meter (Keithley 2400).

The driving voltages of Examples 1 to 8 and Comparative Examples 1 to 6 were calculated as relative values based on Comparative Example 1 and are listed in Table 1.

TABLE 1

| | | | Driving voltage | Efficiency |
|---|---|---|---|---|
| | First host | Second host | ratio (%) | ratio (%) |
| Example 1 | A-17 | B-1 | 94 | 110 |
| Example 2 | A-17 | B-36 | 92 | 109 |
| Example 3 | A-17 | B-71 | 96 | 107 |
| Example 4 | A-17 | B-106 | 93 | 105 |
| Example 5 | A-14 | B-1 | 96 | 109 |
| Example 6 | A-14 | B-36 | 94 | 108 |
| Example 7 | A-27 | B-36 | 93 | 104 |
| Example 8 | A-2 | B-36 | 92 | 107 |
| Comparative | C-1 | B-36 | 100 | 100 |
| Example 1 | | | | |
| Comparative | C-2 | B-71 | 104 | 97 |
| Example 2 | | | | |
| Comparative | C-3 | B-106 | 107 | 95 |
| Example 3 | | | | |
| Comparative | C-4 | B-106 | 109 | 91 |
| Example 4 | | | | |
| Comparative | A-17 | C-5 | 102 | 97 |
| Example 5 | | | | |
| Comparative | A-17 | C-6 | 99 | 100 |
| Example 6 | | | | |

Referring to Table 1 the organic light emitting diodes according to Examples 1 to 8 have significantly improved efficiency while maintaining a low driving voltage compared to the organic light emitting diodes according to Comparative Examples 1 to 6.

While this invention has been described in connection with what is presently considered to be practical example embodiments, it is to be understood that the invention is not limited to the disclosed embodiments. On the contrary, it is intended to cover various modifications and equivalent arrangements included within the spirit and scope of the appended claims.

1. A composition for an organic optoelectronic device, the composition comprising:
a first compound represented by Chemical Formula 1; and
a second compound represented by Chemical Formula 2: 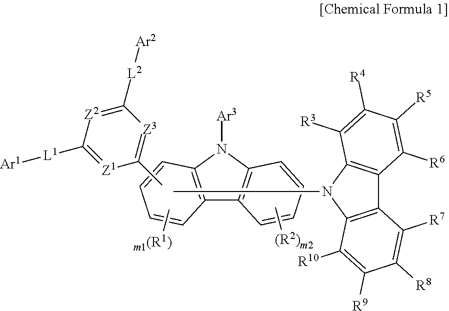
wherein, in Chemical Formula 1,
Z¹ to Z³ are each independently N or C—R^{a},
at least two of Z¹ to Z³ are N,
L¹ and L² are each independently a single bond or a substituted or unsubstituted C6 to C20 arylene group,
Ar¹ and Ar² are each independently a substituted or unsubstituted C6 to C30 aryl group or a substituted or unsubstituted C2 to C30 heterocyclic group,
Ar³ is a substituted or unsubstituted C6 to C20 aryl group,
R¹ and R² are each independently hydrogen, deuterium, a substituted or unsubstituted C1 to C10 alkyl group, a substituted or unsubstituted C6 to C20 aryl group, or a combination thereof,
m1 is an integer of 1 to 3,
m2 is an integer of 1 to 4, and
R^{a} and R³ to R¹⁰ are each independently hydrogen, deuterium, a substituted or unsubstituted C1 to C10 alkyl group, a substituted or unsubstituted C6 to C30 aryl group, a substituted or unsubstituted C2 to C30 heterocyclic group, or a combination thereof, 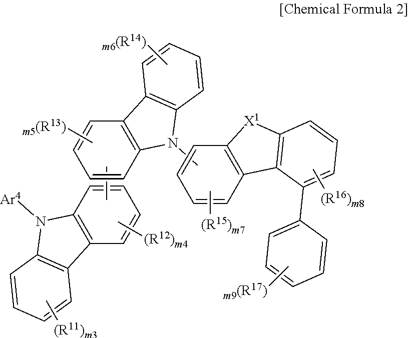
wherein, in Chemical Formula 2,
X¹ is O or S,
Ar⁴ is a substituted or unsubstituted C6 to C30 aryl group, a substituted or unsubstituted C2 to C30 heterocyclic group, or a combination thereof,
R¹¹ to R¹⁴ are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
R¹⁵ to R¹⁷ are each independently hydrogen, deuterium, or a substituted or unsubstituted phenyl group,
m4, m5, m7, and m8 are each independently an integer of 1 to 3, and
m3, m6, and m9 are each independently an integer of 1 to 4.

2. The composition for the organic optoelectronic device of claim 1, wherein:
Chemical Formula 1 is represented by Chemical Formula 1A or Chemical Formula 1B: 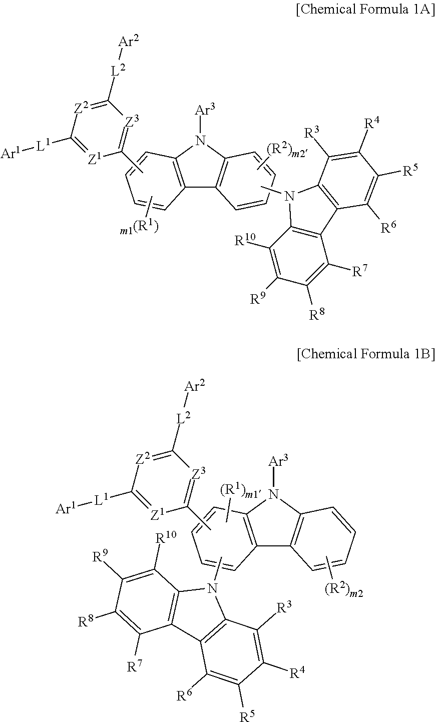
in Chemical Formula 1A and Chemical Formula 1B,
Z¹ to Z³, L¹, L², Ar¹ to Ar³, R¹ to R¹⁰, ml, and m2 are defined the same as those of Chemical Formula 1,
m1′ is an integer of 1 or 2, and
m2′ is an integer of 1 to 3.

3. The composition for the organic optoelectronic device of claim 2, wherein;
Chemical Formula 1A is represented by Chemical Formula 1A-1 to Chemical Formula 1A-4: 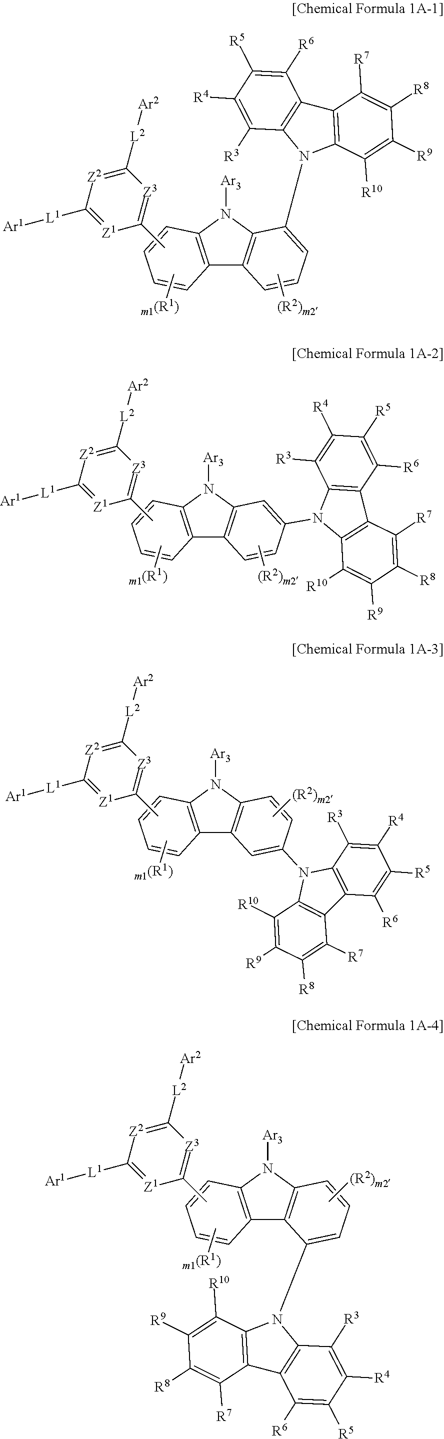
in Chemical Formula 1A-1 to Chemical Formula 1A-4, Z¹ to Z³, L¹, L², Ar¹ to Ar³, R¹ to R¹⁰, ml, and m2′ are defined the same as those of Chemical Formula 1.

4. The composition for the organic optoelectronic device of claim 2, wherein;
Chemical Formula 1B is represented by one of Chemical Formula 1B-1 to Chemical Formula 1B-4: 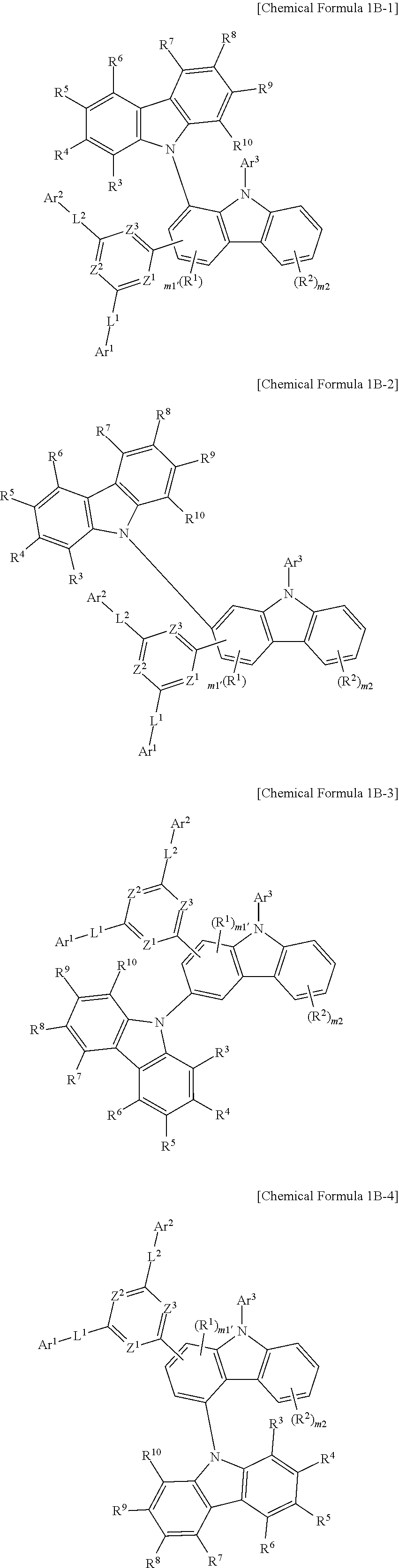
in Chemical Formula 1B-1 to Chemical Formula 1B-4, Z¹ to Z³, L¹, L², Ar¹ to Ar³, R¹ to R¹⁰, m1′, and m2 are defined the same as those of Chemical Formula 1.

5. The composition for the organic optoelectronic device of claim 1, wherein:
Chemical Formula 1 is represented by one of Chemical Formula 1A-1-2, Chemical Formula 1A-2-2, Chemical Formula 1A-3-2, Chemical Formula 1A-4-2, Chemical Formula 1B-1-2, Chemical Formula 1B-2-2, Chemical Formula 1B-3-2, or Chemical Formula 1B-4-2: 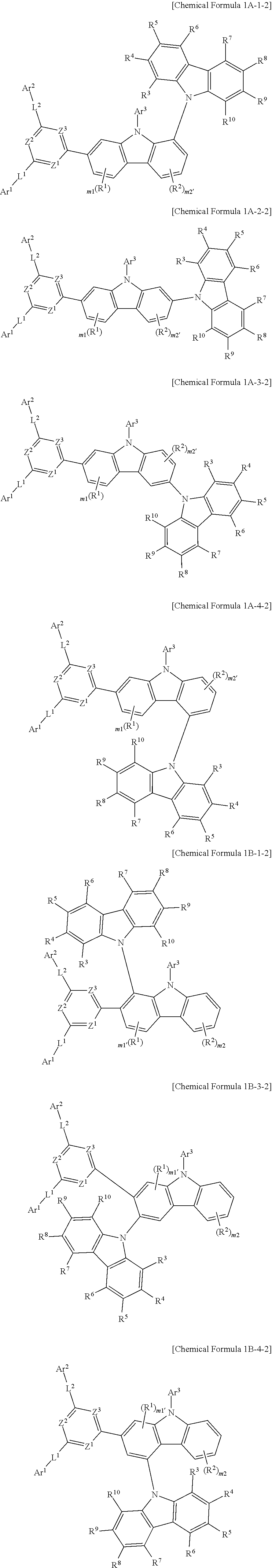
in Chemical Formula 1A-1-2, Chemical Formula 1A-2-2, Chemical Formula 1A-3-2, Chemical Formula 1A-4-2, Chemical Formula 1B-1-2, Chemical Formula 1B-2-2, Chemical Formula 1B-3-2, and Chemical Formula 1B-4-2,
Z¹ to Z³, L¹, L², Ar¹ to Ar³, R¹ to R¹⁰, ml, and m2 are defined the same as those of Chemical Formula 2,
m1′ is an integer of 1 or 2, and
m2′ is an integer of 1 to 3.

6. The composition for the organic optoelectronic device of claim 1, wherein Ar¹ and Ar² in Chemical Formula 1 are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, or a substituted or unsubstituted dibenzothiophenyl group.

7. The composition for the organic optoelectronic device of claim 1, wherein R³ to R⁸ in Chemical Formula 1 are each independently hydrogen, deuterium, or a substituted or unsubstituted C6 to C20 aryl group.

8. The composition for the organic optoelectronic device of claim 1, wherein:
the second compound is represented by one of Chemical Formula 2-1 to Chemical Formula 2-4: 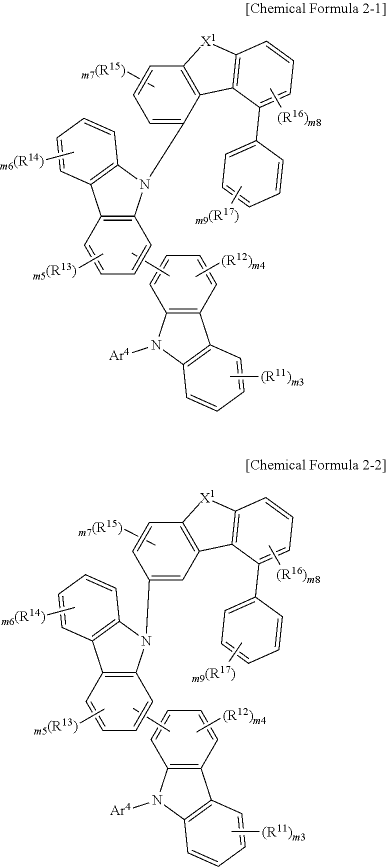 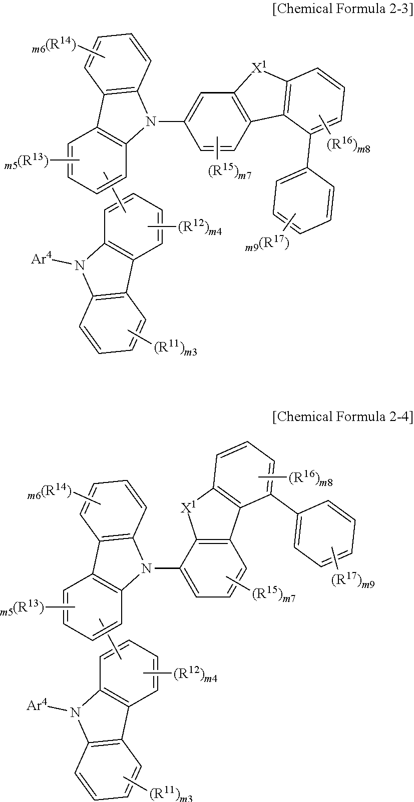
in Chemical Formula 2-1 to Chemical Formula 2-4, X¹, Ar⁴, R¹¹ to R¹⁷, and m3 to m9 are defined the same as those of Chemical Formula 2.

9. The composition for the organic optoelectronic device of claim 8, wherein;
the second compound is represented by one of Chemical Formula 2-1-11, Chemical Formula 2-2-11, Chemical Formula 2-3-11, or Chemical Formula 2-4-11: 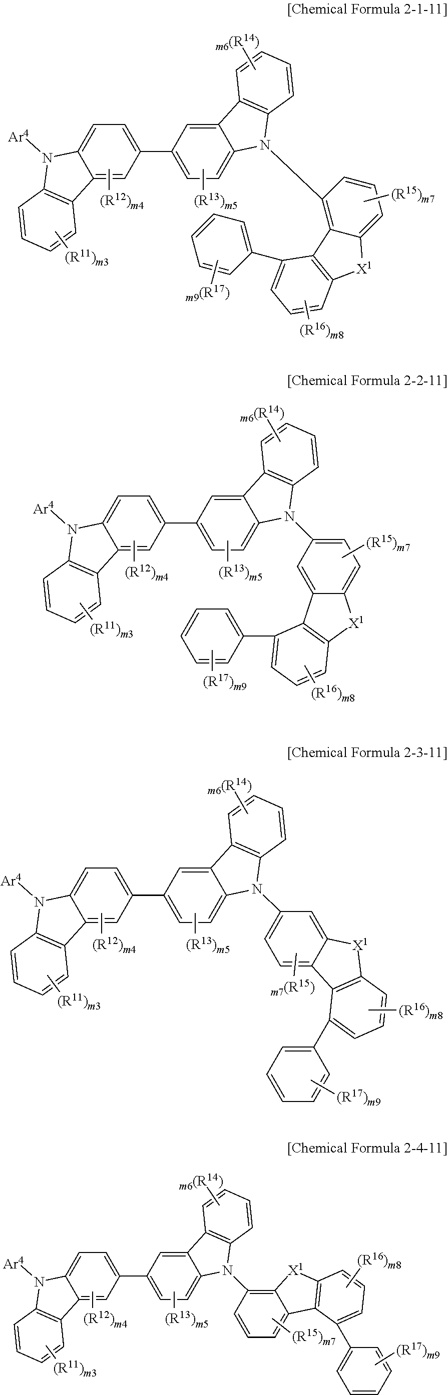
wherein, in Chemical Formula 2-1-11, Chemical Formula 2-2-11, Chemical Formula 2-3-11, and Chemical Formula 2-4-11, X¹, Ar⁴, R¹¹ to R¹⁷, and m3 to m9 are defined the same as those of Chemical Formula 2.

10. The composition for the organic optoelectronic device of claim 1, wherein Ar⁴ in Chemical Formula 2 is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, or a substituted or unsubstituted fluorenyl group.

11. The composition for the organic optoelectronic device of claim 1, wherein:
the first compound is a compound of Group 1, and
the second compound is a compound of Group 2:

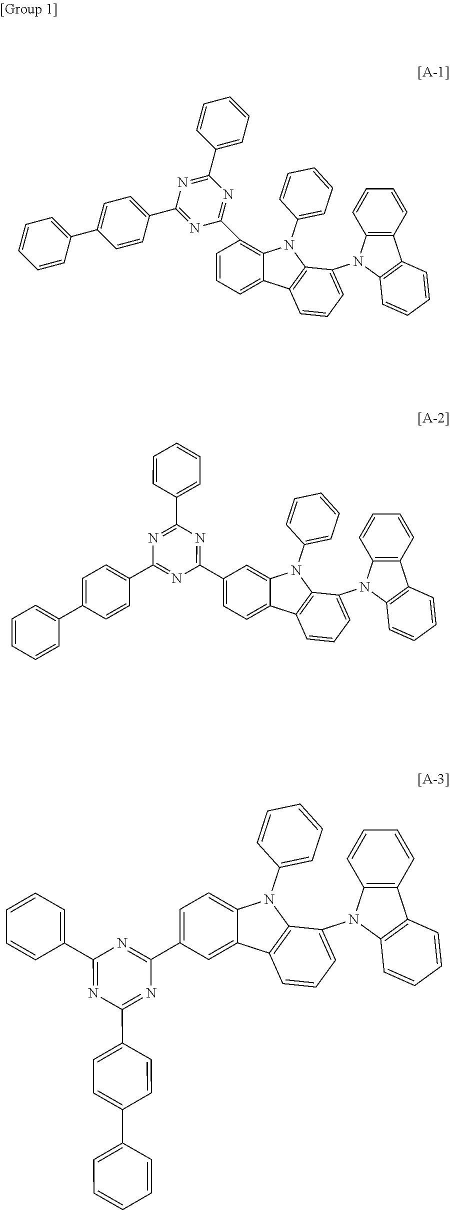 [SMILES: c1ccc(-c2ccc(-c3nc(-c4ccccc4)nc(-c4cccc5c6cccc(-n7c8ccccc8c8ccccc87)c6n(-c6ccccc6)c45)n3)cc2)cc1 ; c1ccc(-c2ccc(-c3nc(-c4ccccc4)nc(-c4ccc5c6cccc(-n7c8ccccc8c8ccccc87)c6n(-c6ccccc6)c5c4)n3)cc2)cc1 ; c1ccc(-c2ccc(-c3nc(-c4ccccc4)nc(-c4ccc5c(c4)c4cccc(-n6c7ccccc7c7ccccc76)c4n5-c4ccccc4)n3)cc2)cc1]

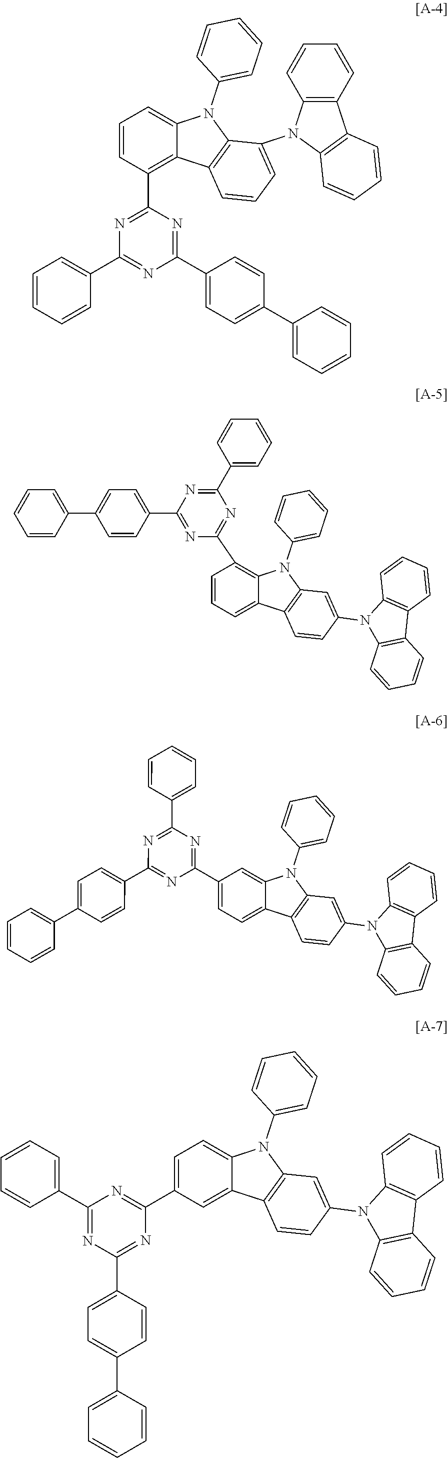 [SMILES: c1ccc(-c2ccc(-c3nc(-c4ccccc4)nc(-c4cccc5c4c4cccc(-n6c7ccccc7c7ccccc76)c4n5-c4ccccc4)n3)cc2)cc1 ; c1ccc(-c2ccc(-c3nc(-c4ccccc4)nc(-c4cccc5c6ccc(-n7c8ccccc8c8ccccc87)cc6n(-c6ccccc6)c45)n3)cc2)cc1 ; c1ccc(-c2ccc(-c3nc(-c4ccccc4)nc(-c4ccc5c6ccc(-n7c8ccccc8c8ccccc87)cc6n(-c6ccccc6)c5c4)n3)cc2)cc1 ; c1ccc(-c2ccc(-c3nc(-c4ccccc4)nc(-c4ccc5c(c4)c4ccc(-n6c7ccccc7c7ccccc76)cc4n5-c4ccccc4)n3)cc2)cc1]

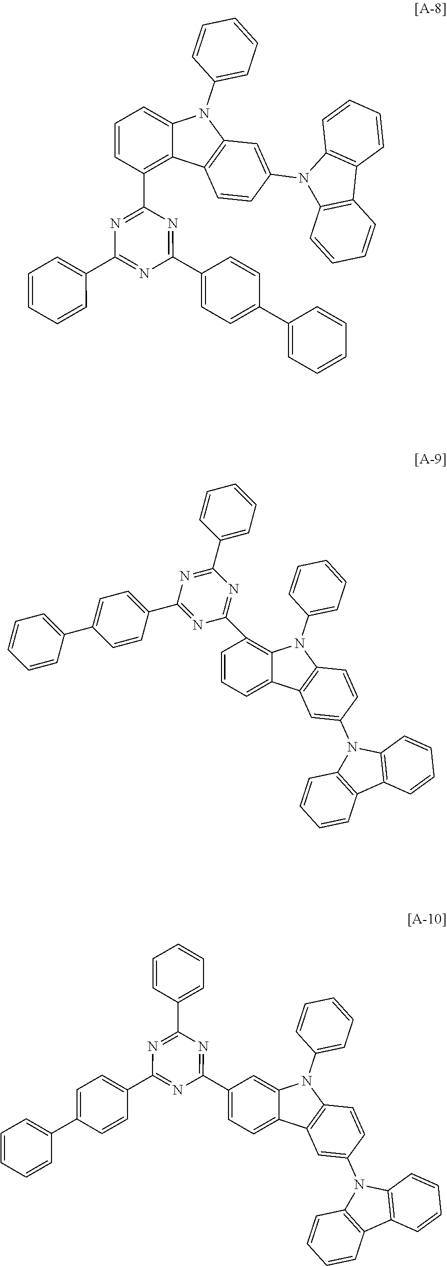 [SMILES: c1ccc(-c2ccc(-c3nc(-c4ccccc4)nc(-c4cccc5c4c4ccc(-n6c7ccccc7c7ccccc76)cc4n5-c4ccccc4)n3)cc2)cc1 ; c1ccc(-c2ccc(-c3nc(-c4ccccc4)nc(-c4cccc5c6cc(-n7c8ccccc8c8ccccc87)ccc6n(-c6ccccc6)c45)n3)cc2)cc1 ; c1ccc(-c2ccc(-c3nc(-c4ccccc4)nc(-c4ccc5c6cc(-n7c8ccccc8c8ccccc87)ccc6n(-c6ccccc6)c5c4)n3)cc2)cc1]

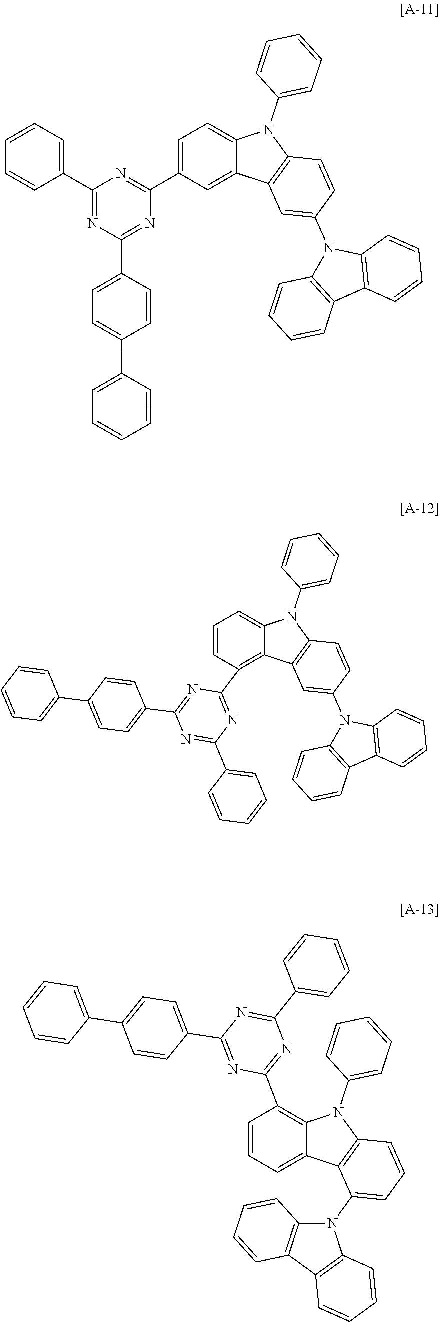 [SMILES: c1ccc(-c2ccc(-c3nc(-c4ccccc4)nc(-c4ccc5c(c4)c4cc(-n6c7ccccc7c7ccccc76)ccc4n5-c4ccccc4)n3)cc2)cc1 ; c1ccc(-c2ccc(-c3nc(-c4ccccc4)nc(-c4cccc5c4c4cc(-n6c7ccccc7c7ccccc76)ccc4n5-c4ccccc4)n3)cc2)cc1 ; c1ccc(-c2ccc(-c3nc(-c4ccccc4)nc(-c4cccc5c6c(-n7c8ccccc8c8ccccc87)cccc6n(-c6ccccc6)c45)n3)cc2)cc1]

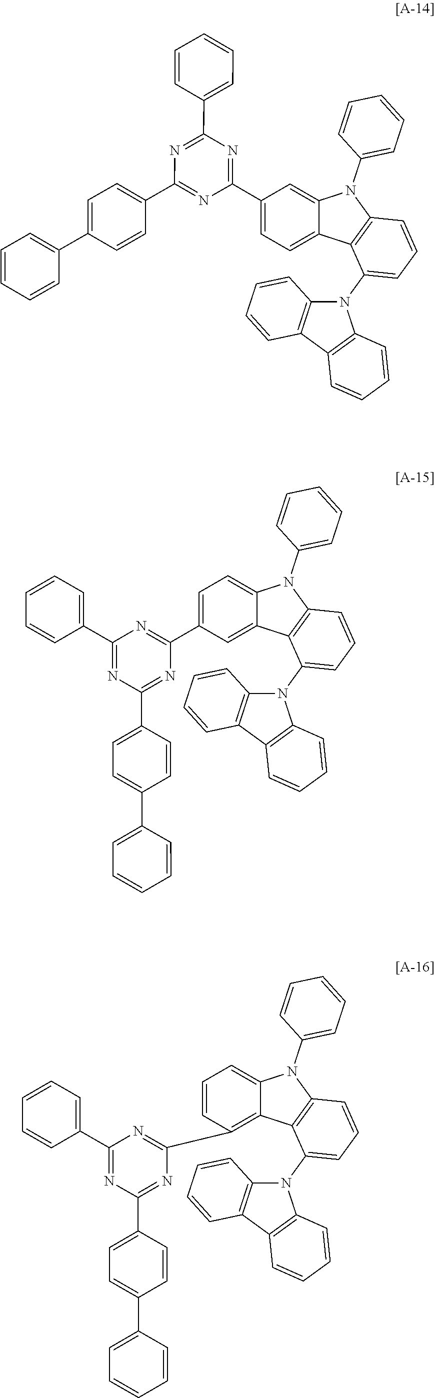 [SMILES: c1ccc(-c2ccc(-c3nc(-c4ccccc4)nc(-c4ccc5c6c(-n7c8ccccc8c8ccccc87)cccc6n(-c6ccccc6)c5c4)n3)cc2)cc1 ; c1ccc(-c2ccc(-c3nc(-c4ccccc4)nc(-c4ccc5c(c4)c4c(-n6c7ccccc7c7ccccc76)cccc4n5-c4ccccc4)n3)cc2)cc1 ; c1ccc(-c2ccc(-c3nc(-c4ccccc4)nc(-c4cccc5c4c4c(-n6c7ccccc7c7ccccc76)cccc4n5-c4ccccc4)n3)cc2)cc1]

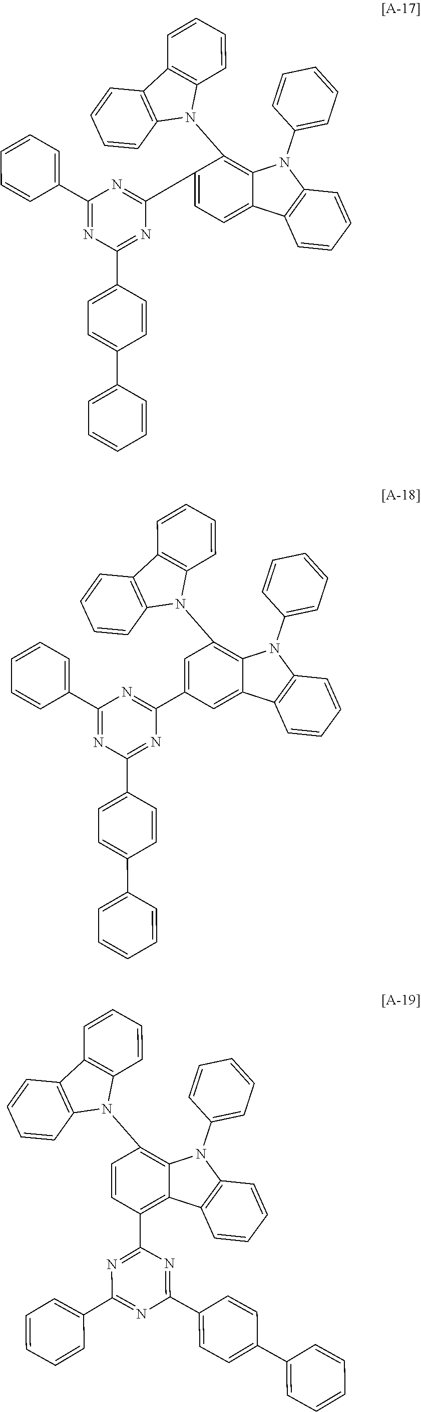 [SMILES: c1ccc(-c2ccc(-c3nc(-c4ccccc4)nc(-c4ccc5c6ccccc6n(-c6ccccc6)c5c4-n4c5ccccc5c5ccccc54)n3)cc2)cc1 ; c1ccc(-c2ccc(-c3nc(-c4ccccc4)nc(-c4cc(-n5c6ccccc6c6ccccc65)c5c(c4)c4ccccc4n5-c4ccccc4)n3)cc2)cc1 ; c1ccc(-c2ccc(-c3nc(-c4ccccc4)nc(-c4ccc(-n5c6ccccc6c6ccccc65)c5c4c4ccccc4n5-c4ccccc4)n3)cc2)cc1]

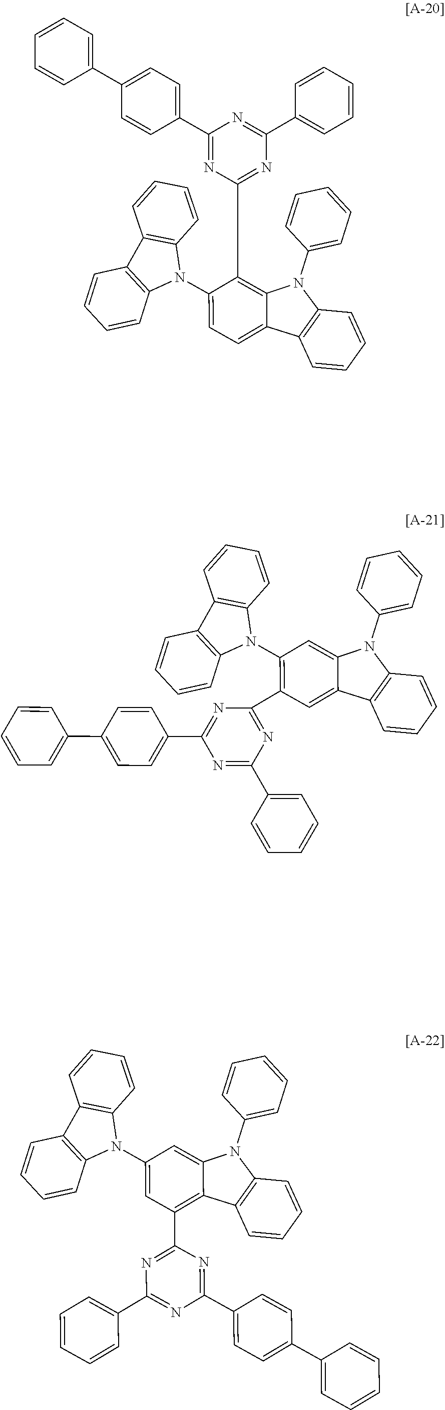 [SMILES: c1ccc(-c2ccc(-c3nc(-c4ccccc4)nc(-c4c(-n5c6ccccc6c6ccccc65)ccc5c6ccccc6n(-c6ccccc6)c45)n3)cc2)cc1 ; c1ccc(-c2ccc(-c3nc(-c4ccccc4)nc(-c4cc5c6ccccc6n(-c6ccccc6)c5cc4-n4c5ccccc5c5ccccc54)n3)cc2)cc1 ; c1ccc(-c2ccc(-c3nc(-c4ccccc4)nc(-c4cc(-n5c6ccccc6c6ccccc65)cc5c4c4ccccc4n5-c4ccccc4)n3)cc2)cc1]

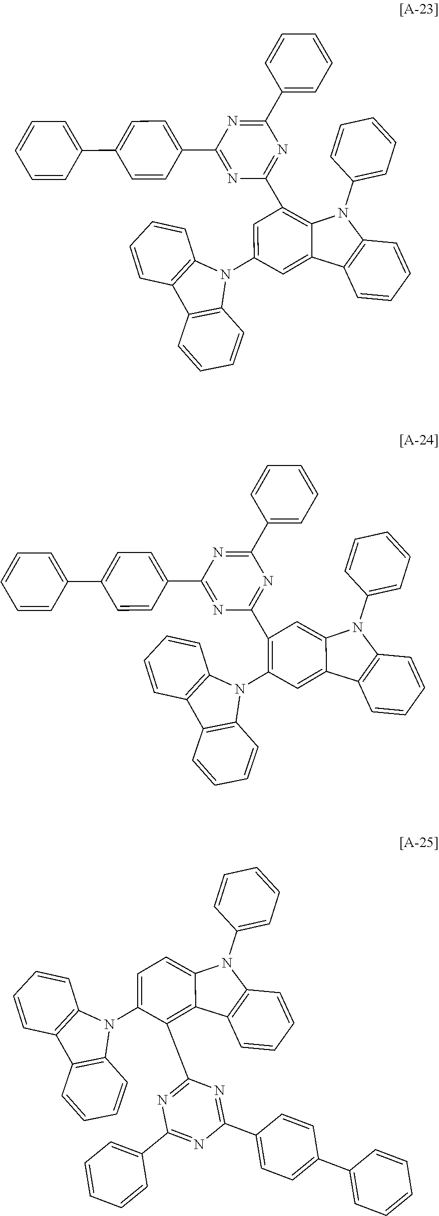 [SMILES: c1ccc(-c2ccc(-c3nc(-c4ccccc4)nc(-c4cc(-n5c6ccccc6c6ccccc65)cc5c6ccccc6n(-c6ccccc6)c45)n3)cc2)cc1 ; c1ccc(-c2ccc(-c3nc(-c4ccccc4)nc(-c4cc5c(cc4-n4c6ccccc6c6ccccc64)c4ccccc4n5-c4ccccc4)n3)cc2)cc1 ; c1ccc(-c2ccc(-c3nc(-c4ccccc4)nc(-c4c(-n5c6ccccc6c6ccccc65)ccc5c4c4ccccc4n5-c4ccccc4)n3)cc2)cc1]

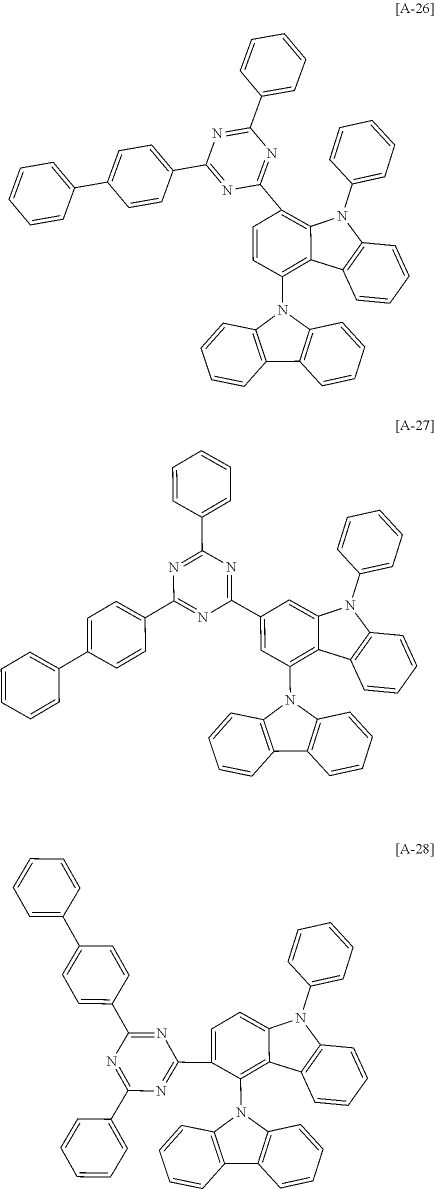 [SMILES: c1ccc(-c2ccc(-c3nc(-c4ccccc4)nc(-c4ccc(-n5c6ccccc6c6ccccc65)c5c6ccccc6n(-c6ccccc6)c45)n3)cc2)cc1 ; c1ccc(-c2ccc(-c3nc(-c4ccccc4)nc(-c4cc(-n5c6ccccc6c6ccccc65)c5c6ccccc6n(-c6ccccc6)c5c4)n3)cc2)cc1 ; c1ccc(-c2ccc(-c3nc(-c4ccccc4)nc(-c4ccc5c(c4-n4c6ccccc6c6ccccc64)c4ccccc4n5-c4ccccc4)n3)cc2)cc1]

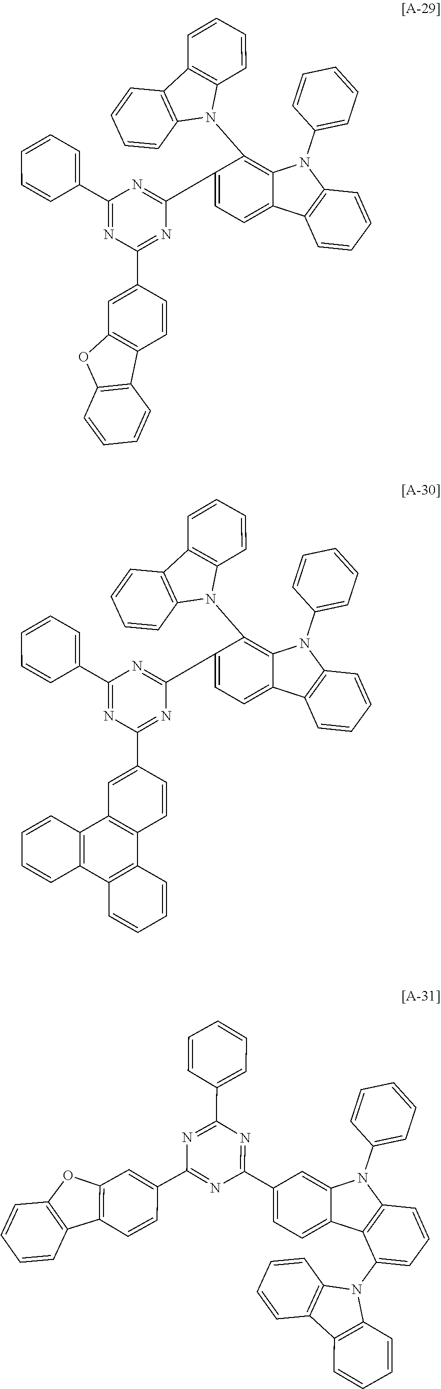 [SMILES: c1ccc(-c2nc(-c3ccc4c(c3)oc3ccccc34)nc(-c3ccc4c5ccccc5n(-c5ccccc5)c4c3-n3c4ccccc4c4ccccc43)n2)cc1 ; c1ccc(-c2nc(-c3ccc4c5ccccc5c5ccccc5c4c3)nc(-c3ccc4c5ccccc5n(-c5ccccc5)c4c3-n3c4ccccc4c4ccccc43)n2)cc1 ; c1ccc(-c2nc(-c3ccc4c(c3)oc3ccccc34)nc(-c3ccc4c5c(-n6c7ccccc7c7ccccc76)cccc5n(-c5ccccc5)c4c3)n2)cc1]

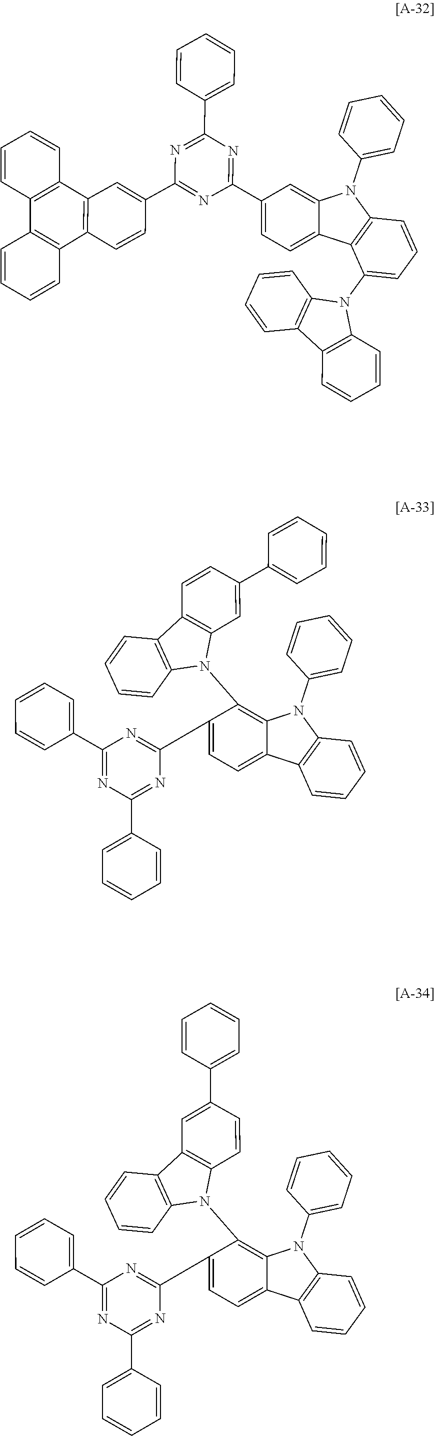 [SMILES: c1ccc(-c2nc(-c3ccc4c5ccccc5c5ccccc5c4c3)nc(-c3ccc4c5c(-n6c7ccccc7c7ccccc76)cccc5n(-c5ccccc5)c4c3)n2)cc1 ; c1ccc(-c2ccc3c4ccccc4n(-c4c(-c5nc(-c6ccccc6)nc(-c6ccccc6)n5)ccc5c6ccccc6n(-c6ccccc6)c45)c3c2)cc1 ; c1ccc(-c2ccc3c(c2)c2ccccc2n3-c2c(-c3nc(-c4ccccc4)nc(-c4ccccc4)n3)ccc3c4ccccc4n(-c4ccccc4)c23)cc1]

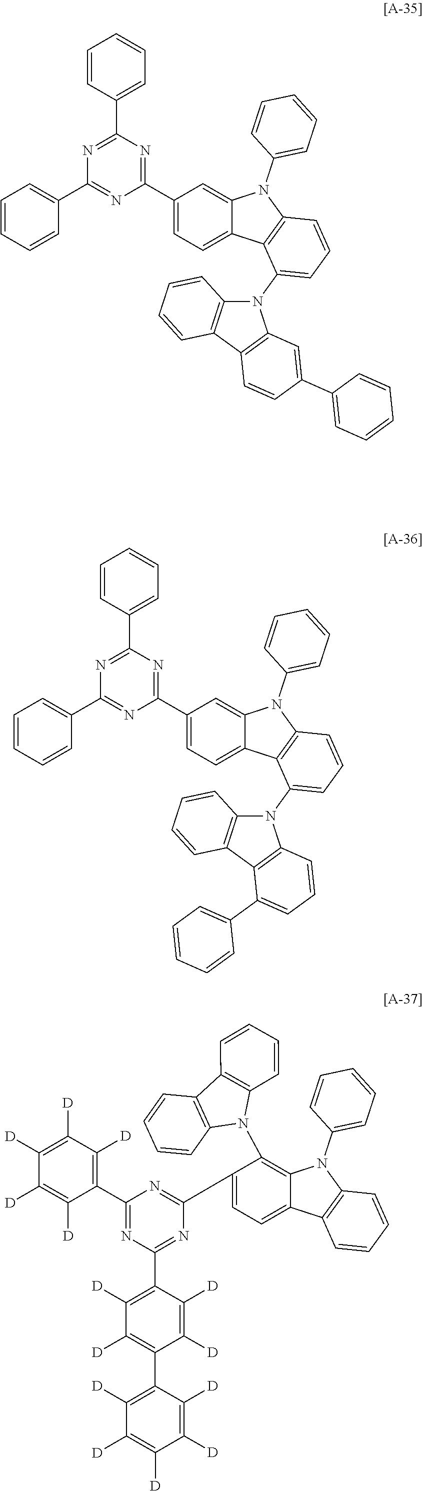 [SMILES: c1ccc(-c2ccc3c4ccccc4n(-c4cccc5c4c4ccc(-c6nc(-c7ccccc7)nc(-c7ccccc7)n6)cc4n5-c4ccccc4)c3c2)cc1 ; c1ccc(-c2nc(-c3ccccc3)nc(-c3ccc4c5c(-n6c7ccccc7c7c(-c8ccccc8)cccc76)cccc5n(-c5ccccc5)c4c3)n2)cc1 ; [2H]c1c([2H])c([2H])c(-c2nc(-c3ccc4c5ccccc5n(-c5ccccc5)c4c3-n3c4ccccc4c4ccccc43)nc(-c3c([2H])c([2H])c(-c4c([2H])c([2H])c([2H])c([2H])c4[2H])c([2H])c3[2H])n2)c([2H])c1[2H]]

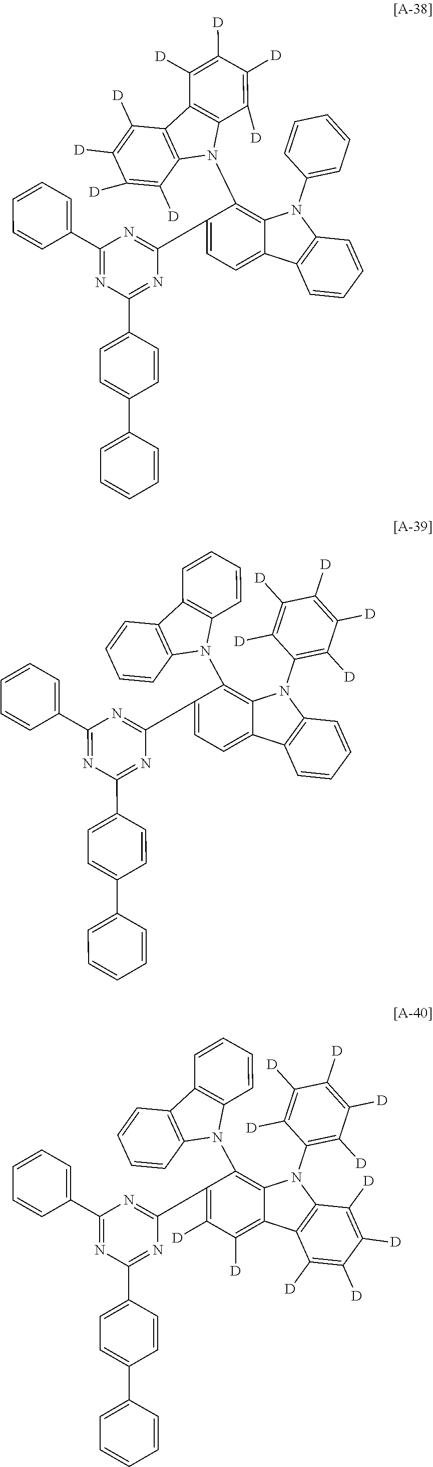 [SMILES: [2H]c1c([2H])c([2H])c2c(c1[2H])c1c([2H])c([2H])c([2H])c([2H])c1n2-c1c(-c2nc(-c3ccccc3)nc(-c3ccc(-c4ccccc4)cc3)n2)ccc2c3ccccc3n(-c3ccccc3)c12 ; [2H]c1c([2H])c([2H])c(-n2c3ccccc3c3ccc(-c4nc(-c5ccccc5)nc(-c5ccc(-c6ccccc6)cc5)n4)c(-n4c5ccccc5c5ccccc54)c32)c([2H])c1[2H] ; [2H]c1c([2H])c([2H])c(-n2c3c([2H])c([2H])c([2H])c([2H])c3c3c([2H])c([2H])c(-c4nc(-c5ccccc5)nc(-c5ccc(-c6ccccc6)cc5)n4)c(-n4c5ccccc5c5ccccc54)c32)c([2H])c1[2H]]

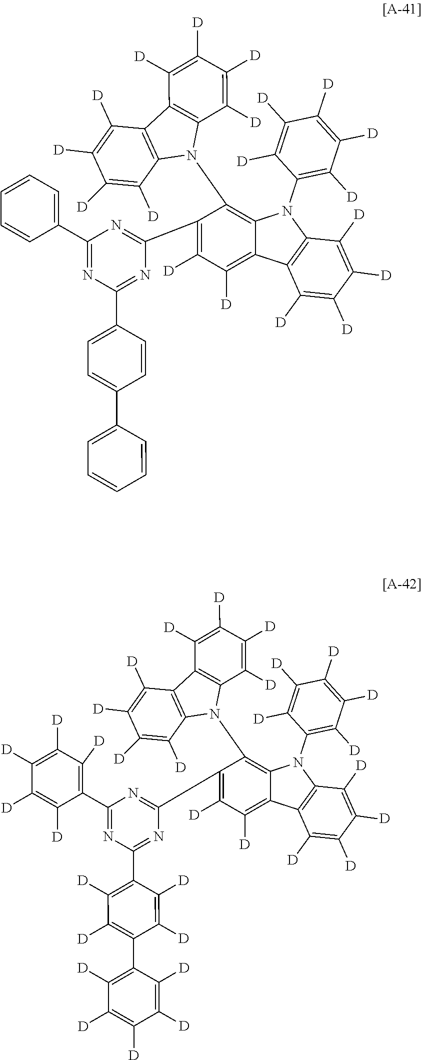 [SMILES: [2H]c1c([2H])c([2H])c(-n2c3c([2H])c([2H])c([2H])c([2H])c3c3c([2H])c([2H])c(-c4nc(-c5ccccc5)nc(-c5ccc(-c6ccccc6)cc5)n4)c(-n4c5c([2H])c([2H])c([2H])c([2H])c5c5c([2H])c([2H])c([2H])c([2H])c54)c32)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c4c5c([2H])c([2H])c([2H])c([2H])c5n(-c5c([2H])c([2H])c([2H])c([2H])c5[2H])c4c3-n3c4c([2H])c([2H])c([2H])c([2H])c4c4c([2H])c([2H])c([2H])c([2H])c43)n2)c([2H])c1[2H]]

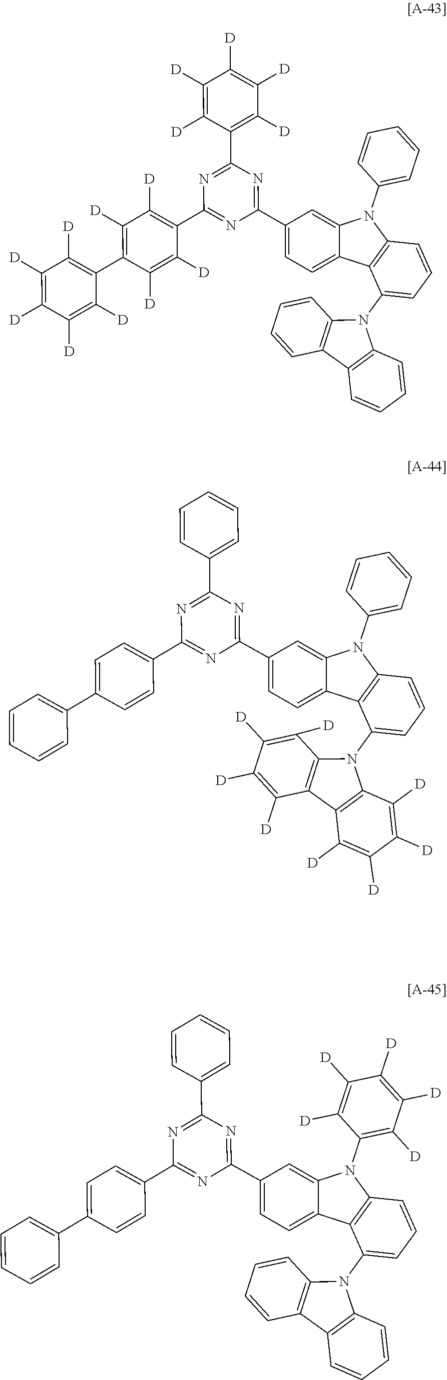 [SMILES: [2H]c1c([2H])c([2H])c(-c2nc(-c3ccc4c5c(-n6c7ccccc7c7ccccc76)cccc5n(-c5ccccc5)c4c3)nc(-c3c([2H])c([2H])c(-c4c([2H])c([2H])c([2H])c([2H])c4[2H])c([2H])c3[2H])n2)c([2H])c1[2H] ; [2H]c1c([2H])c([2H])c2c(c1[2H])c1c([2H])c([2H])c([2H])c([2H])c1n2-c1cccc2c1c1ccc(-c3nc(-c4ccccc4)nc(-c4ccc(-c5ccccc5)cc4)n3)cc1n2-c1ccccc1 ; [2H]c1c([2H])c([2H])c(-n2c3cc(-c4nc(-c5ccccc5)nc(-c5ccc(-c6ccccc6)cc5)n4)ccc3c3c(-n4c5ccccc5c5ccccc54)cccc32)c([2H])c1[2H]]

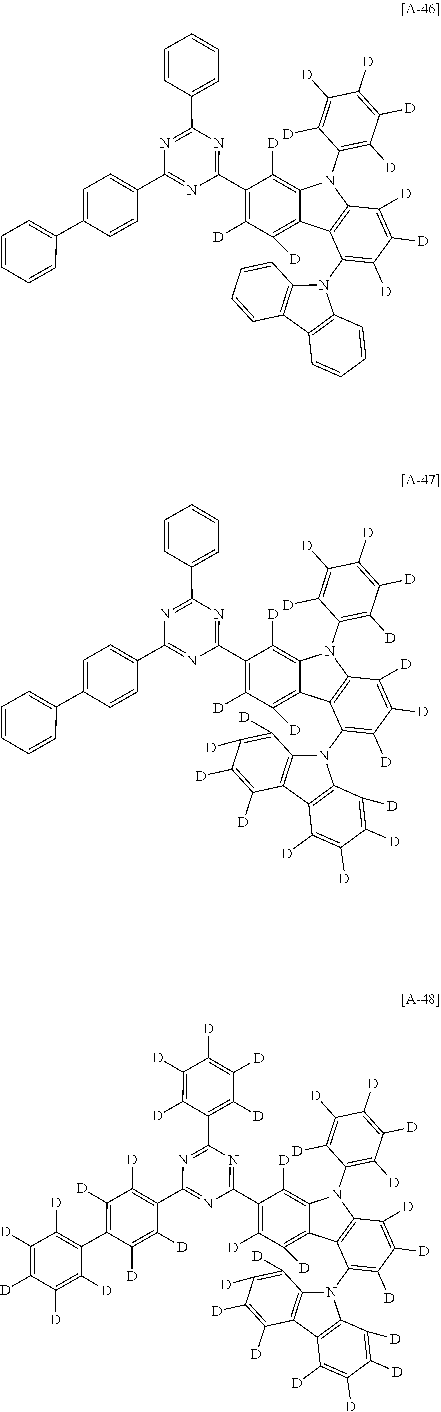

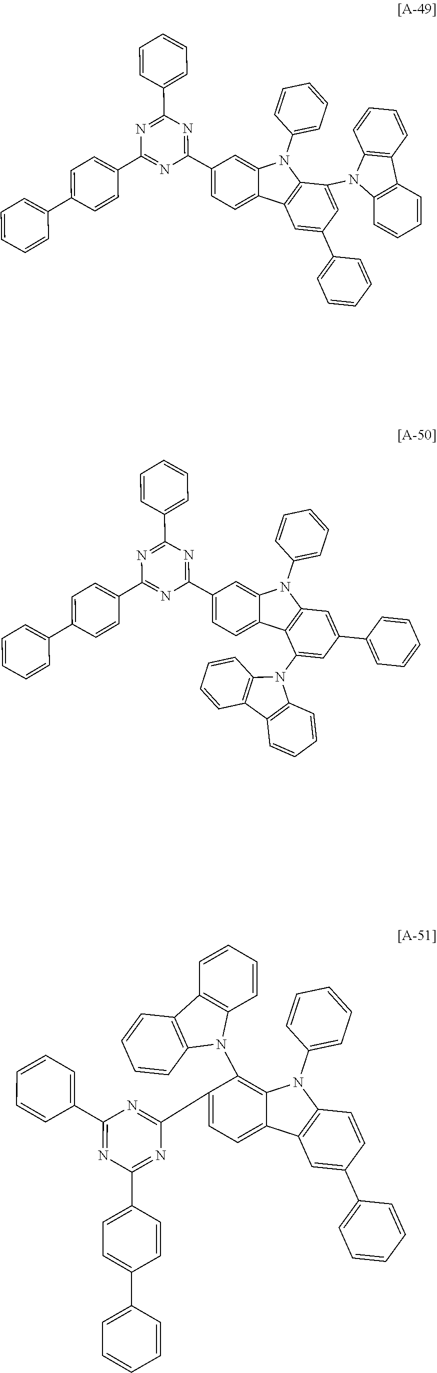 [SMILES: c1ccc(-c2ccc(-c3nc(-c4ccccc4)nc(-c4ccc5c6cc(-c7ccccc7)cc(-n7c8ccccc8c8ccccc87)c6n(-c6ccccc6)c5c4)n3)cc2)cc1 ; c1ccc(-c2ccc(-c3nc(-c4ccccc4)nc(-c4ccc5c6c(-n7c8ccccc8c8ccccc87)cc(-c7ccccc7)cc6n(-c6ccccc6)c5c4)n3)cc2)cc1 ; c1ccc(-c2ccc(-c3nc(-c4ccccc4)nc(-c4ccc5c6cc(-c7ccccc7)ccc6n(-c6ccccc6)c5c4-n4c5ccccc5c5ccccc54)n3)cc2)cc1]

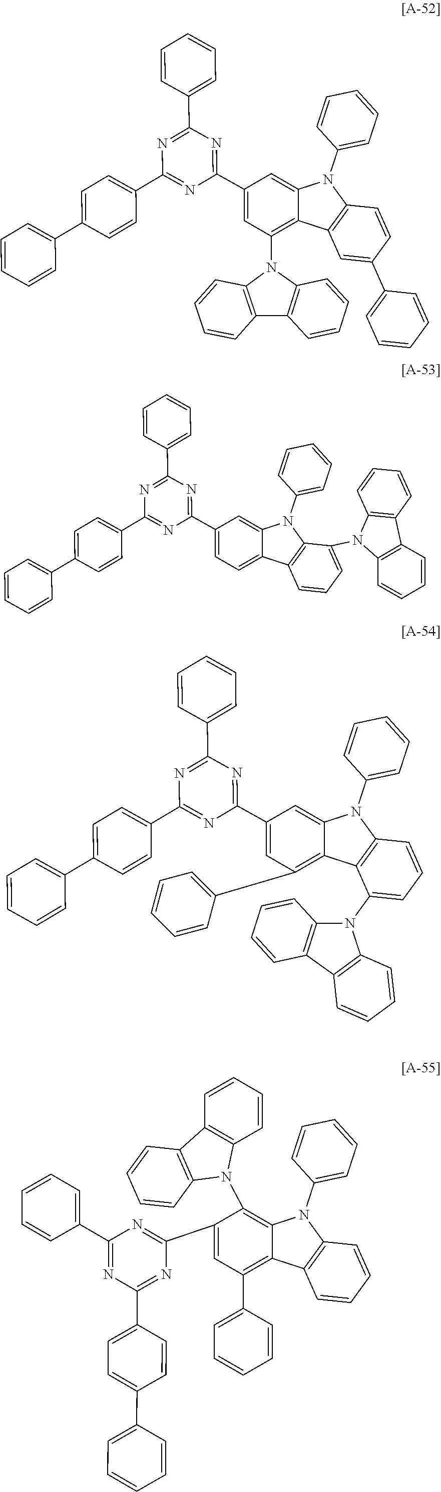 [SMILES: c1ccc(-c2ccc(-c3nc(-c4ccccc4)nc(-c4cc(-n5c6ccccc6c6ccccc65)c5c6cc(-c7ccccc7)ccc6n(-c6ccccc6)c5c4)n3)cc2)cc1 ; c1ccc(-c2ccc(-c3nc(-c4ccccc4)nc(-c4ccc5c6cccc(-n7c8ccccc8c8ccccc87)c6n(-c6ccccc6)c5c4)n3)cc2)cc1 ; c1ccc(-c2ccc(-c3nc(-c4ccccc4)nc(-c4cc(-c5ccccc5)c5c6c(-n7c8ccccc8c8ccccc87)cccc6n(-c6ccccc6)c5c4)n3)cc2)cc1 ; c1ccc(-c2ccc(-c3nc(-c4ccccc4)nc(-c4cc(-c5ccccc5)c5c6ccccc6n(-c6ccccc6)c5c4-n4c5ccccc5c5ccccc54)n3)cc2)cc1]

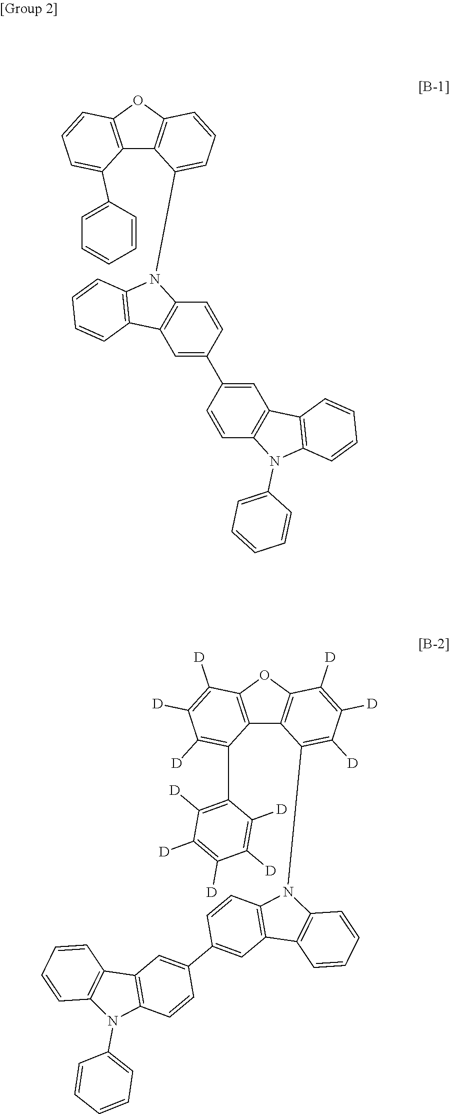 [SMILES: c1ccc(-c2cccc3oc4cccc(-n5c6ccccc6c6cc(-c7ccc8c(c7)c7ccccc7n8-c7ccccc7)ccc65)c4c23)cc1 ; [2H]c1c([2H])c([2H])c(-c2c([2H])c([2H])c([2H])c3oc4c([2H])c([2H])c([2H])c(-n5c6ccccc6c6cc(-c7ccc8c(c7)c7ccccc7n8-c7ccccc7)ccc65)c4c23)c([2H])c1[2H]]

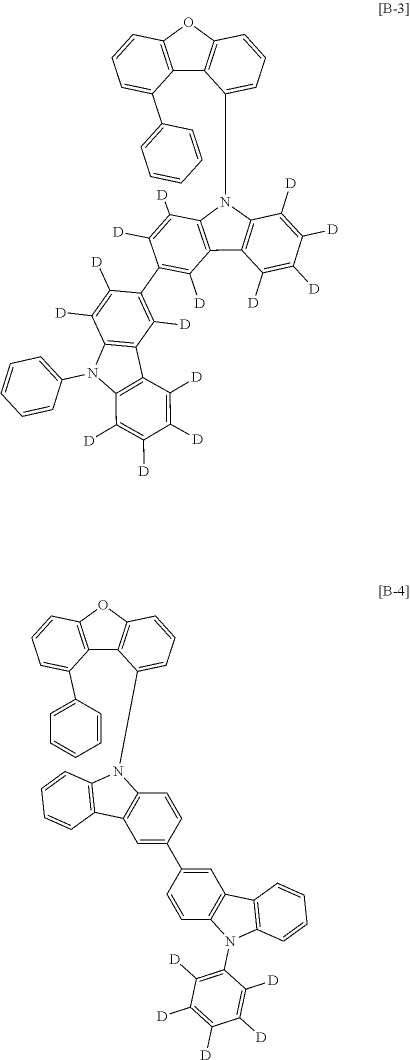 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cccc(-c6ccccc6)c5c34)c([2H])c([2H])c1n2-c1ccccc1 ; [2H]c1c([2H])c([2H])c(-n2c3ccccc3c3cc(-c4ccc5c(c4)c4ccccc4n5-c4cccc5oc6cccc(-c7ccccc7)c6c45)ccc32)c([2H])c1[2H]]

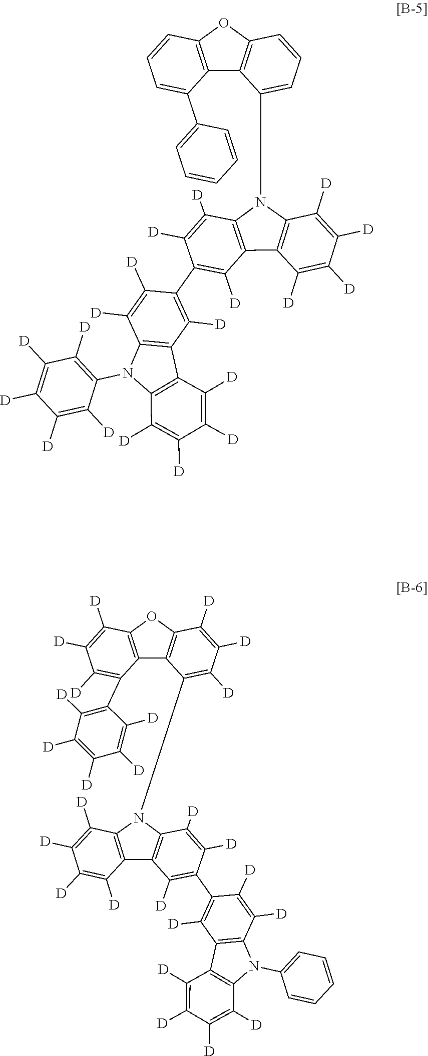 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5oc6cccc(-c7ccccc7)c6c45)c([2H])c([2H])c32)c([2H])c1[2H] ; [2H]c1c([2H])c([2H])c(-c2c([2H])c([2H])c([2H])c3oc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c4c23)c([2H])c1[2H]]

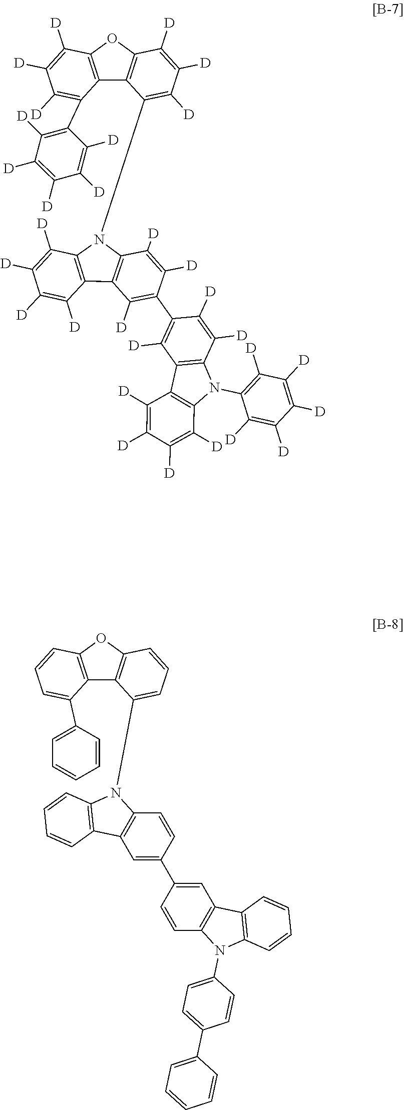 [SMILES: [2H]c1c([2H])c([2H])c(-c2c([2H])c([2H])c([2H])c3oc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c4c23)c([2H])c1[2H] ; c1ccc(-c2ccc(-n3c4ccccc4c4cc(-c5ccc6c(c5)c5ccccc5n6-c5cccc6oc7cccc(-c8ccccc8)c7c56)ccc43)cc2)cc1]

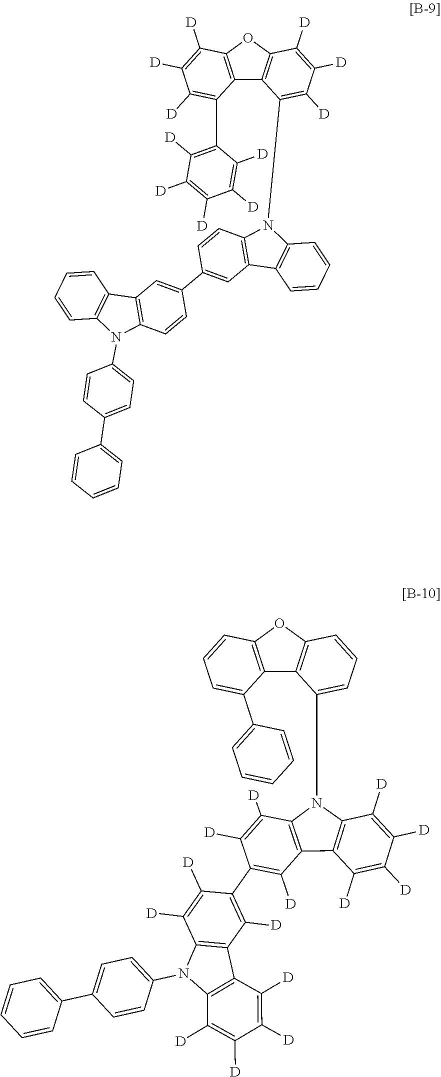 [SMILES: [2H]c1c([2H])c([2H])c(-c2c([2H])c([2H])c([2H])c3oc4c([2H])c([2H])c([2H])c(-n5c6ccccc6c6cc(-c7ccc8c(c7)c7ccccc7n8-c7ccc(-c8ccccc8)cc7)ccc65)c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cccc(-c6ccccc6)c5c34)c([2H])c([2H])c1n2-c1ccc(-c2ccccc2)cc1]

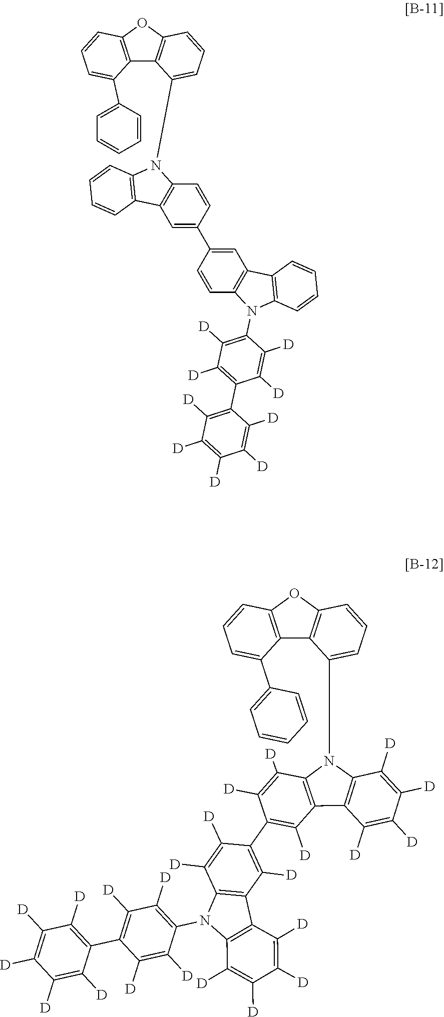 [SMILES: [2H]c1c([2H])c([2H])c(-c2c([2H])c([2H])c(-n3c4ccccc4c4cc(-c5ccc6c(c5)c5ccccc5n6-c5cccc6oc7cccc(-c8ccccc8)c7c56)ccc43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6oc7cccc(-c8ccccc8)c7c56)c([2H])c([2H])c43)c([2H])c2[2H])c([2H])c1[2H]]

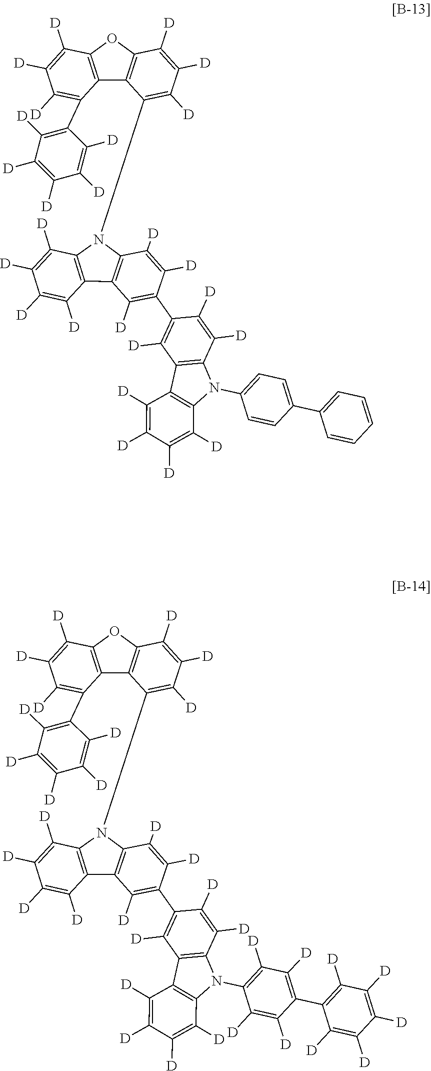

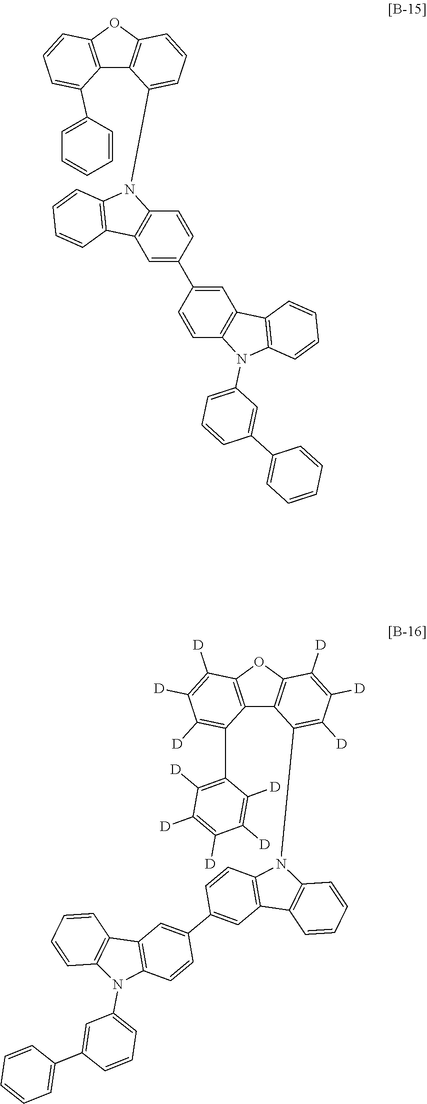 [SMILES: c1ccc(-c2cccc(-n3c4ccccc4c4cc(-c5ccc6c(c5)c5ccccc5n6-c5cccc6oc7cccc(-c8ccccc8)c7c56)ccc43)c2)cc1 ; [2H]c1c([2H])c([2H])c(-c2c([2H])c([2H])c([2H])c3oc4c([2H])c([2H])c([2H])c(-n5c6ccccc6c6cc(-c7ccc8c(c7)c7ccccc7n8-c7cccc(-c8ccccc8)c7)ccc65)c4c23)c([2H])c1[2H]]

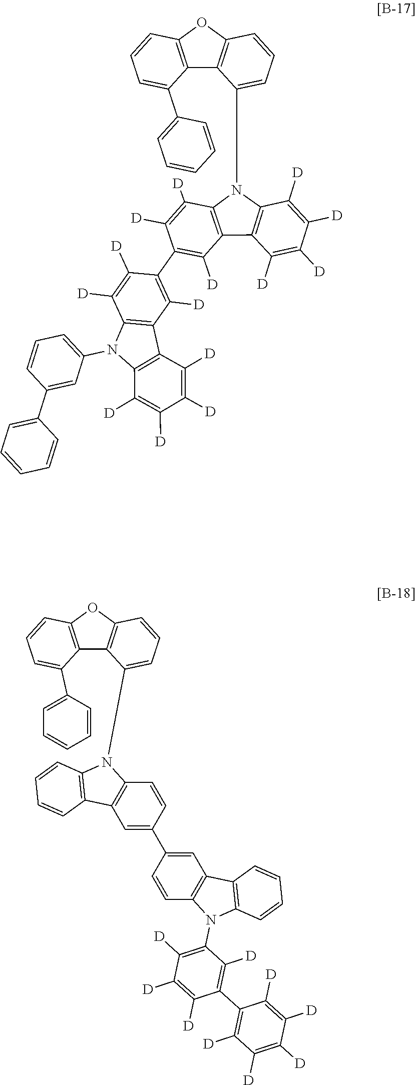 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cccc(-c6ccccc6)c5c34)c([2H])c([2H])c1n2-c1cccc(-c2ccccc2)c1 ; [2H]c1c([2H])c([2H])c(-c2c([2H])c([2H])c([2H])c(-n3c4ccccc4c4cc(-c5ccc6c(c5)c5ccccc5n6-c5cccc6oc7cccc(-c8ccccc8)c7c56)ccc43)c2[2H])c([2H])c1[2H]]

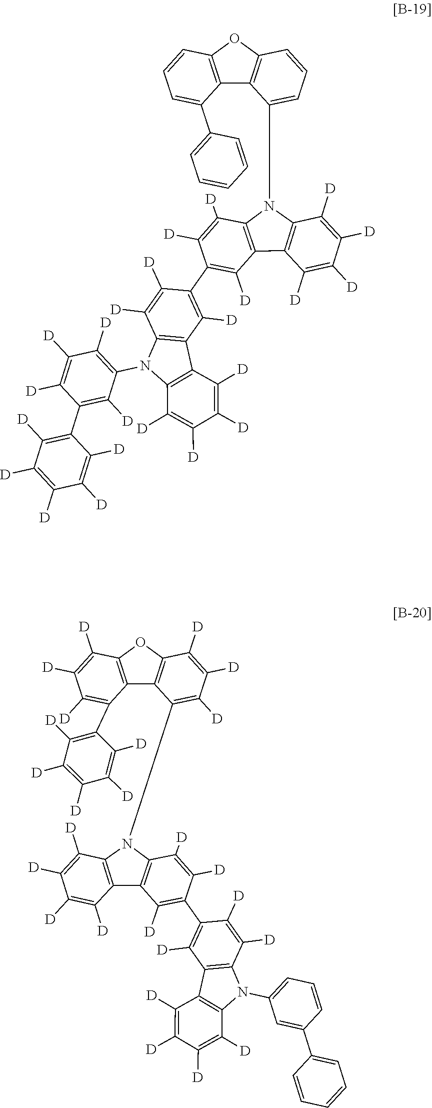 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6oc7cccc(-c8ccccc8)c7c56)c([2H])c([2H])c43)c2[2H])c([2H])c1[2H] ; [2H]c1c([2H])c([2H])c(-c2c([2H])c([2H])c([2H])c3oc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7cccc(-c8ccccc8)c7)c([2H])c([2H])c65)c4c23)c([2H])c1[2H]]

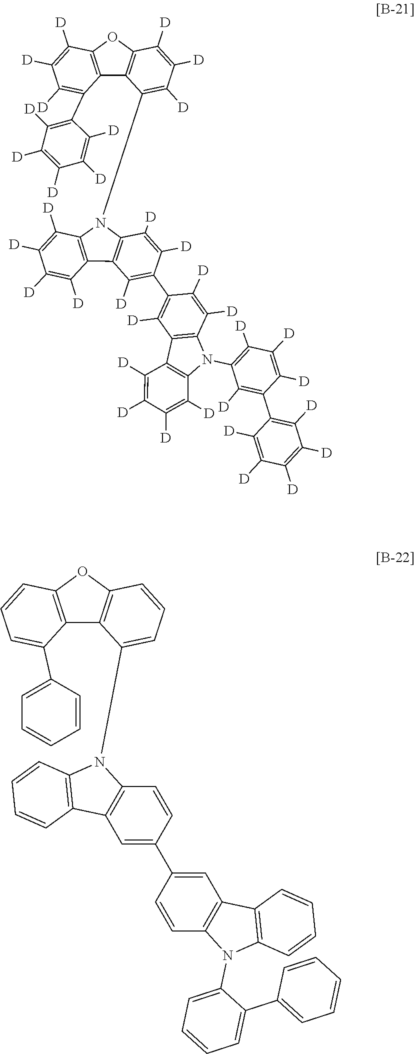 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6oc7c([2H])c([2H])c([2H])c(-c8c([2H])c([2H])c([2H])c([2H])c8[2H])c7c56)c([2H])c([2H])c43)c2[2H])c([2H])c1[2H] ; c1ccc(-c2ccccc2-n2c3ccccc3c3cc(-c4ccc5c(c4)c4ccccc4n5-c4cccc5oc6cccc(-c7ccccc7)c6c45)ccc32)cc1]

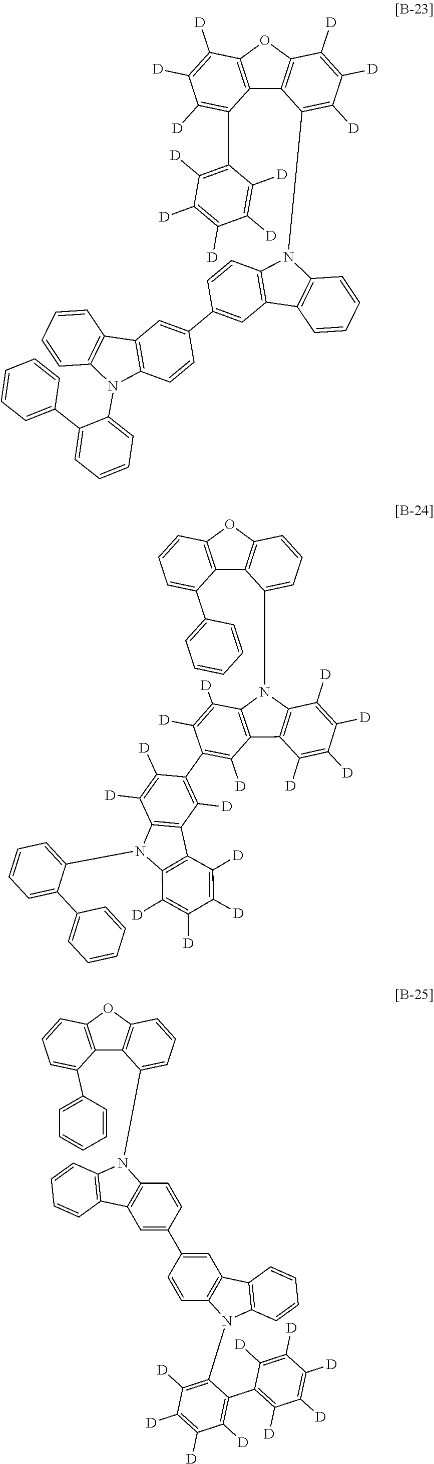 [SMILES: [2H]c1c([2H])c([2H])c(-c2c([2H])c([2H])c([2H])c3oc4c([2H])c([2H])c([2H])c(-n5c6ccccc6c6cc(-c7ccc8c(c7)c7ccccc7n8-c7ccccc7-c7ccccc7)ccc65)c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cccc(-c6ccccc6)c5c34)c([2H])c([2H])c1n2-c1ccccc1-c1ccccc1 ; [2H]c1c([2H])c([2H])c(-c2c([2H])c([2H])c([2H])c([2H])c2-n2c3ccccc3c3cc(-c4ccc5c(c4)c4ccccc4n5-c4cccc5oc6cccc(-c7ccccc7)c6c45)ccc32)c([2H])c1[2H]]

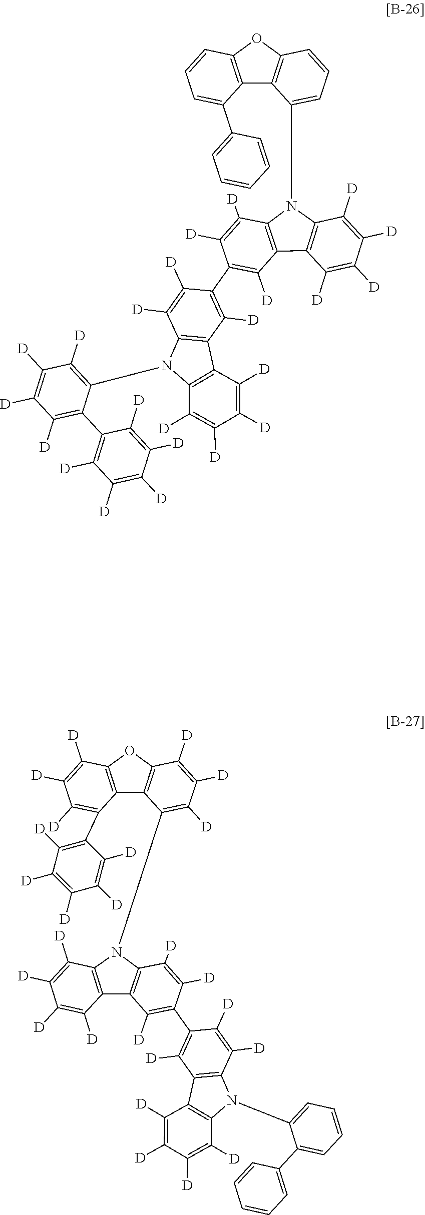 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5oc6cccc(-c7ccccc7)c6c45)c([2H])c([2H])c32)c([2H])c1[2H] ; [2H]c1c([2H])c([2H])c(-c2c([2H])c([2H])c([2H])c3oc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c4c23)c([2H])c1[2H]]

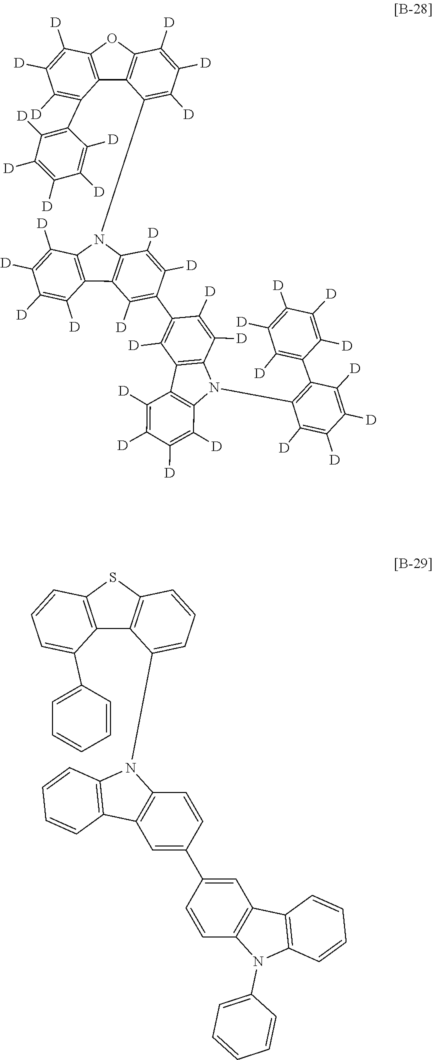 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5oc6c([2H])c([2H])c([2H])c(-c7c([2H])c([2H])c([2H])c([2H])c7[2H])c6c45)c([2H])c([2H])c32)c([2H])c1[2H] ; c1ccc(-c2cccc3sc4cccc(-n5c6ccccc6c6cc(-c7ccc8c(c7)c7ccccc7n8-c7ccccc7)ccc65)c4c23)cc1]

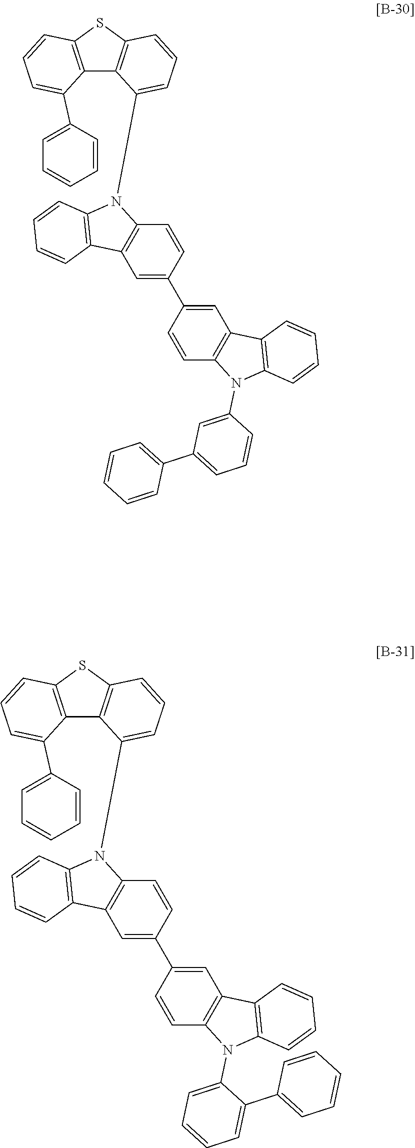 [SMILES: c1ccc(-c2cccc(-n3c4ccccc4c4cc(-c5ccc6c(c5)c5ccccc5n6-c5cccc6sc7cccc(-c8ccccc8)c7c56)ccc43)c2)cc1 ; c1ccc(-c2ccccc2-n2c3ccccc3c3cc(-c4ccc5c(c4)c4ccccc4n5-c4cccc5sc6cccc(-c7ccccc7)c6c45)ccc32)cc1]

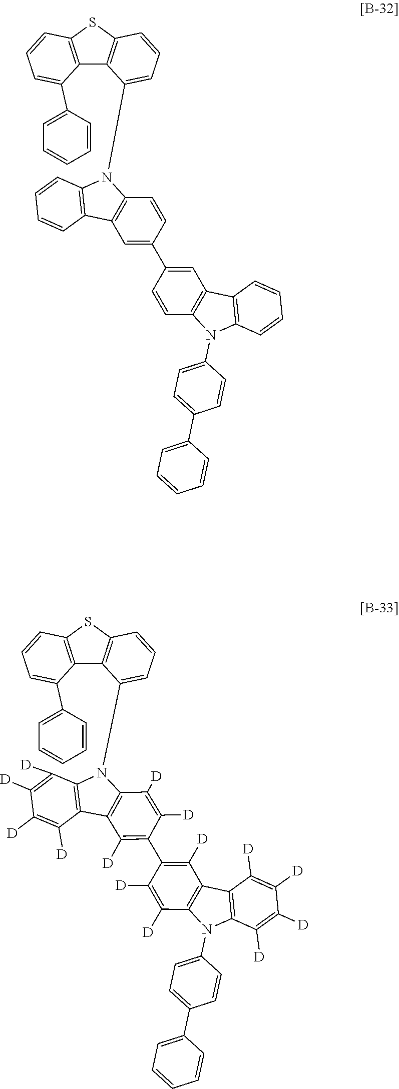 [SMILES: c1ccc(-c2ccc(-n3c4ccccc4c4cc(-c5ccc6c(c5)c5ccccc5n6-c5cccc6sc7cccc(-c8ccccc8)c7c56)ccc43)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4sc5cccc(-c6ccccc6)c5c34)c([2H])c([2H])c1n2-c1ccc(-c2ccccc2)cc1]

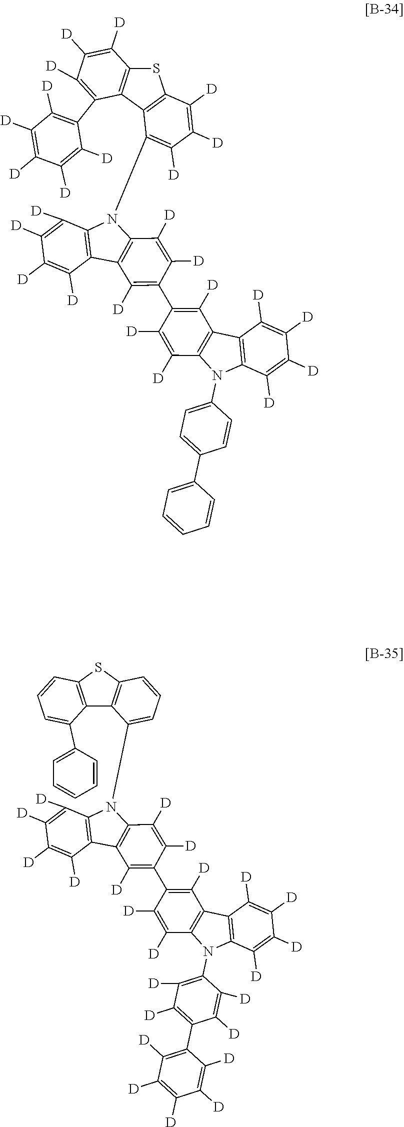 [SMILES: [2H]c1c([2H])c([2H])c(-c2c([2H])c([2H])c([2H])c3sc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c4c23)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6sc7cccc(-c8ccccc8)c7c56)c([2H])c([2H])c43)c([2H])c2[2H])c([2H])c1[2H]]

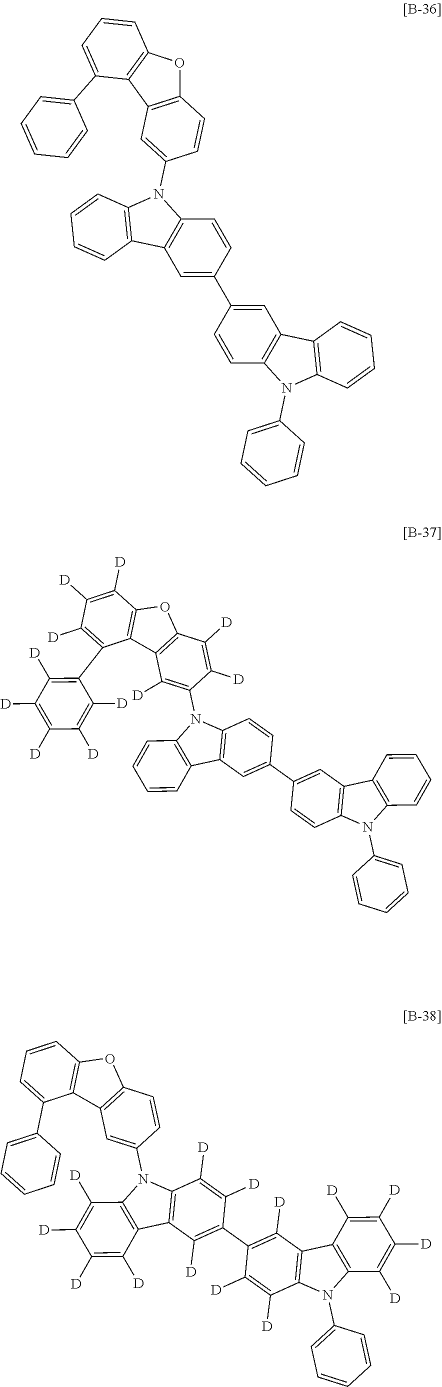 [SMILES: c1ccc(-c2cccc3oc4ccc(-n5c6ccccc6c6cc(-c7ccc8c(c7)c7ccccc7n8-c7ccccc7)ccc65)cc4c23)cc1 ; [2H]c1c([2H])c([2H])c(-c2c([2H])c([2H])c([2H])c3oc4c([2H])c([2H])c(-n5c6ccccc6c6cc(-c7ccc8c(c7)c7ccccc7n8-c7ccccc7)ccc65)c([2H])c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4oc5cccc(-c6ccccc6)c5c4c3)c([2H])c([2H])c1n2-c1ccccc1]

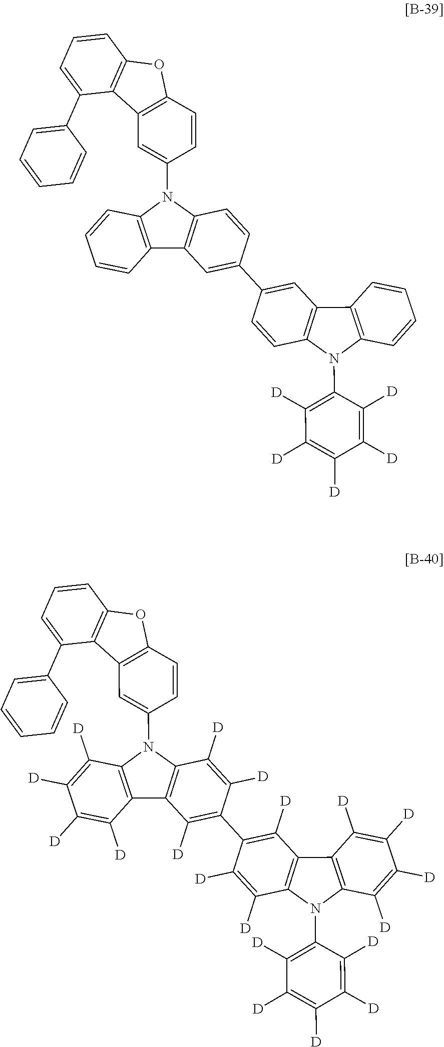 [SMILES: [2H]c1c([2H])c([2H])c(-n2c3ccccc3c3cc(-c4ccc5c(c4)c4ccccc4n5-c4ccc5oc6cccc(-c7ccccc7)c6c5c4)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5oc6cccc(-c7ccccc7)c6c5c4)c([2H])c([2H])c32)c([2H])c1[2H]]

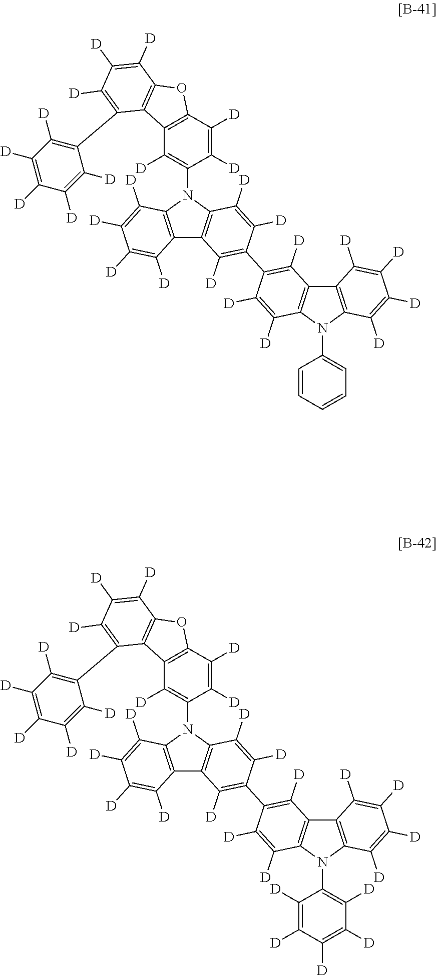 [SMILES: [2H]c1c([2H])c([2H])c(-c2c([2H])c([2H])c([2H])c3oc4c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c([2H])c4c23)c([2H])c1[2H] ; [2H]c1c([2H])c([2H])c(-c2c([2H])c([2H])c([2H])c3oc4c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c4c23)c([2H])c1[2H]]

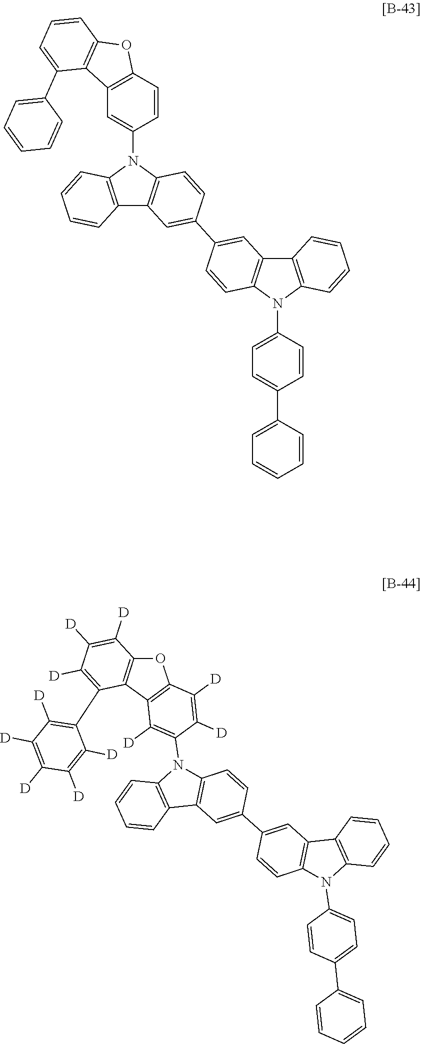 [SMILES: c1ccc(-c2ccc(-n3c4ccccc4c4cc(-c5ccc6c(c5)c5ccccc5n6-c5ccc6oc7cccc(-c8ccccc8)c7c6c5)ccc43)cc2)cc1 ; [2H]c1c([2H])c([2H])c(-c2c([2H])c([2H])c([2H])c3oc4c([2H])c([2H])c(-n5c6ccccc6c6cc(-c7ccc8c(c7)c7ccccc7n8-c7ccc(-c8ccccc8)cc7)ccc65)c([2H])c4c23)c([2H])c1[2H]]

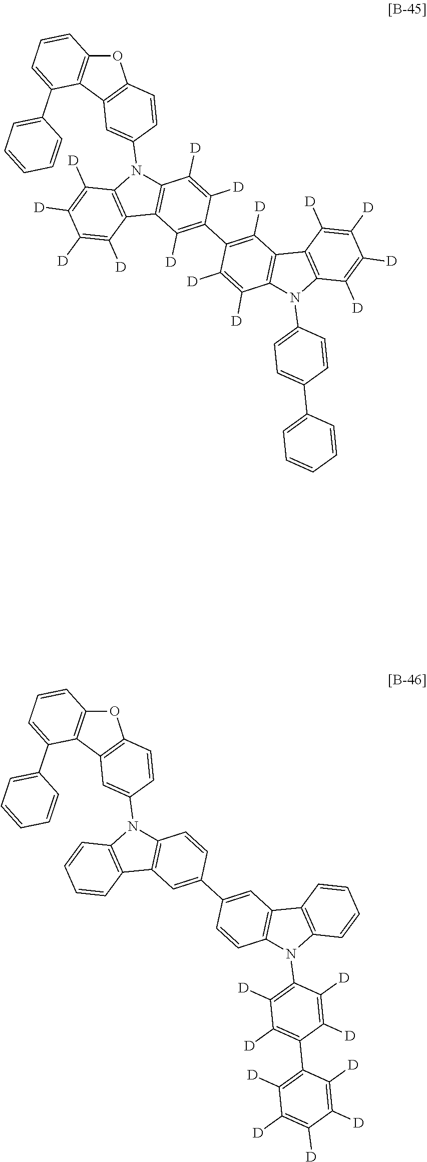 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4oc5cccc(-c6ccccc6)c5c4c3)c([2H])c([2H])c1n2-c1ccc(-c2ccccc2)cc1 ; [2H]c1c([2H])c([2H])c(-c2c([2H])c([2H])c(-n3c4ccccc4c4cc(-c5ccc6c(c5)c5ccccc5n6-c5ccc6oc7cccc(-c8ccccc8)c7c6c5)ccc43)c([2H])c2[2H])c([2H])c1[2H]]

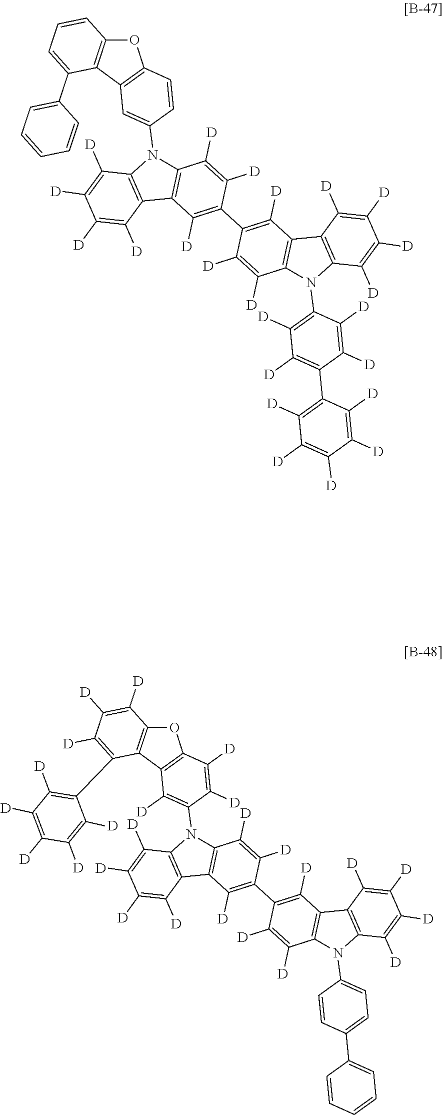 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6oc7cccc(-c8ccccc8)c7c6c5)c([2H])c([2H])c43)c([2H])c2[2H])c([2H])c1[2H] ; [2H]c1c([2H])c([2H])c(-c2c([2H])c([2H])c([2H])c3oc4c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c([2H])c4c23)c([2H])c1[2H]]

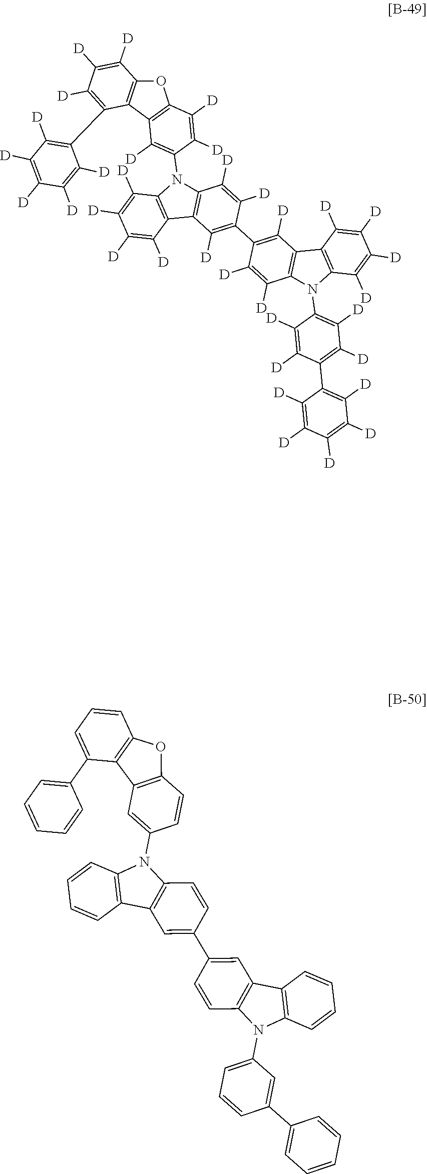 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6oc7c([2H])c([2H])c([2H])c(-c8c([2H])c([2H])c([2H])c([2H])c8[2H])c7c6c5[2H])c([2H])c([2H])c43)c([2H])c2[2H])c([2H])c1[2H] ; c1ccc(-c2cccc(-n3c4ccccc4c4cc(-c5ccc6c(c5)c5ccccc5n6-c5ccc6oc7cccc(-c8ccccc8)c7c6c5)ccc43)c2)cc1]

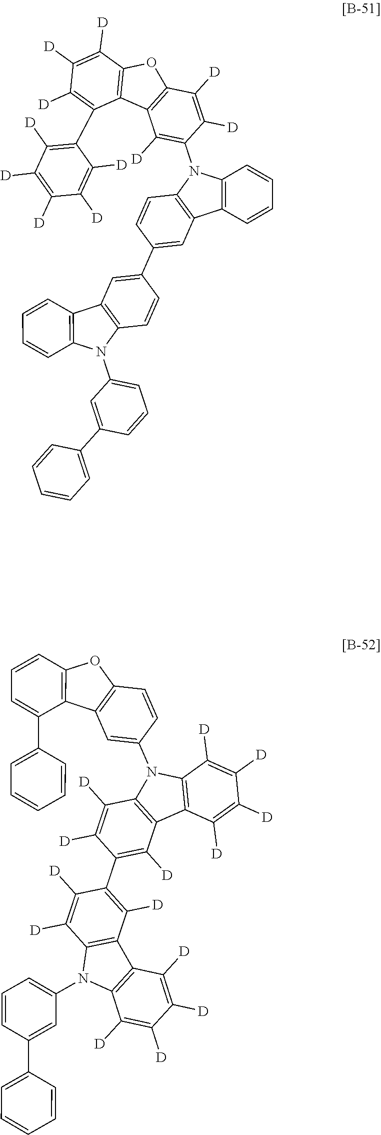 [SMILES: [2H]c1c([2H])c([2H])c(-c2c([2H])c([2H])c([2H])c3oc4c([2H])c([2H])c(-n5c6ccccc6c6cc(-c7ccc8c(c7)c7ccccc7n8-c7cccc(-c8ccccc8)c7)ccc65)c([2H])c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4oc5cccc(-c6ccccc6)c5c4c3)c([2H])c([2H])c1n2-c1cccc(-c2ccccc2)c1]

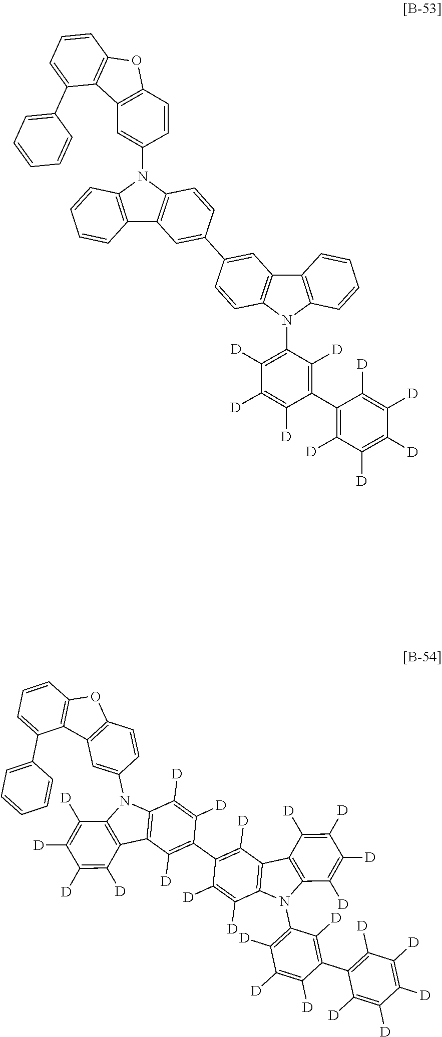 [SMILES: [2H]c1c([2H])c([2H])c(-c2c([2H])c([2H])c([2H])c(-n3c4ccccc4c4cc(-c5ccc6c(c5)c5ccccc5n6-c5ccc6oc7cccc(-c8ccccc8)c7c6c5)ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6oc7cccc(-c8ccccc8)c7c6c5)c([2H])c([2H])c43)c2[2H])c([2H])c1[2H]]

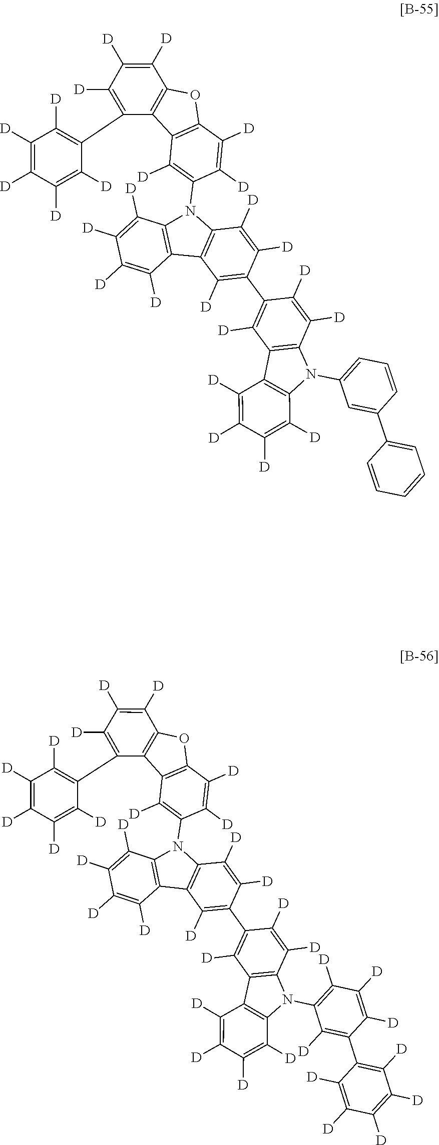

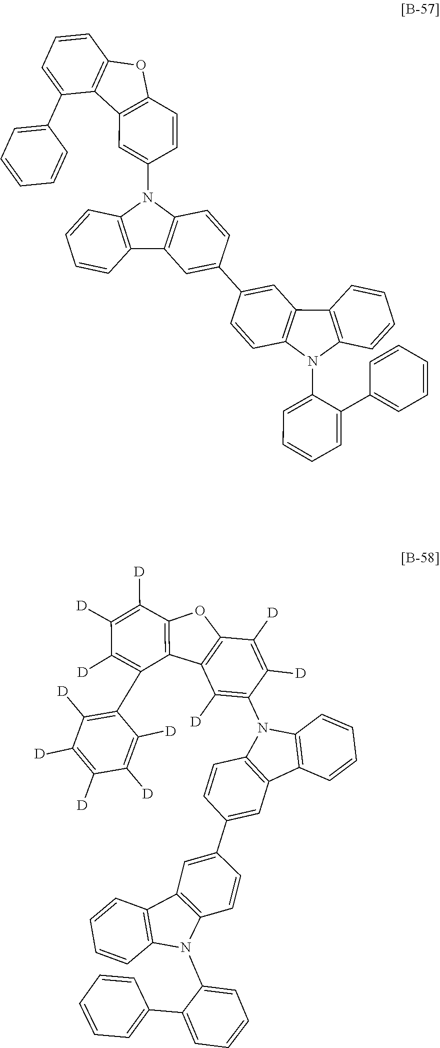 [SMILES: c1ccc(-c2ccccc2-n2c3ccccc3c3cc(-c4ccc5c(c4)c4ccccc4n5-c4ccc5oc6cccc(-c7ccccc7)c6c5c4)ccc32)cc1 ; [2H]c1c([2H])c([2H])c(-c2c([2H])c([2H])c([2H])c3oc4c([2H])c([2H])c(-n5c6ccccc6c6cc(-c7ccc8c(c7)c7ccccc7n8-c7ccccc7-c7ccccc7)ccc65)c([2H])c4c23)c([2H])c1[2H]]

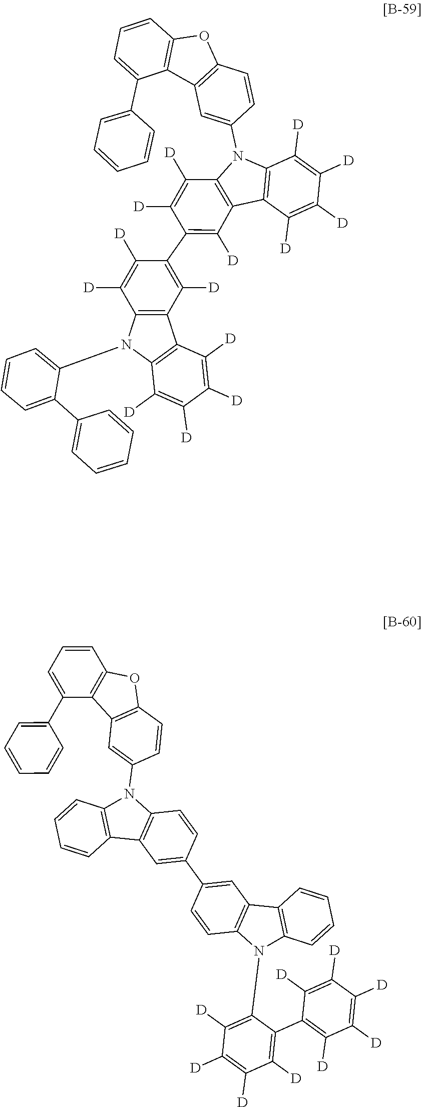 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3-c3ccccc3)c([2H])c([2H])c1n2-c1ccc2oc3cccc(-c4ccccc4)c3c2c1 ; [2H]c1c([2H])c([2H])c(-c2c([2H])c([2H])c([2H])c([2H])c2-n2c3ccccc3c3cc(-c4ccc5c(c4)c4ccccc4n5-c4ccc5oc6cccc(-c7ccccc7)c6c5c4)ccc32)c([2H])c1[2H]]

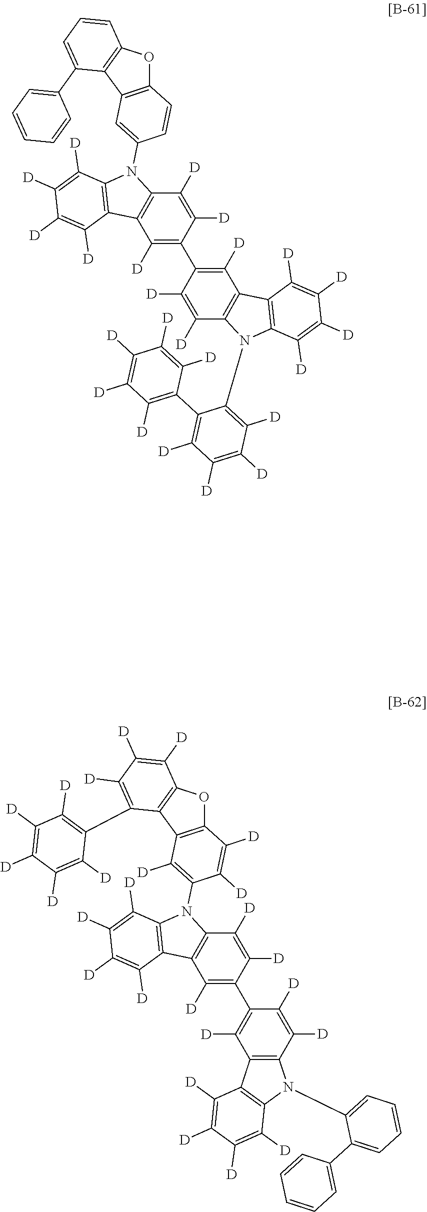 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5oc6cccc(-c7ccccc7)c6c5c4)c([2H])c([2H])c32)c([2H])c1[2H] ; [2H]c1c([2H])c([2H])c(-c2c([2H])c([2H])c([2H])c3oc4c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c4c23)c([2H])c1[2H]]

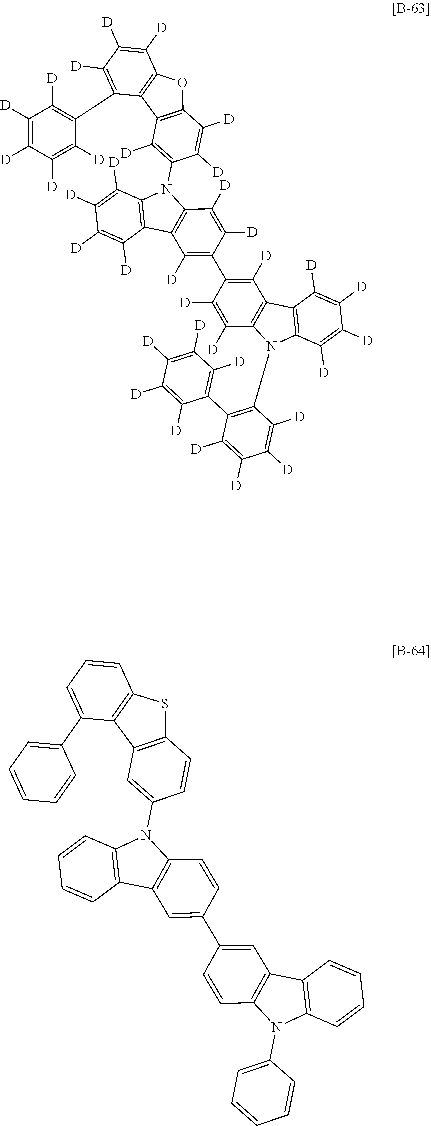 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5oc6c([2H])c([2H])c([2H])c(-c7c([2H])c([2H])c([2H])c([2H])c7[2H])c6c5c4[2H])c([2H])c([2H])c32)c([2H])c1[2H] ; c1ccc(-c2cccc3sc4ccc(-n5c6ccccc6c6cc(-c7ccc8c(c7)c7ccccc7n8-c7ccccc7)ccc65)cc4c23)cc1]

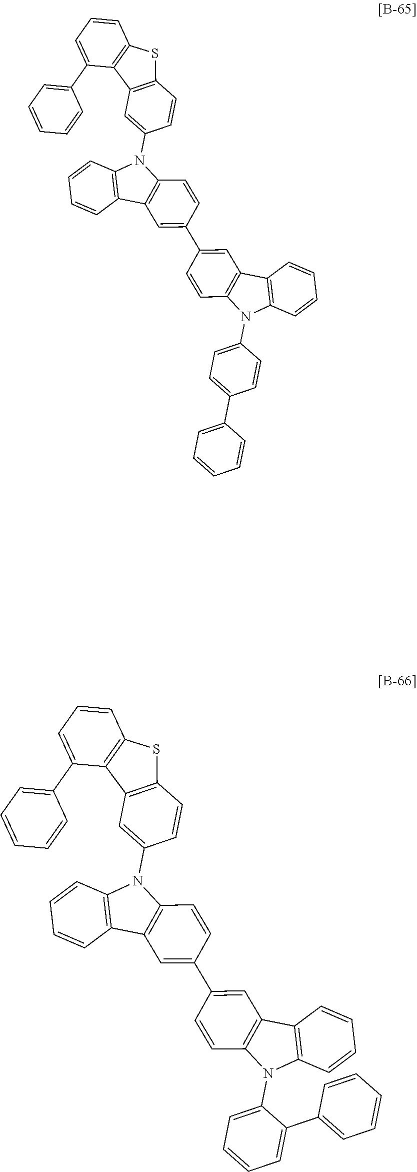 [SMILES: c1ccc(-c2ccc(-n3c4ccccc4c4cc(-c5ccc6c(c5)c5ccccc5n6-c5ccc6sc7cccc(-c8ccccc8)c7c6c5)ccc43)cc2)cc1 ; c1ccc(-c2ccccc2-n2c3ccccc3c3cc(-c4ccc5c(c4)c4ccccc4n5-c4ccc5sc6cccc(-c7ccccc7)c6c5c4)ccc32)cc1]

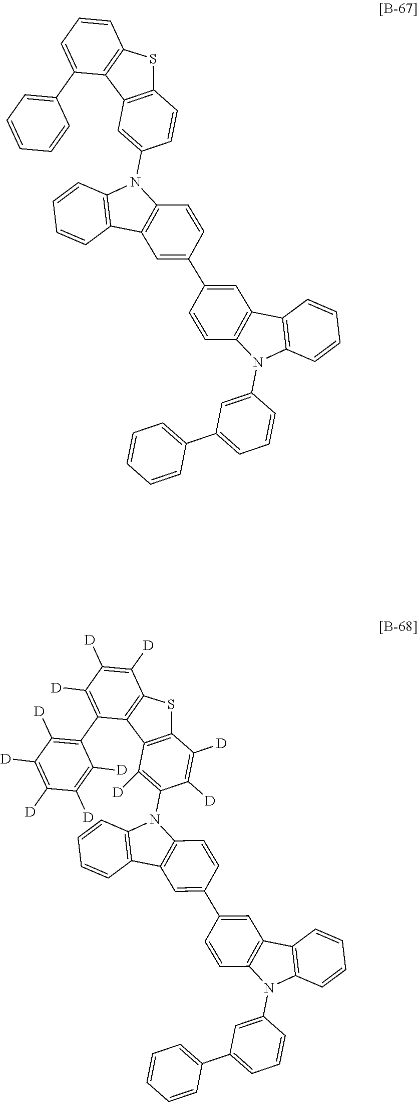 [SMILES: c1ccc(-c2cccc(-n3c4ccccc4c4cc(-c5ccc6c(c5)c5ccccc5n6-c5ccc6sc7cccc(-c8ccccc8)c7c6c5)ccc43)c2)cc1 ; [2H]c1c([2H])c([2H])c(-c2c([2H])c([2H])c([2H])c3sc4c([2H])c([2H])c(-n5c6ccccc6c6cc(-c7ccc8c(c7)c7ccccc7n8-c7cccc(-c8ccccc8)c7)ccc65)c([2H])c4c23)c([2H])c1[2H]]

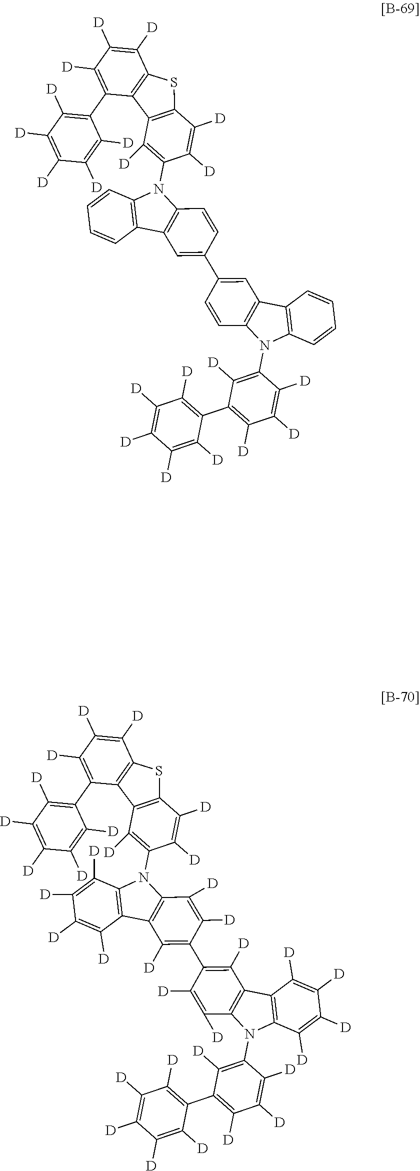 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6sc7c([2H])c([2H])c([2H])c(-c8c([2H])c([2H])c([2H])c([2H])c8[2H])c7c6c5[2H])ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6sc7c([2H])c([2H])c([2H])c(-c8c([2H])c([2H])c([2H])c([2H])c8[2H])c7c6c5[2H])c([2H])c([2H])c43)c2[2H])c([2H])c1[2H]]

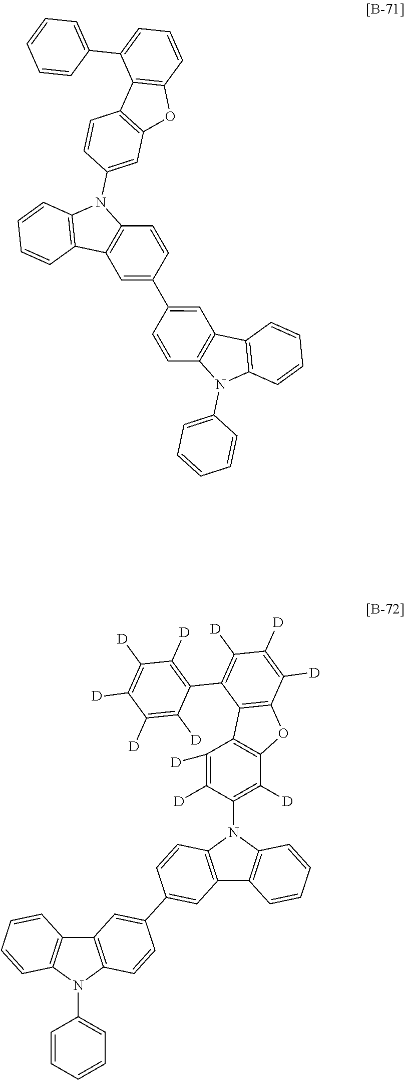 [SMILES: c1ccc(-c2cccc3oc4cc(-n5c6ccccc6c6cc(-c7ccc8c(c7)c7ccccc7n8-c7ccccc7)ccc65)ccc4c23)cc1 ; [2H]c1c([2H])c([2H])c(-c2c([2H])c([2H])c([2H])c3oc4c([2H])c(-n5c6ccccc6c6cc(-c7ccc8c(c7)c7ccccc7n8-c7ccccc7)ccc65)c([2H])c([2H])c4c23)c([2H])c1[2H]]

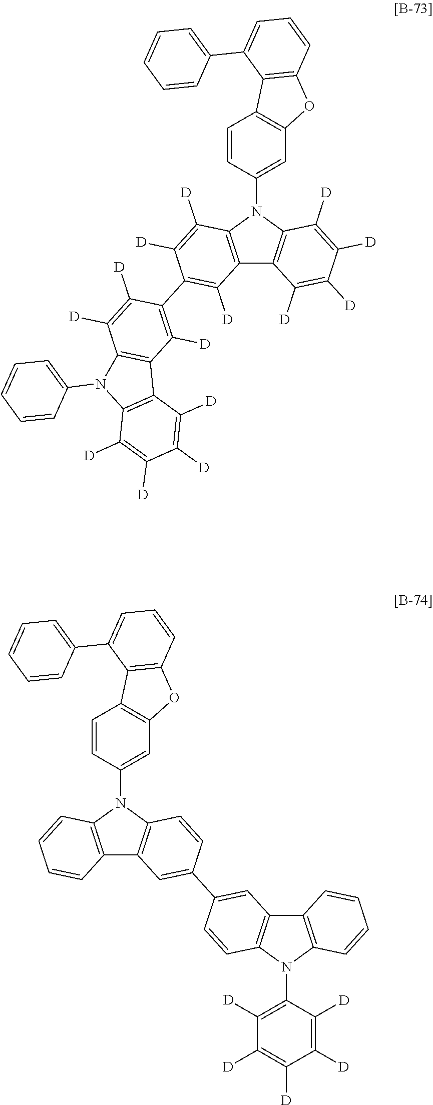 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3cccc(-c5ccccc5)c34)c([2H])c([2H])c1n2-c1ccccc1 ; [2H]c1c([2H])c([2H])c(-n2c3ccccc3c3cc(-c4ccc5c(c4)c4ccccc4n5-c4ccc5c(c4)oc4cccc(-c6ccccc6)c45)ccc32)c([2H])c1[2H]]

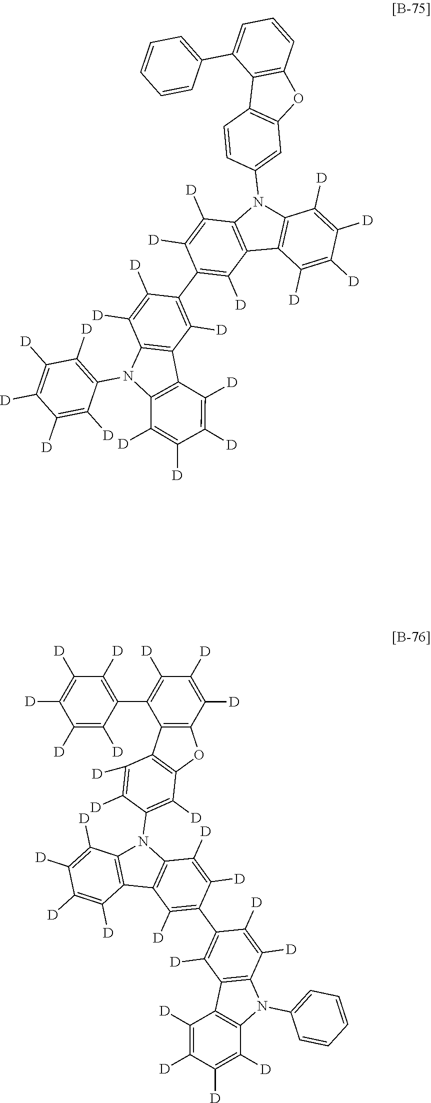 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)oc4cccc(-c6ccccc6)c45)c([2H])c([2H])c32)c([2H])c1[2H] ; [2H]c1c([2H])c([2H])c(-c2c([2H])c([2H])c([2H])c3oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c([2H])c([2H])c4c23)c([2H])c1[2H]]

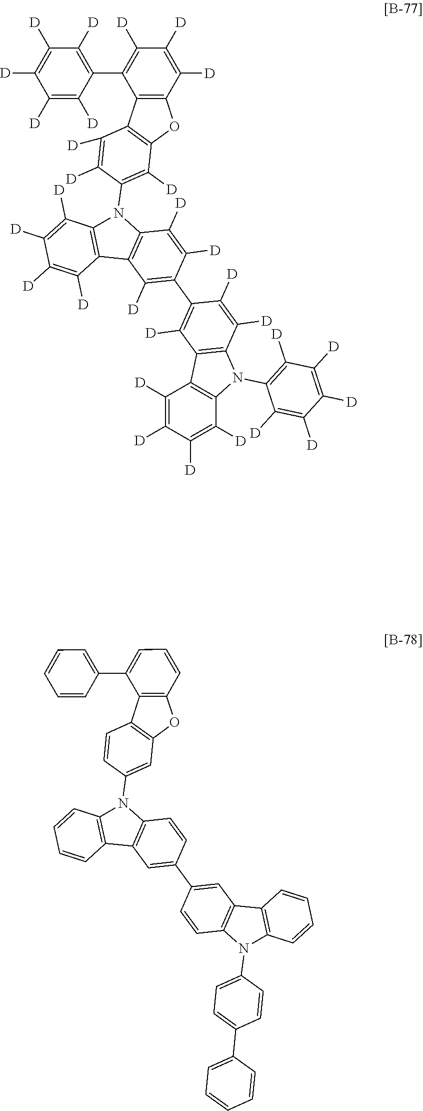 [SMILES: [2H]c1c([2H])c([2H])c(-c2c([2H])c([2H])c([2H])c3oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c([2H])c4c23)c([2H])c1[2H] ; c1ccc(-c2ccc(-n3c4ccccc4c4cc(-c5ccc6c(c5)c5ccccc5n6-c5ccc6c(c5)oc5cccc(-c7ccccc7)c56)ccc43)cc2)cc1]

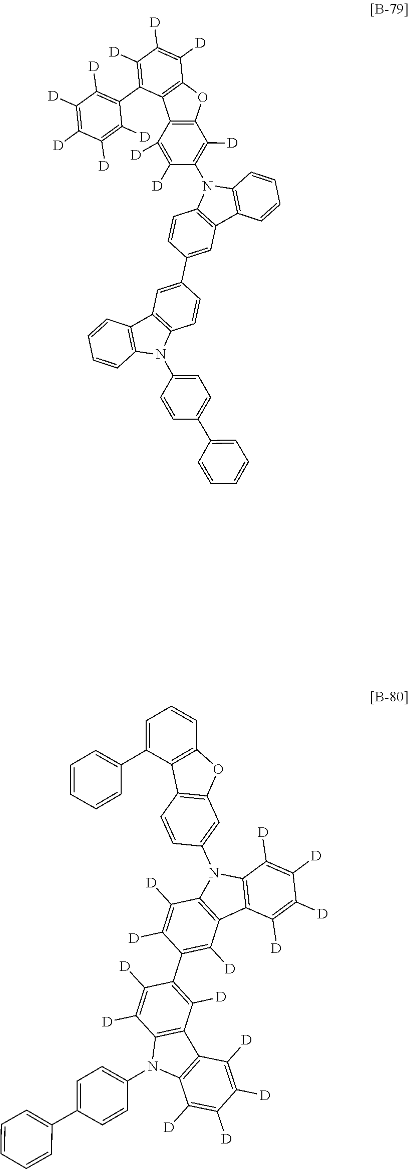 [SMILES: [2H]c1c([2H])c([2H])c(-c2c([2H])c([2H])c([2H])c3oc4c([2H])c(-n5c6ccccc6c6cc(-c7ccc8c(c7)c7ccccc7n8-c7ccc(-c8ccccc8)cc7)ccc65)c([2H])c([2H])c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3cccc(-c5ccccc5)c34)c([2H])c([2H])c1n2-c1ccc(-c2ccccc2)cc1]

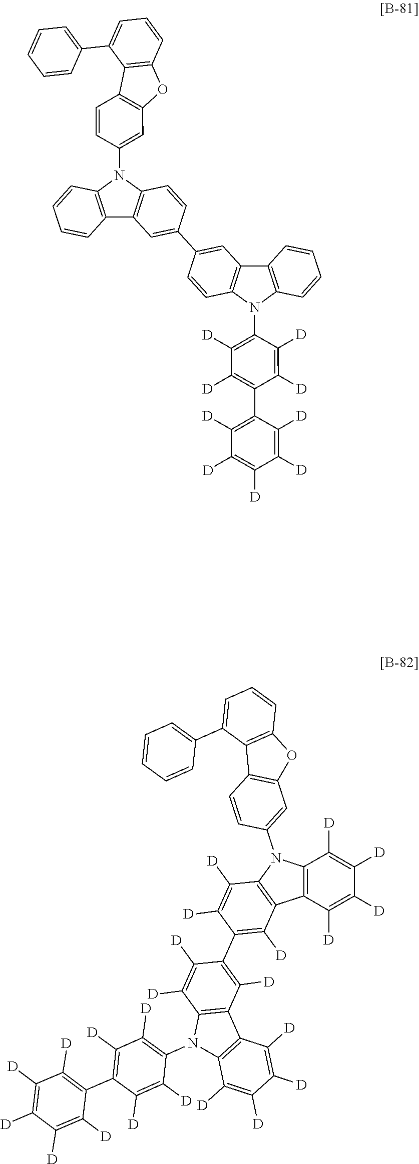 [SMILES: [2H]c1c([2H])c([2H])c(-c2c([2H])c([2H])c(-n3c4ccccc4c4cc(-c5ccc6c(c5)c5ccccc5n6-c5ccc6c(c5)oc5cccc(-c7ccccc7)c56)ccc43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)oc5cccc(-c7ccccc7)c56)c([2H])c([2H])c43)c([2H])c2[2H])c([2H])c1[2H]]

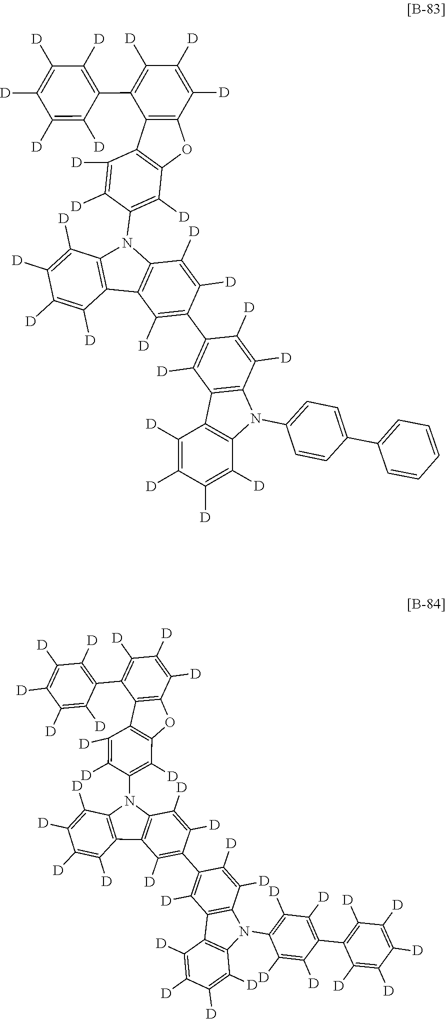

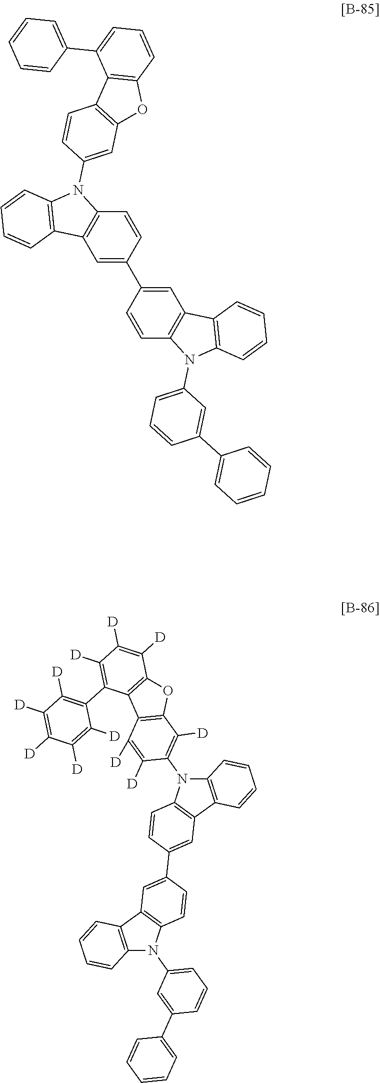 [SMILES: c1ccc(-c2cccc(-n3c4ccccc4c4cc(-c5ccc6c(c5)c5ccccc5n6-c5ccc6c(c5)oc5cccc(-c7ccccc7)c56)ccc43)c2)cc1 ; [2H]c1c([2H])c([2H])c(-c2c([2H])c([2H])c([2H])c3oc4c([2H])c(-n5c6ccccc6c6cc(-c7ccc8c(c7)c7ccccc7n8-c7cccc(-c8ccccc8)c7)ccc65)c([2H])c([2H])c4c23)c([2H])c1[2H]]

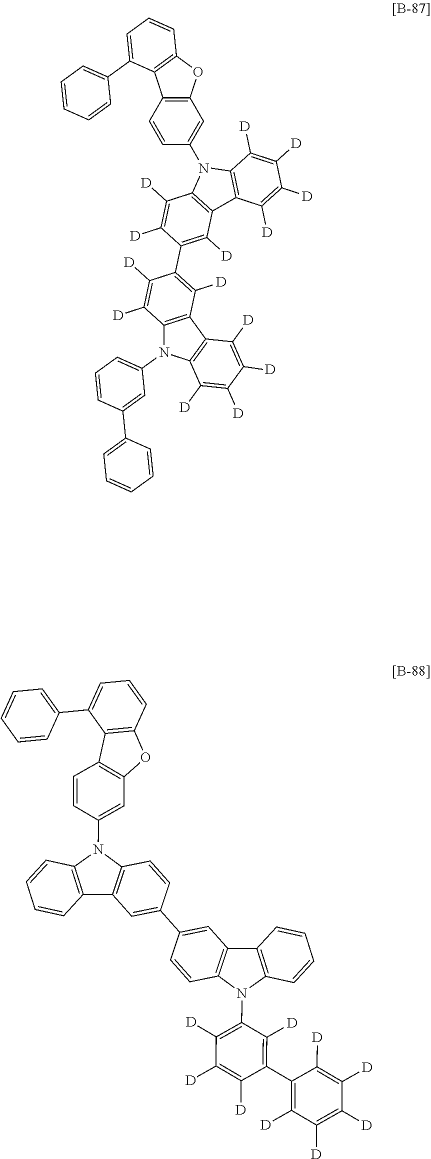 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3cccc(-c5ccccc5)c34)c([2H])c([2H])c1n2-c1cccc(-c2ccccc2)c1 ; [2H]c1c([2H])c([2H])c(-c2c([2H])c([2H])c([2H])c(-n3c4ccccc4c4cc(-c5ccc6c(c5)c5ccccc5n6-c5ccc6c(c5)oc5cccc(-c7ccccc7)c56)ccc43)c2[2H])c([2H])c1[2H]]

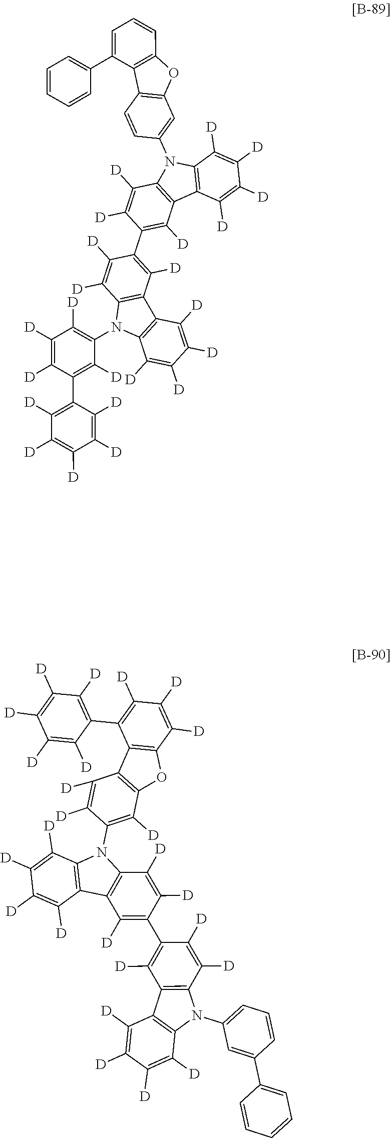 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)oc5cccc(-c7ccccc7)c56)c([2H])c([2H])c43)c2[2H])c([2H])c1[2H] ; [2H]c1c([2H])c([2H])c(-c2c([2H])c([2H])c([2H])c3oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7cccc(-c8ccccc8)c7)c([2H])c([2H])c65)c([2H])c([2H])c4c23)c([2H])c1[2H]]

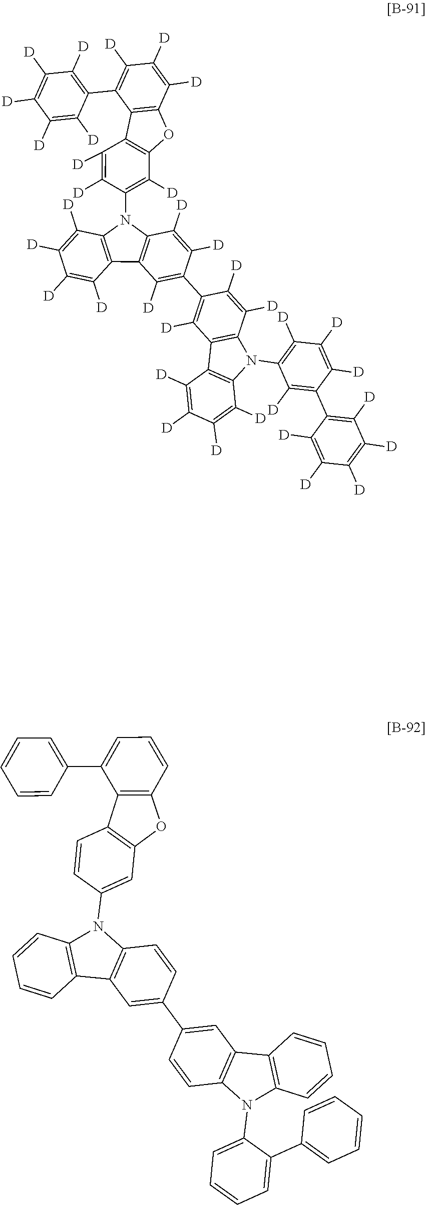 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(oc7c([2H])c([2H])c([2H])c(-c8c([2H])c([2H])c([2H])c([2H])c8[2H])c76)c5[2H])c([2H])c([2H])c43)c2[2H])c([2H])c1[2H] ; c1ccc(-c2ccccc2-n2c3ccccc3c3cc(-c4ccc5c(c4)c4ccccc4n5-c4ccc5c(c4)oc4cccc(-c6ccccc6)c45)ccc32)cc1]

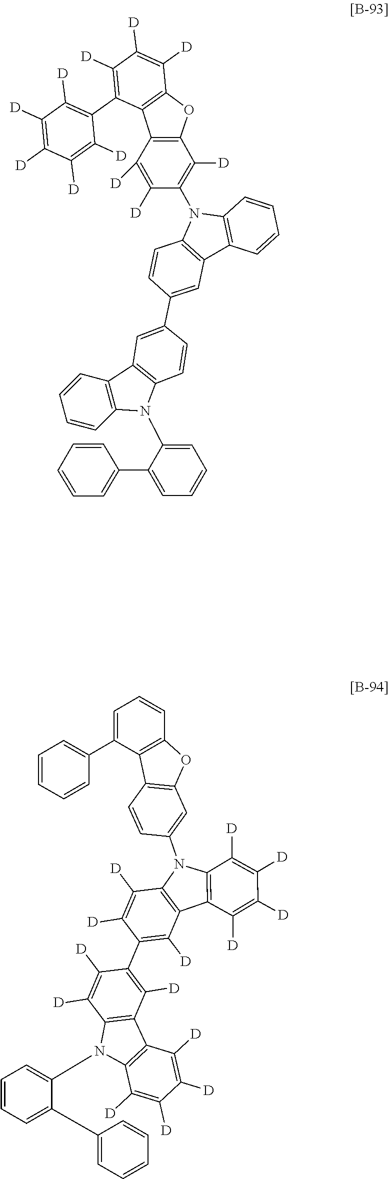 [SMILES: [2H]c1c([2H])c([2H])c(-c2c([2H])c([2H])c([2H])c3oc4c([2H])c(-n5c6ccccc6c6cc(-c7ccc8c(c7)c7ccccc7n8-c7ccccc7-c7ccccc7)ccc65)c([2H])c([2H])c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3-c3ccccc3)c([2H])c([2H])c1n2-c1ccc2c(c1)oc1cccc(-c3ccccc3)c12]

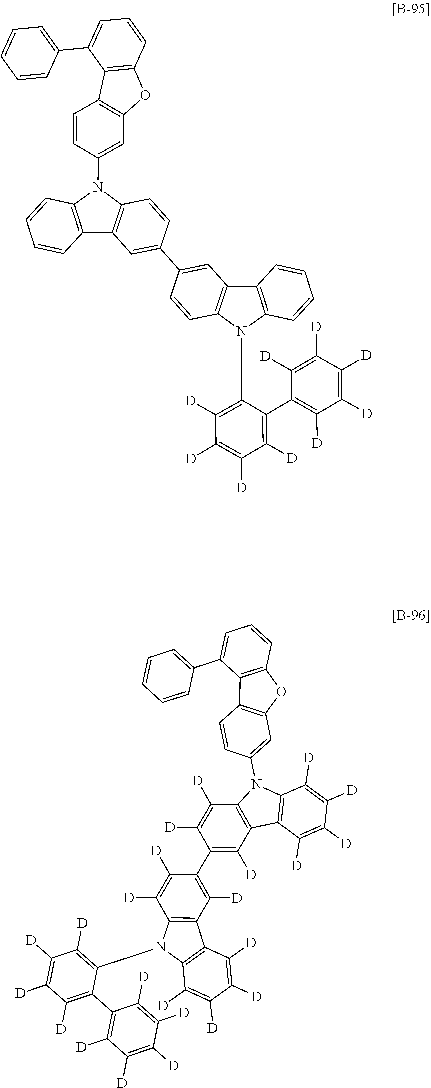 [SMILES: [2H]c1c([2H])c([2H])c(-c2c([2H])c([2H])c([2H])c([2H])c2-n2c3ccccc3c3cc(-c4ccc5c(c4)c4ccccc4n5-c4ccc5c(c4)oc4cccc(-c6ccccc6)c45)ccc32)c([2H])c1[2H] ; [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)oc4cccc(-c6ccccc6)c45)c([2H])c([2H])c32)c([2H])c1[2H]]

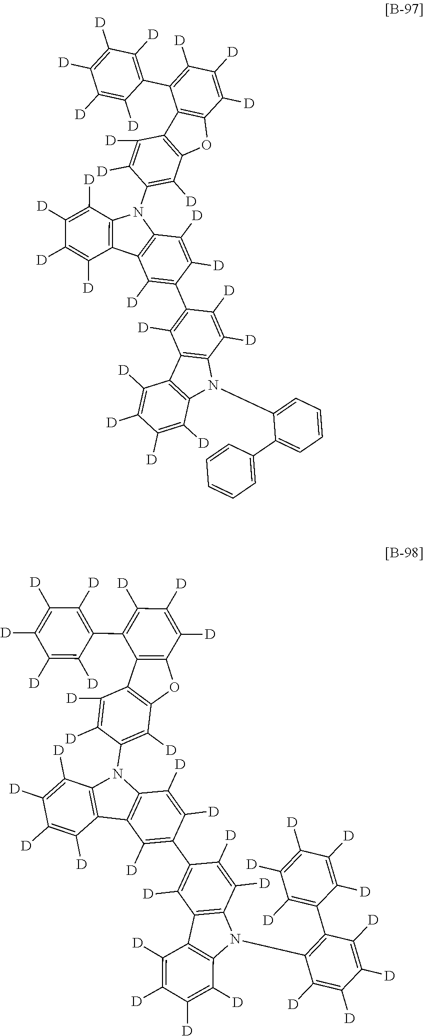

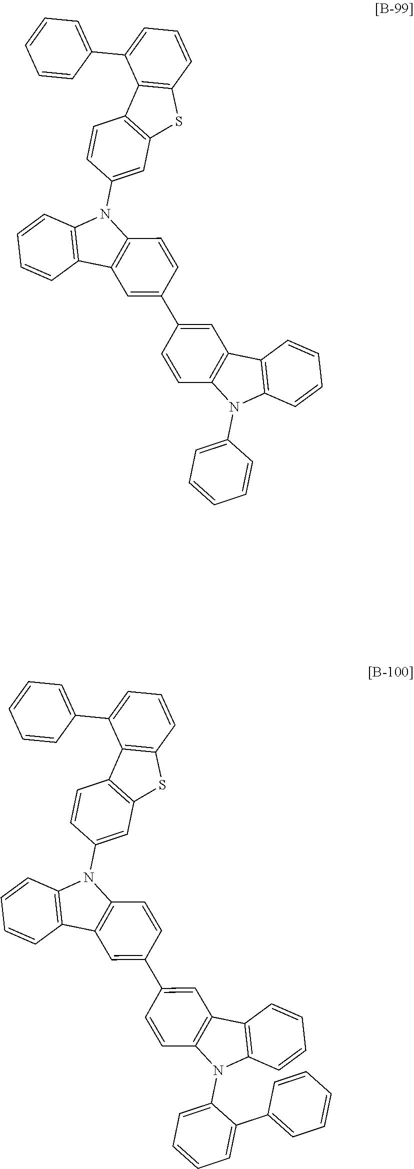 [SMILES: c1ccc(-c2cccc3sc4cc(-n5c6ccccc6c6cc(-c7ccc8c(c7)c7ccccc7n8-c7ccccc7)ccc65)ccc4c23)cc1 ; c1ccc(-c2ccccc2-n2c3ccccc3c3cc(-c4ccc5c(c4)c4ccccc4n5-c4ccc5c(c4)sc4cccc(-c6ccccc6)c45)ccc32)cc1]

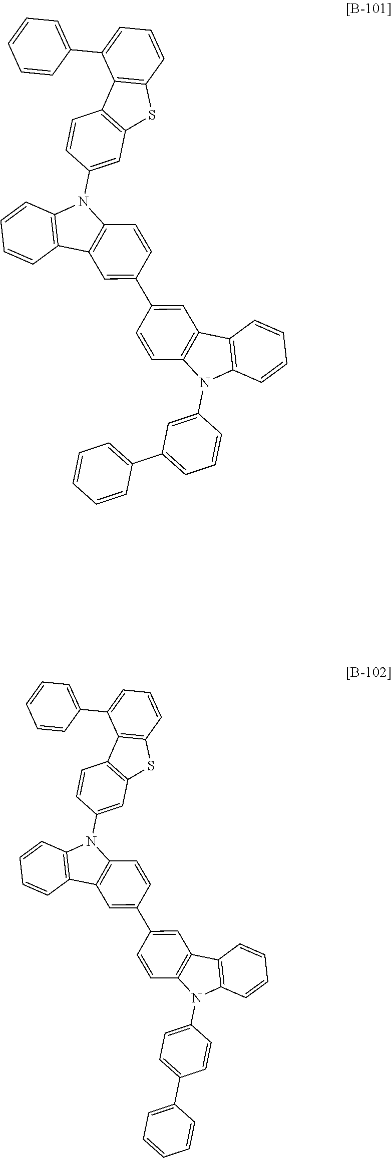 [SMILES: c1ccc(-c2cccc(-n3c4ccccc4c4cc(-c5ccc6c(c5)c5ccccc5n6-c5ccc6c(c5)sc5cccc(-c7ccccc7)c56)ccc43)c2)cc1 ; c1ccc(-c2ccc(-n3c4ccccc4c4cc(-c5ccc6c(c5)c5ccccc5n6-c5ccc6c(c5)sc5cccc(-c7ccccc7)c56)ccc43)cc2)cc1]

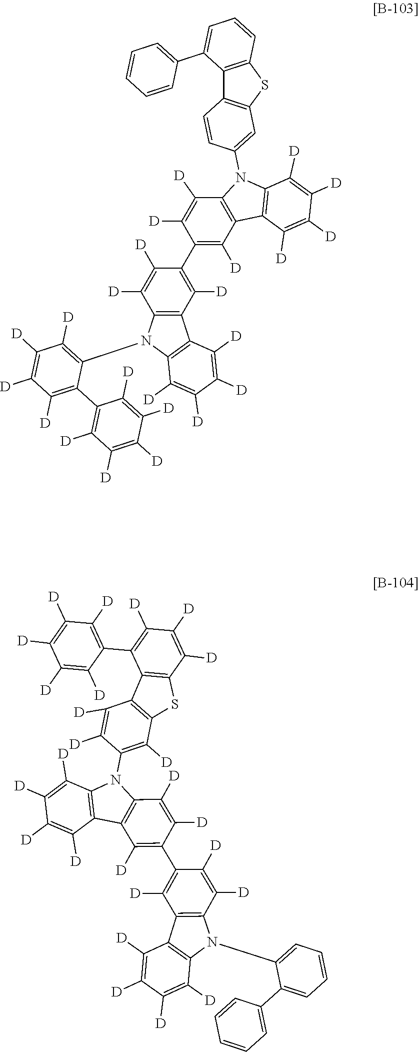 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)sc4cccc(-c6ccccc6)c45)c([2H])c([2H])c32)c([2H])c1[2H] ; [2H]c1c([2H])c([2H])c(-c2c([2H])c([2H])c([2H])c3sc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c([2H])c4c23)c([2H])c1[2H]]

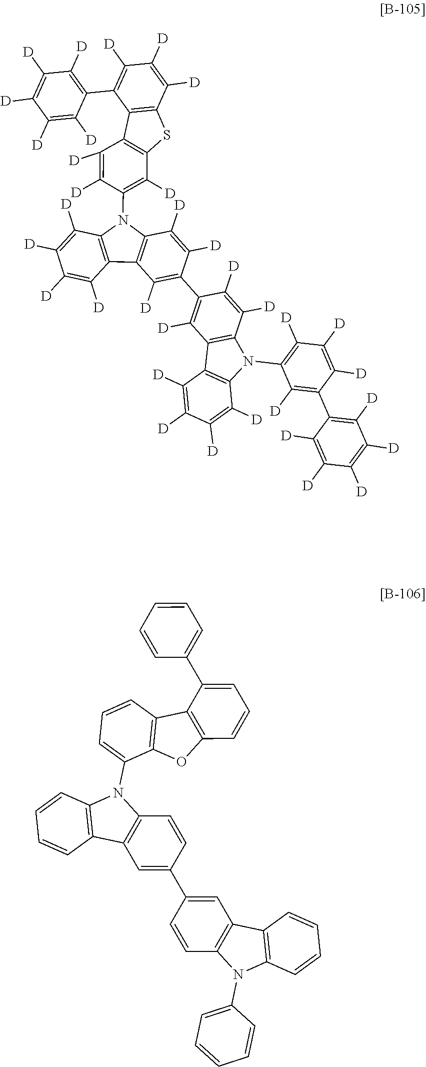 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(sc7c([2H])c([2H])c([2H])c(-c8c([2H])c([2H])c([2H])c([2H])c8[2H])c76)c5[2H])c([2H])c([2H])c43)c2[2H])c([2H])c1[2H] ; c1ccc(-c2cccc3oc4c(-n5c6ccccc6c6cc(-c7ccc8c(c7)c7ccccc7n8-c7ccccc7)ccc65)cccc4c23)cc1]

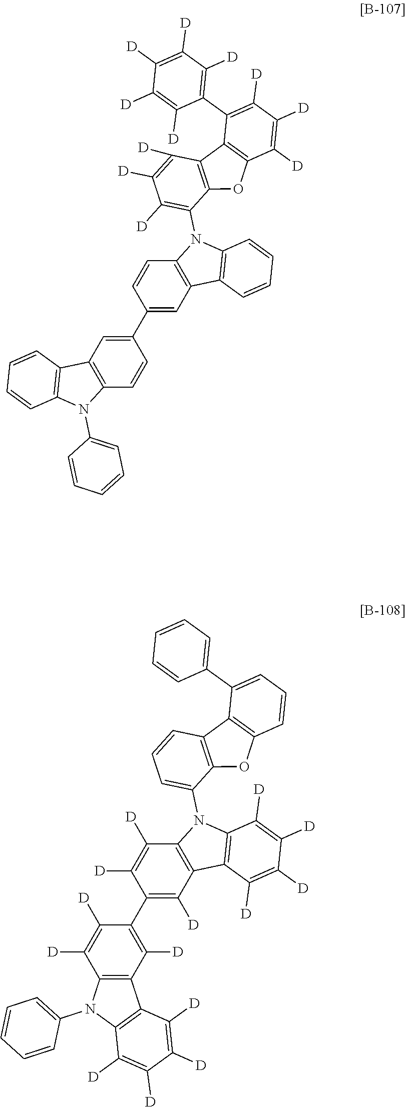 [SMILES: [2H]c1c([2H])c([2H])c(-c2c([2H])c([2H])c([2H])c3oc4c(-n5c6ccccc6c6cc(-c7ccc8c(c7)c7ccccc7n8-c7ccccc7)ccc65)c([2H])c([2H])c([2H])c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cccc(-c5ccccc5)c34)c([2H])c([2H])c1n2-c1ccccc1]

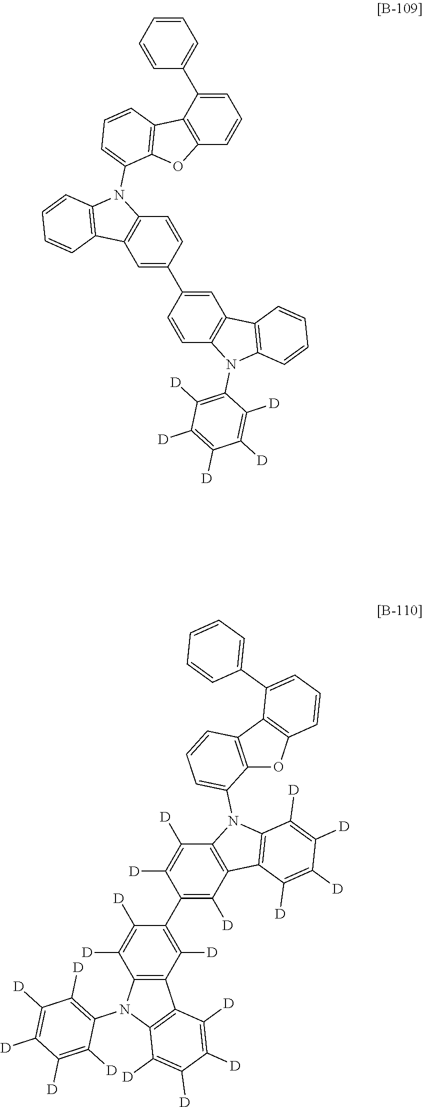 [SMILES: [2H]c1c([2H])c([2H])c(-n2c3ccccc3c3cc(-c4ccc5c(c4)c4ccccc4n5-c4cccc5c4oc4cccc(-c6ccccc6)c45)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4oc4cccc(-c6ccccc6)c45)c([2H])c([2H])c32)c([2H])c1[2H]]

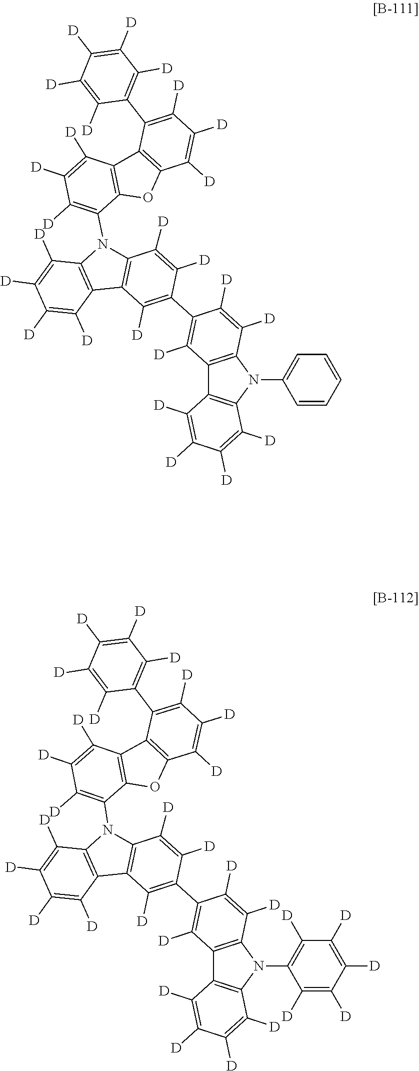 [SMILES: [2H]c1c([2H])c([2H])c(-c2c([2H])c([2H])c([2H])c3oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c([2H])c([2H])c([2H])c4c23)c([2H])c1[2H] ; [2H]c1c([2H])c([2H])c(-c2c([2H])c([2H])c([2H])c3oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c([2H])c([2H])c4c23)c([2H])c1[2H]]

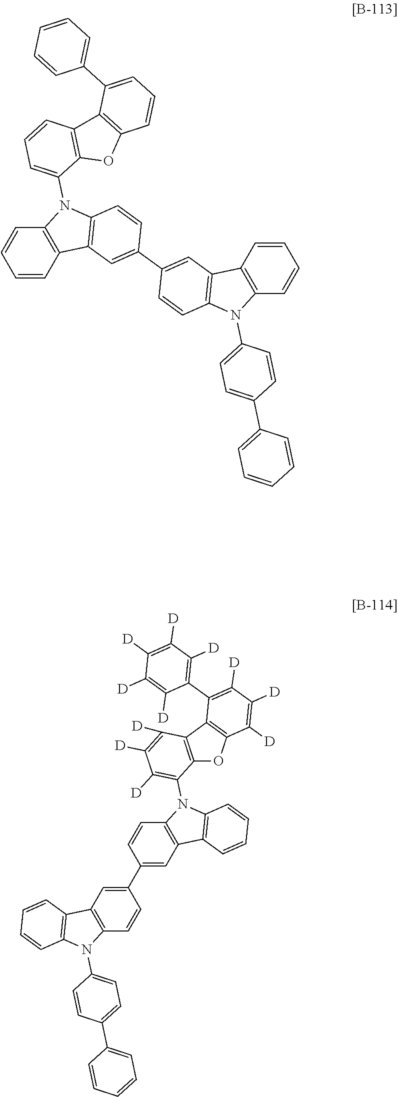 [SMILES: c1ccc(-c2ccc(-n3c4ccccc4c4cc(-c5ccc6c(c5)c5ccccc5n6-c5cccc6c5oc5cccc(-c7ccccc7)c56)ccc43)cc2)cc1 ; [2H]c1c([2H])c([2H])c(-c2c([2H])c([2H])c([2H])c3oc4c(-n5c6ccccc6c6cc(-c7ccc8c(c7)c7ccccc7n8-c7ccc(-c8ccccc8)cc7)ccc65)c([2H])c([2H])c([2H])c4c23)c([2H])c1[2H]]

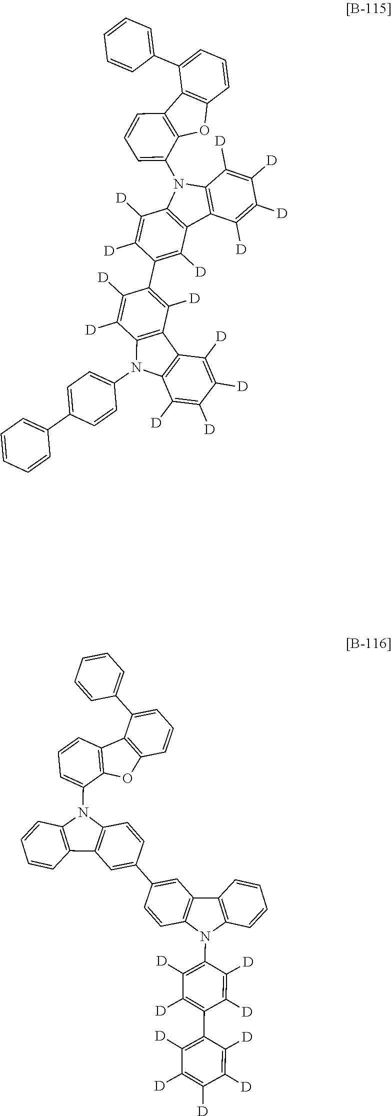 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cccc(-c5ccccc5)c34)c([2H])c([2H])c1n2-c1ccc(-c2ccccc2)cc1 ; [2H]c1c([2H])c([2H])c(-c2c([2H])c([2H])c(-n3c4ccccc4c4cc(-c5ccc6c(c5)c5ccccc5n6-c5cccc6c5oc5cccc(-c7ccccc7)c56)ccc43)c([2H])c2[2H])c([2H])c1[2H]]

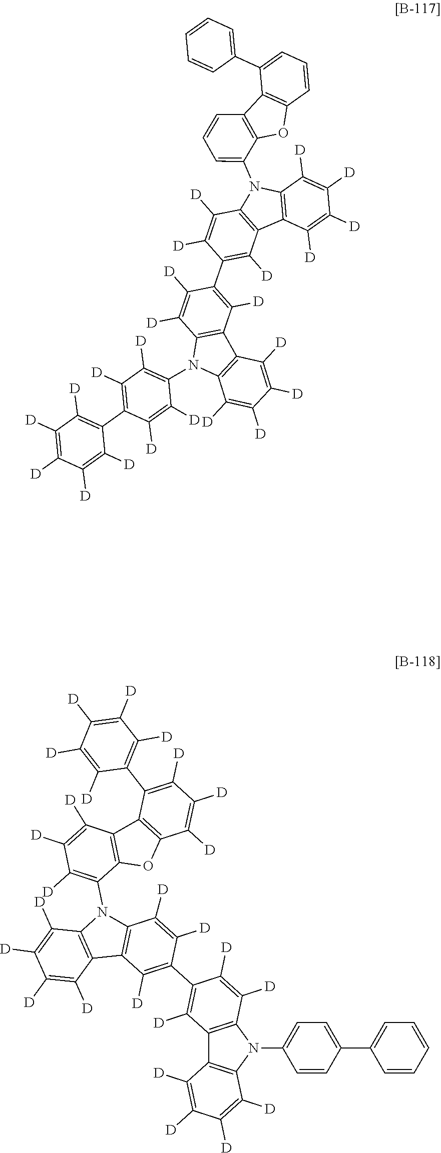 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5oc5cccc(-c7ccccc7)c56)c([2H])c([2H])c43)c([2H])c2[2H])c([2H])c1[2H] ; [2H]c1c([2H])c([2H])c(-c2c([2H])c([2H])c([2H])c3oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c([2H])c([2H])c([2H])c4c23)c([2H])c1[2H]]

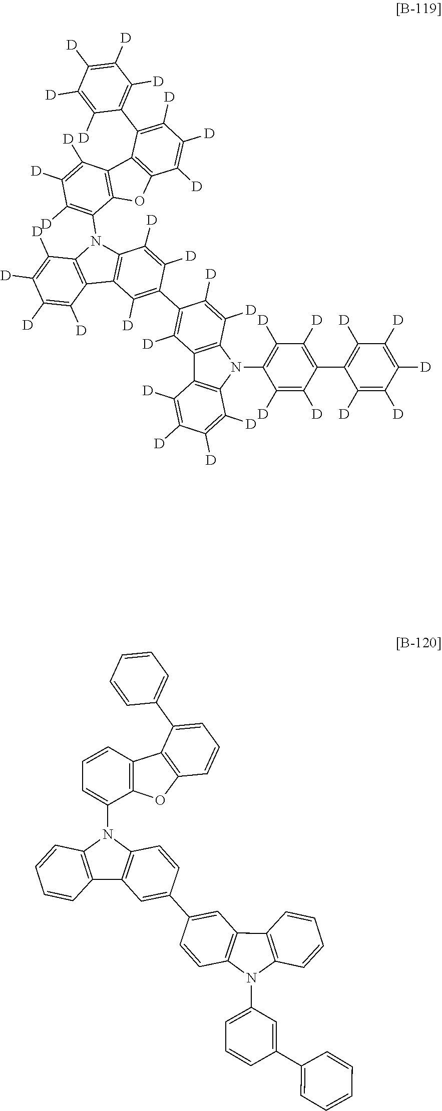 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c5oc5c([2H])c([2H])c([2H])c(-c7c([2H])c([2H])c([2H])c([2H])c7[2H])c56)c([2H])c([2H])c43)c([2H])c2[2H])c([2H])c1[2H] ; c1ccc(-c2cccc(-n3c4ccccc4c4cc(-c5ccc6c(c5)c5ccccc5n6-c5cccc6c5oc5cccc(-c7ccccc7)c56)ccc43)c2)cc1]

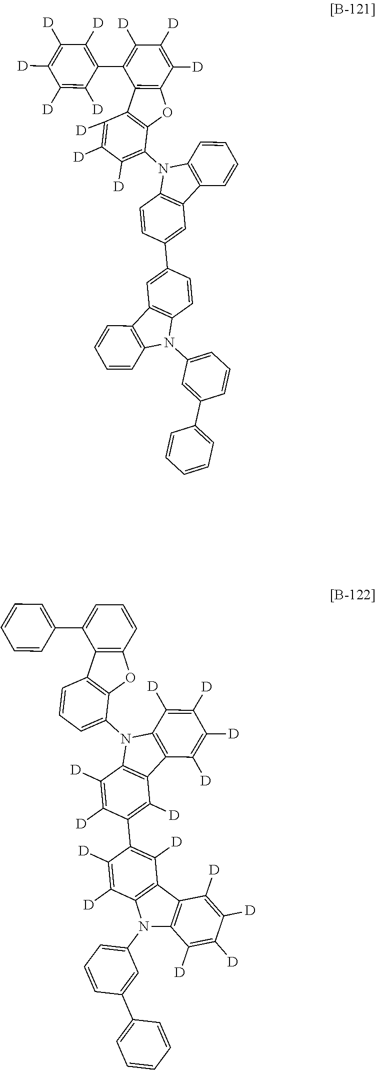 [SMILES: [2H]c1c([2H])c([2H])c(-c2c([2H])c([2H])c([2H])c3oc4c(-n5c6ccccc6c6cc(-c7ccc8c(c7)c7ccccc7n8-c7cccc(-c8ccccc8)c7)ccc65)c([2H])c([2H])c([2H])c4c23)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cccc(-c5ccccc5)c34)c([2H])c([2H])c1n2-c1cccc(-c2ccccc2)c1]

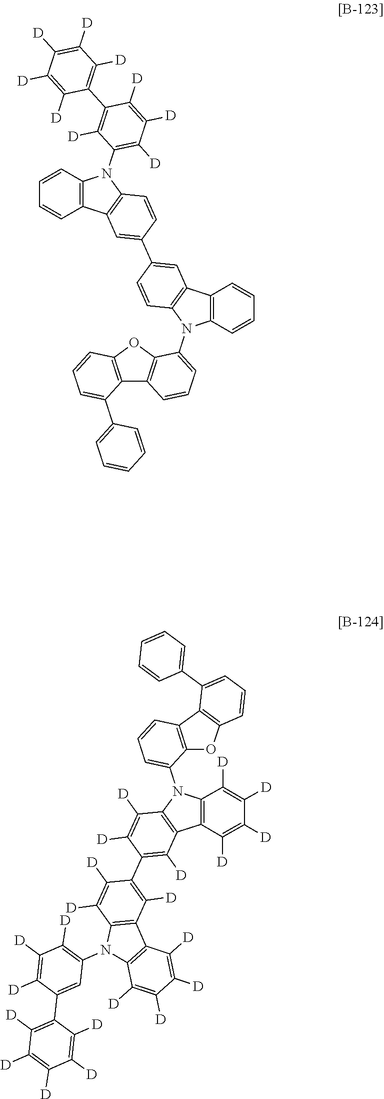 [SMILES: [2H]c1c([2H])c([2H])c(-c2c([2H])c([2H])c([2H])c(-n3c4ccccc4c4cc(-c5ccc6c(c5)c5ccccc5n6-c5cccc6c5oc5cccc(-c7ccccc7)c56)ccc43)c2[2H])c([2H])c1[2H] ; [2H]c1c(-c2c([2H])c([2H])c([2H])c([2H])c2[2H])cc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4oc4cccc(-c6ccccc6)c45)c([2H])c([2H])c32)c([2H])c1[2H]]

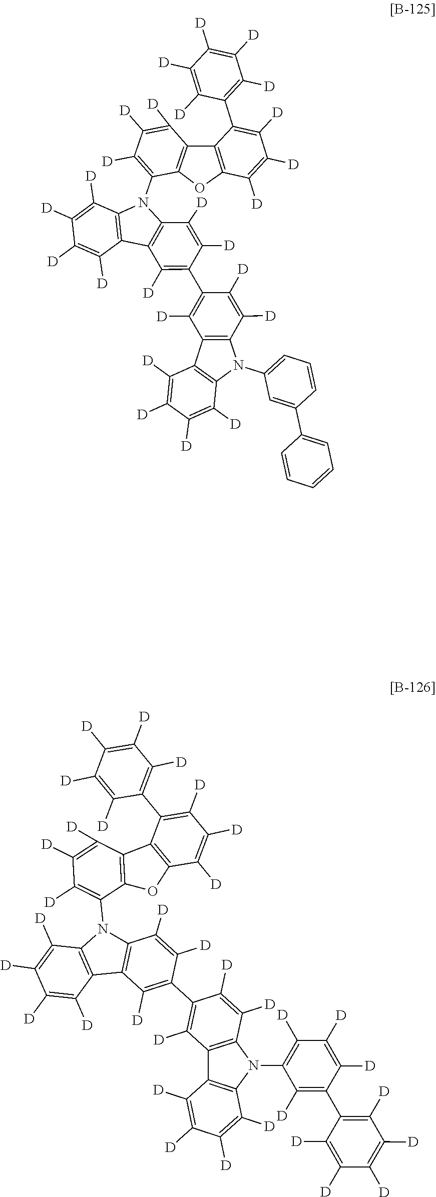

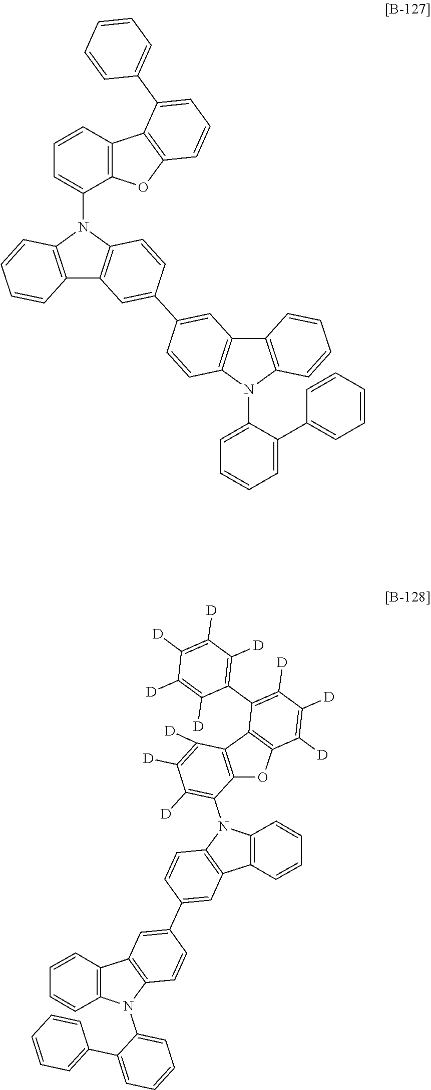 [SMILES: c1ccc(-c2ccccc2-n2c3ccccc3c3cc(-c4ccc5c(c4)c4ccccc4n5-c4cccc5c4oc4cccc(-c6ccccc6)c45)ccc32)cc1 ; [2H]c1c([2H])c([2H])c(-c2c([2H])c([2H])c([2H])c3oc4c(-n5c6ccccc6c6cc(-c7ccc8c(c7)c7ccccc7n8-c7ccccc7-c7ccccc7)ccc65)c([2H])c([2H])c([2H])c4c23)c([2H])c1[2H]]

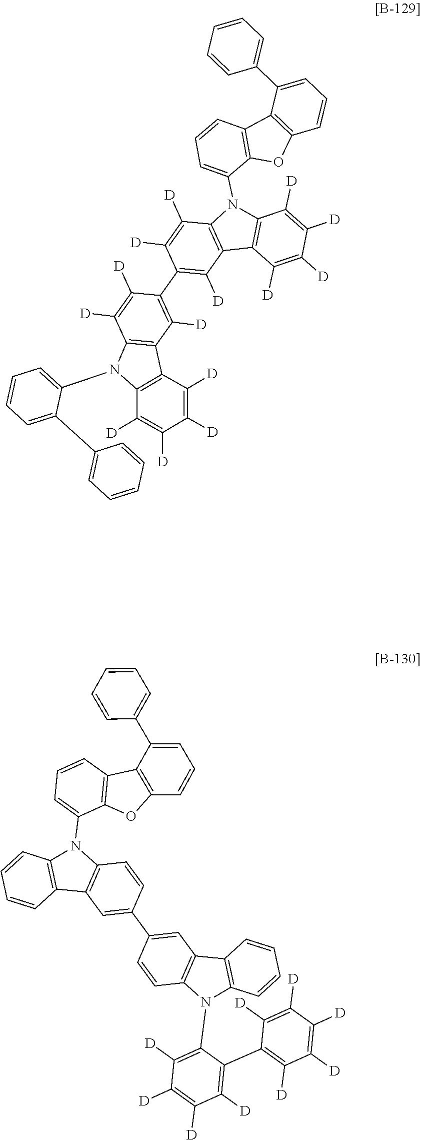 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cccc(-c5ccccc5)c34)c([2H])c([2H])c1n2-c1ccccc1-c1ccccc1 ; [2H]c1c([2H])c([2H])c(-c2c([2H])c([2H])c([2H])c([2H])c2-n2c3ccccc3c3cc(-c4ccc5c(c4)c4ccccc4n5-c4cccc5c4oc4cccc(-c6ccccc6)c45)ccc32)c([2H])c1[2H]]

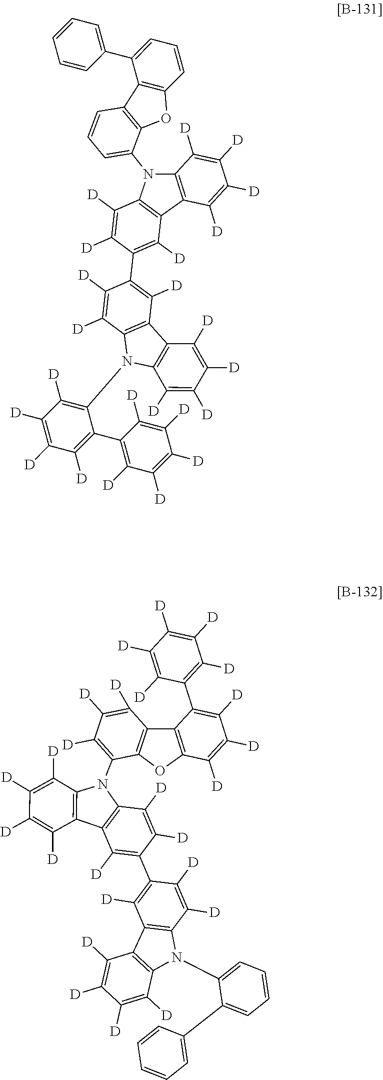 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4oc4cccc(-c6ccccc6)c45)c([2H])c([2H])c32)c([2H])c1[2H] ; [2H]c1c([2H])c([2H])c(-c2c([2H])c([2H])c([2H])c3oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c([2H])c([2H])c4c23)c([2H])c1[2H]]

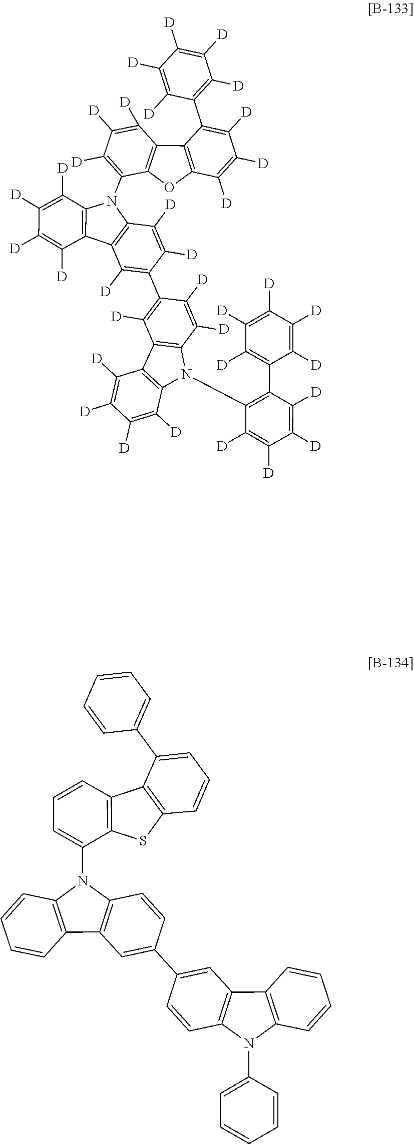 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c4oc4c([2H])c([2H])c([2H])c(-c6c([2H])c([2H])c([2H])c([2H])c6[2H])c45)c([2H])c([2H])c32)c([2H])c1[2H] ; c1ccc(-c2cccc3sc4c(-n5c6ccccc6c6cc(-c7ccc8c(c7)c7ccccc7n8-c7ccccc7)ccc65)cccc4c23)cc1]

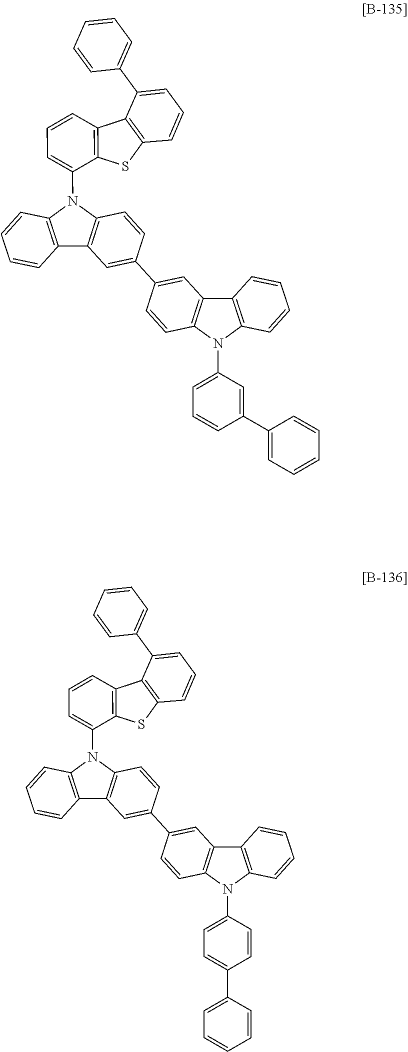 [SMILES: c1ccc(-c2cccc(-n3c4ccccc4c4cc(-c5ccc6c(c5)c5ccccc5n6-c5cccc6c5sc5cccc(-c7ccccc7)c56)ccc43)c2)cc1 ; c1ccc(-c2ccc(-n3c4ccccc4c4cc(-c5ccc6c(c5)c5ccccc5n6-c5cccc6c5sc5cccc(-c7ccccc7)c56)ccc43)cc2)cc1]

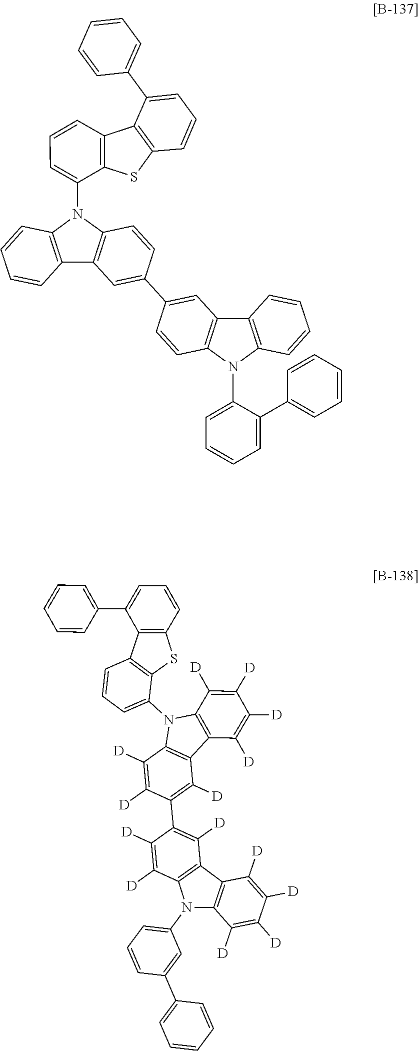 [SMILES: c1ccc(-c2ccccc2-n2c3ccccc3c3cc(-c4ccc5c(c4)c4ccccc4n5-c4cccc5c4sc4cccc(-c6ccccc6)c45)ccc32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3cccc(-c5ccccc5)c34)c([2H])c([2H])c1n2-c1cccc(-c2ccccc2)c1]

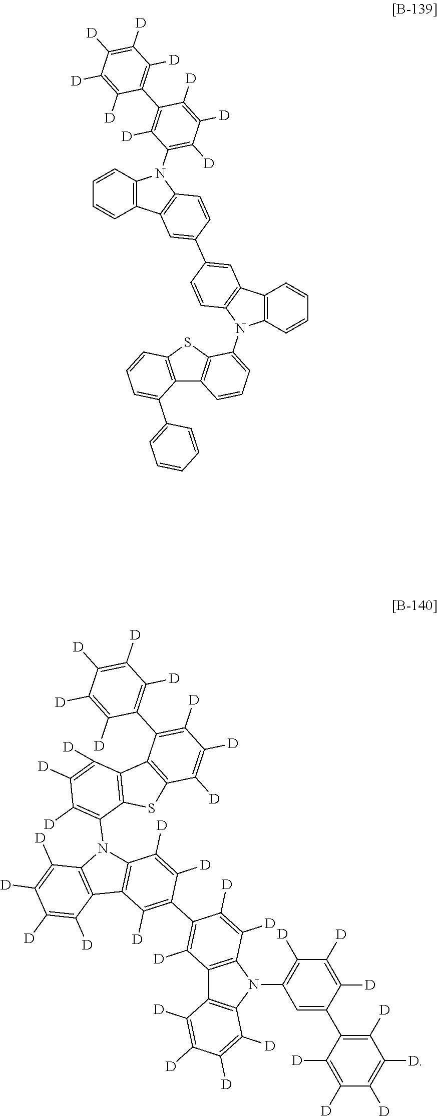

12. An organic optoelectronic device, comprising:
an anode and a cathode facing each other, and
at least one organic layer between the anode and the cathode,
wherein the at least one organic layer includes the composition for the organic optoelectronic device of claim 1.

13. The organic optoelectronic device of claim 12, wherein:
the at least one organic layer includes a light emitting layer, and
the light emitting layer includes the composition for the organic optoelectronic device.

14. The organic optoelectronic device of claim 12, wherein the composition for the organic optoelectronic device further includes a phosphorescent dopant.

15. The organic optoelectronic device of claim 13, wherein the composition for the organic optoelectronic device is a green light-emitting composition.

16. A display device comprising the organic optoelectronic device of claim 12.
